(19) United States
(12) Patent Application Publication
(10) Pub. No.: US 2026/0060974 A1
(43) Pub. Date: Mar. 5, 2026

(54) THERAPEUTIC TYROSINE KINASE INHIBITORS FOR MULTIPLE SCLEROSIS

(71) Applicant: Principia Biopharma Inc., Morristown, NJ (US)

(72) Inventors: Deborah Dukovic, Chester Springs, PA (US); Sana Syed, Chestnut Hill, MA (US); Timothy J. Turner, Belmont, MA (US)

(73) Assignee: Principia Biopharma Inc., Morristown, NJ (US)

(21) Appl. No.: 19/313,165

(22) Filed: Aug. 28, 2025

Related U.S. Application Data
(60) Provisional application No. 63/689,585, filed on Aug. 30, 2024.
(60) Provisional application No. 63/689,584, filed on Aug. 30, 2024.
(60) Provisional application No. 63/695,738, filed on Sep. 17, 2024.
(60) Provisional application No. 63/733,309, filed on Dec. 12, 2024.
(60) Provisional application No. 63/763,718, filed on Feb. 26, 2025.
(60) Provisional application No. 63/770,569, filed on Mar. 12, 2025.

(30) Foreign Application Priority Data
Mar. 24, 2025 (CA) 3268726

(51) Int. Cl. A61K 31/4545; A61P 25/28; A61P 37/00

(52) U.S. Cl. CPC A61K 31/4545; A61P 25/28; A61P 37/00

(57) ABSTRACT

This disclosure relates to the field of therapeutic tyrosine kinase inhibitors, in particular, Bruton tyrosine kinase (“BTK”) inhibitors, for treatment of patients with multiple sclerosis (MS).

### CROSS-REFERENCE TO RELATED APPLICATIONS

This application claims priority to U.S. Provisional Application No. 63/689,584, filed on Aug. 30, 2024, U.S. Provisional Application No. 63/689,585, filed on Aug. 30, 2024, U.S. Provisional Application No. 63/695,738, filed on Sep. 17, 2024, U.S. Provisional Application No. 63/733,309, filed on Dec. 12, 2024, U.S. Provisional Application No. 63/763,718, filed on Feb. 26, 2025, U.S. Provisional Application No. 63/770,569, filed on Mar. 12, 2025, Canadian Application No. 3268726, filed on Mar. 24, 2025, and U.S. Provisional Application No. 63/861,769 filed Aug. 11, 2025, all of which are incorporated by reference herein in their entirety for any purpose.

### FIELD

The present application relates to the use of therapeutic tyrosine kinase inhibitors, in particular, Bruton tyrosine kinase (“BTK”) inhibitors, for treatment of patients with multiple sclerosis (MS).

### INTRODUCTION

This disclosure relates to the field of therapeutic tyrosine kinase inhibitors, in particular, Bruton tyrosine kinase (“BTK”) inhibitors, for treating multiple sclerosis (MS). MS is a chronic, immune-mediated, neurodegenerative disease affecting more than 1 million people worldwide, which results in accumulation of irreversible disabilities over time. The physical and cognitive disability impairments translate into gradual deterioration of health status and lower quality of life, impacting patients' care and life expectancy. It is the most common cause of neurological disability in young and middle-aged adults and has a major physical, psychological, social and financial impact on subjects and their families. MS involves an immune-mediated process in which an abnormal response of the body's immune system is directed against the central nervous system (CNS). In the course of the disease, scleroses, i.e., lesions or scars, appear in the myelin sheath of nerve cells, disrupting transmission of electrical signals. Scleroses accumulate over time and result in the debilitating symptoms experienced by MS patients.

Historically, MS patients have been generally categorized as experiencing one of four clinical courses of disease, each of which might be mild, moderate, or severe: clinically isolated syndrome, relapsing remitting, secondary progressive (e.g., non-relapsing secondary progressive multiple sclerosis (“nrSPMS”)) and primary progressive (“PPMS”). About 85% of MS patients have the relapsing remitting form of the disease, in which they experience clearly defined relapses (also called flare-ups or exacerbations), which are episodes of new or worsening symptoms (i.e., worsening neurologic function), followed by periods of partial or complete recovery (remissions). Within the scope of the present disclosure, “relapsing multiple sclerosis,” “relapsing MS,” “relapsing forms of MS,” or “RMS” may include clinically isolated syndrome (“CIS”), relapsing remitting multiple sclerosis (“RRMS”), and relapsing secondary progressive multiple sclerosis (“R-SPMS”). (See, e.g., Lublin et al., Defining the clinical course of multiple sclerosis; the 2013 revisions, Neurology 2014; 83:278-286.) nrSPMS refers to a disease state in which the patient has stopped experiencing confirmed relapses but continues to experience accumulation of disability, experienced as various symptoms such as gait impairment, fatigue, cognition impairment, balance difficulties, loss of bowel and/or bladder function, sexual disfunction, amongst others. PPMS refers to a disease state in which the patient experiences accumulation of disability, independent of relapses, since the onset of the disease clinical manifestation, but it likely does not have pathophysiologically distinct features from relapsing forms of MS that have entered a progressive course (SPMS). (See, e.g., Lublin et al.)

More recently, a new dogma is emerging in the field of MS, which focuses on relapse-independent disability accumulation or accrual as the key component for disease progression verses frequency of relapses (See, e.g., Kappos et al., JAMA Neurology 2020; 77(9):1132-1140). The authors analyzed the relative contributions of progression independent of relapse activity (PIRA) and relapse-associated worsening (RAW) to overall accumulation or accrual of disability in 1656 patients with relapsing multiple sclerosis from two randomized phase 3 clinical trials (OPERA I and II). The results demonstrated that approximately 80% to 90% of confirmed disability events occur in the absence of relapses, challenging the current clinical distinction of relapsing and progressive forms of multiple sclerosis.

Immunomodulatory drugs have been the mainstay of MS therapy. Recent results from clinical studies have demonstrated efficacy of agents that target B lymphocytes, especially B-cell-depleting agents like ocrelizumab (anti-CD20) (Hauser et al., N Engl J Med. 2017; 376(3):221-34).

Targeting B-cells represents a departure from the prevailing dogma based on animal models that demonstrated therapeutic benefits from modulating T-cell activity and positions the B cell as the centerpiece of current MS drug development (Lehmann-Horn K et al., Int J Mol Sci.2017; 18(10):2048). The importance of immune cells residing in the CNS is also well known and needs to be considered in MS pathogenesis (Hemmer B et al, Nat Clin Pract Neurol. 2006; 2(4):201-11).

Tolebrutinib is a BTK inhibitor that is being investigated as a disease modifying therapy to treat MS. The Bruton's tyrosine kinase pathway is critical to signaling in B lymphocytes and myeloid cells including CNS microglia. Each of these cell types has been implicated in the pathophysiology of multiple sclerosis. Further, as BTK signaling is vital for maturation of B cells into antibody-secreting plasma cells, BTK inhibition can modulate both cellular and humoral immunity.

Accordingly, an inhibitor of BTK signaling represents a dual mechanism targeting both aspects of the immune system.

Accordingly, compounds that inhibit BTK that are able to both inhibit antigen-induced B-cell activation responsible for focal inflammation and modulate maladaptive, CNS-resident microglial cells linked to neuroinflammation in the brain and spinal cord may be useful in treating both relapsing and progressive forms of MS, as traditionally categorized, and/or reducing the risk of disability accumulation with superior benefits when compared to currently available therapies.

Drug-induced liver injury has been identified in the ongoing tolebrutinib Phase 3 trials. The reported events occurred between months 2 to 3 after the start of tolebrutinib administration, and the elevation of liver enzymes appears reversible after tolebrutinib discontinuation. Thus, there is a need to mitigate the risk of hepatic injury and provide safe treatment for patients with MS.

### SUMMARY

Disclosed herein is a method of reducing disability accumulation independent of relapse activity in a patient with secondary progressive MS in need thereof comprising administering to the patient in need thereof a therapeutically effective amount of tolebrutinib or pharmaceutically acceptable salt thereof.

Provided herein is a method of treating a patient with non-relapsing secondary progressive multiple sclerosis (nrSPMS) in need thereof, comprising administering to the patient in need thereof a therapeutically effective amount of tolebrutinib or pharmaceutically acceptable salt thereof, wherein at least one outcome chosen from the following occurs: (a) the risk of confirmed disability progression (CDP) is reduced; (b) the total number of new and/or enlarging T2-hyperintense lesions as detected by MRI, defined as the sum of the individual number of new and/or enlarging T2 lesions, are reduced; (c) the risk of sustained 20% increase in the timed 25-foot walk (T25-FW) test confirmed over at least 3 months is reduced; and (d) the chance of confirmed disability improvement (CDI) is improved.

Also disclosed herein is a method of treating a patient with non-relapsing secondary progressive multiple sclerosis (nrSPMS) in need thereof, comprising administering to the patient in need thereof a therapeutically effective amount of tolebrutinib or pharmaceutically acceptable salt thereof, wherein the risk of confirmed disability progression (CDP) is reduced.

In some embodiments, disclosed herein is a method of reducing the risk of confirmed disability progression (CDP) in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

In some embodiments, disclosed herein is a method of reducing the total number of new and/or enlarging T2-hyperintense lesions as detected by MRI, in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

In some embodiments, disclosed herein is a method of reducing the risk of sustained 20% increase in the timed 25-foot walk (T25-FW) test confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

In some embodiments, disclosed herein is a method of reducing the risk of sustained 20% increase in the 9-hole peg test (9-HPT) confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

In some embodiments, disclosed herein is a method of improving the chance of confirmed disability improvement (CDI) in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

In some embodiments, disclosed herein is a method of improving time to onset of confirmed disability progression (CDP) in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

In some embodiments, disclosed herein is a method of improving time to onset of sustained 20% increase in the timed 25-foot walk (T25-FW) test confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

In some embodiments, disclosed herein is a method of improving time to onset of sustained 20% increase in the 9-hole peg test (9-HPT) confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

In some embodiments, disclosed herein is a method of improving time to onset of confirmed disability improvement (CDI) in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

In some embodiments, disclosed herein is tolebrutinib or a pharmaceutically acceptable salt thereof for use in at least one of: (i) treating a patient with non-relapsing secondary progressive multiple sclerosis (nrSPMS); (ii) reducing the risk of confirmed disability progression (CDP) in a patient with non-relapsing secondary progressive multiple sclerosis (nrSPMS); (iii) reducing the total number of new and/or enlarging T2-hyperintense lesions as detected by MRI in a patient with non-relapsing secondary progressive multiple sclerosis (nrSPMS); (iv) reducing the risk of sustained 20% increase in the timed 25-foot walk (T25-FW) test confirmed over at least 3 months in a patient with non-relapsing secondary progressive multiple sclerosis (nrSPMS); (v) reducing the risk of sustained 20% increase in the 9-hole peg test (9-HPT) confirmed over at least 3 months in a patient with non-relapsing secondary progressive multiple sclerosis (nrSPMS); (vi) improving the chance of confirmed disability improvement (CDI) in a patient with non-relapsing secondary progressive multiple sclerosis (nrSPMS); (vii) improving time to onset of confirmed disability progression (CDP) in a patient with non-relapsing secondary progressive multiple sclerosis (nrSPMS); (viii) in improving time to onset of sustained 20% increase in the timed 25-foot walk (T25-FW) test confirmed over at least 3 months in a patient with non-relapsing secondary progressive multiple sclerosis (nrSPMS); (ix) in improving time to onset of sustained 20% increase in the 9-hole peg test (9-HPT) confirmed over at least 3 months in a patient with non-relapsing secondary progressive multiple sclerosis (nrSPMS); and (x) improving time to onset of confirmed disability improvement (CDI) in a patient with non-relapsing secondary progressive multiple sclerosis (nrSPMS), in certain methods described herein.

Also disclosed herein is a method of reducing the risk of confirmed disability worsening (CDW) in a patient with a relapsing form of multiple sclerosis (RMS) in need thereof, comprising administering to the patient in need thereof a therapeutically effective amount of tolebrutinib or pharmaceutically acceptable salt thereof.

In some embodiments, disclosed herein is a method of improving time to onset of confirmed disability worsening (CDW) in a patient with a relapsing form of multiple sclerosis (RMS) in need thereof, comprising administering to the patient in need thereof a therapeutically effective amount of tolebrutinib or pharmaceutically acceptable salt thereof.

In some embodiments, provided herein a method of improving percent change in brain volume loss (BVL) as detected by brain MRI in a patient with relapsing forms of multiple sclerosis (RMS), comprising administering to the patient in need thereof a therapeutically effective amount of tolebrutinib or pharmaceutically acceptable salt thereof.

In some embodiments, disclosed herein is tolebrutinib or a pharmaceutically acceptable salt thereof for use in at least one of (i) reducing the risk of confirmed disability worsening (CDW) in a patient with a relapsing form of multiple sclerosis (RMS); (ii) improving time to onset of confirmed disability worsening (CDW) in a patient with a relapsing form of multiple sclerosis (RMS); and (iii) improving percent change in brain volume loss (BVL) as detected by brain MRI in a patient with a relapsing form of multiple sclerosis (RMS), in certain methods described herein.

Still further disclosed herein is a method of treating a patient with primary progressive multiple sclerosis (PPMS) in need thereof, comprising administering to the patient in need thereof a therapeutically effective amount of tolebrutinib or pharmaceutically acceptable salt thereof, wherein at least one outcome chosen from the following occurs: (a) the risk of confirmed disability progression (CDP) is reduced; (b) the total number of new and/or enlarging T2-hyperintense lesions as detected by MRI, defined as the sum of the individual number of new and/or enlarging T2 lesions, are reduced; (c) the risk of sustained 20% increase in the timed 25-foot walk (T25-FW) test confirmed over at least 3 months is reduced; (d) the risk of sustained 20% increase in the 9-hole peg test (9-HPT) confirmed over at least 3 months is reduced; (e) the chance of confirmed disability improvement (CDI) is improved; and (f) the percent change in brain volume loss (BVL) as detected by brain MRI is improved.

In some embodiments, disclosed herein is a method of treating a patient with primary progressive multiple sclerosis (PPMS) in need thereof, comprising administering to the patient in need thereof a therapeutically effective amount of tolebrutinib or pharmaceutically acceptable salt thereof, wherein the risk of confirmed disability progression (CDP) or composite confirmed disability progression (cCDP) is reduced, wherein the cCDP comprises progression in at least one of: (a) CDP; (b) sustained 20% increase in the timed 25-foot walk (T25-FW) test confirmed over at least 3 months; and (c) sustained 20% increase in the 9-hole peg test (9-HPT) confirmed over at least 3 months.

In some embodiments, disclosed herein is a method of reducing the risk of confirmed disability progression (CDP) or composite confirmed disability progression (cCDP) in a patient with primary progressive multiple sclerosis (PPMS) in need thereof, comprising administering to the patient in need thereof a therapeutically effective amount of tolebrutinib or pharmaceutically acceptable salt thereof, wherein the cCDP comprises progression in at least one of: (a) CDP; (b) sustained 20% increase in the timed 25-foot walk (T25-FW) test confirmed over at least 3 months; and (c) sustained 20% increase in the 9-hole peg test (9-HPT) confirmed over at least 3 months.

In some embodiments, disclosed herein is a method of improving time to onset of confirmed disability progression (CDP) or composite confirmed disability progression (cCDP) in a patient with primary progressive multiple sclerosis (PPMS) in need thereof, comprising administering to the patient in need thereof a therapeutically effective amount of tolebrutinib or pharmaceutically acceptable salt thereof, wherein the cCDP comprises progression in at least one of: (a) CDP; (b) sustained 20% increase in the timed 25-foot walk (T25-FW) test confirmed over at least 3 months; and (c) sustained 20% increase in the 9-hole peg test (9-HPT) confirmed over at least 3 months.

In some embodiments, disclosed herein is tolebrutinib or a pharmaceutically acceptable salt thereof for use in at least one of (i) treating a patient with primary progressive multiple sclerosis (PPMS); (ii) reducing the risk of confirmed disability progression (CDP) or composite confirmed disability progression (cCDP) in a patient with primary progressive multiple sclerosis (PPMS); and (iii) improving time to onset of confirmed disability progression (CDP) or composite confirmed disability progression (cCDP) in a patient with primary progressive multiple sclerosis (PPMS), in certain methods described herein.

The present disclosure also relates to a method of treating disability accumulation in a patient with multiple sclerosis comprising administering to the patient in need thereof a therapeutically effective amount of tolebrutinib or pharmaceutically acceptable salt thereof.

In some embodiments, provided herein is a method of treating disability progression independent of relapse activity in a patient with multiple sclerosis comprising administering to the patient in need thereof a therapeutically effective amount of tolebrutinib or pharmaceutically acceptable salt thereof.

In some embodiments, disclosed herein is tolebrutinib or a pharmaceutically acceptable salt thereof for use in at least one of (i) treating disability accumulation in a patient with multiple sclerosis; and (ii) treating disability progression independent of relapse activity in a patient with multiple sclerosis, in certain methods described herein.

### BRIEF DESCRIPTION OF DRAWINGS

FIG. 1A provides the study design of Example 1. ^{¶}The frequency of LFT monitoring in the first 90 days increased from monthly to once every 2 weeks from week 4 following a protocol amendment on May 23, 2022. Further monitoring at week 2 and weekly from week 4-12 was added after an additional amendment on Sep. 13, 2022. Week 3 monitoring was added in the Nov. 10, 2023 amendment. ^{‡}The 28-day screening period was considered Month −1. ^{∥}EOS safety follow-up visit occurred 4 weeks after the last dose of study treatment for participants not entering the long-term safety study. ^{§} All participants who completed the trial were offered enrollment in a long-term safety study with open-label tolebrutinib. CDP, confirmed disability progression; EDSS, Expanded Disability Status Scale; EOS, end of study; LFT, liver function test; MRI, magnetic resonance imaging; nrSPMS, non-relapsing secondary progressive MS; SPMS, secondary progressive MS; R, randomisation.

FIG. 1B provides the study design of Example 2. ^{¶}The frequency of LFT monitoring in the first 90 days increased from monthly to once every 2 weeks from week 4 following a protocol amendment on May 23, 2022. Further monitoring at week 2 and weekly from week 4-12 was added after an additional amendment on Sep. 13, 2022. Week 3 monitoring was added in the Dec. 20, 2023 amendment. ^{‡}The 28-day screening period was considered Month −1. ^{∥}EOS safety follow-up visit occurred 4 weeks after the last dose of study treatment for participants not entering the long-term safety study. ^{§}All participants who completed the trial were offered enrollment in a long-term safety study with open-label tolebrutinib. *Approximate number of events in both treatment arms for the pooled data from GEMINI 1 and 2. CDW, confirmed disability worsening; EDSS, Expanded Disability Status Scale; EOS, end of study; MRI, magnetic resonance imaging; R, randomisation.

FIG. 1C provides the study design of Example 3.

FIG. 2 provides suggested actions and follow-up assessments in the event of neutropenia. *For individuals of African descent, the relevant value of concern is <1000/mm³.

FIG. 3 provides suggested actions and follow-up assessments in the event of thrombocytopenia. aPTT: activated partial thromboplastin time; EDTA: Ethylene-diaminetetraacetic acid; INR: international normalized ratio; PK: pharmacokinetic; PT: prothrombin time.

FIG. 4 provides suggested actions and follow-up assessments in the event of increased alanine aminotransferase (ALT) algorithm. *If unable to reset in 72 hours, use original laboratory results to decide on further reporting/monitoring/discontinuation. ALT: alanine aminotransferase; AST: aspartate aminotransferase; CRF: case report form; IMP: investigational medicinal product; INR: international normalized ratio; ULN: upper limit of normal.

FIG. 5 provides suggested actions and follow-up assessments in the event of serum creatinine. ARF, acute renal failure; ULN, upper limit of normal; DIC, disseminated intravascular coagulation; CPK, creatine phosphokinase; ECG, electrocardiogram; PK, pharmacokinetic(s).

FIG. 6 provides suggested actions and follow-up assessments in the event of increase in CPK of non-cardiac origin and not related to intensive physical activity. CK-MB, creatine kinase-MB; CK-MM, creatine kinase-MM; ECG, electrocardiogram; PK, pharmacokinetic(s); ULN, upper limit of normal.

FIG. 7 provides suggested actions and follow-up assessments when progressive multifocal leukoencephalopathy (PML) is suspected. CSF, cerebrospinal fluid; Gd, gadolinium; IMP, investigational medicinal product; JCV, John Cunningham virus; MRI, magnetic resonance imaging; PCR, polymerase chain reaction; PML, progressive multifocal leukoencephalopathy

FIG. 8 provides a description of the expanded disability status scale score (EDSS) in view of the level of disability.

FIG. 9 shows the participant disposition for 1131 randomized patients entering the study of Example 1. Note: A participant could have failed screening due to not meeting multiple eligibility criteria. ITT, intention-to-treat.

FIG. 10A is a plot showing the 6-month confirmed disability progression (CDP) for patients treated with tolebrutinib or placebo in the study of Example 1. ^{a}6-month CDP is defined as an increase of ≥1.0 point from baseline EDSS score when baseline score is ≤5.0 or an increase of ≥0.5 points when baseline score is >5.0, confirmed over ≥6 months. CDP, confirmed disability progression; CI, confidence interval; EDSS, Expanded Disability Status Scale; HR, hazard ratio. ^{b}P-value is from Cox proportional hazards model. FIG. 10B is a plot showing the 6-month CDP from FIG. 10A without 6-month confirmation. FIG. 10C is a plot showing the 6-month CDP from FIG. 10A without imputation. FIG. 10D is a Forest plot of hazard ratios and corresponding 95% confidence intervals comparing tolebrutinib to placebo for time to onset of 6-month CDP within each subgroup.

FIG. 11 is a plot showing the 3-month confirmed disability progression (CDP) for patients treated with tolebrutinib or placebo in the study of Example 1. ^{a}3-month CDP is defined as an increase of ≥1.0 point from baseline EDSS score when baseline score is ≤5.0 or an increase of ≥0.5 points when baseline score is >5.0, confirmed over ≥3 months. CDP, confirmed disability progression; CI, confidence interval; EDSS, Expanded Disability Status Scale; HR, hazard ratio. ^{b}P-value is from Cox proportional hazards model.

FIG. 12 shows the total number of new and/or enlarging T2-hyperintense lesions as detected by MRI for patients treated with tolebrutinib or placebo in the study of Example 1. CI, confidence interval.

FIG. 13 is a plot showing the 3-month confirmed 20% increase in 9-hole peg test (9-HPT) for patients treated with tolebrutinib or placebo in the study of Example 1.

FIG. 14 is a plot showing the 3-month confirmed 20% increase in the timed 25-foot walk test (T25-FW) for patients treated with tolebrutinib or placebo in the study of Example 1.

FIG. 15 is a plot showing the 6-month confirmed disability improvement (CDI) for patients treated with tolebrutinib or placebo in the study of Example 1. ^{a}6-month CDI is defined as a decrease of ≥1.0 point from baseline EDSS score confirmed over ≥6 months. ^{b}Nominal p-value from Cox proportional hazards model. CDI, confirmed disability improvement; CI, confidence interval; EDSS, Expanded Disability Status Scale; HR, hazard ratio.

FIG. 16 shows the percent change in brain volume loss (BVL) for patients treated with tolebrutinib or placebo in the study of Example 1. ^{a}Nominal p-value. ^{b}The mean trial duration was 27 months. Note: BVL is measured as percentage change in brain volume from Month 6 to exclude the potential confounding effect of pseudoatrophy. At baseline, the median normalized brain volume was 1467.8 cm³ (IQR, 1404.2-1529.3) for 746 participants in the tolebrutinib group and 1462.0 cm3 (1401.5-1515.0) for 375 participants in the placebo group.BL, baseline; LSM, least squares mean; EOS, end of study; IQR, interquartile range; SE, standard error.

FIG. 17A shows a summary of percent adverse events for patients treated with tolebrutinib or placebo in the study of Example 1. FIG. 17B shows a summary of percent treatment emergent serious adverse events (TE SAEs) for patients treated with tolebrutinib or placebo. FIG. 17C shows a summary of percent adverse events of special interest (AESI) for patients treated with tolebrutinib or placebo. FIG. 17D shows a line plot subgroup analysis of patients with an absolute difference of less than 1% for patients treated with tolebrutinib or placebo.

FIG. 18 shows a summary of percent peak on-treatment alanine aminotransferase (ALT) enzyme and biochemical Hy's law for patients treated with tolebrutinib or placebo in the study of Example 1. ^{a}One participant on tolebrutinib received a liver transplant and died due to post-operative complications. This case occurred prior to the implementation of a revised protocol with more stringent monitoring. ALT, alanine aminotransferase; BILI, bilirubin; ULN, upper limit of normal.

FIGS. 19A-19B shows the participant disposition for 974 randomized patients entering Gemini 1 (EFC16033) (FIG. 19A) and 899 randomized patients entering Gemini 2 (EFC16034) (FIG. 19B), as described in Example 2. Note: A participant could have failed screening due to not meeting multiple eligibility criteria. ECG, electrocardiogram; ITT, intention-to-treat.

FIG. 20 shows the annualized (adjudicated) relapse rate for patients treated with tolebrutinib or teriflunomide, where the patients were treated in either the Gemini 1 study (EFC16033, left), the Gemini 2 study (EFC16034, middle), or in a pooled analysis (right), of Example 2. ARR, annualized (adjudicated) relapse rate; CI, confidence interval.

FIG. 21 is a plot showing the 6-month confirmed disability worsening (CDW) for a pooled population of patients treated with tolebrutinib or teriflunomide in the studies of Example 2. Risk reduction is based on imputation for confirmed 3M-CDW without 6M confirmation due to end of study. p-value from stratified log-rank test. ^{a}6-month CDW is defined as a sustained increase from baseline in EDSS score of ≥1.5 points when baseline score is 0, ≥1.0 points when baseline score is 0.5 to ≤5.5 or ≥0.5 points when baseline score is >5.5, confirmed over ≥6 months. ^{b}Nominal p-value (stratified log-rank test). CDW, confirmed disability worsening; CI, confidence interval; EDSS, Expanded Disability Status Scale; HR, hazard ratio.

FIG. 22 is a plot showing the 3-month confirmed disability worsening (CDW) for a pooled population of patients treated with tolebrutinib or teriflunomide in the studies of Example 2. ^{a}3-month CDW is defined as a sustained increase from baseline in EDSS score of ≥1.5 points when baseline score is 0, ≥1.0 points when baseline score is 0.5 to ≤5.5 or ≥0.5 points when baseline score is >5.5, confirmed over ≥3 months. ^{b}Nominal p-value (stratified log-rank test). CDW, confirmed disability worsening; CI, confidence interval; EDSS, Expanded Disability Status Scale; HR, hazard ratio.

FIG. 23 is a plot showing the 6-month confirmed disability improvement (CDI) for a pooled population of patients treated with tolebrutinib or teriflunomide in the studies of Example 2. ^{a}6-month CDI is defined as a decrease of ≥1.0 point from baseline EDSS score confirmed over ≥6 months. ^{b}Nominal p-value (stratified log-rank test). CDI, confirmed disability improvement; CI, confidence interval; EDSS, Expanded Disability Status Scale; HR, hazard ratio.

FIG. 24 shows the total number of new Gd-enhancing T1-hyperintense lesions as detected by MRI for patients treated with tolebrutinib or teriflunomide, where the patients were treated in either the Gemini 1 study (EFC16033, left) or the Gemini 2 study (EFC16034, right) of Example 2. ^{a}Nominal p-value. Note: Data are mean (95% CI). CI, confidence interval; Gd, gadolinium; MRI, magnetic resonance imaging.

FIG. 25 shows the total number of new and/or enlarging T2-hyperintense lesions as detected by MRI for patients treated with tolebrutinib or teriflunomide, where the patients were treated in either the Gemini 1 study (EFC16033, left) or the Gemini 2 study (EFC16034, right) of Example 2. ^{a}Nominal p-value. Note: Data are mean (95% CI). CI, confidence interval; Gd, gadolinium; MRI, magnetic resonance imaging.

FIGS. 26A-26B show the percent change in brain volume loss (BVL) for patients treated with tolebrutinib or teriflunomide, where the patients were treated in either the Gemini 1 study (EFC16033, FIG. 26A) or the Gemini 2 study (EFC16034, FIG. 26B) of Example 2. ^{a}Nominal p-value. ^{b}The mean trial duration was 30 months for GEMINI 1 and 29 months for GEMINI 2. Note: n indicates the number of participants with EOS data. BVL is measured as percentage change in brain volume from Month 6 to exclude the potential confounding effect of pseudoatrophy. In GEMINI 1, mean (SD) normalized brain volume at baseline was 1533.0 (103.7) cm³ in the tolebrutinib group and 1538.1 (92.4) cm³ in the teriflunomide group. In GEMINI 2, mean (SD) normalized brain volume at baseline was 1547.1 (81.1) cm3 in the tolebrutinib group and 1551.4 (81.5) cm³ in the teriflunomide group. BL, baseline; LSM, least squares mean; EOS, end of study; SE, standard error.

FIGS. 27A-27D show safety parameters associated with tolebrutinib treatment for a pooled population of patients from the Gemini 1 (EFC16033) and Gemini 2 (EFC16034) studies of Example 2. FIG. 27A shows a summary of percent adverse events for patients treated with tolebrutinib or teriflunomide. FIG. 27B shows a summary of percent treatment emergent serious adverse events (TE SAEs) for patients treated with tolebrutinib or teriflunomide. FIG. 27C shows a summary of percent adverse events of special interest (AESI) for patients treated with tolebrutinib or teriflunomide. FIG. 27D shows a line plot subgroup analysis of patients (PTs) with an absolute difference of less than 1% for patients treated with tolebrutinib or teriflunomide.

FIG. 28 shows a summary of percent peak on-treatment alanine aminotransferase (ALT) enzyme and biochemical Hy's law for patients treated with tolebrutinib or teriflunomide for a pooled population of patients from the Gemini 1 (EFC16033) and Gemini 2 (EFC16034) studies of Example 2.

FIG. 29 is a summary of primary and hierarchically ordered secondary end points and results for the Hercules study in Example 1. 9-HPT, 9-HPT, 9-Hole Peg Test; CDI, confirmed disability improvement; CDP, confirmed disability progression; CI, confidence interval; diff, difference; EOS, end of study; HR, hazard ratio; LSM, least squares mean; T25-FW=timed 25-foot walk.

FIG. 30 is a summary of the hierarchical testing procedure for the Gemini 1 and Gemini 2 studies of Example 2, including pooled secondary endpoints. ARR, annualized (adjudicated) relapse rate; CDI, confirmed disability improvement; CDW, confirmed disability worsening; CVLT-II, California Verbal Learning Test-Second Edition; SDMT, Symbol Digit Modalities Test.

FIGS. 31A-31B show plots of the change in SDMT number of correct substitutions z-score from baseline over time for patients treated with tolebrutinib or teriflunomide in the Gemini 1 (EFC16033, FIG. 31A) and Gemini 2 (EFC16034, FIG. 31B) studies of Example 2. *Data are the number of correct substitutions z-score. EOS, end of study; LSM, least squares mean; SDMT, Symbol Digit Modalities Test; SE, standard error.

FIGS. 32A-32B show plots of the change in CVLT-II total correct standardized score from baseline over time for patients treated with tolebrutinib or teriflunomide in the Gemini 1 (EFC16033, FIG. 32A) and Gemini 2 (EFC16034, FIG. 32B) studies of Example 2. *Data are the total correct standardized score. CVLT-II, California Verbal Learning Test-Second Edition; EOS, end of study; LSM, least squares mean; SE, standard error.

FIGS. 33A-33B show plots of the number of new gadolinium-enhancing T1-weighted lesions over time for patients treated with tolebrutinib or teriflunomide in the Gemini 1 (EFC16033, FIG. 33A) and Gemini 2 (EFC16034, FIG. 33B) studies of Example 2. EOS, end of study; Gd, gadolinium; SE, standard error.

FIGS. 34A-34B is shows plots of the number of new/enlarging T2 lesions over time for patients treated with tolebrutinib or teriflunomide in the Gemini 1 (EFC16033, FIG. 34A) and Gemini 2 (EFC16034, FIG. 34B) studies of Example 2. EOS, end of study; SE, standard error.

FIG. 35 is a Forest plot of hazard ratios and corresponding 95% confidence intervals comparing tolebrutinib to teriflunomide for time to onset of 6-month CDP within each subgroup for the intention-to-treat population pooled from the Gemini 1 and Gemini 2 studies of Example 2.

FIGS. 36A-36B show plots of the percentage of patients (left axes) in subgroups based on the number of baseline phase rim lesions (PRL) (0, 1-3, or ≥4) having 6-month CDP (FIG. 36A) or 6-month CDW (FIG. 36B) in the study of Example 1 and Example 2, respectively. Hazard ratios (HR) are shown on the right axes.

FIGS. 37A-37B show plots of Kaplan-Meier estimates of time to onset of 6-month CDW, progression independent of relapse activity (PIRA), and relapse-associated worsening (RAW) for for a pooled population of patients treated with teriflunomide (FIG. 37A) or tolebrutinib (FIG. 37B) in the studies of Example 2.

FIG. 38 is a Forest plot of hazard ratios (HR) and corresponding 95% confidence intervals (CI) comparing tolebrutinib to placebo for time ot onset of 6-month CDP within subgroups defined by plasma exposure for tolebrutinib, M2, or tolebrutinib+M2 area-under-the curve (AUC) above or below median.

FIG. 39 is a plot of mean MSQoL-54 physical and mental health composite scores and subscale scores at baseline of the study of Example 1.

FIGS. 40A-40B are plots of the least squares mean (LSM) change from baseline in the (FIG. 40A) physical health composite score and (FIG. 40B) mental health composite scores at assessments over the course of the study, at end of study (EOS), and averaged across visits. ^{a}EOS assessment occurred between 18-43 months post-baseline (median [IQR]: 32 [28-36] months). P-values are nominal. B, baseline; SE, standard error; CI, confidence interval.

FIGS. 41A-41D show spider plots and Forest plots for the least squares mean (LSM) change from baseline and LSM difference versus placebo, respectively, at (FIG. 41A) Month 12, (FIG. 41B) Month 24, (FIG. 41C) end of study (EOS), and (FIG. 41D) averaged across visits. P-values are nominal (*P<0.05; **P<0.01; **P<0.001). CI, confidence interval.

### DETAILED DESCRIPTION

Reference will now be made in detail to certain embodiments, examples of which are illustrated in the accompanying drawings. While the disclosure provides various embodiments, it will be understood that they are not intended to limit the disclosure to those embodiments. On the contrary, the disclosure is intended to encompass alternatives, modifications, and equivalents, as will be appreviated by those of skill in the art, and may be included within the disclosure as defined by the appended claims.

The section headings used herein are for organizational purposes only and are not to be construed as limiting the desired subject matter in any way. In the event that any literature incorporated by reference contradicts any term defined in this specification, this specification controls.

### I. Definitions

Unless otherwise stated, the following terms used in the specification and claims are defined for the purposes of this disclosure and have the following meaning.

As used herein, “the BTK inhibitor,” “the BTK inhibitor compound,” and “the compound,” and “tolebrutinib” refers to (R)-1-(1-acryloylpiperidin-3-yl)-4-amino-3-(4-phenoxyphenyl)-1H-imidazo[4,5-c]pyridin-2(3H)-one having the following structure:

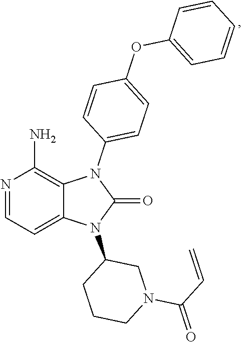 [SMILES: C=CC(=O)N1CCC[C@@H](n2c(=O)n(-c3ccc(Oc4ccccc4)cc3)c3c(N)nccc32)C1]

which is also known as 4-amino-3-(4-phenoxyphenyl)-1-[(3R)-1-(prop-2-enoyl)piperidin-3-yl]-1,3-dihydro-2H-imidazo[4,5-c]pyridin-2-one having the following structure:

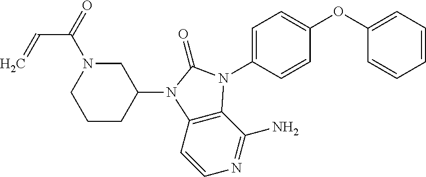 [SMILES: C=CC(=O)N1CCCC(n2c(=O)n(-c3ccc(Oc4ccccc4)cc3)c3c(N)nccc32)C1]

or a pharmaceutically acceptable salt thereof.

As used herein, the term “about” means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In some embodiments, the term “about” is used herein to modify a numerical value above and below the stated value by a variance of 5%.

A “pharmaceutically acceptable carrier” or a “pharmaceutically acceptable excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Optional” or “optionally” means that the subsequently described event or circumstance may but need not occur, and that the description includes instances where the event or circumstance occurs and instances in which it does not.

Within the scope of the present disclosure, “multiple sclerosis” or “MS” includes four clinical courses of disease, each of which might be mild, moderate, or severe: clinically isolated syndrome, relapsing remitting, secondary progressive (e.g., non-relapsing secondary progressive multiple sclerosis (“nrSPMS”)) and primary progressive (“PPMS”). Within the scope of the present disclosure, “relapsing multiple sclerosis,” “relapsing MS,” “relapsing forms of MS,” or “RMS” includes clinically isolated syndrome (“CIS”), relapsing remitting multiple sclerosis (“RRMS”), and relapsing secondary progressive multiple sclerosis (“R-SPMS”). RRMS refers to a disease state where patients experience clearly defined relapses (also called flare-ups or exacerbations), which are episodes of new or worsening symptoms (i.e., worsening neurologic function), followed by periods of partial or complete recovery (remissions). nrSPMS refers to a disease state in which the patient has stopped experiencing confirmed relapses but continues to experience accumulation of disability, experienced as various symptoms such as gait impairment, fatigue, cognition impairment, balance difficulties, loss of bowel and/or bladder function, sexual disfunction, amongst others. nrSPMS includes “non-active SPMS” in which the patient has stopped experiencing confirmed relapses and inflammatory activity as detected by MRI (e.g., Gd-enhancing T1-hyperintense lesions). PPMS refers to a disease state in which the patient experiences accumulation of disability, independent of relapses, since the onset of the disease clinical manifestation, but it likely does not have pathophysiologically distinct features from relapsing forms of MS that have entered a progressive course (SPMS).

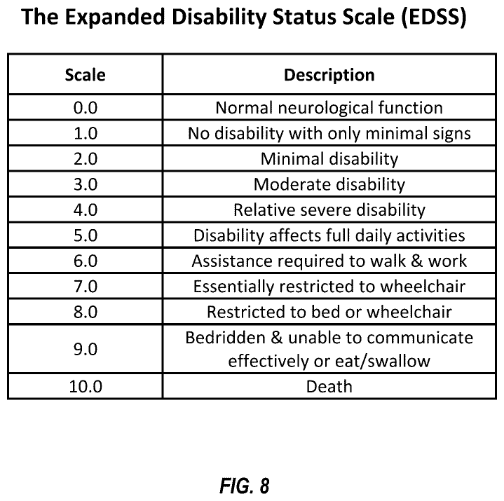

“Expanded disability status scale (EDSS) score” is a method of quantifying disability in multiple sclerosis and monitoring changes in the level of disability over time. The EDSS scale ranges from 0 to 10 in 0.5 unit increments that represent higher levels of disability. EDSS steps 1.0 to 4.5 refer to people with MS who are able to walk without any aid and is based on measures of impairment in eight functional systems (FS): pyramidal—muscle weakness or difficulty moving limbs; cerebellar—ataxia, loss of balance, coordination or tremor; brainstem—problems with speech, swallowing and nystagmus; sensory—numbness or loss of sensations; bowel and bladder function; visual function—problems with sight; cerebral functions—problems with thinking and memory. EDSS steps 5.0 to 9.5 are defined by the impairment to walking. See, e.g., FIG. 8. Information about this score is found at Kurtzke et al. Neurology 1983, 33, 1444-1452.

As used herein, “disability accumulation” or “disability accrual” refer to the change in disability in patients with multiple sclerosis. It includes both the concepts of “disability worsening,” which refers to the typically stepwise increase in disability in patients with relapsing forms of MS, and “disability progression,” which refers to the typically more continuous increase in disability in patients with progressive forms of MS (e.g., nrSPMS, PPMS). A patient's disability accumulation or accrual may also be impacted by “disability improvement,” which refers to a decrease in disability, should it occur, e.g., in response to a therapy. Disability accumulation (or accrual) that is associated with incomplete recovery after relapse is referred to as “relapse-associated worsening” or “RAW,” and typically occurs in patients with RMS. Disability accumulation (or accrual) that is not associated with relapse is referred to as “disability progression independent of relapse activity,” “progression independent of relapse activity,” or “PIRA.” See, e.g., Kappos, et al., JAMA Neurology 2020; 77:1-9; Ciccarelli, et al., Neurology 2024; 103:e209444.

As used herein, “confirmed disability accumulation,” “confirmed disability accrual,” or “CDA” refer to measurements such as confirmed disability progression (CDP), confirmed disability improvement (CDI), and confirmed disability worsening (CDW), which are used to assess disability change in patients. CDA may be assessed by a single metric such as progression or worsening in an EDSS score, or may be assessed as a composite metric of progression or worsening in one or more of multiple metrics such as an EDSS score, timed 25-foot walk (T25-FW) test, and 9-hole peg test (9-HPT). When assessed as a composite, CDA may be referred to as “composite Confirmed Disability Accumulation,” “composite Confirmed Disability Accrual,” or “cCDA.”

As used herein “administering” means providing an agent (e.g., active ingredient) or a composition containing the agent to a subject in a manner that results in the agent being inside the subject's body, or prescribing, instructing, managing, or supervising another, including the subject, to so provide said agent or composition.

As used herein, “treating” or “treatment” of a disease includes (1) inhibiting the disease, e.g., arresting, reducing, or slowing the development of the disease or its clinical symptoms; and/or (2) relieving the disease, e.g., causing regression of the disease or its clinical symptoms.”

Within the scope of the present disclosure, treatment may refer to arresting, reducing, or slowing disability accumulation (e.g., disability progression or worsening) or causing disability improvement.

For example, treatment may refer to reducing the risk of an increase in disability accumulation (e.g., disability progression or disability worsening) or to improving the chance of a decrease in disability (e.g., disability improvement). “Reducing the risk” refers to decreasing the likelihood of an increase in disability or occurrence of a disability event, which may be assessed by a particular measure such as confirmed disability accumulation (CDA), confirmed disability progression (CDP), confirmed disability worsening (CDW), sustained increase in 25-foot-walk (T25-FW), or sustained increase in 9-hole peg test (9-HPT). “Improving the chance” refers to increasing the likelihood of a decrease in disability or occurrence of a (beneficial) disability event, which may be assessed by a particular measure such as confirmed disability improvement (CDI). A risk reduction (or improvement in chance) may be statistically quantified as a hazard ratio comparing the rate of occurrence of the outcome with treatment (e.g., tolebrutinib) versus the rate of occurrence of the outcome with no treatment (e.g., placebo) or treatment with a comparator (e.g., teriflunomide). For example, a risk reduction of about 30% may correspond to a hazard ratio of about 0.70 for tolebrutinib versus placebo or a comparator. As another example, an improvement in chance of about 90% may correspond to a hazard ratio of about 1.9 for tolebrutinib versus placebo or another comparator.

As another example, treatment may refer to improving the time to onset of an increase in disability accumulation (e.g., disability progression or disability worsening), wherein improving means lengthening the time to onset, i.e., slowing disability accumulation, or to improving the time to onset of a decrease in disability (e.g., disability improvement), wherein improving means shortening the time to onset. Improvements may be assessed by a particular measure for the onset of an event such as confirmed disability accumulation (CDA), confirmed disability progression (CDP), confirmed disability worsening (CDW), sustained increase in 25-foot-walk (T25-FW), or sustained increase in 9-hole peg test (9-HPT), confirmed disability improvement (CDI).

As used herein, “placebo” refers to a composition which contains no active ingredient, for example, no tolebrutinib. A placebo may be administered in a clinical trial to patients in a control group for comparison with patients receiving a composition which contains the active ingredient, for example, tolebrutinib. As will be understood by the skilled artisan, an effect observed for tolebrutinib versus placebo is likely to also be observed for tolebrutinib versus no treatment, with the possibility that placebo or nocebo effects account for differences between comparison with placebo and with no treatment.

Before describing the present teachings in detail, it is to be understood that the disclosure is not limited to specific compositions or process steps, as such may vary.

It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range, e.g., a range of “between 1.0 and 2.0” or “from 1.0 to 2.0” are equivalent and include the endpoints “1.0” and “2.0”. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims.)

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “and/or,” unless the context requires otherwise.

“Ceasing” or “cessation” when used regarding administration of a BTK inhibitor compound means that the BTK inhibitor compound is no longer being administered to the patient on either a temporary or permanent basis.

“Monitoring” with reference to assessment of the level of ALT in a patient means checking, and/or detecting the level of ALT in a patient over at least two points in time; in some embodiments, over a period of time; in some embodiments, monthly; in some embodiments, at least monthly; in some embodiments, weekly; in some embodiments, at least weekly; in some embodiments, every 5 days; in some embodiments, every 3 days; in some embodiments, every 2 to 3 days; in some embodiments, every 2 days; in some embodiments, daily.

### II. Administered BTK Inhibitor Compound

In some embodiments, the BTK inhibitor compound (tolebrutinib), (R)-1-(1-acryloylpiperidin-3-yl)-4-amino-3-(4-phenoxyphenyl)-1H-imidazo[4,5-c]pyridin-2(3H)-one is administered for treating MS in a patient in need thereof. In some embodiments, the BTK inhibitor compound is a pharmaceutically acceptable salt of (R)-1-(1-acryloylpiperidin-3-yl)-4-amino-3-(4-phenoxyphenyl)-1H-imidazo[4,5-c]pyridin-2(3H)-one. In some embodiments, a therapeutically effective amount of the BTK inhibitor compound is administered. In some embodiments, a dose of 5 to 60 mg of the BTK inhibitor compound, measured in base form, is administered. In some embodiments, a dose of 60 mg of the BTK inhibitor compound, measured in base form, is administered. In some embodiments, a dose of 60 mg once daily of the BTK inhibitor compound, measured in base form, is administered.

In some embodiments, a therapeutically effective amount of tolebrutinib is provided for use in a method for treating nrSPMS in a patient in need thereof.

The BTK inhibitor compound can be prepared according to the methods and schemes described in, e.g., U.S. Pat. No. 9,688,676 B2, in particular the content of column 62, line 8 to column 65 line 32, and column 67, line 28 to column 69, which is incorporated herein by reference.

The following preparation of the compound of (R)-1-(1-acryloylpiperidin-3-yl)-4-amino-3-(4-phenoxyphenyl)-1H-imidazo[4,5-c]pyridin-2(3H)-one, is given to enable those skilled in the art to prepare the BTK inhibitor compound. The synthetic route should not be considered as limiting the scope of the disclosure, but merely as being illustrative and representative thereof.

Exemplary synthesis of (R)-1-(1-acryloylpiperidin-3-yl)-4-amino-3-(4-phenoxyphenyl)-1H-imidazo[4,5-c]pyridin-2(3H)-one:

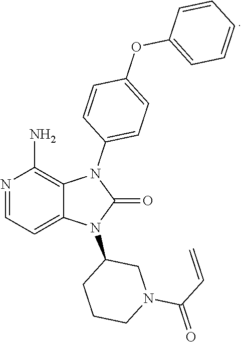 [SMILES: C=CC(=O)N1CCC[C@@H](n2c(=O)n(-c3ccc(Oc4ccccc4)cc3)c3c(N)nccc32)C1]

Into a 100 mL round-bottom flask was placed (R)-4-amino-3-(4-phenoxyphenyl)-1-(piperidin-3-yl)-1H-imidazo[4,5-c]pyridin-2(3H)-one (150 mg, 0.37 mmol, 1.00 equiv), DCM-CH3OH (6 mL), TEA (113 mg, 1.12 mmol, 3.00 equiv). This was followed by the addition of prop-2-enoyl chloride (40.1 mg, 0.44 mmol, 1.20 equiv) dropwise with stirring at 0° C. in 5 min. The resulting solution was stirred for 2 h at 0° C. The resulting mixture was concentrated under vacuum. The residue was applied onto a silica gel column with dichloromethane/methanol (30:1). The crude product (100 mg) was purified by Prep-HPLC under the following conditions (Column, XBridge Prep C18 OBD Column, 5 μm, 19*150 mm; mobile phase, water with 0.05% TFA and ACN (25.0% ACN up to 45.0% in 8 min). 54.5 mg product of (R)-1-(1-acryloylpiperidin-3-yl)-4-amino-3-(4-phenoxyphenyl)-1H-imidazo[4,5-c]pyridin-2(3H)-one was obtained as a white solid. LC-MS m/z: 465.2 (M+1).

The BTK inhibitor compound can also be prepared according to the methods and schemes described in, for example, WO2022/0121670. In addition, it can be prepared as described in WO2022/257845. Further, the BTK inhibitor compound can be prepared as described in WO2023/122072.

In some embodiments, provided herein is a solid form of the BTK inhibitor, or a salt thereof. In some embodiments, provided is a solid form of the salt of the BTK inhibitor. In some embodiments, provided herein is a solid form of the BTK inhibitor. In some embodiments, provided is an amorphous form of the BTK inhibitor, or a salt thereof. In some embodiments, provided is an amorphous form of the salt of the BTK inhibitor. In some embodiments, provided is an amorphous form of the BTK inhibitor. In some embodiments, provided is a crystalline form of the BTK inhibitor, or a salt thereof. In some embodiments, provided is a crystalline form of the salt of the BTK inhibitor. In some embodiments, provided is a crystalline form of the BTK inhibitor.

In some embodiments, provided is a crystalline form of tolebrutinib, wherein an X-ray powder diffraction pattern comprises one or two or three characteristic peaks at 2theta values of 7.7°±0.2°, 11.0°±0.2° and 22.8°±0.2° using CuKα radiation. In some embodiments, provided is a crystalline form of tolebrutinib, wherein an X-ray powder diffraction pattern comprises one or two or three characteristic peaks at 2theta values of 12.0°±0.2°, 16.1°±0.2° and 18.5°±0.2° using CuKα radiation. In at least one embodiment, provided is a crystalline form of tolebrutinib, wherein an X-ray powder diffraction pattern comprises characteristic peaks at 2theta values of 7.7°±0.2°, 11.0°±0.2°, 22.8°±0.2°, 12.0°±0.2°, 16.1°±0.2°, and 18.5°±0.2° using CuKα radiation. In some embodiments, provided is a crystalline form of tolebrutinib, wherein an X-ray powder diffraction pattern comprises one or two or three characteristic peaks at 2theta values of 713.6°±0.2°, 20.1°±0.2° and 24.8°±0.2° using CuKα radiation.

In some embodiments, provided is a crystalline form of tolebrutinib, wherein an X-ray powder diffraction pattern comprises one or two or three characteristic peaks at 2theta values of 4.1°±0.2°, 10.2°±0.2° and 22.6°±0.2° using CuKα radiation. In some embodiments, provided is a crystalline form of tolebrutinib, wherein an X-ray powder diffraction pattern comprises one or two or three characteristic peaks at 2theta values of 11.3°±0.2°, 16.5°±0.2° and 17.8°±0.2° using CuKα radiation. In some embodiments, provided is a crystalline form of tolebrutinib, wherein an X-ray powder diffraction pattern comprises one or two or three characteristic peaks at 2theta values of 8.2°±0.2°, 10.8°±0.2° and 24.7°±0.2° using CuKα radiation. In some embodiments, provided is a crystalline form of tolebrutinib, wherein an X-ray powder diffraction pattern comprises one or two or three or four or five or six or seven or eight or nine or ten characteristic peaks at 2theta values of 4.10°±0.2°, 10.2°±0.2°, 22.6°±0.2°, 11.3°±0.2°, 16.5°±0.2°, 17.8°±0.2°, 8.2°±0.2°, 10.8°±0.2°, 24.7°±0.2° and 20.5°±0.2° using CuKα radiation.

In some embodiments, provided is a crystalline form of tolebrutinib, wherein an X-ray powder diffraction pattern comprises one or more characteristic peaks at 2theta values at about 7.66°2θ, 7.86°2θ, 10.03°2θ, 10.51°2θ, 10.97°2θ, 11.99°2θ, 13.19°2θ, 13.59°2θ and 13.96°2θ using CuKα radiation. In some embodiments, provided is a crystalline form of tolebrutinib, wherein an X-ray powder diffraction pattern comprises one or more characteristic peaks at 2theta values at about 4.15°2θ, 10.22°2θ, 10.41°2θ, 11.03°2θ, 14.41°2θ, 14.85°2θ, 15.63°2θ, 16.55°2θ and 17.73°2θ using CuKα radiation.

In some embodiments, provided is a crystalline form of tolebrutinib HCl salt, wherein an X-ray powder diffraction pattern comprises one or more characteristic peaks at 2theta values at about 6.309°2θ, 9.480°2θ, 10.933°2θ, 12.261°2θ, 12.647°2θ, 14.482°2θ, 14.918°2θ, 16.253°2θ and 16.425°2θ using CuKα radiation. In some embodiments, provided is a crystalline form of tolebrutinib HCl salt, wherein an X-ray powder diffraction pattern comprises one or more characteristic peaks at 2theta values at about 8.00°2θ, 10.11°2θ, 11.98°2θ, 13.33°2θ, 14.40°2θ, 14.92°2θ, 15.66°2θ, 16.05°2θ, 16.72°2θ, and 17.28°2θ using CuKα radiation.

In some embodiments, the tolebrutinib, pharmaceutically acceptable salt thereof, or crystalline form thereof is provided as a pharmaceutical composition in association with at least one pharmaceutically acceptable excipient.

### III. Therapeutic Methods

Provided herein are methods of reducing disability accumulation independent of relapse activity in a patient with MS in need thereof comprising administering to the patient in need thereof a therapeutically effective amount of tolebrutinib or pharmaceutically acceptable salt thereof.

In some embodiments, provided herein are methods useful for treating patients having non-relapsing secondary progressive multiple sclerosis (nrSPMS) by administration of tolebrutinib or pharmaceutically acceptable salt thereof.

In an embodiment, provided herein are methods of treating a patient with nrSPMS, comprising administering to the patient in need thereof a therapeutically effective amount of tolebrutinib or pharmaceutically acceptable salt thereof. However, in some embodiments, the administration of the compound will only occur when certain patient outcomes are met.

In one embodiment, the outcome is the patient's risk of confirmed disability progression (CDP) is reduced. In at least one embodiment, the risk is reduced is relative to placebo. In at least one embodiment, the risk is reduced is relative to no treatment. In some embodiments, the risk is reduced about 5%, 10%, 15%, 20%, 25%, 30%, 35%, 40%, 45%, or 50% for tolebrutinib versus placebo. In at least one embodiment, the risk of 6-month CDP is reduced about 31% for tolebrutinib versus placebo. In at least one embodiment, the risk of 3-month CDP is reduced about 24% for tolebrutinib versus placebo. In one embodiment, the outcome is the patient's total number of new and/or enlarging T2-hyperintense lesions as detected by MRI, defined as the sum of the individual number of new and/or enlarging T2 lesions, are reduced. In at least one embodiment, the annualized rate is reduced relative to placebo. In at least one embodiment, the annualized rate is reduced relative to no treatment. In some embodiments, the annualized rate is reduced about 5%, 10%, 15%, 20%, 25%, 30%, 35%, 40%, 45%, or 50% for tolebrutinib versus placebo. In at least one embodiment, the annualized rate is reduced about 38% for tolebrutinib versus placebo. In one embodiment, the outcome is the patient's risk of sustained 20% increase in the timed 25-foot walk (T25-FW) test confirmed over at least 3 months is reduced. In at least one embodiment, the risk reduction is relative to placebo. In at least one embodiment, the risk reduction is relative to no treatment. In some embodiments, the risk is reduced about 5%, 10%, 15%, 20%, 25%, 30%, 35%, 40%, 45%, or 50% for tolebrutinib versus placebo. In at least one embodiment, the risk of sustained 20% increase in T25-FW test confirmed over 3 months is reduced about 23% for tolebrutinib versus placebo. In one embodiment, the outcome is the patient's chance of confirmed disability improvement (CDI) is improved. In at least one embodiment, the chance is improved relative to placebo. In at least one embodiment, the chance is improved relative to treatment. In some embodiments, the chance is improved about 10%, 20%, 30%, 40%, 50%, 60%, 70%, 80%, 90%, or 100% for tolebrutinib versus placebo. In at least one embodiment, the chance of 6-month CDI is improved about 88% for tolebrutinib versus placebo.

Provided herein are methods of treating a patient with nrSPMS in need thereof, comprising administering to the patient in need thereof a therapeutically effective amount of tolebrutinib or pharmaceutically acceptable salt thereof, wherein the risk of confirmed disability progression (CDP) is reduced. In one embodiment, the patient's CDP is measured over at least three months, such as at least six months. In some embodiments, the CDP is measured over at least one month, at least two months, at least three months, at least four months, at least five months, at least six months, at least seven months, at least eight months, at least nine months, at least ten months, at least eleven months, or at least a year. In at least one embodiment, the risk is reduced is relative to placebo. In at least one embodiment, the risk is reduced is relative to no treatment. In some embodiments, the risk is reduced about 5%, 10%, 15%, 20%, 25%, 30%, 35%, 40%, 45%, or 50% for tolebrutinib versus placebo. In at least one embodiment, the risk of 6-month CDP is reduced about 310% for tolebrutinib versus placebo. In at least one embodiment, the risk of 3-month CDP is reduced about 24% for tolebrutinib versus placebo.

In one embodiment, the patient's CDP increases at least 1.0 point from a baseline expanded disability status scale score (EDSS) when the baseline score is less than or equal to 5.0. In one embodiment, the patient's CDP in increases at least 0.5 points when the baseline EDSS score is greater than 5.0.

In one embodiment, the patient with nrSPMS has a SPMS diagnosis with an expanded disability status scale score (EDSS) between 3.0 and 6.5. In one embodiment, the patient with nrSPMS has no clinical relapse in the previous 24 months. In one embodiment, the patient with nrSPMS has disability accumulation in the previous 12 months. In one embodiment, the patient has a SPMS diagnosis with an expanded disability status scale score (EDSS) between 3.0 and 6.5, no clinical relapse in the previous 24 months, and has disability accumulation in the previous 12 months.

Also provided herein are methods of reducing the risk of confirmed disability progression (CDP) in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

In one embodiment, the patient's CDP is measured over at least three months, such as at least six months. In some embodiments, the CDP is measured over at least one month, at least two months, at least three months, at least four months, at least five months, at least six months, at least seven months, at least eight months, at least nine months, at least ten months, at least eleven months, or at least a year. In one embodiment, the confirmed disability progression (CDP) comprises: increasing at least 1.0 point from a baseline expanded disability status scale score (EDSS) when the baseline score is less than or equal to 5.0; or increasing at least 0.5 points when the baseline EDSS score is greater than 5.0. In one embodiment, the patient with nrSPMS has: a SPMS diagnosis with an expanded disability status scale score (EDSS) between 3.0 and 6.5; no clinical relapse in the previous 24 months; and disability accumulation in the previous 12 months. In at least one embodiment, the risk reduction is relative to placebo. In at least one embodiment, the risk reduction is relative to no treatment. In some embodiments, the risk is reduced about 5%, 10%, 15%, 20%, 25%, 30%, 35%, 40%, 45%, or 50% for tolebrutinib versus placebo. In at least one embodiment, the risk of 6-month CDP is reduced about 310% for tolebrutinib versus placebo. In at least one embodiment, the risk of 3-month CDP is reduced about 24% for tolebrutinib versus placebo.

Also provided herein is a method of reducing the total number of new and/or enlarging T2-hyperintense lesions as detected by MRI, in a patient with non-relapsing secondary progressive multiple sclerosis (nrSPMS) in need thereof, comprising administering to the patient in need thereof a therapeutically effective amount of tolebrutinib or pharmaceutically acceptable salt thereof. In one embodiment, reducing comprises reducing the annualized rate of new and/or enlarging T2-hyperintense lesions. In one embodiment, the patient with nrSPMS has: a SPMS diagnosis with an expanded disability status scale score (EDSS) between 3.0 and 6.5; no clinical relapse in the previous 24 months; and disability accumulation in the previous 12 months. In one embodiment, the annualized rate of new and/or enlarging T2-hyperintense lesions is less than 2. In one embodiment, the annualized rate of new and/or enlarging T2-hyperintense lesions is about 1.8. In at least one embodiment, the annualized rate is reduced relative to placebo. In at least one embodiment, the annualized rate is reduced relative to no treatment. In some embodiments, the annualized rate is reduced about 5%, 10%, 15%, 20%, 25%, 30%, 35%, 40%, 45%, or 50% for tolebrutinib versus placebo. In at least one embodiment, the annualized rate is reduced about 38% for tolebrutinib versus placebo.

Provided herein is a method of reducing the risk of sustained 20% increase in the timed 25-foot walk (T25-FW) test confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof. In one embodiment, the T25-FW test is confirmed over at least at least four months, at least five months, at least six months, at least seven months, at least eight months, at least nine months, at least ten months, at least eleven months, or at least a year. In one embodiment, the patient with nrSPMS has: a SPMS diagnosis with an expanded disability status scale score (EDSS) between 3.0 and 6.5; no clinical relapse in the previous 24 months; and disability accumulation in the previous 12 months. In at least one embodiment, the risk reduction is relative to placebo. In at least one embodiment, the risk reduction is relative to no treatment. In some embodiments, the risk is reduced about 5%, 10%, 15%, 20%, 25%, 30%, 35%, 40%, 45%, or 50% for tolebrutinib versus placebo. In at least one embodiment, the risk of of sustained 20% increase in the T25-FW test confirmed over at least 3 months reduced about 23% for tolebrutinib versus placebo.

Also provided herein is a method of improving the chance of confirmed disability improvement (CDI), in a patient with non-relapsing secondary progressive multiple sclerosis (nrSPMS) in need thereof, comprising administering to the patient in need thereof a therapeutically effective amount of tolebrutinib or pharmaceutically acceptable salt thereof. In one embodiment, the patient's CDI is measured over at least three months, such as at least six months. In some embodiments, the CDI is measured over at least one month, at least two months, at least three months, at least four months, at least five months, at least six months, at least seven months, at least eight months, at least nine months, at least ten months, at least eleven months, or at least a year. In at least one embodiment, the confirmed disability improvement (CDI) comprises decreasing at least 1.0 point from a baseline expanded disability status scale score (EDSS). In some embodiments, the patient with nrSPMS has: a SPMS diagnosis with an expanded disability status scale score (EDSS) between 3.0 and 6.5; no clinical relapse in the previous 24 months; and disability accumulation in the previous 12 months. In at least one embodiment, the chance is improved relative to placebo. In at least one embodiment, the chance is improved relative to no treatment. In some embodiments, the chance is improved about 10%, 20%, 30%, 40%, 50%, 60%, 70%, 80%, 90%, or 100% for tolebrutinib versus placebo. In at least one embodiment, the chance of 6-month CDI is improved about 88% for tolebrutinib versus placebo.

Provided herein is a method of improving time to onset of confirmed disability progression (CDP) in a patient with non-relapsing secondary progressive multiple sclerosis (nrSPMS) in need thereof, comprising administering to the patient in need thereof a therapeutically effective amount of tolebrutinib or pharmaceutically acceptable salt thereof. In one embodiment, the patient's CDP is measured over at least three months, such as at least six months. In some embodiments, the CDP is measured over at least one month, at least two months, at least three months, at least four months, at least five months, at least six months, at least seven months, at least eight months, at least nine months, at least ten months, at least eleven months, or at least a year. In one embodiment, the confirmed disability progression (CDP) comprises: increasing at least 1.0 point from a baseline expanded disability status scale score (EDSS) when the baseline score is less than or equal to 5.0; or increasing at least 0.5 points when the baseline EDSS score is greater than 5.0. In some embodiments, the patient with nrSPMS has: a SPMS diagnosis with an expanded disability status scale score (EDSS) between 3.0 and 6.5; no clinical relapse in the previous 24 months; and disability accumulation in the previous 12 months. In some embodiments, the improvement is relative to placebo. In some embodiments, the improvement is relative to no treatment. In some embodiments, the improvement is a delay in time of onset of about 5%, 10%, 15%, 20%, 25%, 30%, 35%, 40%, 45%, or 50% for tolebrutinib versus placebo. In at least one embodiment, the improvement is a delay in time of onset of 6-month CDP of about 31% for tolebrutinib versus placebo. In at least one embodiment, the improvement is a delay in time of onset of 3-month CDP of about 24% for tolebrutinib versus placebo.

Further provided herein is a method of improving time to onset of sustained 20% increase in the timed 25-foot walk (T25-FW) test confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof. In one embodiment, the patient with nrSPMS has: a SPMS diagnosis with an expanded disability status scale score (EDSS) between 3.0 and 6.5; no clinical relapse in the previous 24 months; and disability accumulation in the previous 12 months. In some embodiments, the improvement is relative to placebo. In some embodiments, the improvement is relative to no treatment. In some embodiments, the improvement is a delay in time of onset of about 5%, 10%, 15%, 20%, 25%, 30%, 35%, 40%, 45%, or 50% for tolebrutinib versus placebo. In at least one embodiment, the improvement is a delay in time of onset of sustained 20% increase in the T25-FW test confirmed over at least 3 months about 23% for tolebrutinib versus placebo.

Provided herein is a method of improving time to onset of confirmed disability improvement (CDI) in a patient with non-relapsing secondary progressive multiple sclerosis (nrSPMS) in need thereof, comprising administering to the patient in need thereof a therapeutically effective amount of tolebrutinib or pharmaceutically acceptable salt thereof. In one embodiment, the patient's CDI is measured over at least three months, such as at least six months. In some embodiments, the CDI is measured over at least one month, at least two months, at least three months, at least four months, at least five months, at least six months, at least seven months, at least eight months, at least nine months, at least ten months, at least eleven months, or at least a year. In at least one embodiment, the CDI comprises decreasing at least 1.0 point from a baseline expanded disability status scale score (EDSS). In some embodiments the patient with nrSPMS has: a SPMS diagnosis with an expanded disability status scale score (EDSS) between 3.0 and 6.5; no clinical relapse in the previous 24 months; and disability accumulation in the previous 12 months. In some embodiments, the improvement is relative to placebo. In some embodiments, the improvement is relative to no treatment. In some embodiments, the improvement is a shortening of time of onset by about 10%, 20%, 30%, 40%, 50%, 60%, 70%, 80%, 90%, or 100% for tolebrutinib versus placebo. In at least one embodiment, the improvement is a shortening of time of onset of 6-month CDP of about 88% for tolebrutinib versus placebo.

Disclosed herein is tolebrutinib or a pharmaceutically acceptable salt thereof for use in treating a patient with non-relapsing secondary progressive multiple sclerosis (nrSPMS) in certain methods disclosed herein. In some embodiments, treating a patient with nrSPMS comprises at least one of reducing the risk of confirmed disability progression (CDP); reducing the total number of new and/or enlarging T2-hyperintense lesions as detected by MRI; reducing the risk of sustained 20% increase in the timed 25-foot walk (T25-FW) test confirmed over at least 3 months; and improving the chance of confirmed disability improvement (CDI).

Also disclosed herein is tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof reduces the risk of confirmed disability progression (CDP) in certain methods disclosed herein.

Disclosed herein is tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof reduces the total number of new and/or enlarging T2-hyperintense lesions as detected by MRI in certain methods disclosed herein.

Further disclosed herein is tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof reduces the risk of sustained 20% increase in the timed 25-foot walk (T25-FW) test confirmed over at least 3 months in certain methods disclosed herein.

Still further disclosed herein is tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves the chance of confirmed disability improvement (CDI) in certain methods disclosed herein.

Disclosed herein is tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves time to onset of confirmed disability progression (CDP) in certain methods disclosed herein.

Also disclosed herein it tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves time to onset of sustained 20% increase in the timed 25-foot walk (T25-FW) test confirmed over at least 3 months in certain methods disclosed herein.

Disclosed herein is tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves time to onset of confirmed disability improvement (CDI) in certain methods disclosed herein.

Disclosed herein is the use of tolebrutinib or a pharmaceutically acceptable salt thereof for the manufacture of a medicament for a patient with nrSPMS in certain methods disclosed herein. In some embodiments, treating a patient with nrSPMS comprises at least one of reducing the risk of confirmed disability progression (CDP); reducing the total number of new and/or enlarging T2-hyperintense lesions as detected by MRI; reducing the risk of sustained 20% increase in the timed 25-foot walk (T25-FW) test confirmed over at least 3 months; and improving the chance of confirmed disability improvement (CDI).

Disclosed herein is the use of tolebrutinib or a pharmaceutically acceptable salt thereof for the manufacture of a medicament for treating a patient with nrSPMS, wherein the tolebrutinib or a pharmaceutically acceptable salt thereof reduces the risk of confirmed disability progression (CDP), in certain methods disclosed herein.

Also disclosed herein is the use of tolebrutinib or a pharmaceutically acceptable salt thereof for the manufacture of a medicament for treating a patient with nrSPMS, wherein the tolebrutinib or a pharmaceutically acceptable salt thereof reduces the total number of new and/or enlarging T2-hyperintense lesions as detected by MRI, in certain methods disclosed herein.

Further disclosed herein is the use of tolebrutinib or a pharmaceutically acceptable salt thereof for the manufacture of a medicament for treating a patient with nrSPMS, wherein the tolebrutinib or a pharmaceutically acceptable salt thereof reduces the risk of sustained 20% increase in the timed 25-foot walk (T25-FW) test confirmed over at least 3 months, in certain methods disclosed herein.

Still further disclosed herein is the use of tolebrutinib or a pharmaceutically acceptable salt thereof for the manufacture of a medicament for treating a patient with nrSPMS, wherein the tolebrutinib or a pharmaceutically acceptable salt thereof improves the chance of confirmed disability improvement (CDI), in certain methods disclosed herein.

Disclosed herein is the use of tolebrutinib or a pharmaceutically acceptable salt thereof for the manufacture of a medicament for treating a patient with nrSPMS, wherein the tolebrutinib or a pharmaceutically acceptable salt thereof improves time to onset of confirmed disability progression (CDP), in certain methods disclosed herein.

Also disclosed herein is the use of tolebrutinib or a pharmaceutically acceptable salt thereof for the manufacture of a medicament for treating a patient with nrSPMS, wherein the tolebrutinib or a pharmaceutically acceptable salt thereof improves time to onset of sustained 20% increase in the timed 25-foot walk (T25-FW) test confirmed over at least 3 months, in certain methods disclosed herein.

Further disclosed herein is the use of tolebrutinib or a pharmaceutically acceptable salt thereof for the manufacture of a medicament for treating a patient with nrSPMS, wherein the tolebrutinib or a pharmaceutically acceptable salt thereof improves time to onset of confirmed disability improvement (CDI), in certain methods disclosed herein.

Provided herein is a method of reducing the risk of confirmed disability worsening (CDW) in a patient with a relapsing form of multiple sclerosis (RMS) in need thereof, comprising administering to the patient in need thereof a therapeutically effective amount of tolebrutinib or pharmaceutically acceptable salt thereof. In some embodiments, CDW is measured over at least 1, 2, 3, 4, 5, 6, 7, 8, 9, 10, 11, 12, 13, 14, 15, 16, 17, 18, 19, or 20 weeks. In some embodiments, CDW is measured over at least 1, 2, 3, 4, 5, 6, 7, 8, 9, 10, 12, 16, 20, 24, 28, 32, 36, or 48 months. In some embodiments, CDW is measured over about 1, 2, 3, 4, 5, 6, 7, 8, 9, 10, or 12 months. In some embodiments, CDW is measured over three months. In some embodiments, CDW is measured over at least three months, such as at least six months. In some embodiments, CDW is measured over six months.

In some embodiments, CDW comprises: (a) increasing at least 1.5 point from a baseline expanded disability status scale score (EDSS) when the baseline score is 0; (b) increasing at least 1.0 point from the baseline EDSS when the baseline score is greater than or equal to 0.5 and less than or equal to 5.5; or (c) increasing at least 0.5 points when the baseline EDSS score is greater than 5.5. In some embodiments, CDW comprises increasing at least 1.5 point from the baseline EDSS when the baseline score is 0. In some embodiments, CDW comprises increasing at least 1.0 point from the baseline EDSS when the baseline score is greater than or equal to 0.5 and less than or equal to 5.5. In some embodiments, CDW comprises increasing at least 0.5 points when the baseline EDSS score is greater than 5.5.

In some embodiments, the risk reduction is relative to treatment with teriflunomide. In some embodiments, the risk is reduced about 5%, 10%, 15%, 20%, 25%, 30%, 35%, 40%, 45%, or 50% for tolebrutinib versus teriflunomide. In at least one embodiment, the risk of 6-month CDW is reduced about 29% for tolebrutinib versus teriflunomide. In at least one embodiment, the risk of 3-month CDW is reduced about 27% for tolebrutinib versus teriflunomide.

Also provided herein is a method of improving time to onset of confirmed disability worsening (CDW) in a patient with a relapsing form of multiple sclerosis (RMS) in need thereof, comprising administering to the patient in need thereof a therapeutically effective amount of tolebrutinib or pharmaceutically acceptable salt thereof. In some embodiments, CDW is measured over at least 1, 2, 3, 4, 5, 6, 7, 8, 9, 10, 11, 12, 13, 14, 15, 16, 17, 18, 19, or 20 weeks. In some embodiments, CDW is measured over at least 1, 2, 3, 4, 5, 6, 7, 8, 9, 10, 12, 16, 20, 24, 28, 32, 36, or 48 months. In some embodiments, CDW is measured over about 1, 2, 3, 4, 5, 6, 7, 8, 9, 10, or 12 months. In some embodiments, CDW is measured over three months. In some embodiments, CDW is measured over at least three months, such as at least six months. In some embodiments, CDW is measured over six months.

In some embodiments, CDW comprises: (a) increasing at least 1.5 point from a baseline expanded disability status scale score (EDSS) when the baseline score is 0; (b) increasing at least 1.0 point from the baseline EDSS when the baseline score is greater than or equal to 0.5 and less than or equal to 5.5; or (c) increasing at least 0.5 points when the baseline EDSS score is greater than 5.5.

In some embodiments, the improvement in time to onset of CDW is relative to treatment with teriflunomide. In some embodiments, the improvement is a delay in time of onset of about 5%, 10%, 15%, 20%, 25%, 30%, 35%, 40%, 45%, or 50% for tolebrutinib versus teriflunomide. In at least one embodiment, the improvement is a delay in time of onset of 6-month CDW of about 29% for tolebrutinib versus teriflunomide. In at least one embodiment, improvement is a delay in time of onset of 3-month CDW of about 27% for tolebrutinib versus teriflunomide.

Also provided herein is a method of improving percent change in brain volume loss (BVL) as detected by brain MRI in a patient with relapsing forms of multiple sclerosis (RMS), comprising administering to the patient in need thereof a therapeutically effective amount of tolebrutinib or pharmaceutically acceptable salt thereof.

In some embodiments, the percent change BVL improvement is relative to treatment with teriflunomide. In some embodiments, the percent change BVL decreases by about 5%, 10%, 15%, 20%, 25%, 30%, 35%, 40%, 45%, or 50% for tolebrutinib versus teriflunomide. In at least one embodiment, the percent change BVL decreases by about 20% for tolebrutinib versus teriflunomide.

In some embodiments, the patient has an RMS diagnosis with an EDSS less than or equal to 5.5 prior to initial administration of tolebrutinib. In some embodiments, the patient has one or more of the following: (a) one or more documented relapses in the previous year; (b) two or more documented relapses in the previous 2 years; and (c) one or more Gd-enhancing lesions on an MRI scan in the previous year. In some embodiments, the patient has two or more of the following: (a) one or more documented relapses in the previous year; (b) two or more documented relapses in the previous 2 years; and (c) one or more Gd-enhancing lesions on an MRI scan in the previous year.

Also provided herein is tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof reduces the risk of confirmed disability worsening (CDW) in certain methods described herein.

Also provided herein is tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves time to onset of confirmed disability worsening (CDW) in certain methods described herein.

Also provided herein is tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves percent change in brain volume loss (BVL) as detected by brain MRI in certain methods described herein.

Also provided herein is the use of tolebrutinib or a pharmaceutically acceptable salt thereof in the manufacture of a medicament for treating a patient with RMS, wherein tolebrutinib or a pharmaceutically acceptable salt thereof reduces the risk of confirmed disability worsening (CDW), in certain methods described herein.

Also provided herein is the use of tolebrutinib or a pharmaceutically acceptable salt thereof in the manufacture of a medicament for treating a patient with RMS, wherein tolebrutinib or a pharmaceutically acceptable salt thereof improves time to onset of confirmed disability worsening (CDW), in certain methods described herein.

Also provided herein is the use of tolebrutinib or a pharmaceutically acceptable salt thereof in the manufacture of a medicament for treating a patient with RMS, wherein tolebrutinib or a pharmaceutically acceptable salt thereof improves percent change in brain volume loss (BVL) as detected by brain MRI, in certain methods described herein.

Provided herein is a method of treating a patient with non-relapsing secondary progressive multiple sclerosis (nrSPMS) in need thereof, comprising administering to the patient in need thereof a therapeutically effective amount of tolebrutinib or pharmaceutically acceptable salt thereof, wherein at least one outcome chosen from the following occurs: (a) the risk of confirmed disability progression (CDP) is reduced; (b) the total number of new and/or enlarging T2-hyperintense lesions as detected by MRI, defined as the sum of the individual number of new and/or enlarging T2 lesions, are reduced; (c) the risk of sustained 20% increase in the timed 25-foot walk (T25-FW) test confirmed over at least 3 months is reduced; and (d) the chance of confirmed disability improvement (CDI) is improved. In some embodiments, one or more of the reduction in risk of CDP, the reduction in total number of new and/or enlarging T2-hyperintense lesions, the reduction in risk of sustained 20% increase in T25-FW test, or the improvement of chance of CDI is relative to placebo. In some embodiments, one or more of the reduction in risk of CDP, the reduction in total number of new and/or enlarging T2-hyperintense lesions, the reduction in risk of sustained 20% increase in T25-FW test, or the improvement of chance of CDI is relative to no treatment. In some embodiments, the risk of CDP is reduced about 5%, 10%, 15%, 20%, 25%, 30%, 35%, 40%, 45%, or 50% for tolebrutinib versus placebo. In at least one embodiment, the risk of 6-month CDP is reduced about 31% for tolebrutinib versus placebo. In at least one embodiment, the risk of 3-month CDP is reduced about 24% for tolebrutinib versus placebo. In some embodiments, the annualized rate of new and/or enlarging T2-hyperintense lesions is reduced about 5%, 10%, 15%, 20%, 25%, 30%, 35%, 40%, 45%, or 50% for tolebrutinib versus placebo. In at least one embodiment, the annualized rate is reduced about 38% for tolebrutinib versus placebo. In some embodiments, the risk of sustained 20% increase in T25-FW test is reduced about 5%, 10%, 15%, 20%, 25%, 30%, 35%, 40%, 45%, or 50% for tolebrutinib versus placebo. In at least one embodiment, the risk of sustained 20% increase in T25-FW test confirmed over 3 months is reduced about 23% for tolebrutinib versus placebo. In some embodiments, the chance of CDI is improved about 10%, 20%, 30%, 40%, 50%, 60%, 70%, 80%, 90%, or 100% for tolebrutinib versus placebo. In at least one embodiment, the chance of 6-month CDI is improved about 88% for tolebrutinib versus placebo.

Also provided herein is a method of treating a patient with non-relapsing secondary progressive multiple sclerosis (nrSPMS) in need thereof, comprising administering to the patient in need thereof a therapeutically effective amount of tolebrutinib or pharmaceutically acceptable salt thereof, wherein the risk of confirmed disability progression (CDP) is reduced. In some embodiments, the CDP is measured over at least three months, such as at least six months. In some embodiments, the confirmed disability progression (CDP) comprises: (a) increasing at least 1.0 point from a baseline expanded disability status scale score (EDSS) when the baseline score is less than or equal to 5.0; or (b) increasing at least 0.5 points when the baseline EDSS score is greater than 5.0. In some embodiments, the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.

In some embodiments, provided herein is a method of reducing the risk of confirmed disability progression (CDP) in a patient with non-relapsing secondary progressive multiple sclerosis (nrSPMS) in need thereof, comprising administering to the patient in need thereof a therapeutically effective amount of tolebrutinib or pharmaceutically acceptable salt thereof. In some embodiments, the CDP is measured over at least three months, such as at least six months. In some embodiments, confirmed disability progression (CDP) comprises: (a) increasing at least 1.0 point from a baseline expanded disability status scale score (EDSS) when the baseline score is less than or equal to 5.0; or (b) increasing at least 0.5 points when the baseline EDSS score is greater than 5.0. In some embodiments, the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months. In some embodiments, the risk reduction is relative to placebo. In some embodiments, the risk reduction is relative to no treatment. In some embodiments, the risk is reduced about 5%, 10%, 15%, 20%, 25%, 30%, 35%, 40%, 45%, or 50% for tolebrutinib versus placebo. In at least one embodiment, the risk of 6-month CDP is reduced about 31% for tolebrutinib versus placebo. In at least one embodiment, the risk of 3-month CDP is reduced about 24% for tolebrutinib versus placebo.

In some embodiments, provided herein is a method of reducing the total number of new and/or enlarging T2-hyperintense lesions as detected by MRI, in a patient with non-relapsing secondary progressive multiple sclerosis (nrSPMS) in need thereof, comprising administering to the patient in need thereof a therapeutically effective amount of tolebrutinib or pharmaceutically acceptable salt thereof. In some embodiments, reducing comprises reducing the annualized rate of new and/or enlarging T2-hyperintense lesions. In some embodiments, the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months. In some embodiments, the annualized rate is reduced relative to placebo. In some embodiments, the annualized rate is reduced relative to no treatment. In some embodiments, the annualized rate is reduced about 5%, 10%, 15%, 20%, 25%, 30%, 35%, 40%, 45%, or 50% for tolebrutinib versus placebo. In at least one embodiment, the annualized rate is reduced about 38% for tolebrutinib versus placebo.

In some embodiments, provided herein is a method of reducing the risk of sustained 20% increase in the timed 25-foot walk (T25-FW) test confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof. In some embodiments, the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months. In some embodiments, the risk reduction is relative to placebo. In some embodiments, the risk reduction is relative to no treatment. In some embodiments, the risk is reduced about 5%, 10%, 15%, 20%, 25%, 30%, 35%, 40%, 45%, or 50% for tolebrutinib versus placebo. In at least one embodiment, the risk of sustained 20% increase in the T25-FW test confirmed over 3 months is reduced about 23% for tolebrutinib versus placebo.

In some embodiments, provided herein is a method of reducing the risk of sustained 20% increase in the 9-hole peg test (9-HPT) confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof. In some embodiments, the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months. In some embodiments, the risk reduction is relative to placebo. In some embodiments, the risk reduction is relative to no treatment. In some embodiments, the risk is reduced about 5%, 10%, 15%, 20%, 25%, 30%, 35%, 40%, 45%, or 50% for tolebrutinib versus placebo.

In some embodiments, provided herein is a method of improving the chance of confirmed disability improvement (CDI) in a patient with non-relapsing secondary progressive multiple sclerosis (nrSPMS) in need thereof, comprising administering to the patient in need thereof a therapeutically effective amount of tolebrutinib or pharmaceutically acceptable salt thereof. In some embodiments, the CDI is measured over at least three months, such as at least six months. In some embodiments, the confirmed disability improvement (CDI) comprises decreasing at least 1.0 point from a baseline expanded disability status scale score (EDSS). In some embodiments, the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months. In some embodiments, the chance is improved relative to placebo. In some embodiments, the chance is improved relative to no treatment. In some embodiments, the chance is improved about 10%, 20%, 30%, 40%, 50%, 60%, 70%, 80%, 90%, or 100% for tolebrutinib versus placebo. In at least one embodiment, the chance of 6-month CDI is improved about 88% for tolebrutinib versus placebo.

In some embodiments, provided herein is a method of improving time to onset of confirmed disability progression (CDP) in a patient with non-relapsing secondary progressive multiple sclerosis (nrSPMS) in need thereof, comprising administering to the patient in need thereof a therapeutically effective amount of tolebrutinib or pharmaceutically acceptable salt thereof. In some embodiments, the CDP is measured over at least three months, such as at least six months. In some embodiments, the confirmed disability progression (CDP) comprises: (a) increasing at least 1.0 point from a baseline expanded disability status scale score (EDSS) when the baseline score is less than or equal to 5.0; or (b) increasing at least 0.5 points when the baseline EDSS score is greater than 5.0. In some embodiments, the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months. In some embodiments, the improvement is relative to placebo. In some embodiments, the improvement is relative to no treatment. In some embodiments, the improvement is a delay in time of onset of about 5%, 10%, 15%, 20%, 25%, 30%, 35%, 40%, 45%, or 50% for tolebrutinib versus placebo. In at least one embodiment, the improvement is a delay in time of onset of 6-month CDP of about 31% for tolebrutinib versus placebo. In at least one embodiment, the improvement is a delay in time of onset of 3-month CDP of about 24% for tolebrutinib versus placebo.

In some embodiments, provided herein is a method of improving time to onset of sustained 20% increase in the timed 25-foot walk (T25-FW) test confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof. In some embodiments, the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months. In some embodiments, the improvement is relative to placebo. In some embodiments, the improvement is relative to no treatment. In some embodiments, the improvement is a delay in time of onset of about 5%, 10%, 15%, 20%, 25%, 30%, 35%, 40%, 45%, or 50% for tolebrutinib versus placebo. In at least one embodiment, the improvement is a delay in time of onset of sustained 20% increase in the T25-FW test confirmed over at least 3 months of about 23% for tolebrutinib versus placebo.

In some embodiments, provided herein is a method of improving time to onset of sustained 20% increase in the 9-hole peg test (9-HPT) confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof. In some embodiments, the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months. In some embodiments, the improvement is relative to placebo. In some embodiments, the improvement is relative to no treatment. In some embodiments, the improvement is a delay in time of onset of about 5%, 10%, 15%, 20%, 25%, 30%, 35%, 40%, 45%, or 50% for tolebrutinib versus placebo.

In some embodiments, provided herein is a method of improving time to onset of confirmed disability improvement (CDI) in a patient with non-relapsing secondary progressive multiple sclerosis (nrSPMS) in need thereof, comprising administering to the patient in need thereof a therapeutically effective amount of tolebrutinib or pharmaceutically acceptable salt thereof. In some embodiments, the CDI is measured over at least three months, such as at least six months. In some embodiments, the confirmed disability improvement (CDI) comprises decreasing at least 1.0 point from a baseline expanded disability status scale score (EDSS). In some embodiments, the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months. In some embodiments, the improvement is relative to placebo. In some embodiments, the improvement is relative to no treatment. In some embodiments, the improvement is a shortening of time of onset of about 10%, 20%, 30%, 40%, 50%, 60%, 70%, 80%, 90%, or 100% for tolebrutinib versus placebo. In at least one embodiment, the improvement is a shortening of time of onset of 6-month CDP of about 88% for tolebrutinib versus placebo.

In some embodiments, provided herein is tolebrutinib or a pharmaceutically acceptable salt thereof for use in treating a patient with non-relapsing secondary progressive multiple sclerosis (nrSPMS) in certain methods described herein. In some embodiments, treating a patient with nrSPMS comprises at least one of the following: reducing the risk of confirmed disability progression (CDP); reducing the total number of new and/or enlarging T2-hyperintense lesions as detected by MRI; reducing the risk of sustained 20% increase in the timed 25-foot walk (T25-FW) test confirmed over at least 3 months; improving the chance of confirmed disability improvement (CDI); improving time to onset of confirmed disability progression (CDP); improving time to onset of sustained 20% increase in the timed 25-foot walk (T25-FW) test confirmed over at least 3 months; improving time to onset of sustained 20% increase in the 9-hole peg test (9-HPT) confirmed over at least 3 months; and improving time to onset of confirmed disability improvement (CDI).

In some embodiments, provided herein is tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof reduces the risk of confirmed disability progression (CDP) in certain methods described herein.

In some embodiments, provided herein is tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof reduces the total number of new and/or enlarging T2-hyperintense lesions as detected by MRI in certain methods described herein.

In some embodiments, provided herein is tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof reduces the risk of sustained 20% increase in the timed 25-foot walk (T25-FW) test confirmed over at least 3 months in certain methods described herein.

In some embodiments, provided herein is tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof reduces the risk of sustained 20% increase in the 9-hole peg test (9-HPT) confirmed over at least 3 months in certain methods described herein.

In some embodiments, provided herein is tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves the chance of confirmed disability improvement (CDI) in certain methods described herein.

In some embodiments, provided herein is tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves time to onset of confirmed disability progression (CDP) in certain methods described herein.

In some embodiments, provided herein is tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves time to onset of sustained 20% increase in the timed 25-foot walk (T25-FW) test confirmed over at least 3 months in certain methods described herein.

In some embodiments, provided herein is tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves time to onset of sustained 20% increase in the 9-hole peg test (9-HPT) confirmed over at least 3 months in certain methods described herein.

In some embodiments, provided herein is tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves time to onset of confirmed disability improvement (CDI) in certain methods described herein.

In some embodiments, provided herein is the use of tolebrutinib or a pharmaceutically acceptable salt thereof in the manufacture of a medicament for treating a patient with nrSPMS in certain methods described herein. In some embodiments, treating a patient with nrSPMS comprises at least one of the following: reducing the risk of confirmed disability progression (CDP); reducing the total number of new and/or enlarging T2-hyperintense lesions as detected by MRI; reducing the risk of sustained 20% increase in the timed 25-foot walk (T25-FW) test confirmed over at least 3 months; improving the chance of confirmed disability improvement (CDI); improving time to onset of confirmed disability progression (CDP); improving time to onset of sustained 20% increase in the timed 25-foot walk (T25-FW) test confirmed over at least 3 months; improving time to onset of sustained 20% increase in the 9-hole peg test (9-HPT) confirmed over at least 3 months; and improving time to onset of confirmed disability improvement (CDI).

In some embodiments, provided herein is the use of tolebrutinib or a pharmaceutically acceptable salt thereof in the manufacture of a medicament for treating a patient with nrSPMS, wherein tolebrutinib or a pharmaceutically acceptable salt thereof reduces the risk of confirmed disability progression (CDP), in certain methods described herein.

In some embodiments, provided herein is the use of tolebrutinib or a pharmaceutically acceptable salt thereof in the manufacture of a medicament for treating a patient with nrSPMS, wherein tolebrutinib or a pharmaceutically acceptable salt thereof reduces the total number of new and/or enlarging T2-hyperintense lesions as detected by MRI, in certain methods described herein.

In some embodiments, provided herein is the use of tolebrutinib or a pharmaceutically acceptable salt thereof in the manufacture of a medicament for treating a patient with nrSPMS, wherein tolebrutinib or a pharmaceutically acceptable salt thereof reduces the risk of sustained 20% increase in the timed 25-foot walk (T25-FW) test confirmed over at least 3 months, in certain methods described herein.

In some embodiments, provided herein is the use of tolebrutinib or a pharmaceutically acceptable salt thereof in the manufacture of a medicament for treating a patient with nrSPMS, wherein tolebrutinib or a pharmaceutically acceptable salt thereof reduces the risk of sustained 20% increase in the 9-hole peg test (9-HPT) confirmed over at least 3 months, in certain methods described herein.

In some embodiments, provided herein is the use of tolebrutinib or a pharmaceutically acceptable salt thereof in the manufacture of a medicament for treating a patient with nrSPMS, wherein tolebrutinib or a pharmaceutically acceptable salt thereof improves the chance of confirmed disability improvement (CDI), in certain methods described herein.

In some embodiments, provided herein is the use of tolebrutinib or a pharmaceutically acceptable salt thereof in the manufacture of a medicament for treating a patient with nrSPMS, wherein tolebrutinib or a pharmaceutically acceptable salt thereof improves time to onset of confirmed disability progression (CDP), in certain methods described herein.

In some embodiments, provided herein is the use of tolebrutinib or a pharmaceutically acceptable salt thereof in the manufacture of a medicament for treating a patient with nrSPMS, wherein tolebrutinib or a pharmaceutically acceptable salt thereof improves time to onset of sustained 20% increase in the timed 25-foot walk (T25-FW) test confirmed over at least 3 months, in certain methods described herein.

In some embodiments, provided herein is the use of tolebrutinib or a pharmaceutically acceptable salt thereof in the manufacture of a medicament for treating a patient with nrSPMS, wherein tolebrutinib or a pharmaceutically acceptable salt thereof improves time to onset of sustained 20% increase in the 9-hole peg test (9-HPT) confirmed over at least 3 months, in certain methods described herein.

In some embodiments, provided herein is the use of tolebrutinib or a pharmaceutically acceptable salt thereof in the manufacture of a medicament for treating a patient with nrSPMS, wherein tolebrutinib or a pharmaceutically acceptable salt thereof improves time to onset of confirmed disability improvement (CDI), in certain methods described herein.

In some embodiments, provided herein is a method of reducing the risk of confirmed disability worsening (CDW) in a patient with a relapsing form of multiple sclerosis (RMS) in need thereof, comprising administering to the patient in need thereof a therapeutically effective amount of tolebrutinib or pharmaceutically acceptable salt thereof. In some embodiments, the CDW is measured over at least three months, such as at least six months. In some embodiments, CDW comprises: (a) increasing at least 1.5 point from a baseline expanded disability status scale score (EDSS) when the baseline score is 0; (b) increasing at least 1.0 point from the baseline EDSS when the baseline score is greater than or equal to 0.5 and less than or equal to 5.5; or (c) increasing at least 0.5 points when the baseline EDSS score is greater than 5.5. In some embodiments, the patient has an RMS diagnosis with an EDSS less than or equal to 5.5 prior to initial administration of tolebrutinib, and one or more of the following: (a) one or more documented relapses in the previous year; (b) two or more documented relapses in the previous 2 years; and (c) one or more Gd-enhancing lesions on an MRI scan in the previous year. In some embodiments, the risk reduction is relative to treatment with teriflunomide. In some embodiments, the risk is reduced about 5%, 10%, 15%, 20%, 25%, 30%, 35%, 40%, 45%, or 50% for tolebrutinib versus teriflunomide. In at least one embodiment, the risk of 6-month CDW is reduced about 29% for tolebrutinib versus teriflunomide. In at least one embodiment, the risk of 3-month CDW is reduced about 27% for tolebrutinib versus teriflunomide.

In some embodiments, provided herein is a method of improving time to onset of confirmed disability worsening (CDW) in a patient with a relapsing form of multiple sclerosis (RMS) in need thereof, comprising administering to the patient in need thereof a therapeutically effective amount of tolebrutinib or pharmaceutically acceptable salt thereof. In some embodiments, the CDW is measured over at least three months, such as at least six months. In some embodiments, CDW comprises: (a) increasing at least 1.5 point from a baseline expanded disability status scale score (EDSS) when the baseline score is 0; (b) increasing at least 1.0 point from the baseline EDSS when the baseline score is greater than or equal to 0.5 and less than or equal to 5.5; or (c) increasing at least 0.5 points when the baseline EDSS score is greater than 5.5. In some embodiments, the patient has an RMS diagnosis with an EDSS less than or equal to 5.5 at the first visit, and one or more of the following: (a) one or more documented relapses in the previous year; (b) two or more documented relapses in the previous 2 years; and (c) one or more Gd-enhancing lesions on an MRI scan in the previous year. In some embodiments, the improvement is relative to treatment with teriflunomide. In some embodiments, the improvement is a delay in time to onset of about 5%, 10%, 15%, 20%, 25%, 30%, 35%, 40%, 45%, or 50% for tolebrutinib versus teriflunomide. In at least one embodiment, the improvement is a delay in time of onset of 6-month CDW of about 29% for tolebrutinib versus teriflunomide. In at least one embodiment, improvement is a delay in time of onset of 3-month CDW of about 27% for tolebrutinib versus teriflunomide.

In some embodiments, provided herein is a method of improving percent change in brain volume loss (BVL) as detected by brain MRI in a patient with relapsing forms of multiple sclerosis (RMS), comprising administering to the patient in need thereof a therapeutically effective amount of tolebrutinib or pharmaceutically acceptable salt thereof. In some embodiments, the patient has an RMS diagnosis with an EDSS less than or equal to 5.5 at the first visit, and one or more of the following: (a) one or more documented relapses in the previous year; (b) two or more documented relapses in the previous 2 years; and (c) one or more Gd-enhancing lesions on an MRI scan in the previous year. In some embodiments, the percent change BVL improvement is relative to treatment with teriflunomide. In some embodiments, the percent change BVL decreases by about 5%, 10%, 15%, 20%, 25%, 30%, 35%, 40%, 45%, or 50% for tolebrutinib versus teriflunomide. In at least one embodiment, the percent change BVL decreases by about 20% for tolebrutinib versus teriflunomide.

In some embodiments, provided herein is tolebrutinib or a pharmaceutically acceptable salt thereof for use in treating a patient with a relapsing form of multiple sclerosis (RMS) in certain methods described herein. In some embodiments, treating a patient with RMS comprises at least one of: reducing the risk of confirmed disability worsening (CDW); improving time to onset of confirmed disability worsening (CDW); and improving percent change in brain volume loss (BVL) as detected by brain MRI.

In some embodiments, provided herein is tolebrutinib or a pharmaceutically acceptable salt thereof for use in treating a patient with a relapsing form of multiple sclerosis (RMS), wherein tolebrutinib or a pharmaceutically acceptable salt thereof reduces the risk of confirmed disability worsening (CDW) in certain methods described herein.

In some embodiments, provided herein is tolebrutinib or a pharmaceutically acceptable salt thereof for use in treating a patient with a relapsing form of multiple sclerosis (RMS), wherein tolebrutinib or a pharmaceutically acceptable salt thereof improves time to onset of confirmed disability worsening (CDW) in certain methods described herein.

In some embodiments, provided herein is tolebrutinib or a pharmaceutically acceptable salt thereof for use in treating a patient with a relapsing form of multiple sclerosis (RMS), wherein tolebrutinib or a pharmaceutically acceptable salt thereof improves percent change in brain volume loss (BVL) as detected by brain MRI in certain methods described herein.

In some embodiments, provided herein is the use of tolebrutinib or a pharmaceutically acceptable salt thereof in the manufacture of a medicament for use in treating a patient with RMS in certain methods described herein. In some embodiments, treating a patient with RMS comprises at least one of: reducing the risk of confirmed disability worsening (CDW); improving time to onset of confirmed disability worsening (CDW); and improving percent change in brain volume loss (BVL) as detected by brain MRI.

In some embodiments, provided herein is the use of tolebrutinib or a pharmaceutically acceptable salt thereof in the manufacture of a medicament for use in reducing the risk of confirmed disability worsening (CDW), in certain methods described herein.

In some embodiments, the use of tolebrutinib or a pharmaceutically acceptable salt thereof in the manufacture of a medicament for use in treating a patient with RMS, wherein tolebrutinib or a pharmaceutically acceptable salt thereof improves time to onset of confirmed disability worsening (CDW), in certain methods described herein.

In some embodiments, the use of tolebrutinib or a pharmaceutically acceptable salt thereof in the manufacture of a medicament for use in treating a patient with RMS, wherein tolebrutinib or a pharmaceutically acceptable salt thereof improves percent change in brain volume loss (BVL) as detected by brain MRI, in certain methods described herein.

In some embodiments, provided herein is a method of treating a patient with primary progressive multiple sclerosis (PPMS) in need thereof, comprising administering to the patient in need thereof a therapeutically effective amount of tolebrutinib or pharmaceutically acceptable salt thereof, wherein at least one outcome chosen from the following occurs: (a) the risk of composite confirmed disability progression (cCDP) is reduced, wherein the cCDP comprises progression in at least one of confirmed disability progression (CDP), sustained 20% increase in the timed 25-foot walk (T25-FW) test), and sustained 20% increase in the 9-hole peg test (9-HPT); (b) the risk of confirmed disability progression (CDP) is reduced; (c) the total number of new and/or enlarging T2-hyperintense lesions as detected by MRI, defined as the sum of the individual number of new and/or enlarging T2 lesions, are reduced; (d) the risk of sustained 20% increase in the timed 25-foot walk (T25-FW) test is reduced; (e) the risk of sustained 20% increase in the 9-hole peg test (9-HPT) is reduced; (f) the chance of confirmed disability improvement (CDI) is improved; and (g) the percent change in brain volume loss (BVL) as detected by brain MRI is improved. In some embodiments, the CDP, cCDP, sustained 20% increase in the timed 25-foot walk (T25-FW) test, or sustained 20% increase in the 9-hole peg test (9-HPT) is measured over at least three months, such as at least six months. In some embodiments, one or more of the reduction in risk of CDP or cCDP, the reduction in total number of new and/or enlarging T2-hyperintense lesions, the reduction in risk of sustained 20% increase in T25-FW test, the reduction in risk of sustained 20% increase in the 9-hole peg test (9-HPT), the improvement of chance of CDI, or the improvement percent change in BVL is relative to placebo. In some embodiments, one or more of the reduction in risk of CDP or cCDP, the reduction in total number of new and/or enlarging T2-hyperintense lesions, the reduction in risk of sustained 20% increase in T25-FW test, the reduction in risk of sustained 20% increase in the 9-hole peg test (9-HPT), the improvement of chance of CDI, or the improvement percent change in BVL is relative to no treatment. In some embodiments, the risk of CDP or cCDP is reduced about 5%, 10%, 15%, 20%, 25%, 30%, 35%, 40%, 45%, or 50% for tolebrutinib versus placebo. In some embodiments, the risk of CDP or cCDP is reduced about 55%, 60%, 65%, 70%, 75%, 80%, 85%, 90%, 95% for tolebrutinib versus placebo. In some embodiments, the annualized rate of or new and/or enlarging T2-hyperintense lesions is reduced about 5%, 10%, 15%, 20%, 25%, 30%, 35%, 40%, 45%, or 50% for tolebrutinib versus placebo. In some embodiments, the annualized rate is reduced about 55%, 60%, 55%, 60%, 65%, 70%, 75%, 80%, 85%, 90%, or 95% for tolebrutinib versus placebo. In some embodiments, the risk of sustained 20% increase in T25-FW test is reduced about 5%, 10%, 15%, 20%, 25%, 30%, 35%, 40%, 45%, or 50% for tolebrutinib versus placebo. In some embodiments, the risk of sustained 20% increase in T25-FW test is reduced about 55%, 60%, 55%, 60%, 65%, 70%, 75%, 80%, 85%, 90%, or 95% for tolebrutinib versus placebo. In some embodiments, the risk of sustained 20% increase in 9-HPT is reduced about 5%, 10%, 15%, 20%, 25%, 30%, 35%, 40%, 45%, or 50% for tolebrutinib versus placebo. In some embodiments, the risk of sustained 20% increase in 9-HPT is reduced about 55%, 60%, 55%, 60%, 65%, 70%, 75%, 80%, 85%, 90%, or 95% for tolebrutinib versus placebo. In some embodiments, the chance of CDI is improved about 10%, 20%, 30%, 40%, 50%, 60%, 70%, 80%, 90%, or 100% for tolebrutinib versus placebo. In some embodiments, the chance of CDI is improved about 110%, 120%, 130%, 140%, 150%, ¹60%, 170%, 180%, 190%, or 200% for tolebrutinib versus placebo. In some embodiments, the percent change BVL decreases by about 5%, 10%, 15%, 20%, 25%, 30%, 35%, 40%, 45%, or 50% for tolebrutinib versus placebo. In some embodiments, the percent change BVL decreases by about 55%, 60%, 55%, 60%, 65%, 70%, 75%, 80%, 85%, 90%, or 95% for tolebrutinib versus placebo.

In some embodiments, provided herein is a method of treating a patient with primary progressive multiple sclerosis (PPMS) in need thereof, comprising administering to the patient in need thereof a therapeutically effective amount of tolebrutinib or pharmaceutically acceptable salt thereof, wherein the risk of confirmed disability progression (CDP) or composite confirmed disability progression (cCDP) is reduced, wherein the cCDP comprises progression in at least one of: (a) CDP; (b) sustained 20% increase in the timed 25-foot walk (T25-FW) test; and (c) sustained 20% increase in the 9-hole peg test (9-HPT). In some embodiments, the CDP or cCDP is measured over at least three months, such as at least six months. In some embodiments, the CDP comprises: (a) increasing at least 1.0 point from a baseline expanded disability status scale score (EDSS) when the baseline score is less than or equal to 5.5; or (b) increasing at least 0.5 points when the baseline EDSS score is greater than 5.5. In some embodiments, the patient with PPMS has: (a) a PPMS diagnosis with an expanded disability status scale score (EDSS) between 2.0 and 6.5; and (b) a cerebrospinal fluid (CSF) analysis that is positive for isoelectric focusing evidence of oligoclonal bands or elevated IgG index. In some embodiments, the risk of CDP is reduced. In some embodiments, the risk of cCDP is reduced. In some embodiments, the risk of CDP or cCDP is reduced relative to placebo. In some embodiments, the risk of CDP or cCDP is reduced relative to no treatment. In some embodiments, the risk is reduced about 5%, 10%, 15%, 20%, 25%, 30%, 35%, 40%, 45%, or 50% for tolebrutinib versus placebo. In some embodiments, the risk of CDP or cCDP is reduced about 55%, 60%, 55%, 60%, 65%, 70%, 75%, 80%, 85%, 90%, or 95% for tolebrutinib versus placebo.

In some embodiments, provided herein is a method of reducing the risk of confirmed disability progression (CDP) or composite confirmed disability progression (cCDP) in a patient with primary progressive multiple sclerosis (PPMS) in need thereof, comprising administering to the patient in need thereof a therapeutically effective amount of tolebrutinib or pharmaceutically acceptable salt thereof, wherein the cCDP comprises progression in at least one of: (a) CDP; (b) sustained 20% increase in the timed 25-foot walk (T25-FW) test; and (c) sustained 20% increase in the 9-hole peg test (9-HPT). In some embodiments, the CDP or cCDP is measured over at least three months, such as at least six months. In some embodiments, confirmed disability progression (CDP) comprises: (a) increasing at least 1.0 point from a baseline expanded disability status scale score (EDSS) when the baseline score is less than or equal to 5.5; or (b) increasing at least 0.5 points when the baseline EDSS score is greater than 5.5. In some embodiments, the patient with PPMS has: (a) a PPMS diagnosis with an expanded disability status scale score (EDSS) between 2.0 and 6.5; and (b) a cerebrospinal fluid (CSF) analysis that is positive for isoelectric focusing evidence of oligoclonal bands or elevated IgG index. In some embodiments, the risk reduction is relative to placebo. In some embodiments, the risk reduction is relative to no treatment. In some embodiments, the risk of CDP is reduced. In some embodiments, the risk of cCDP is reduced. In some embodiments, the risk of CDP or cCDP is reduced about 5%, 10%, 15%, 20%, 25%, 30%, 35%, 40%, 45%, or 50% for tolebrutinib versus placebo. In some embodiments, the risk of CDP or cCDP is reduced about 55%, 60%, 55%, 60%, 65%, 70%, 75%, 80%, 85%, 90%, or 95% for tolebrutinib versus placebo.

In some embodiments, provided herein is a method of improving time to onset of confirmed disability progression (CDP) or composite confirmed disability progression (cCDP) in a patient with primary progressive multiple sclerosis (PPMS) in need thereof, comprising administering to the patient in need thereof a therapeutically effective amount of tolebrutinib or pharmaceutically acceptable salt thereof, wherein the cCDP comprises progression in at least one of: (a) CDP; (b) sustained 20% increase in the timed 25-foot walk (T25-FW) test; and (c) sustained 20% increase in the 9-hole peg test (9-HPT). In some embodiments, the CDP or cCDP is measured over at least three months, such as at least six months. In some embodiments, the CDP comprises: (a) increasing at least 1.0 point from a baseline expanded disability status scale score (EDSS) when the baseline score is less than or equal to 5.5; or (b) increasing at least 0.5 points when the baseline EDSS score is greater than 5.5. In some embodiments, the patient with PPMS has: (a) a PPMS diagnosis with an expanded disability status scale score (EDSS) between 2.0 and 6.5; and (b) a cerebrospinal fluid (CSF) analysis that is positive for isoelectric focusing evidence of oligoclonal bands or elevated IgG index. In some embodiments, the improvement is relative to placebo. In some embodiments, the improvement is relative to no treatment. In some embodiments, the time to onset of CDP is improved. In some embodiments, the time to onset of cCDP is improved. In some embodiments, the improvement is a delay in time to onset of CDP or cCDP about 5%, 10%, 15%, 20%, 25%, 30%, 35%, 40%, 45%, or 50% for tolebrutinib versus placebo. In some embodiments, the improvement is a delay in time to onset of CDP or cCDP of about 55%, 60%, 55%, 60%, 65%, 70%, 75%, 80%, 85%, 90%, or 95% for tolebrutinib versus placebo.

In some embodiments, provided herein is a method of reducing the total number of new and/or enlarging T2-hyperintense lesions as detected by MRI, in a patient with primary progressive multiple sclerosis (PPMS) in need thereof, comprising administering to the patient in need thereof a therapeutically effective amount of tolebrutinib or pharmaceutically acceptable salt thereof. In some embodiments, reducing comprises reducing the annualized rate of new and/or enlarging T2-hyperintense lesions. In some embodiments, the patient with PPMS has: (a) a PPMS diagnosis with an expanded disability status scale score (EDSS) between 2.0 and 6.5; and (b) a cerebrospinal fluid (CSF) analysis that is positive for isoelectric focusing evidence of oligoclonal bands or elevated IgG index. In some embodiments, the annualized rate is reduced relative to placebo. In some embodiments, the annualized rate is reduced relative to no treatment. In some embodiments, the annualized rate is reduced about 5%, 10%, 15%, 20%, 25%, 30%, 35%, 40%, 45%, or 50% for tolebrutinib versus placebo. In some embodiments, the annualized rate is reduced about 55%, 60%, 55%, 60%, 65%, 70%, 75%, 80%, 85%, 90%, or 95% for tolebrutinib versus placebo.

In some embodiments, provided herein is a method of reducing the risk of sustained 20% increase in the timed 25-foot walk (T25-FW) test, in a patient with primary progressive multiple sclerosis (PPMS) in need thereof, comprising administering to the patient in need thereof a therapeutically effective amount of tolebrutinib or pharmaceutically acceptable salt thereof. In some embodiments, the sustained 20% increase in the T25-FW test is measured over at least three months, such as at least six months In some embodiments, the patient with PPMS has: (a) a PPMS diagnosis with an expanded disability status scale score (EDSS) between 2.0 and 6.5; and (b) a cerebrospinal fluid (CSF) analysis that is positive for isoelectric focusing evidence of oligoclonal bands or elevated IgG index. In some embodiments, the risk reduction is relative to placebo. In some embodiments, the risk reduction is relative to no treatment. In some embodiments, the risk is reduced about 5%, 10%, 15%, 20%, 25%, 30%, 35%, 40%, 45%, or 50% for tolebrutinib versus placebo. In some embodiments, the risk is reduced about 55%, 60%, 55%, 60%, 65%, 70%, 75%, 80%, 85%, 90%, or 95% for tolebrutinib versus placebo.

In some embodiments, provided herein is a method of reducing the risk of sustained 20% increase in the 9-hole peg test (9-HPT), in a patient with primary progressive multiple sclerosis (PPMS) in need thereof, comprising administering to the patient in need thereof a therapeutically effective amount of tolebrutinib or pharmaceutically acceptable salt thereof. In some embodiments, the sustained 20% increase in the 9-HPT is measured over at least three months, such as at least six months In some embodiments, the patient with PPMS has: (a) a PPMS diagnosis with an expanded disability status scale score (EDSS) between 2.0 and 6.5; and (b) a cerebrospinal fluid (CSF) analysis that is positive for isoelectric focusing evidence of oligoclonal bands or elevated IgG index. In some embodiments, the risk reduction is relative to placebo. In some embodiments, the risk reduction is relative to no treatment. In some embodiments, the risk is reduced about 5%, 10%, 15%, 20%, 25%, 30%, 35%, 40%, 45%, or 50% for tolebrutinib versus placebo. In some embodiments, the risk is reduced about 55%, 60%, 55%, 60%, 65%, 70%, 75%, 80%, 85%, 90%, or 95% for tolebrutinib versus placebo.

In some embodiments, provided herein is a method of improving the chance of confirmed disability improvement (CDI) in a patient with primary progressive multiple sclerosis (PPMS) in need thereof, comprising administering to the patient in need thereof a therapeutically effective amount of tolebrutinib or pharmaceutically acceptable salt thereof. In some embodiments, the CDI is measured over at least three months, such as at least six months. In some embodiments, the confirmed disability improvement (CDI) comprises decreasing at least 1.0 point from a baseline expanded disability status scale score (EDSS). In some embodiments, the patient with PPMS has: (a) a PPMS diagnosis with an expanded disability status scale score (EDSS) between 2.0 and 6.5; and (b) a cerebrospinal fluid (CSF) analysis that is positive for isoelectric focusing evidence of oligoclonal bands or elevated IgG index. In some embodiments, the chance is improved relative to placebo. In some embodiments, the chance is improved relative to no treatment. In some embodiments, the chance is improved about 10%, 20%, 30%, 40%, 50%, 60%, 70%, 80%, 90%, or 100% for tolebrutinib versus placebo. In some embodiments, the chance of CDI is improved about 110%, 120%, ¹30%, 140%, 150%, 160%, 170%, 180%, 190%, or 200% for tolebrutinib versus placebo.

In some embodiments, provided herein is a method of improving time to onset of sustained 20% increase in the timed 25-foot walk (T25-FW) test, in a patient with primary progressive multiple sclerosis (PPMS) in need thereof, comprising administering to the patient in need thereof a therapeutically effective amount of tolebrutinib or pharmaceutically acceptable salt thereof. In some embodiments, the sustained 20% increase in the T25-FW test is measured over at least three months, such as at least six months In some embodiments, the patient with PPMS has: (a) a PPMS diagnosis with an expanded disability status scale score (EDSS) between 2.0 and 6.5; and (b) a cerebrospinal fluid (CSF) analysis that is positive for isoelectric focusing evidence of oligoclonal bands or elevated IgG index. In some embodiments, the improvement is relative to placebo. In some embodiments, the improvement is a delay in time of onset of about 5%, 10%, 15%, 20%, 25%, 30%, 35%, 40%, 45%, or 50% for tolebrutinib versus placebo. In some embodiments, the improvement is relative to placebo. In some embodiments, the improvement is relative to no treatment. In some embodiments, the improvement is a delay in time of onset of about 10%, 20%, 30%, 40%, 50%, 60%, 70%, 80%, 90%, or 100% for tolebrutinib versus placebo.

In some embodiments, provided herein is a method of improving time to onset of sustained 20% increase in the 9-hole peg test (9-HPT), in a patient with primary progressive multiple sclerosis (PPMS) in need thereof, comprising administering to the patient in need thereof a therapeutically effective amount of tolebrutinib or pharmaceutically acceptable salt thereof. In some embodiments, the sustained 20% increase in the 9-HPT is measured over at least three months, such as at least six months In some embodiments, the patient with PPMS has: (a) a PPMS diagnosis with an expanded disability status scale score (EDSS) between 2.0 and 6.5; and (b) a cerebrospinal fluid (CSF) analysis that is positive for isoelectric focusing evidence of oligoclonal bands or elevated IgG index. In some embodiments, the improvement is relative to placebo. In some embodiments, the improvement is relative to no treatment. In some embodiments, the improvement is a delay in time of onset of about 5%, 10%, 15%, 20%, 25%, 30%, 35%, 40%, 45%, or 50% for tolebrutinib versus placebo. In some embodiments, the improvement is a delay in time of onset of about 10%, 20%, 30%, 40%, 50%, 60%, 70%, 80%, 90%, or 100% for tolebrutinib versus placebo.

In some embodiments, provided herein is a method of improving time to onset of confirmed disability improvement (CDI) in a patient with primary progressive multiple sclerosis (PPMS) in need thereof, comprising administering to the patient in need thereof a therapeutically effective amount of tolebrutinib or pharmaceutically acceptable salt thereof. In some embodiments, the CDI is measured over at least three months, such as at least six months. In some embodiments, the confirmed disability improvement (CDI) comprises decreasing at least 1.0 point from a baseline expanded disability status scale score (EDSS). In some embodiments, the patient with PPMS has: (a) a PPMS diagnosis with an expanded disability status scale score (EDSS) between 2.0 and 6.5; and (b) a cerebrospinal fluid (CSF) analysis that is positive for isoelectric focusing evidence of oligoclonal bands or elevated IgG index. In some embodiments, the improvement is relative to placebo. In some embodiments, the improvement is relative to no treatment. In some embodiments, the improvement is a shortening of time of onset of about 10%, 20%, 30%, 40%, 50%, 60%, 70%, 80%, 90%, or 100% for tolebrutinib versus placebo.

In some embodiments, provided herein is tolebrutinib or a pharmaceutically acceptable salt thereof for use in treating a patient with primary progressive multiple sclerosis (PPMS) in certain methods described herein. In some embodiments, treating a patient with PPMS comprises at least one of: reducing the risk of confirmed disability progression (CDP) or composite confirmed disability progression (cCDP) and improving time to onset of confirmed disability progression (CDP) or composite confirmed disability progression (cCDP).

In some embodiments, provided herein is tolebrutinib or a pharmaceutically acceptable salt thereof for use in treating a patient with primary progressive multiple sclerosis (PPMS), wherein tolebrutinib or a pharmaceutically acceptable salt reduces the risk of confirmed disability progression (CDP) or composite confirmed disability progression (cCDP) in certain methods described herein.

In some embodiments, provided herein is tolebrutinib or a pharmaceutically acceptable salt thereof for use in treating a patient with primary progressive multiple sclerosis (PPMS), wherein tolebrutinib or a pharmaceutically acceptable salt improves time to onset of confirmed disability progression (CDP) or composite confirmed disability progression (cCDP) in certain methods described herein.

In some embodiments, provided herein is the use of tolebrutinib or a pharmaceutically acceptable salt thereof in the manufacture of a medicament for use in treating a patient with PPMS in certain methods described herein. In some embodiments, treating a patient with PPMS comprises at least one of: reducing the risk of confirmed disability progression (CDP) or composite confirmed disability progression (cCDP) and improving time to onset of confirmed disability progression (CDP) or composite confirmed disability progression (cCDP).

In some embodiments, provided herein is the use of tolebrutinib or a pharmaceutically acceptable salt thereof in the manufacture of a medicament for use in treating a patient with PPMS, wherein tolebrutinib or a pharmaceutically acceptable salt reduces the risk of confirmed disability progression (CDP) or composite confirmed disability progression (cCDP), in certain methods described herein.

In some embodiments, provided herein is the use of tolebrutinib or a pharmaceutically acceptable salt thereof in the manufacture of a medicament for use in treating a patient with PPMS, wherein tolebrutinib or a pharmaceutically acceptable salt improves time to onset of confirmed disability progression (CDP) or composite confirmed disability progression (cCDP) in a patient with primary progressive multiple sclerosis (PPMS) in certain methods described herein.

In some embodiments, provided herein is a method of treating disability accumulation in a patient with multiple sclerosis comprising administering to the patient in need thereof a therapeutically effective amount of tolebrutinib or pharmaceutically acceptable salt thereof.

In some embodiments, provided herein is a method of treating disability progression independent of relapse activity in a patient with multiple sclerosis comprising administering to the patient in need thereof a therapeutically effective amount of tolebrutinib or pharmaceutically acceptable salt thereof. In some embodiments, treating disability accumulation or disability progression independent of relapse activity comprises at least one of: (a) reducing the risk of disability accumulation in the patient in need thereof; (b) delaying disability accumulation in the patient in need thereof; or (c) improving time to onset of disability accumulation in the patient in need thereof.

In some embodiments, the reduction of risk, delay, or improvement of time to onset of disability accumulation is relative to placebo. In some embodiments, the reduction of risk, delay, or improvement of time to onset of disability accumulation is relative to no treatment. In some embodiments, the risk of disability accumulation is reduced about 5%, 10%, 15%, 20%, 25%, 30%, 35%, 40%, 45%, or 50% for tolebrutunib versus placebo. In at least one embodiment, the risk of 6-month CDP is reduced about 310% for tolebrutinib versus placebo. In at least one embodiment, the risk of 3-month CDP is reduced about 24% for tolebrutinib versus placebo. In at least one embodiment, the risk of sustained 20% increase in the T25-FW test confirmed over at least 3 months is reduced about 23% for tolebrutinib versus placebo. In some embodiments, the disability accumulation is delayed about 5%, 10%, 15%, 20%, 25%, 30%, 35%, 40%, 45%, or 50% for tolebrutunib versus placebo. In at least one embodiment, 6-month CDP is delayed by about 31% for tolebrutinib versus placebo. In at least one embodiment, 3-month CDP is delayed by about 24% for tolebrutinib versus placebo. In at least one embodiment, sustained 20% increase in the T25-FW test confirmed over at least 3 months is delayed by about 23% for tolebrutinib versus placebo. In some embodiments, the improvement in time to onset of disability accumulation is a delay of about 5%, 10%, 15%, 20%, 25%, 30%, 35%, 40%, 45%, or 50% for tolebrutunib versus placebo. In at least one embodiment, the improvement is a delay in time of onset of 6-month CDP of about 31% for tolebrutinib versus placebo. In at least one embodiment, the improvement is a delay in time of onset of 3-month CDP of about 24% for tolebrutinib versus placebo. In at least one embodiment, the improvement is a delay in time ot onset of sustained 20% increase in the T25-FW test confirmed over at least 3 months of about 23% for tolebrutinib versus placebo.

In some embodiments the In some embodiments, the reduction of risk, delay, or improvement of time to onset of disability accumulation is relative to treatment with teriflunomide. In some embodiments, the risk of disability accumulation is reduced about 5%, 10%, 15%, 20%, 25%, 30%, 35%, 40%, 45%, or 50% for tolebrutinib versus teriflunomide. In at least one embodiment, the risk of 6-month CDW is reduced about 29% for tolebrutinib versus teriflunomide. In at least one embodiment, the risk of 3-month CDW is reduced about 27% for tolebrutinib versus teriflunomide. In some embodiments, the disability accumulation is delayed about 5%, 10%, 15%, 20%, 25%, 30%, 35%, 40%, 45%, or 50% for tolebrutunib versus teriflunomide. In at least one embodiment, 6-month CDW is delayed about 29% for tolebrutinib versus teriflunomide. In at least one embodiment, 3-month CDW is delayed reduced about 27% for tolebrutinib versus teriflunomide. In some embodiments, the improvement in time to onset of disability accumulation is a delay of about 5%, 10%, 15%, 20%, 25%, 30%, 35%, 40%, 45%, or 50% for tolebrutunib versus teriflunomide. In at least one embodiment, the improvement is a delay in time of onset of 6-month CDW of about 29% for tolebrutinib versus teriflunomide. In at least one embodiment, improvement is a delay in time of onset of 3-month CDW of about 27% for tolebrutinib versus teriflunomide.

In some embodiments, the disability accumulation or disability progression independent of relapse activity is confirmed disability accumulation (CDA). In some embodiments, the confirmed disability accumulation (CDA) is confirmed disability progression (CDP). In some embodiments, the confirmed disability accumulation (CDA) is confirmed disability worsening (CDW). In some embodiments, the confirmed disability accumulation (CDA) is a composite confirmed disability accumulation (cCDA) comprising progression in one or more of: (a) CDW or CDP; (b) sustained 20% increase in the timed 25-foot walk test (T25-FW); and (c) sustained 20% increase in the 9-hole peg test (9-HPT). In some embodiments, the CDP comprises: (a) increasing at least 1.0 point from a baseline expanded disability status scale score (EDSS) when the baseline score is less than or equal to 5.0; or (b) increasing at least 0.5 points when the baseline EDSS score is greater than 5.0. In some embodiments, the CDW comprises: (a) increasing at least 1.5 point from the baseline expanded disability status scale score (EDSS) when the baseline score is 0; (b) increasing at least 1.0 point from the baseline EDSS when the baseline score is greater than or equal to 0.5 and less than or equal to 5.5; or (c) increasing at least 0.5 points when the baseline EDSS score is greater than 5.5. In some embodiments, the CDA or cCDA is measured over at least 3 months, such as at least 6 months.

In some embodiments, the patient in need thereof has secondary progressive multiple sclerosis (SPMS). In some embodiments, the SPMS is non-relapsing secondary progressive multiple sclerosis (nrSPMS). In some embodiments, the SPMS is relapsing secondary progressive multiple sclerosis (R-SPMS). In some embodiments, the patient in need thereof has a relapsing form of multiple sclerosis (RMS). In some embodiments, the patient in need thereof has primary progressive multiple sclerosis (PPMS).

In some embodiments, provided herein is tolebrutinib or a pharmaceutically acceptable salt thereof for use in treating disability accumulation in a patient with multiple sclerosis in certain methods described herein.

In some embodiments, provided herein is tolebrutinib or a pharmaceutically acceptable salt thereof for use in treating disability progression independent of relapse activity in a patient with multiple sclerosis in certain methods described herein.

In some embodiments, provided herein is the use of tolebrutinib or a pharmaceutically acceptable salt thereof for use in the manufacture of a medicament for use in treating disability accumulation in a patient with multiple sclerosis in certain methods described herein.

In some embodiments, provided herein is the use of tolebrutinib or a pharmaceutically acceptable salt thereof for use in the manufacture of a medicament for use in treating disability progression independent of relapse activity in a patient with multiple sclerosis in certain methods described herein.

In some embodiments, the patient in need thereof is an adult. In some embodiments, the patient is at least 18 years old. In some embodiments, the patient ranges in age from 18 to 90 years old, for example, 20 to 85 years old, 25 to 80 years old, 30 to 75 years old, 35 to 70 years old, 40 to 65 years old, 45 to 60 years old or 50 to 55 years old.

In some embodiments, the therapeutically effective amount of tolebrutinib or pharmaceutically acceptable salt thereof is about 5 mg to about 60 mg, measured in base form.

In some embodiments, the therapeutically effective amount of tolebrutinib or pharmaceutically acceptable salt thereof is 60 mg, measured in base form.

For certain of the methods disclosed herein, the observed benefit (e.g., treatment effect) may be greater in patients in a first subgroup compared to patients in one or more second subgroups of patients or to patients not in the first subgroup.

In certain methods disclosed herein, wherein tolebrutinib is administered to a patient in need thereof having non-relapsing secondary progressive multiple sclerosis (nrSPMS), the treatment effect (e.g., improved time to onset of 6-month confirmed disability progression (CDP)) is greater in a first subgroup of patients than in patients not in the first subgroup.

In some embodiments, the patient in need thereof has one or more Gd-enhancing T1-hyperintense lesions as detected by MRI before administration of tolebrutinib. In some embodiments, the time to onset of 6-month CDP is improved about 65% for the patient having one or more Gd-enhancing T1-hyperintense lesions compared to about 22% for a patient not having a Gd-enhancing T1-hyperintense lesion before administration of tolebrutinib, wherein the improvements are relative to placebo.

In some embodiments, the patient in need thereof having non-relapsing SPMS has non-active SPMS. In some embodiments, the patient in need therof with non-active SPMS has no Gd-enhancing T1-hyperintense lesions as detected by MRI before administration of tolebrutinib, and had no relapse in the 2 years before administration of tolebrutinib. In some embodiments, the time to onset of 6-month CDP is improved about 23% for the patient having no Gd-enhancing T1-hyperintense lesions as detected by MRI before administration of tolebrutinib, and having no relapse in the 2 years before administration of tolebrutinib, wherein the improvement is relative to placebo.

In some embodiments, the patient in need thereof has an EDSS score of 4.5 or less before administration of tolebrutinib. In some embodiments, the time to onset of 6-month CDP is improved about 40% for the patient having an EDSS score of 4.5 or less compared to 28% for a patient having an EDSS score greater than 4.5 before administration of tolebrutinib, wherein the improvements are relative to placebo.

In some embodiments, the patient in need thereof has an EDSS score of 5.5 or less before administration of tolebrutinib. In some embodiments, the time to onset of 6-month CDP is improved about 43% for the patient having an EDSS score of 5.5 or less compared to 20% for a patient having an EDSS score greater than 5.5 before administration of tolebrutinib, wherein the improvements are relative to placebo.

In some embodiments, the patient in need thereof has received one or fewer disease modifying therapies before administration of tolebrutinib. In some embodiments, the patient in need thereof has not received a disease modifying therapy before administration of tolebrutinib. In some embodiments, the time to onset of 6-month CDP is improved about 610% for the patient who has not received a disease modifying therapy compared to about 35% for the patient having received one disease modifying therapy and compared to about 10% for a patient having received two or more disease modifying therapies before administration of tolebrutinib, wherein the improvements are relative to placebo. In some embodiments, the patient in need thereof has received one prior disease modifying therapy. In some embodiments, the one prior disease modifying therapy is selected from the group consisting of an interferon, glatiramer acetate, dimethyl fumarate, ocrelizumab, teriflunomide, natalizumab, fingolimod, and rituximab.

In some embodiments, the patient in need thereof had onset of RMS symptoms less than 10 years before administration of tolebrutinib. In some embodiments, the patient in need thereof had onset of RMS symptoms more than 5 years and 10 years or less before administration of tolebrutinib. In some embodiments, the patient in need thereof had onset of RMS symptoms 5 years or less before administration of tolebrutinib. In some embodiments, the time to onset of 6-month CDP is improved about 49% for the patient having onset of RMS symptoms 5 years or less or about 46% for the patient having onset of RMS symptoms more than 5 years and 10 years or less compared to about 26% for a patient having onset of RMS symptoms more than 10 years before administration of tolebrutinib, wherein the improvements are relative to placebo.

In some embodiments, the patient in need thereof had one or more phase rim lesions (PRL) as detected by MRI before administration of tolebrutinib. In some embodiments, the patient in need thereof had one, two, or three phase rim lesions (PRL) as detected by MRI before administration of tolebrutinib. In some embodiments, the patient in need thereof has four or more phase rim lesions (PRL) as detected by MRI before administration of tolebrutinib. In some embodiments, the time to onset of 6-month CDP is improved about 54% for the patient having four or more PRL compared to about 15% for the patient having one, two, or three PRLs and compared to about −17% for a patient having no PRL before administration of tolebrutinib, wherein the improvements are relative to placebo.

In certain methods disclosed herein, wherein tolebrutinib is administered to a patient in need thereof having a relapsing form of multiple sclerosis (RMS), the treatment effect (e.g., improved time to onset of 6-month confirmed disability progression (CDW)) is greater in a first subgroup of patients than in patients not in the first subgroup.

In some embodiments, the patient in need thereof has an EDSS score of less than 4 before administration of tolebrutinib. In some embodiments, the time to onset of 6-month CDW is improved about 33% for the patient having an EDSS score of less than 4 compared to about −4% for a patient having an EDSS score of 4 or more before administration of tolebrutinib, wherein the improvements are relative to teriflunomide.

In some embodiments, the patient in need thereof has one or more Gd-enhancing T1-hyperintense lesions as detected by MRI before administration of tolebrutinib. In some embodiments, the time to onset of 6-month CDW is improved about 39% for the patient having one or more Gd-enhancing T1-hyperintense lesions compared to about 24% for a patient not having a Gd-enhancing T1-hyperintense lesion before administration of tolebrutinib, wherein the improvements are relative to teriflunomide.

In some embodiments, the patient in need thereof has not received a disease modifying therapy before administration of tolebrutinib. In some embodiments, the time to onset of 6-month CDW is improved about 46% for the patient who has not received a disease modifying therapy compared to about −5% for a patient having received one disease modifying therapy before administration of tolebrutinib, wherein the improvements are relative to teriflunomide.

In some embodiments, the patient in need thereof experienced onset of RMS symptoms less than 10 years before administration of tolebrutinib. In some embodiments, the patient in need thereof experienced onset of RMS symptoms less than 5 years before administration of tolebrutinib. In some embodiments, the patient in need thereof experienced onset of RMS symptoms 5 or more years and less than 10 years before administration of tolebrutinib. In some embodiments, the time to onset of 6-month CDW is improved about 44% for the patient having onset of RMS symptoms less than 5 years or about 44% for the patient having onset of RMS symptoms 5 or more years and less than 10 years compared to about−10% for a patient having onset of RMS symptoms 10 years or more before administration of tolebrutinib, wherein the improvements are relative to teriflunomide.

In some embodiments, the patient in need thereof had 2 or more relapses in the one year before administration of tolebrutinib. In some embodiments, the time to onset of 6-month CDW is improved about 51% for the patient having 2 or more relapses in the one year before administration of tolebrutinib compared to about 22% for a patient having less than 2 relapses in the one year before administration of tolebrutinib, wherein the improvements are relative to teriflunomide.

In some embodiments, the patient in need thereof had one or more phase rim lesions (PRL) as detected by MRI before administration of tolebrutinib. In some embodiments, the patient in need thereof had one, two, or three phase rim lesions (PRL) as detected by MRI before administration of tolebrutinib. In some embodiments, the patient in need thereof has four or more phase rim lesions (PRL) as detected by MRI before administration of tolebrutinib. In some embodiments, the time to onset of 6-month CDW is improved about 49% for the patient having four or more PRL or about 46% for the patient having one, two, or three PRLs compared to about 18% for a patient having no PRL before administration of tolebrutinib, wherein the improvements are relative to teriflunomide.

### IV. Therapeutic Methods to Mitigate Risk of Hepatic Injury

Provided herein are methods of treating MS comprising administering to a patient in need thereof a therapeutically effective amount of (R)-1-(1-acryloylpiperidin-3-yl)-4-amino-3-(4-phenoxyphenyl)-1H-imidazo[4,5-c]pyridin-2(3H)-one, a pharmaceutically acceptable salt thereof, or a crystalline form thereof. However, in some embodiments, the administration of the compound will (in certain embodiments) only occur if certain pre-conditions are met.

In one embodiment, the patient's levels of transferrin or ferritin are measured. If it is found that the patient's levels of transferrin or ferritin are not elevated, then the patient will receive tolebrutinib. However, if is found that the patient's transferrin or ferritin are elevated, the patient will not receive the compound. Elevated transferrin levels are determined by examining the saturation level of transferrin. Particularly an elevated transferrin level is where the patient's transferrin saturation level is >50% in males and >40% in females. Stated differently, if the patient's transferrin saturation level is >50% in males and >40% in females, the patient will not receive tolebrutinib, whereas if the patient's transferrin saturation level is measured and it is found that the saturation is less than or equal to 50% in males or less than or equal to 40% in females, then the patient can receive study drug.

In some embodiments, the patient's ferritin levels are measured. If it is found that the patient's levels of ferritin are not elevated, then the patient will receive tolebrutinib. Elevated ferritin levels means a level of >500 μg/L. In some embodiments, the patient's level of ferritin is measured, and if it is found to be higher than 500 μg/L, the patient is not administered the BTK compound. But if it is found to be less than or equal to 500 μg/L, then the patient will receive the BTK compound.

In some embodiments, the patient's iron panel will be measured, which includes both iron levels in blood and serum, ferritin levels and transferrin saturation. If either the ferritin level or the transferrin level is above the thresholds listed above, the patient will not receive the BTK inhibitor. But if both the ferritin level and the transferrin level are below the thresholds listed above, the patient will receive the BTK inhibitor.

In some embodiments, there are methods of treating MS comprising determining the patient's alcohol consumption, and when the patient is found to have a suitable alcohol consumption, administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one. In some embodiments, suitable alcohol consumption means that patient does not have an active alcohol use disorder or a history of alcohol or drug abuse within 1 year prior to the first visit. In other embodiments, suitable alcohol consumption means that the patient has a current alcohol intake of >2 drinks per day for men and >1 drink per day for women (1 drink=approximately 14 grams of alcohol=350 mL beer=140 mL wine=40 mL of spirits). Thus, the patient needs to have an alcohol consumption less than 2 drinks/day for men and 1 drink/day for women in order for the BTK compound to be administered.

In some embodiments, there are methods for treating MS in which it is measured whether the patient is concomitantly taking inducers of the CYP3A enzyme. If the patient has taken such an inhibitor, the patient will not receive the BTK compound. Likewise, the patient will not be given the BTK compound if patient is concomitantly taking an inhibitor of CYP3C8.

In some embodiments, it will be determined if the patient has elevated ALT enzymes, and if so, the patient will not receive the BTK inhibitor. This refers to the alanine aminotransferase (ALT) enzyme that has been increased and/or has a starting value that is >3×upper limit of normal (ULN). Such patients will not receive the BTK compound.

In one embodiment, the patient's levels of transferrin or ferritin are measured. If it is found that the patient's levels of transferrin or ferritin are not elevated, then the patient will continue to receive tolebrutinib. However, if is found that the patient's transferrin or ferritin are elevated, the patient discontinues receiving the compound (or the compound is no longer administered to the patient). Specifically, in some embodiments if the patient's transferrin saturation level is >50% in males and >40% in females, the patient will discontinue receiving the compound.

In some embodiments, the patient's ferritin levels are measured. If it is found that the patient's levels of ferritin are not elevated, then the patient will continue to receive tolebrutinib. However, if is found that the patient's ferritin is elevated, the patient discontinues receiving the compound (or the compound is no longer administered to the patient). Specifically, the patient's level of ferritin is measured, and if it is found to be higher than >500 μg/L, the patient is not administered the BTK compound and/or the patient stops receiving the compound. But if it is found to be less than or equal to 500 μg/L, then the patient will continue to receive the BTK compound.

In some embodiments, the patient's iron panel will be measured, which includes both iron levels in blood and serum, ferritin levels and transferrin saturation. If either the ferritin level or the transferrin level is above the thresholds listed above, the patient will not receive the BTK inhibitor or will stop receiving the compound. If both the ferritin level or the transferrin level is above the thresholds listed above, the patient will also not receive the BTK inhibitor or will stop receiving the compound.

In some embodiments, there are methods of treating MS comprising determining the patient's alcohol consumption, and when the patient is found to have a suitable alcohol consumption, administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one. In some embodiments, suitable alcohol consumption means that patient does not have an active alcohol use disorder or a history of alcohol or drug abuse within 1 year prior to the first visit. In other embodiments, suitable alcohol consumption means that the patient has a current alcohol intake of >2 drinks per day for men and >1 drink per day for women (1 drink=approximately 14 grams of alcohol=350 mL beer=140 mL wine=40 mL of spirits). Thus, the patient needs to have an alcohol consumption less than 2 drinks/day for men and 1 drink/day for women in order for the BTK compound to be administered.

In some embodiments, there are methods of treating MS comprising determining the patient's alcohol consumption, and when the patient is found to have a higher than suitable alcohol consumption, the patient is not administered tolebrutinib or the administration of the compound is discontinued. In some embodiments, higher than suitable alcohol consumption means that patient has an active alcohol use disorder or a history of alcohol or drug abuse within 1 year prior to the first visit. In other embodiments, higher than suitable alcohol consumption means that the patient has a current alcohol intake of >2 drinks per day for men and >1 drink per day for women (1 drink=approximately 14 grams of alcohol=350 mL beer=140 mL wine=40 mL of spirits). Thus, the patient needs to have an alcohol consumption less than 2 drinks/day for men and 1 drink/day for women in order for tolebrutinib to be administered or for the patient to continue to receive tolebrutinib.

In some embodiments, there are methods for treating MS in which it is measured whether the patient is concomitantly taking inducers of the CYP3A enzyme. If the patient has taken such an inhibitor, the patient will not receive tolebrutinib and/or the administration of the compound will be discontinued. Likewise, the patient will not be given tolebrutinib (or the administration of the compound will be discontinued) if patient is concomitantly taking an inhibitor of CYP3C8.

In some embodiments, it will be determined if the patient has elevated ALT enzymes, and if so, the patient will not receive tolebrutinib or will discontinue receiving the compound. This refers to the alanine aminotransferase (ALT) enzyme that has been increased and/or has a starting value that is >3×upper limit of normal (ULN). Such patients will not receive tolebrutinib or the administration of the compound will cease.

In one embodiment, the patient will be classified as having mild, moderate or severe hepatic impairment as measured by the Child-Pugh class scale. In some embodiments, if the patient is determined to have severe hepatic impairment, the patient will not receive the compound or the administration of the compound to said patient will cease. In some embodiments, if the patient is determined to have moderate hepatic impairment, the patient will not receive the compound or the administration of the compound to said patient will cease. In some embodiments, if the patient is determined to have mild hepatic impairment, the patient will not receive the compound or the administration of the compound to said patient will cease.

In some embodiments, the patient will be screened to determine if the ALT>1.5×ULN OR AST>1.5×ULN OR alkaline phosphatase >2×ULN (unless caused by non-liver-related disorder or explained by a stable chronic liver disorder) OR total bilirubin >1.5×ULN (unless due to Gilbert syndrome or non-liver-related disorder). Such patient's that have enzymes above these levels will not be administered the compound or if they have already received the compound, will discontinue receiving the compound.

In some embodiments, the dose is once daily. In some embodiments, the dose is administered once daily with food. In some embodiments, the dose is 60 mg, measured in base form, and is administered once daily with food. In some embodiments, tolebrutinib is administered as monotherapy.

In some embodiments, the patient is a mammal. In some embodiments, the mammal is a human. In some embodiments, the patient is a human subject ranging in age from 12 to 55 years old. In some embodiments, the patient is a human subject at least 18 years old. In some embodiments, the patient is a human subject ranging in age from 18 to 90 years old, for example, 20 to 85 years old, 25 to 80 years old, 30 to 75 years old, 35 to 70 years old, 40 to 65 years old, 45 to 60 years old or 50 to 55 years old.

In some embodiments the therapeutically effective amount is about 5 to about 60 mg, measured in base form. In some embodiments, the patient is a mammal. In some embodiments, the mammal is a human. In some embodiments, the patient is a human patient ranging in age from 12 to 55 years old. In some embodiments, the patient is a human subject at least 18 years old. In some embodiments, the patient is a human patient ranging in age from 18 to 90 years old, for example, 20 to 85 years old, 25 to 80 years old, 30 to 75 years old, 35 to 70 years old, 40 to 65 years old, 45 to 60 years old or 50 to 55 years old.

In some embodiments, tolebrutinib is administered as monotherapy. In some embodiments, tolebrutinib is administered as monotherapy in 60 mg doses. In some embodiments, tolebrutinib is administered as monotherapy in 60 mg doses once daily. In some embodiments, tolebrutinib is administered as monotherapy in 60 mg doses once daily with food.

In some embodiments, a dose of about 5-10 mg, 10-15 mg, 15-20 mg, 20-25 mg, 25-30 mg, 30-35 mg, 35-40 mg, 40-45 mg, 45-50 mg, 50-55 mg, or 55-60 mg of tolebrutinib or a pharmaceutically acceptable salt thereof, measured in base form, is administered. In some embodiments, the dose is 5 mg, 10 mg, 15 mg, 20 mg, 25 mg, 30 mg, 35 mg, 40 mg, 45 mg, 50 mg, 55 mg, or 60 mg of tolebrutinib or a pharmaceutically acceptable salt thereof, measured in base form. In some embodiments, the dose is 5 mg of tolebrutinib or a pharmaceutically acceptable salt thereof, measured in base form. In some embodiments, the dose is 15 mg of tolebrutinib or a pharmaceutically acceptable salt thereof, measured in base form. In some embodiments, the dose is 30 mg. In some embodiments, the dose is 60 mg of tolebrutinib or a pharmaceutically acceptable salt thereof, measured in base form.

In some embodiments, the dose is administered daily. The daily dose can be delivered as a single dose or split into multiple parts. For example, in some embodiments, the dose is administered once a day (e.g., about every 24 hours). In some embodiments, the dose is administered twice daily. In some embodiments, the dose is subdivided in two parts to be administered twice per day (e.g., about every 12 hours). In some embodiments, the dose is subdivided in three parts to be administered three times per day (e.g., about every 8 hours). In some embodiments, the dose is subdivided in four parts to be administered four times per day (e.g., about every 6 hours).

In some embodiments, the dose is administered orally. In some embodiments, the dose is administered in a form of tablets. In some embodiments, the dose is administered in the form of pills, capsules, semisolids, powders, sustained release formulations, solutions, suspensions, elixirs, aerosols, or any other appropriate compositions.

In some embodiments, the patient is administered tolebrutinib for a period of about 1, 2, 3, 4, 5, 6, 7, 8, 9, 10, 11, or 12 months, or for life. In some embodiments, the patient is administered tolebrutinib for a period of about 12 months. In some embodiments, the dose is once daily. In one embodiment, a method of treating MS is provided, the method comprising administering to a patient in need thereof 60 mg tolebrutinib or a pharmaceutically acceptable salt thereof, measured in base form.

In some embodiments, tolebrutinib is administered as monotherapy. In some embodiments, the method comprises administering tolebrutinib and at least one additional therapeutic agent. The additional therapeutic agent may be administered concurrently or sequentially with tolebrutinib.

Determination of the frequency of administration can be made by persons skilled in the art, such as an attending physician based on considerations of the condition being treated, age of the subject being treated, severity of the condition being treated, general state of health of the subject being treated and the like. In some embodiments, BTK inhibitor compounds are administered in a therapeutically effective amount for treatment of MS. The therapeutically effective amount is typically dependent on the weight of the subject being treated, his or her physical or health condition, the extensiveness of the condition to be treated, or the age of the subject being treated, pharmaceutical formulation methods, or administration methods (e.g., administration time and administration route).

In some embodiments, a method of treating MS is provided, comprising the steps of performing an iron panel test using a patient's blood or serum, and if the patient has a suitable iron panel, administering a therapeutically acceptable amount of tolebrutinib to the patient. In some embodiments the iron panel test measures any one or more of levels of iron, ferritin, transferrin saturation, and total iron-binding capacity (TIBC) in a patient's blood or serum. In some embodiments, a suitable iron panel includes one or more of the following: (i) an iron level of 60 to 170 μg/dL, (ii) a ferritin level of ≤500 μg/L (iii) a transferrin saturation level ≤50% in a male patient or ≤40% in a female patient, and (iv) a TIBC of 240 to 450 μg/dL.

In some embodiments, a method of treating MS is provided, comprising the steps of performing an iron panel test in a patient's blood or serum, detecting levels of the iron panel test that are within normal ranges, and administering a therapeutically acceptable amount of tolebrutinib to the patient. In some embodiments the iron panel test measures any one or more of levels of iron, ferritin, transferrin saturation, and total iron-binding capacity (TIBC) in a patient's blood or serum. In some embodiments, the normal ranges of the iron panel test include one or more of (i) an iron level of 60 to 170 μg/dL, (ii) a ferritin level of ≤500 μg/L (iii) a transferrin saturation level ≤50% in a male patient or ≤40% in a female patient, and (iv) a TIBC of 240 to 450 μg/dL.

In some embodiments, the present disclosure provides tolebrutinib for use in a method of treating MS, comprising the steps of performing an iron panel test in a patient's blood or serum, detecting levels of the iron panel test that are within normal ranges, and administering a therapeutically acceptable amount of tolebrutinib to the patient. In some embodiments the iron panel test measures any one or more of levels of iron, ferritin, transferrin saturation, and total iron-binding capacity (TIBC) in a patient's blood or serum. In some embodiments, the normal ranges of the iron panel test include one or more of (i) an iron level of 60 to 170 μg/dL, (ii) a ferritin level of ≤500 μg/L (iii) a transferrin saturation level ≤50% in a male patient or ≤40% in a female patient, and (iv) a TIBC of 240 to 450 μg/dL.

In some embodiments, a method of treating MS is provided, comprising the steps of determining the level of transferrin saturation in a patient's blood or serum, and if the level of transferrin saturation is suitable, administering a therapeutically effective amount of tolebrutinib to the patient. In some embodiments, a suitable transferrin saturation level in the blood or serum of a male patient is a transferrin saturation of ≤50%. In some embodiments, a suitable transferrin saturation level in the blood or serum of a female patient is a transferrin saturation of ≤40%.

In some embodiments, a method of treating MS is provided, comprising the steps of detecting a level of transferrin saturation in a patient's blood or serum that is within normal range, and administering a therapeutically effective amount of tolebrutinib to the patient. In some embodiments, a transferrin saturation level that is within normal range in the blood or serum of a male patient is a transferrin saturation of ≤50%. In some embodiments, a transferrin saturation level that is within normal range in the blood or serum of a female patient is a transferrin saturation of ≤40%.

In some embodiments, the present disclosure provides tolebrutinib for use in a method of treating MS, comprising the steps of detecting a level of transferrin saturation in a patient's blood or serum that is within normal range, and administering a therapeutically effective amount of tolebrutinib to the patient. In some embodiments, a transferrin saturation level that is within normal range in the blood or serum of a male patient is a transferrin saturation of ≤50%. In some embodiments, a transferrin saturation level that is within normal range in the blood or serum of a female patient is a transferrin saturation of ≤40%. In some embodiments, detecting a level of transferrin saturation is done in a previously collected patient's blood or serum sample.

In some embodiments, a method of treating MS is provided, comprising the steps of determining the level of ferritin in a patient's blood or serum, and if the level of ferritin is suitable, administering a therapeutically acceptable amount of tolebrutinib to the patient. In some embodiments, a suitable ferritin level in the blood or serum of a patient is ≤500 μg/L.

In some embodiments, a method of treating MS is provided, comprising the steps of detecting a level of ferritin in a patient's blood or serum that is within normal range, and administering a therapeutically acceptable amount of tolebrutinib to the patient. In some embodiments, a ferritin level that is within normal range in the blood or serum of a patient is ≤500 μg/L.

In some embodiments, the present disclosure provides tolebrutinib for use in a method of treating MS, comprising the steps of detecting a level of ferritin in a patient's blood or serum that is within normal range, and administering a therapeutically acceptable amount of tolebrutinib to the patient. In some embodiments, a ferritin level that is within normal range in the blood or serum of a patient is ≤500 μg/L. In some embodiments, detecting a level of ferritin is done in a previously collected patient's blood or serum sample.

In some embodiments, a method of treating MS is provided, comprising the steps of performing liver function tests in a patient, and if the patient has suitable liver function, administering a therapeutically acceptable amount of tolebrutinib to the patient. In some embodiments, the liver function tests measure one or more of the levels of aspartate transaminase (AST), alanine transaminase (ALT), albumin, alkaline phosphatase, total and direct bilirubin, and total protein in a patient's blood. In some embodiments a patient having a suitable liver function has one or more of ALT levels of ≤1.5×upper limit of normal (ULN), AST levels of ≤1.5×ULN, and alkaline phosphatase ≤2×ULN (unless caused by non-liver related disorder or explained by a stable chronic liver disorder) and total bilirubin ≤1.5×ULN (unless due to Gilbert syndrome or non-liver-related disorder).

In some embodiments, a method of treating MS is provided, comprising the steps of performing liver function tests in a patient, detecting suitable liver function, and administering a therapeutically acceptable amount of tolebrutinib to the patient. In some embodiments, the liver function tests measure one or more of the levels of aspartate transaminase (AST), alanine transaminase (ALT), albumin, alkaline phosphatase, total and direct bilirubin, and total protein in a patient's blood. In some embodiments a patient having a suitable liver function has one or more of ALT levels of ≤1.5×upper limit of normal (ULN), AST levels of ≤1.5×ULN, and alkaline phosphatase ≤2×ULN (unless caused by non-liver related disorder or explained by a stable chronic liver disorder) and total bilirubin ≤1.5×ULN (unless due to Gilbert syndrome or non-liver-related disorder).

In some embodiments, the present disclosure provides tolebrutinib for use in a method of treating MS, comprising the steps of performing liver function tests in a patient, detecting suitable liver function, and administering a therapeutically acceptable amount of tolebrutinib to the patient. In some embodiments, the liver function tests measure one or more of the levels of aspartate transaminase (AST), alanine transaminase (ALT), albumin, alkaline phosphatase, total and direct bilirubin, and total protein in a patient's blood. In some embodiments a patient having a suitable liver function has one or more of ALT levels of ≤1.5×upper limit of normal (ULN), AST levels of ≤1.5×ULN, and alkaline phosphatase ≤2×ULN (unless caused by non-liver related disorder or explained by a stable chronic liver disorder) and total bilirubin ≤1.5×ULN (unless due to Gilbert syndrome or non-liver-related disorder). In some embodiments, measuring of aspartate transaminase (AST), alanine transaminase (ALT), albumin, alkaline phosphatase, total and direct bilirubin, and total protein is done in a previously collected patient's blood sample.

In some embodiments, the liver function tests are performed at least about every 6 months, at least about every 5 months, at least about every 4 months, at least about every 3 months, at least about every 2 months, or at least about monthly. In some embodiments, the liver function tests are performed at least about every 12 weeks, at least about every 11 weeks, at least about every 10 weeks, at least about every 9 weeks, at least about every 8 weeks, at least about every 7 weeks, at least about every 6 weeks, at least about every 5 weeks, at least about every 4 weeks, at least about every 3 weeks, at least about every 2 weeks, or at least about weekly.

In some embodiments, a method of treating MS is provided, comprising the steps of:
a) administering a therapeutically effective amount of tolebrutinib to a patient in need thereof;
b) measuring the level of alanine aminotransferase (ALT) in the patient;
c) detecting a level of ALT of >8×upper limit of normal (ULN);
d) ceasing administration of tolebrutinib to the patient; and optionally
e) monitoring the level of ALT in the patient; and
f) resuming administration of a therapeutically effective amount of tolebrutinib to the patient when the patient's level of ALT is determined to be <1.5×ULN.

In some embodiments, the present disclosure provides tolebrutinib for use in a method of treating MS, comprising the steps of:
a) administering a therapeutically effective amount of tolebrutinib to a patient in need thereof;
b) measuring the level of alanine aminotransferase (ALT) in the patient;
c) detecting a level of ALT of >8×upper limit of normal (ULN);
d) ceasing administration of the tolebrutinib to the patient; and optionally
e) monitoring the level of ALT in the patient; and
f) resuming administration of a therapeutically effective amount of tolebrutinib to the patient when the patient's level of ALT is determined to be <1.5×ULN.

In some embodiments, a method of treating MS is provided, comprising the steps of:
a) administering a therapeutically effective amount of tolebrutinib to a patient in need thereof;
b) measuring the level of alanine aminotransferase (ALT) in the patient;
c) detecting a level of ALT of >5×upper limit of normal (ULN) during a period
of at least two weeks;
d) ceasing administration of tolebrutinib to the patient; and optionally
e) monitoring the level of ALT in the patient; and
f) resuming administration of a therapeutically effective amount of tolebrutinib to the patient when the patient's level of ALT is determined to be <1.5×ULN.

In some embodiments, the present disclosure provides tolebrutinib for use in a method of treating MS, comprising the steps of:
a) administering a therapeutically effective amount of tolebrutinib to a patient in need thereof;
b) measuring the level of alanine aminotransferase (ALT) in the patient;
c) detecting a level of ALT of >5×upper limit of normal (ULN) during a period of at least two weeks;
d) ceasing administration of tolebrutinib to the patient; and optionally
e) monitoring the level of ALT in the patient; and
f) resuming administration of a therapeutically effective amount of tolebrutinib to the patient when the patient's level of ALT is determined to be <1.5×ULN.

In some embodiments, a method of treating MS is provided, comprising the steps of:
a) administering a therapeutically effective amount of tolebrutinib to a patient in need thereof;
b) measuring the level of alanine aminotransferase (ALT) in the patient;
c) detecting a level of ALT of >3×upper limit of normal (ULN);
d) measuring one or more of total bilirubin and international normalized ratio (INR) in a patient;
e) detecting one or more of total bilirubin >2×ULN and INR>1.5;
f) ceasing administration of tolebrutinib to the patient; and optionally
g) monitoring the level of ALT in the patient; and
h) resuming administration of a therapeutically effective amount of tolebrutinib to the patient when the patient's level of ALT is determined to be <1.5×ULN.

In some embodiments, the present disclosure provides tolebrutinib for use in a method of treating MS, comprising the steps of:
a) administering a therapeutically effective amount of tolebrutinib to a patient in need thereof;
b) measuring the level of alanine aminotransferase (ALT) in the patient;
c) detecting a level of ALT of >3×upper limit of normal (ULN);
d) measuring one or more of total bilirubin and international normalized ratio (INR) in a patient;
e) detecting one or more of total bilirubin >2×ULN and INR>1.5;
f) ceasing administration of tolebrutinib to the patient; and optionally
g) monitoring the level of ALT in the patient; and
h) resuming administration of a therapeutically effective amount of tolebrutinib to the patient when the patient's level of ALT is determined to be <1.5×ULN.

In some embodiments, a method of treating MS is provided, comprising the steps of:
a) administering a therapeutically effective amount of tolebrutinib to a patient in need thereof;
b) measuring the level of alanine aminotransferase (ALT) in the patient;
c) detecting a level of ALT of >3×upper limit of normal (ULN);
d) ceasing administration of tolebrutinib to the patient if the patient experiences one or more of fatigue, nausea, vomiting, right upper quadrant pain or tenderness, fever, rash, and eosinophilia >5%; and optionally
e) monitoring the level of ALT in the patient; and
f) resuming administration of a therapeutically effective amount of tolebrutinib to the patient when the patient's level of ALT is determined to be <1.5×ULN.

In some embodiments, the present disclosure provides tolebrutinib for use in a method of treating MS, comprising the steps of:
a) administering a therapeutically effective amount of tolebrutinib to a patient in need thereof;
b) measuring the level of alanine aminotransferase (ALT) in the patient;
c) detecting a level of ALT of >3×upper limit of normal (ULN);
d) ceasing administration of tolebrutinib to the patient if the patient experiences one or more of fatigue, nausea, vomiting, right upper quadrant pain or tenderness, fever, rash, and eosinophilia >5%; and optionally
e) monitoring the level of ALT in the patient; and
f) resuming administration of a therapeutically effective amount of tolebrutinib to the patient when the patient's level of ALT is determined to be <1.5×ULN.

In some embodiments, the steps of measuring, detecting, and monitoring a level of ALT or total bilirubin are done on a previously collected blood or serum sample of the patient in need thereof.

In some embodiments, the ALT level in a patient is measured at least about every 6 months, at least about every 5 months, at least about every 4 months, at least about every 3 months, at least about every 2 months, or at least about monthly. In some embodiments, the ALT level in a patient is measured at least about every 12 weeks, at least about every 11 weeks, at least about every 10 weeks, at least about every 9 weeks, at least about every 8 weeks, at least about every 7 weeks, at least about every 6 weeks, at least about every 5 weeks, at least about every 4 weeks, at least about every 3 weeks, at least about every 2 weeks, or at least about weekly.

In some embodiments, after ceasing administration of the compound, the ALT level in a patient is monitored about every 2 to 3 days, about every 3 days, about every 2 days, or about daily.

In some embodiments, a method of treating MS is provided, comprising administering to a therapeutically acceptable amount of tolebrutinib to a patient, wherein the patient is not receiving potent and moderate inducers of cytochrome P450 3A (CYP3A) or potent inhibitors of CYP2C8 hepatic enzymes. In some embodiments the potent CYP3A inducers are selected from rifampin, carbamazepine, phenobarbital, St John's Wort extract, avasimibe, lumacaftor, rifapentine, rifabutin, and phenytoin. In some embodiments the moderate CYP3A inducers are selected from semagacestat, asunaprevir, beclabuvir, daclatasvir, cenobamate, nafcillin, lesinurad, bosentan, telotristat ethyl, thioridazine, elagolix and rifabutin. In some embodiments, potent CYP2C8 inhibitors are selected from Gemfibrozil and Clopidogrel.

In some embodiments, the present disclosure provides tolebrutinib for use in a method of treating MS, comprising administering to a therapeutically acceptable amount of tolebrutinib to a patient, wherein the patient is not receiving potent and moderate inducers of cytochrome P450 3A (CYP3A) or potent inhibitors of CYP2C8 hepatic enzymes. In some embodiments the potent CYP3A inducers are selected from rifampin, carbamazepine, phenobarbital, St John's Wort extract, avasimibe, lumacaftor, rifapentine, rifabutin, and phenytoin. In some embodiments the moderate CYP3A inducers are selected from semagacestat, asunaprevir, beclabuvir, daclatasvir, cenobamate, nafcillin, lesinurad, bosentan, telotristat ethyl, thioridazine, elagolix and rifabutin. In some embodiments, potent CYP2C8 inhibitors are selected from Gemfibrozil and Clopidogrel.

In some embodiments, a method of treating MS is provided, comprising administering to a therapeutically acceptable amount of tolebrutinib to a patient, wherein the patient is not receiving potent, moderate, or mild inhibitor of cytochrome P450 2C8 (CYP2C8), potent or moderate CYP3A inducers, or potent, moderate, or mild inhibitors of CYP3A hepatic enzymes. In some embodiments, potent CYP2C8 inhibitors are selected from Gemfibrozil and Clopidogrel. In some embodiments, moderate CYP2C8 inhibitor is Trimethoprim. In some embodiments, mild CYP2C8 inhibitors are selected from sulfamethoxazole, trimethoprim, and fluvoxamine. In some embodiments the potent CYP3A inducers are selected from avasimibe, rifampin, carbamazepine, lumacaftor, phenobarbital, phenytoin, rifapentine, and St John's Wort extract. In some embodiments the moderate CYP3A inducers are selected from elagolix, cenobamate, nafcillin, asunaprevir, beclabuvir, daclatasvir, lesinurad, bosentan, thioridazine, and rifabutin. In some embodiments the potent CYP3A inhibitors are selected from clarithromycin, itraconazole, ketoconazole, nirmatrelvir and ritonavir, fluoxetine, and grapefruit juice. In some embodiments the moderate CYP3A inhibitors are selected from ciprofloxacin, diltiazem, erythromycin, fluconazole, verapamil, and sertraline. In some embodiments the mild CYP3A inhibitors are selected from alprazolam, atorvastatin, amlodipine, cimetidine, ranitidine, roxithromycin, Ginkgo biloba, and isoniazid.

In some embodiments, the present disclosure provides tolebrutinib for use in a method of treating MS, comprising administering to a therapeutically acceptable amount of tolebrutinib to a patient, wherein the patient is not receiving potent, moderate, or mild inhibitor of cytochrome P450 2C8 (CYP2C8), potent or moderate CYP3A inducers, or potent, moderate, or mild inhibitors of CYP3A hepatic enzymes. In some embodiments, potent CYP2C8 inhibitors are selected from Gemfibrozil and Clopidogrel. In some embodiments, moderate CYP2C8 inhibitor is Trimethoprim. In some embodiments, mild CYP2C8 inhibitors are selected from sulfamethoxazole, trimethoprim, and fluvoxamine. In some embodiments the potent CYP3A inducers are selected from avasimibe, rifampin, carbamazepine, lumacaftor, phenobarbital, phenytoin, rifapentine, and St John's Wort extract. In some embodiments the moderate CYP3A inducers are selected from elagolix, cenobamate, nafcillin, asunaprevir, beclabuvir, daclatasvir, lesinurad, bosentan, thioridazine, and rifabutin. In some embodiments the potent CYP3A inhibitors are selected from clarithromycin, itraconazole, ketoconazole, nirmatrelvir and ritonavir, fluoxetine, and grapefruit juice. In some embodiments the moderate CYP3A inhibitors are selected from ciprofloxacin, diltiazem, erythromycin, fluconazole, verapamil, and sertraline. In some embodiments the mild CYP3A inhibitors are selected from alprazolam, atorvastatin, amlodipine, cimetidine, ranitidine, roxithromycin, Ginkgo biloba, and isoniazid.

In some embodiments, a method of treating MS is provided, comprising the steps of advising the patient to limit alcohol consumption during treatment, and administering a therapeutically acceptable amount of tolebrutinib to the patient. In some embodiments, the patient is female and is advised to limit alcohol consumption to 1 drink per day or less. In some embodiments, 1 drink is approximately 14 grams of alcohol (e.g., 350 mL beer, 140 mL wine, or 40 mL spirits). In some embodiments, the patient is male and is advised to limit alcohol consumption to 2 drinks per day or less. In some embodiments, 2 drinks is approximately 28 grams of alcohol.

In some embodiments, the present disclosure provides tolebrutinib for use in a method of treating MS, comprising the steps of advising the patient to limit alcohol consumption during treatment, and administering a therapeutically acceptable amount of tolebrutinib to the patient. In some embodiments, the patient is female and is advised to limit alcohol consumption to 1 drink per day or less. In some embodiments, 1 drink is approximately 14 grams of alcohol (e.g., 350 mL beer, 140 mL wine, or 40 mL spirits). In some embodiments, the patient is male and is advised to limit alcohol consumption to 2 drinks per day or less. In some embodiments, 2 drinks is approximately 28 grams of alcohol.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability. Bioavailability of drugs that decompose at stomach pH can be increased by administration of such drugs in a formulation that releases the drug intraduodenally.

The compositions are comprised of in general, tolebrutinib or a pharmaceutically acceptable salt thereof in combination with a pharmaceutically acceptable excipient such as binders, surfactants, diluents, buffering agents, antiadherents, glidants, hydrophilic or hydrophobic polymers, retardants, stabilizing agents or stabilizers, disintegrants or superdisintegrants, antioxidants, antifoaming agents, fillers, flavors, colors, lubricants, sorbents, preservatives, plasticizers, or sweeteners, or mixtures thereof, which facilitate processing of tolebrutinib or a pharmaceutically acceptable salt thereof into preparations which can be used pharmaceutically. Any of the well-known techniques and excipients may be used as suitable and as understood in the art, see for example, Remington: The Science and Practice of Pharmacy, Twenty-first Ed., (Pharmaceutical Press, 2005); Liberman, H. A., Lachman, L., and Schwartz, J. B. Eds., Pharmaceutical Dosage Forms, Vol. 1-2 Taylor & Francis 1990; and R. I. Mahato, Ansel's Pharmaceutical Dosage Forms and Drug Delivery Systems, Second Ed. (Taylor & Francis, 2012).

In certain embodiments, the formulations may include one or more pH adjusting agents or buffering agents, for example, acids such as acetic, boric, citric, fumaric, maleic, tartaric, malic, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate, ammonium chloride, and the like. Such buffers used as bases may have other counterions than sodium, for example, potassium, magnesium, calcium, ammonium, or other counterions. Such acids, bases and buffers are included in an amount required to maintain pH of the composition in an acceptable range.

In certain embodiments, the formulations may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate, or bisulfite anions; suitable salts include sodium chloride, potassium chloride, sodium thiosulfate, sodium bisulfite and ammonium sulfate.

In certain embodiments, the formulations may also include one or more antifoaming agents to reduce foaming during processing which can result in coagulation of aqueous dispersions, bubbles in the finished film, or generally impair processing. Exemplary anti-foaming agents include silicon emulsions or sorbitan sesquoleate.

In certain embodiments, the formulations may also include one or more antioxidants, such as non-thiol antioxidants, for example, butylated hydroxytoluene (BHT), sodium ascorbate, ascorbic acid or its derivative, and tocopherol or its derivatives. In certain embodiments, antioxidants enhance chemical stability where required. Other agents such as citric acid or citrate salts or EDTA may also be added to slow oxidation.

In certain embodiments, the formulations may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide, and cetylpyridinium chloride.

In certain embodiments, the formulations may also include one or more binders. Binders impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinyl-pyrrolidone/vinyl acetate copolymer; crosspovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum mucilage of isapol husks, polyvinylpyrrolidone (e.g., Polyvidone® CL, Kollidon® CL, Polyplasdone® XL-10), larch arabogalactan, Veegum®, polyethylene glycol, polyethylene oxide, waxes, sodium alginate, and the like.

In certain embodiments, the formulations may also include dispersing agents or viscosity modulating agents. Dispersing agents or viscosity modulating agents include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween®60 or 80, PEG, polyvinylpyrrolidone (PVP; commercially known as Plasdone®), and the carbohydrate-based dispersing agents such as, for example, hydroxypropyl celluloses (e.g., HPC, H-PC-SL, and HPC-L), hydroxypropyl methylcelluloses (e.g., HPMC K100, RPMC K4M, HPMC K15M, and HPMC K100M), carboxymethylcellulose sodium, methylcellulose, hydroxyethyl-cellulose, hydroxypropyl-cellulose, hydroxypropylmethylcellulose phthalate, hydroxypropyl-methylcellulose acetate stearate (HPMCAS), noncrystalline cellulose, polyethylene oxides, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F10®8, which are block copolymers of ethylene oxide and propylene oxide); and poloxamines (e.g., Tetronic 908®, also known as Poloxamine 908®, which is a tetrafonctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizcers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate. In general, binder levels of about 10 to about 70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 90% and more typically up to 70% in tablet formulations is common.

In certain embodiments, the formulations may also include one or more diluents which refer to chemical compounds that are used to dilute the compound of interest prior to delivery.

Diluents can also be used to stabilize compounds because they can provide a more stable environment Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); hydroxypropyl-methylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In certain embodiments, the formulations may also include one or more disintegrants which includes both the dissolution and dispersion of the dosage form when contacted with gastrointestinal fluid. Disintegration agents or disintegrants facilitate the breakup or disintegration of a substance. Examples of disintegration agents include a starch, e.g., e.g., a natural starch such as corn starch or potato starch, a pregelatinized starch such as National 1551 or sodium starch glycolate such as Promogel® or Explotab®, a cellulose such as a wood product, methylcrystalline cellulose, e.g., e.g., Avicel®, Avicel® PH101, Avicel® PH 102, Avicel® PH105, Elceme® P100, Emcocel®, Vivacel®, and Solka-Floc®, methylcellulose, croscarmellose, or a cross-linked cellulose, such as cross-linked sodium carboxymethyl-cellulose (Ac-Di-Sol*), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In certain embodiments, the formulations may also include erosion facilitators. Erosion facilitators include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

In certain embodiments, the formulations may also include one or more filling agents which include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

In certain embodiments, the formulations may also include one or more flavoring agents or sweeteners, e.g., acacia syrup, acesulfame K, alitame, anise, apple, aspartame, banana, Bavarian cream berry, black currant, butterscotch, calcium citrate, camphor, caramel, cherry, cherry cream chocolate, cinnamon, bubble gum, citrus, citrus punch, citrus cream, cotton candy, cocoa, cola, cool cherry, cool citrus, cyclamate, cyclamate, dextrose, eucalyptus, eugenol, fructose, fruit punch, ginger, glycyrrhizinate, glycyrrhiza (licorice) syrup, grape, grapefruit, honey, isomalt, lemon, lime, lemon cream, monoammonium glyrrhizinate, maltol, mannitol, maple, marshmallow, menthol, mint cream, mixed berry, neohesperidine DC, neotame, orange, pear, peach, peppermint, peppermint cream, Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, xylitol, sucralose, sorbitol, Swiss cream, tagatose, tangerine, thaumatin, tutti frutti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

In certain embodiments, the formulations may also include one or more lubricants and glidants which are compounds that prevent, reduce or inhibit adhesion or friction of materials.

Exemplary lubricants include, e.g., stearic acid, calcium hydroxide, talc, sodium stearyl lumerate, a hydrocarbon such as mineral oil, or hydrogenated vegetable oil such as hydrogenated soybean oil, higher fatty acids and their alkali-metal and alkaline earth metal salts, such as aluminum, calcium, magnesium, zinc, stearic acid, sodium stearates, glycerol, talc, waxes, boric acid, sodium benzoate, sodium acetate, sodium chloride, leucine, a polyethylene glycol (e.g., PEG4000) or a methoxypolyethylene glycol such as Carbowax®, sodium oleate, sodium benzoate, glyceryl behenate, polyethylene glycol, magnesium or sodium lauryl sulfate, colloidal silica such as Syloid®, Cab-O-Sil®, a starch such as corn starch, silicone oil, a surfactant, and the like.

In certain embodiments, the formulations may also include one or more plasticizers which are compounds used to soften the enteric or delayed release coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl citrate, dibutyl sebacate, triethyl cellulose and triacetin. In some embodiments, plasticizers can also function as dispersing agents or wetting agents.

In certain embodiments, the formulations may also include one or more solubilizers which include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins for example Captisol®, ethanol, n-butanol, isopropyl alcohol, cholesterol, bile salts, polyethylene glycol 200-600, glycofurol, transcutol, propylene glycol, and dimethyl isosorbide and the like. In one embodiment, the solubilizer is vitamin E TPGS or Captisol® or ß-hydroxypropylcyclodextrin.

In certain embodiments, the formulations may also include one or more suspending agents which include compounds such as polyvinylpyrrolidone, e.g., polyvinylpyrrolidone K112, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monoleate, povidone and the like.

In certain embodiments, the formulations may also include one or more surfactants which include compounds such as sodium lauryl sulfate, sodium docusate, Tween 20, 60 or 80, triacetin, vitamin E TPGS, sorbitan monooleate, polyoxyethylene sorbitan monooleate, polyoxyethylene sorbitan monolaurate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

In certain embodiments, the formulations may also include one or more viscosity enhancing agents which include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol alginates, acacia, chitosans and combinations thereof.

In certain embodiments, the formulations may also include one or more wetting agents which include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

Pharmaceutical preparations disclosed herein can be obtained by mixing one or more solid excipient such as carrier, binder, filling agent, suspending agent, flavoring agent, sweetening agent, disintegrating agent, dispersing agent, surfactant, lubricant, colorant diluent, solubilizer, moistening agent, plasticizer, stabilizer, penetration enhancer, wetting agent, anti-foaming agent, antioxidant, preservative, or one or more combination thereof with one or more of the compounds described herein, optionally grinding the resulting mixture, and processing the mixture of granules, after adding suitable excipients, if desired, to obtain tablets.

Pharmaceutical preparations disclosed herein also include capsules made of gelatin, as well as soft, sealed capsules made of gelatin and a plasticizer, such as glycerol or sorbitol. Capsules may also be made of polymers such as hypromellose. The capsules can contain the active ingredients in admixture with filler such as lactose, binders such as starches, or lubricants such as talc or magnesium stearate and, optionally, stabilizers. In soft capsules, the active compounds may be dissolved or suspended in suitable liquids, such as fatty oils, liquid paraffin, lipids, solubilizers, or liquid polyethylene glycols. In addition, stabilizers may be added. All formulations for oral administration should be in dosages suitable for such administration.

These formulations can be manufactured by conventional pharmacological techniques. Conventional pharmacological techniques include, e.g., one or a combination of methods: (1) dry mixing, (2) direct compression, (3) milling, (4) dry or non-aqueous granulation, (5) wet granulation, (6) fusion, or (7) extrusion. See, e.g., Lachman et al., The Theory and Practice of Industrial Pharmacy, 3^{rd} ed. (1986). Other methods include, e.g., spray drying, pan coating, melt granulation, granulation, fluidized bed spray drying or coating (e.g., wurster coating), tangential coating, top spraying, tableting, extruding, extrusion/spheronization, and the like.

It should be appreciated that there is considerable overlap between excipients used in the solid dosage forms described herein. Thus, the above-listed additives should be taken as merely exemplary, and not limiting, of the types of excipients that can be included in solid dosage forms described herein. The type and amounts of such excipient can be readily determined by one skilled in the art, according to the particular properties desired.

In some embodiments, the solid dosage forms described herein are enteric coated oral dosage forms, i.e., as an oral dosage form of a pharmaceutical composition as described herein which utilizes an enteric coating to affect the release of the compound in the intestine of the gastrointestinal tract. An “enterically coated” drug or tablet refers to a drug or tablet that is coated with a substance that remains intact in the stomach but dissolves and releases the drug once the intestine (in one embodiment small intestine) is reached. As used herein “enteric coating”, is a material, such as a polymer material or materials which encase the therapeutically active agent core either as a dosage form or as particles. Typically, a substantial amount or all of the enteric coating material is dissolved before the therapeutically active agent is released from the dosage form, so as to achieve delayed dissolution of the therapeutically active agent core or particles in the small or large intestine. Enteric coatings are discussed, for example, Loyd, V. Allen, Remington: The Science and Practice of Pharmacy, Twenty-first Ed., (Pharmaceutical Press, 2005; and P. J. Tarcha, Polymers for Controlled Drug Delivery, Chapter 3, CRC Press, 1991. Methods for applying enteric coatings to pharmaceutical compositions are well known in the art, and include for example, U.S. Patent Publication No. 2006/0045822.

The enteric coated dosage form may be a compressed or molded or extruded tablet (coated or uncoated) containing granules, powder, pellets, beads, or particles of tolebrutinib or a pharmaceutically acceptable salt thereof or other excipients, which are themselves coated or uncoated provided at least the tablet or tolebrutinib is coated. The enteric coated oral dosage form may also be a capsule (coated or uncoated) containing pellets, beads, or granules of tolebrutinib or a pharmaceutically acceptable salt thereof or other excipients, which are themselves coated or uncoated provided at least one of them is coated. Some examples of coatings that were originally used as enteric coatings are beeswax and glyceryl monostearate; beeswax, shellac, and cellulose; and cetyl alcohol, mastic, and shellac as well as shellac and stearic acid (U.S. Pat. No. 2,809,918); polyvinylacetate and ethyl cellulose (U.S. Pat. No. 3,835,221). More recently, the coatings used are neutral copolymers of polymethacrylic acid esters (Eudragit L30D). (F. W. Goodhart et al, Pharm. Tech., p. 64-71, April 1984); copolymers of methacrylic acid and methacrylic acid methyl ester (Eudragit S), or a neutral copolymer of polymethacrylic acid esters containing metallic stearates (Mehta et al U.S. Pat. Nos. 4,728,512 and 4,794,001), cellulose acetate succinate, and hypromellose phthalate.

Any anionic polymer exhibiting a pH-dependent solubility profile can be used as an enteric coating in the methods and compositions described herein to achieve delivery to the intestine. In one embodiment, delivery can be to the small intestine. In another embodiment, delivery can be to the duodenum. In some embodiments the polymers described herein are anionic carboxylic polymers. In other embodiments, the polymers, and compatible mixtures thereof, and some of their properties, include, but are not limited to the following.

Shellac: Also called purified lac, it is a refined product obtained from the resinous secretion of an insect. This coating dissolves in media of pH>7.

Acrylic polymers: The performance of acrylic polymers (primarily their solubility in biological fluids) can vary based on the degree and type of substitution. Examples of suitable acrylic polymers include methacrylic acid copolymers and ammonium methacrylate copolymers. The Eudragit series L, S, and RS (manufactured Rohm Pharma and known as Evonik®) are available as solubilized in organic solvent, aqueous dispersion, or dry powders. The Eudragit series RL, NE, and RS are insoluble in the gastrointestinal tract but are permeable and are used primarily for colonic targeting. The Eudragit series L, L-30D and S are insoluble in stomach and dissolve in the intestine and may be selected and formulated to dissolve at a value of pH greater than 5.5 or as low as greater than 5 or as high as greater than 7.

Cellulose Derivatives: Examples of suitable cellulose derivatives are ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP pseudolatex with particles <1 μm. Other components in Aquateric can include pluronics, Tweens, and acetylated monoglycerides. Other suitable cellulose derivatives include cellulose acetate tritnellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (HPMCAS e.g., AQOAT (Shin Etsu)). The performance can vary based on the degree and type of substitution. For example, HPMCP such as, HP-50, HP-55, HP-555, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions.

Poly Vinyl Acetate Phthalate (PVAP): PVAP dissolves in pH>5, and it is much less permeable to water vapor and gastric fluids. Detailed description of above polymers and their pH-dependent solubility can be found at in the article titled “Enteric coated hard gelatin capsules” by Professor Karl Thoma and Karoline Bechtold at http://pop.www.capsugel.com/media/library/enteric-coated-hard-gelatin-capsules.pdf. In some embodiments, the coating can, and usually does, contain a plasticizer and possibly other coating excipients such as colorants, talc, or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually contain 10-25% by weight of a plasticizer, especially dibutyl phthalate, polyethylene glycol, triethyl citrate and triacetin. Conventional coating techniques such as fluid bed or Wurster coaters, or spray or pan coating are employed to apply coatings. The coating thickness must be sufficient to ensure that the oral dosage form remains intact until the desired site of topical delivery in the intestinal tract is reached.

Colorants, surfactants, anti-adhesion agents, antifoaming agents, lubricants (e.g., carnauba wax or PEG) and other additives may be added to the coatings besides plasticizers to solubilize or disperse the coating material, and to improve coating performance and the coated product.

To accelerate the dissolution of the enteric coat, a half-thickness, double coat of enteric polymer (for instance, Eudragit L30 D-55) may be applied, and the inner enteric coat may have a buffer up to pH 6.0 in the presence of 10% citric acid, followed by a final layer of standard Eudragit L 30 D-55. Applying two layers of enteric coat, each half the thickness of a typical enteric coat, Liu and Basit were able to accelerate enteric coating dissolution compared to a similar coating system applied, unbuffered, as a single layer (Liu, F. and Basit, A. Journal of Controlled Release. 147 (2010) 242-245.)

The intactness of the enteric coating may be measured, for example, by the degradation of the drug within the micropellets. The enteric coated dosage forms or pellets may be tested in dissolution testing first in gastric fluid and separately in intestinal fluid as described in USP to determine its function.

The enteric coated tablets and capsules formulation containing the disclosed compounds can be made by methods well known in the art. For example, tablets containing a compound disclosed herein can be enterically coated with a coating solution containing Eudragit®, diethylphthlate, isopropyl alcohol, talc, and water using a side vented coating pan (Freund Hi-Coater).

Alternatively, a multi-unit dosage form comprising enteric-coated pellets that can be incorporated into a tablet or into a capsule can be prepared as follows.

Core material: The core material for the individually enteric coating layered pellets can be constituted according to different principles. Seeds layered with the active agent (i.e., tolebrutinib or a pharmaceutically acceptable sale thereof), optionally mixed with alkaline substances or buffer, can be used as the core material for the further processing. The seeds which are to be layered with the active agent can be water insoluble seeds comprising different oxides, celluloses, organic polymers, and other materials, alone or in mixtures or water-soluble seeds comprising different inorganic salts, sugars, non-pareils and other materials, alone or in mixtures.

Further, the seeds may comprise the active agent in the form of crystals, agglomerates, compacts etc. The size of the seeds is not essential for the present disclosure but may range in size approximately from 0.1 to 2 mm. The seeds layered with the active agent are produced either by powder or solution/suspension layering using for instance granulation or spray coating layering equipment. Before the seeds are layered, active agent may be mixed with further components.

Such components can be binders, surfactants, fillers, disintegrating agents, alkaline additives or other or pharmaceutically acceptable ingredients alone or in mixtures. The binders are for example polymers such as hydroxypropyl methylcellulose (HPMC), hydroxypropyl-cellulose (HPC), carboxymethylcellulose sodium, polyvinyl pyrrolidone (PVP), or sugars, starches, or other pharmaceutically acceptable substances with cohesive properties. Suitable surfactants are found in the groups of pharmaceutically acceptable non-ionic or ionic surfactants such as for instance sodium lauryl sulfate.

Alternatively, the active agent optionally mixed with suitable constituents can be formulated into a core material. Said core material may be produced by extrusion/spheronization, balling or compression utilizing conventional process equipment. The size of the formulated core material is approximately from 0.1 to 4 mm, and for example, from 0.1 to 2 mm. The manufactured core material can further be layered with additional ingredients comprising the active agent or be used for further processing.

The active agent is mixed with pharmaceutical constituents to obtain preferred handling and processing properties and a suitable concentration of the active agent in the final preparation. Pharmaceutical constituents such as fillers, binders, lubricants, disintegrating agents, surfactants, and other pharmaceutically acceptable additives may be used.

Alternatively, the aforementioned core material can be prepared by using spray drying or spray congealing technique.

Enteric Coating Layer(s): Before applying the enteric coating layer(s) onto the core material in the form of individual pellets, the pellets may optionally be covered with one or more separating layer(s) comprising pharmaceutical excipients optionally including alkaline compounds such as pH-buffering compounds. This/these separating layer(s), separate(s) the core material from the outer layers being enteric coating layer(s). This/these separating layer(s) protecting the core material of active agent should be water soluble or rapidly disintegrating in water.

A separating layer(s) can be optionally applied to the core material by coating or layering procedures in suitable equipment such as coating pan, coating granulator or in a fluidized bed apparatus using water or organic solvents for the coating process. As an alternative the separating layer(s) can be applied to the core material by using powder coating technique. The materials for the separating layers are pharmaceutically acceptable compounds such as, for instance, sugar, polyethylene glycol, polyvinylpyrrolidone, polyvinyl alcohol, polyvinyl acetate, hydroxypropyl cellulose, methylcellulose, ethylcellulose, hydroxypropyl methyl cellulose, carboxymethylcellulose sodium, water soluble salts of enteric coating polymers and others, used alone or in mixtures. Additives such as plasticizers, colorants, pigments, fillers anti-tacking and anti-static agents, such as for instance magnesium stearate, titanium dioxide, talc and other additives may also be included into the separating layer(s).

When the optional separating layer is applied to the core material it may constitute a variable thickness. The maximum thickness of the separating layer(s) is normally only limited by processing conditions. The separating layer may serve as a diffusion barrier and may act as a pH-buffering zone. The optionally applied separating layer(s) is not essential for the embodiments of the present disclosure. However, the separating layer(s) may improve the chemical stability of the active substance or the physical properties of the novel multiple unit tableted dosage form.

Alternatively, the separating layer may be formed in situ by a reaction between an enteric coating polymer layer applied on the core material and an alkaline reacting compound in the core material. Thus, the separating layer formed comprises a water-soluble salt formed between the enteric coating layer polymer(s) and an alkaline reacting compound which is in the position to form a salt.

One or more enteric coating layers are applied onto the core material or onto the core material covered with separating layer(s) by using a suitable coating technique. The enteric coating layer material may be dispersed or dissolved in either water or in suitable organic solvents. As enteric coating layer polymers, one or more, separately or in combination, of the following can be used, e.g., solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating polymer(s).

The enteric coating layers contain pharmaceutically acceptable plasticizers to obtain the desired mechanical properties, such as flexibility and hardness of the enteric coating layers. Such plasticizers are for instance, but not restricted to triacetin, citric acid esters, phthalic acid esters, dibutyl sebacate, cetyl alcohol, polyethylene glycols, polysorbates or other plasticizers.

The amount of plasticizer is optimized for each enteric coating layer formula, in relation to the selected enteric coating layer polymer(s), selected plasticizer(s) and the applied amount of said polymer(s), in such a way that the mechanical properties, i.e., flexibility and hardness of the enteric coating layer(s), for instance exemplified as Vickers hardness, are adjusted so that if a tablet is desired the acid resistance of the pellets covered with enteric coating layer(s) does not decrease significantly during compression of pellets into tablets. The amount of plasticizer is usually above 5% by weight of the enteric coating layer polymer(s), such as 15-50% and further such as 20-50%. Additives such as dispersants, colorants, pigments polymers e.g., poly(ethylacrylate, methylmethacrylate), anti-tacking and anti-foaming agents may also be included into the enteric coating layer(s). Other compounds may be added to increase film thickness and to decrease diffusion of acidic gastric juices into the acid susceptible material. The maximum thickness of the applied enteric coating is normally only limited by processing conditions and the desired dissolution profile.

Over-Coating Layer: Pellets covered with enteric coating layer(s) may optionally further be covered with one or more over-coating layer(s). The over-coating layer(s) should be water soluble or rapidly disintegrating in water. The over-coating layer(s) can be applied to the enteric coating layered pellets by coating or layering procedures in suitable equipment such as coating pan, coating granulator or in a fluidized bed apparatus using water or organic solvents for the coating or layering process. The materials for over-coating layers are chosen among pharmaceutically acceptable compounds such as, for instance sugar, polyethylene glycol, polyvinylpyrrolidone, polyvinyl alcohol, polyvinyl acetate, hydroxypropyl cellulose, methylcellulose, ethylcellulose, hydroxypropyl methyl cellulose, carboxymethylcellulose sodium and others, used alone or in mixtures. Additives such as plasticizers, colorants, pigments, fillers, anti-tacking, and anti-static agents, such for instance magnesium stearate, titanium dioxide, talc and other additives may also be included into the over-coating layer(s). The over-coating layer may further prevent potential agglomeration of enteric coating layered pellets, further it may protect the enteric coating layer towards cracking during the compaction process and enhance the tableting process. The maximum thickness of the applied over-coating layer(s) is normally limited by processing conditions and the desired dissolution profile. The over-coating layer may also be used as a tablet film coating layer.

Enteric coating of soft gelatin capsules may contain an emulsion, oil, microemulsion, self-emulsifying system, lipid, triglycerides, polyethylene glycol, surfactants, other solubilizers and the like, and combinations thereof, to solubilize the active agent. The flexibility of the soft gelatin capsule is maintained by residual water and plasticizer. Moreover, for gelatin capsules the gelatin may be dissolved in water so that spraying must be accomplished at a rate with relatively low relative humidity such as can be accomplished in a fluid bed or Wurster. In addition, drying should be accomplished without removing the residual water or plasticizer causing cracking of the capsule shell. Commercially available blends optimized for enteric coating of soft gelatin capsules such as Instamodel EPD (Enteric Polymeric Dispersion), available from Ideal Cures, Pvt. Ltd. (Mumbai, India). On a laboratory scale enteric coated capsules may be prepared by: a) rotating capsules in a flask or dipping capsules in a solution of the gently heated enteric coating material with plasticizer at the lowest possible temperature or b) in a lab scale sprayer/fluid bed and then drying.

For aqueous active agents, it can be especially desirable to incorporate the drug in the water phase of an emulsion. Such “water-in-oil” emulsion provides a suitable biophysical environment for the drug and can provide an oil-water interface that can protect the drug from adverse effects of pH or enzymes that can degrade the drug. Additionally, such water-in-oil formulations can provide a lipid layer, which can interact favorably with lipids in cells of the body, and can increase the partition of the formulation onto the membranes of cells. Such partition can increase the absorption of drugs in such formulations into the circulation and therefore can increase the bioavailability of the drug.

In some embodiments the water-in-oil emulsion contains an oily phase composed of medium or long chain carboxylic acids or esters or alcohols thereof, a surfactant or a surface-active agent, and an aqueous phase containing primarily water and the active agent.

Medium and long chain carboxylic acids are those ranging from C8 to C22 with up to three unsaturated bonds (also branching). Examples of saturated straight chain acids are n-dodecanoic acid, n-tetradecanoic acid, n-hexadecanoic acid, caproic acid, caprylic acid, capric acid, lauric acid, myristic acid, palmitic acid, stearic acid, arachidic acid, behenic acid, montanic acid and melissic acid. Also useful are unsaturated monoolefinic straight chain monocarboxylic acids.

Examples of these are oleic acid, gadoleic acid and erucic acid. Also useful are unsaturated (polyolefinic) straight chain monocarboxylic acids. Examples of these are linoleic acid, ricinoleic acid, linolenic acid, arachidonic acid and behenolic acid. Useful branched acids include, for example, diacetyl tartaric acid. Unsaturated olefinic chains may also be hydroxylated or ethoxylated to prevent oxidation or to alter the surface properties.

Examples of long chain carboxylic acid esters include, but are not limited to, those from the group of: glyceryl monostearates; glyceryl monopalmitates; mixtures of glyceryl monostearate and glyceryl monopalmitate; glyceryl monolinoleate; glyceryl monooleate; mixtures of glyceryl monopalmitate, glyceryl monostearate, glyceryl monooleate and glyceryl monolinoleate; glyceryl monolinolenate; glyceryl monogadoleate; mixtures of glyceryl monopalmitate, glyceryl monostearate, glyceryl monooleate, glyceryl monolinoleate, glyceryl monolinolenate and glyceryl monogadoleate; acetylated glycerides such as distilled acetylated monoglycerides; mixtures of propylene glycol monoesters, distilled monoglycerides, sodium steroyl lactylate and silicon dioxide; d-alpha tocopherol polyethylene glycol 1000 succinate; mixtures of mono- and di-glyceride esters such as Atmul; calcium stearoyl lactylate; ethoxylated mono- and di-glycerides; lactated mono- and di-glycerides; lactylate carboxylic acid ester of glycerol and propylene glycol; lactylic esters of long chain carboxylic acids; polyglycerol esters of long chain carboxylic acids, propylene glycol mono- and di-esters of long chain carboxylic acids; sodium stearoyl lactylate; sorbitan monostearate; sorbitan monooleate; other sorbitan esters of long chain carboxylic acids; succinylated monoglycerides; stearyl monoglyceryl citrate; stearyl heptanoate; cetyl esters of waxes; stearyl octanoate; C8-C30 cholesterol/lavosterol esters; and sucrose long chain carboxylic acid esters.

Examples of the self-emulsifying long chain carboxylic acid esters include those from the groups of stearates, palmitates, ricinoleates, oleates, behenates, ricinolenates, myristates, laurates, caprylates, and caproates. In some embodiments the oily phase may comprise a combination of 2 or more of the long chain carboxylic acids or esters or alcohols thereof. In some embodiments medium chain surfactants may be used and the oil phase may comprise a mixture of caprylic/capric triglyceride and C8/C10 mono-/di-glycerides of caprylic acid, glyceryl caprylate or propylene glycol monocaprylate or their mixtures.

The alcohols that can be used are exemplified by the hydroxyl forms of the carboxylic acids exemplified above and also stearyl alcohol.

Surface active agents or surfactants are long chain molecules that can accumulate at hydrophilic/hydrophobic (water/oil) interfaces and lower the surface tension at the interface. As a result, they can stabilize an emulsion. In some embodiments, the surfactant may comprise: Tween® (polyoxyethylene sorbate) family of surfactants, Span® (sorbitan long chain carboxylic acid esters) family of surfactants, Pluronic® (ethylene or propylene oxide block copolymers) family of surfactants, Labrasol®, Labrafil® and Labrafac®(each polyglycolyzed glycerides) families of surfactants, sorbitan esters of oleate, stearate, laurate or other long chain carboxylic acids, poloxamers (polyethylene-polypropylene glycol block copolymers or Pluronic®.), other sorbitan or sucrose long chain carboxylic acid esters, mono and diglycerides, PEG derivatives of caprylic/capric triglycerides and mixtures thereof or mixture of two or more of the above. In some embodiments the surfactant phase may comprise a mixture of Polyoxyethylene (20) sorbitan monooleate (Tween 80®) and sorbitan monooleate (Span 80®).

The aqueous phase may optionally comprise the active agent suspended in water and a buffer.

In some embodiments, such emulsions are coarse emulsions, microemulsions and liquid crystal emulsions. In other embodiments such emulsion may optionally comprise a permeation enhancer. In other embodiments, spray-dried dispersions or microparticles or nanoparticles containing encapsulated microemulsion, coarse emulsion or liquid crystal can be used.

In some embodiments, the solid dosage forms described herein are non-enteric time-delayed release dosage forms. The term “non-enteric time-delayed release” as used herein refers to the delivery so that the release of the drug can be accomplished at some generally predictable location in the intestinal tract more distal to that which would have been accomplished if there had been no delayed release alterations. In some embodiments the method for delay of release is a coating that becomes permeable, dissolves, ruptures, or is no longer intact after a designed duration. The coating in the time-delayed release dosage forms can have a fixed time to erode after which the drug is released (suitable coating include polymeric coating such as HPMC, PEO, and the like) or has a core comprised of a superdisintegrant(s) or osmotic agent(s) or water attractant such as a salt, hydrophilic polymer, typically polyethylene oxide or an alkylcellulose, salts such as sodium chloride, magnesium chloride, sodium acetate, sodium citrate, sugar, such as glucose, lactose, or sucrose, or the like, which draw water through a semi-permeable membrane or a gas generating agent such as citric acid and sodium bicarbonate with or without an acid such as citric acid or any of the aforementioned acids incorporated in dosage forms. The semi-permeable membrane, while mostly not permeable to the drug nor the osmotic agent, is permeable to water that permeates at a near constant rate to enter the dosage form to increase the pressure and ruptures after the swelling pressure exceeds a certain threshold over a desired delay time. The permeability through this membrane of the drug should be less than 1/10 than water and in one embodiment less than 1/100 the water permeability. Alternatively, a membrane could become porous by leaching an aqueous extractable over a desired delay time.

Osmotic dosage forms have been described in Theeuwes U.S. Pat. No. 3,760,984, and an osmotic bursting dosage form is described in Baker U.S. Pat. No. 3,952,741. This osmotic bursting dosage form can provide a single pulse of release or multiple pulses if different devices with different timings are employed. The timing of the osmotic burst may be controlled by the choice of polymer and the thickness or the area of the semipermeable membrane surrounding the core that contains both the drug and the osmotic agent or attractant. As the pressure in the dosage form increase with additional permeated water, the membrane elongates until its breaking point, and then the drug is released. Alternatively, specific areas of rupture can be created in the membrane by having a thinner, weaker area in the membrane or by adding a weaker material to an area of the coating membrane. Some preferred polymers with high water permeabilities that may be used as semipermeable membranes are cellulose acetate, cellulose acetate butyrate, cellulose nitrate, crosslinked polyvinyl, alcohol, polyurethanes, nylon 6, nylon 6.6, and aromatic nylon. Cellulose acetate is an especially preferred polymer.

In another embodiment, the time-delayed coating that begins its delay to releasing drug after the enteric coating is at least partially dissolved is comprised of hydrophilic, erodible polymers that upon contact with water begin to gradually erode over time. Examples of such polymers include cellulose polymers and their derivatives including, but not limited to, hydroxyalkyl celluloses, hydroxymethyl cellulose, hydroxyethyl cellulose, hydroxypropyl cellulose, hydroxypropylmethyl cellulose, carboxymethylcellulose, microcrystalline cellulose; polysaccharides and their derivatives; polyalkylene oxides, such as polyethylene oxide or polyethylene glycols, particularly high molecular weight polyethylene glycols; chitosan; poly(vinyl alcohol); xanthan gum; maleic anhydride copolymers; poly(vinyl pyrrolidone); starch and starch-based polymers; maltodextrins; poly (2-ethyl-2-oxazoline); poly(ethyleneimine); polyurethane; hydrogels; crosslinked polyacrylic acids; and combinations or blends of any of the foregoing.

Some preferred erodible hydrophilic polymers suitable for forming the erodible coating are poly(ethylene oxide), hydroxypropyl methyl cellulose, and combinations of poly(ethylene oxide) and hydroxypropyl methyl cellulose. Poly(ethylene oxide) is used herein to refer to a linear polymer of unsubstituted ethylene oxide. The molecular weight of the poly(ethylene oxide) polymers can range from about 10⁵ Daltons to about 10⁷ Daltons. A preferred molecular weight range of poly(ethylene oxide) polymers is from about 2×10⁵ to 2×10⁶ Daltons and is commercially available from The Dow Chemical Company (Midland, Mich.) referred to as SENTRYR POLYOX™ water-soluble resins, NF (National Formulary) grade. When higher molecular weights of polyethylene oxide are used, other hydrophilic agents, such as salts or sugars, like glucose, sucrose, or lactose, that promote erosion or disintegration of this coating, are also included.

The time-delayed dosage form can be a mechanical pill such as an Enterion® capsule or pH sensitive capsule which can release the drug after a pre-programmed time or when it receives a signal which can be transmitted or once it leaves the stomach.

The amount of the compound of the disclosure in a formulation can vary within the full range employed by those skilled in the art. Typically, the formulation will contain, on a weight percent (wt %) basis, from about 0.01-99.99 wt % of the BTK inhibitor compound based on the total formulation, with the balance being one or more suitable pharmaceutical excipients. In one embodiment, the compound is present at a level of about 1-80 wt %.

The foregoing disclosure has been described in some detail by way of illustration and example, for purposes of clarity and understanding. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled. Additionally, the attached examples provide exemplary study protocols showing how a clinical study may be implemented.

### EXAMPLES

The following examples are provided to illustrate certain disclosed embodiments and are not to be construed as limiting the scope of this disclosure in any way. In the Examples discussed below, the BTK inhibitor, as defined above, may be also referred as “the compound” or “the drug” interchangeably.

### Example 1—A Phase 3, Randomized, Double-Blind, Efficacy and Safety Study Comparing Tolebrutinib to Placebo in Participants with Nonrelapsing Secondary Progressive Multiple Sclerosis (HERCULES)

Overall design: This is a Phase 3, randomized, double-blind, 2-arm, placebo-controlled, parallel group, multicenter, event-driven (6-month CDP) trial with a variable treatment duration ranging from approximately 24 to 48 months in participants with nrSPMS. A graphical scheme of the study design is shown in FIG. TA. Abbreviations used in FIG. TA: CDP, confirmed disability progression; EOS, end of study; MRI, magnetic resonance imaging; R, randomization. “Month-1 (D-28-D-1)” refers to screening period as “Day-28 to Day-1”; “Month 0 (D1)” refers to randomization on Day 1.

Table 1A shown below describe the schedule of activities during the course of study. Table 1B that follows describes the objective and endpoints of the overall study.

TABLE 1A
Schedule of Activities (SOA)

| | Procedure | | | | | | | | | | | |
|---|---|---|---|---|---|---|---|---|---|---|---|---|
| | | Randomization/ | | | | | | | | | | |
| | Screening^{a} | start of IMP | To Year 2 (M 24)^{b} | | | | | | | | | |
| | Visit (a window of ±7 days is allowed for all visits after D 1) | | | | | | | | | | | |
| | | | | | M 1.25 (W 5) | | M 2.25 (W 9) | | | | | |
| | D −28 | | M 0.5 (W 2) | | M 1.5 (W 6) | | M 2.5 (W 10) | | | | | |
| | to D −1 | D 1 | M 0.75 (W 3) | M 1(W 4)^{d} | M 1.75 (W 7) | M 2(W 8)^{d} | M 2.75 (W 11) | M 3 | M 4, M 5^{d} | M 6 | M 7 M 8 | M 9 |
| | Visit Number | | | | | | | | | | | |
| | V1 | V2 | | V3 | | V4 | | V5 | V6, V7 | V8 | | V9 |
| Informed | X | | | | | | | | | | | |
| consent | | | | | | | | | | | | |
| Demography | X | | | | | | | | | | | |
| Inclusion/ | X | X | | | | | | | | | | |
| exclusion criteria | | | | | | | | | | | | |
| Medical/surgical | X | | | | | | | | | | | |
| history | | | | | | | | | | | | |
| Prior/concomitant | <====================================================================================================> | | | | | | | | | | | |
| medications^{g} | | | | | | | | | | | | |
| Randomization | | X | | | | | | | | | | |
| IRT contact | X | X | | | | | | X | | X | | X |

Study treatment administration

| | | | | |
|---|---|---|---|---|
| IMP | X | X | X | X |
| dispensation | | | | |
| IMP Compliance | | X | X | X |
| Paper diary | X | X | X | X |
| dispensation/ | | | | |
| collection | | | | |

Safety^{y}

| Physical | X | X | | | | | | X | | X | | X |
|---|---|---|---|---|---|---|---|---|---|---|---|---|
| examination^{h} | | | | | | | | | | | | |
| and vital signs | | | | | | | | | | | | |
| Height | X | | | | | | | | | | | |
| Body weight | X | X | | | | | | X | | X | | |
| Serology tests | X | | | | | | | | | | | |
| for hepatitis B | | | | | | | | | | | | |
| and C | | | | | | | | | | | | |
| HIV and other | X | | | | | | | | | | | |
| infectious | | | | | | | | | | | | |
| diseases, if | | | | | | | | | | | | |
| required locally | | | | | | | | | | | | |
| TB/QuantiFERON ® | X | | | | | | | | | | | |
| TB Gold | | | | | | | | | | | | |
| test or | | | | | | | | | | | | |
| equivalentⁱ | | | | | | | | | | | | |
| Body | X | X | | | | | | X | | X | | X |
| temperature | | | | | | | | | | | | |
| 12-lead ECG^{j} | X | | | | | | | X | | X | | X |
| Hematology, | X | X^{z} | | X | | X | | X | X | X | | X |
| biochemistry^{k} | | | | | | | | | | | | |
| Liver function | | | X | | X | | X | | | | X | |
| tests^{l} | | | | | | | | | | | | |
| Iron panel | X | | | | | | | | | | | |
| (serum): iron, | | | | | | | | | | | | |
| ferritin, transferrin | | | | | | | | | | | | |
| saturation, TIBC; | | | | | | | | | | | | |
| to be repeated | | | | | | | | | | | | |
| during the study if | | | | | | | | | | | | |
| needed | | | | | | | | | | | | |
| Coagulation: | X | | | | | | | | | | | |
| PT/INR, aPTT | | | | | | | | | | | | |
| (to be repeated | | | | | | | | | | | | |
| during the study, | | | | | | | | | | | | |
| if needed) | | | | | | | | | | | | |
| Urinalysis | X | | | | | | | X | | | | |
| Pregnancy test | X | X | | | | | | X | | X | | X |
| (if applicable)^{m} | | | | | | | | | | | | |
| Serum FSHⁿ | X | | | | | | | | | | | |
| Suicidality | X | X | | | | | | X | | X | | X |
| assessment by | | | | | | | | | | | | |
| C-SSRS | | | | | | | | | | | | |
| Adverse event | <====================================================================================================> | | | | | | | | | | | |
| collection | | | | | | | | | | | | |

Efficacy

| EDSS | X | X | X | X | X |
|---|---|---|---|---|---|
| Timed 25-foot | | X | X | X | X |
| walk test | | | | | |
| 9-hole peg test | | X | X | X | X |
| SDMT and | | X | X | X | X |
| CVLT-II, where | | | | | |
| available^{o} | | | | | |
| Basic or | X^{q} | | | X | |
| expanded MRI^{p} | | | | | |
| Actigraphy | X | | | | |
| (optional for | | | | | |
| subset of | | | | | |
| participants)^{r} | | | | | |

Clinical outcome assessments^{s}

| | | |
|---|---|---|
| MSQoL-54 | X | X |
| EQ-5D-5L | X | X |

Pharmacokinetics^{s}

| | | |
|---|---|---|
| Tolebrutinib and | X^{u} | X^{u} |
| relevant | | |
| metabolite(s) | | |
| pharmacokinetic | | |
| plasma samples^{t} | | |

Pharmacogenetics^{s}

| | |
|---|---|
| DNA sample | X |
| (optional)^{w} | |

Pharmacodynamics/biomarkers^{s}

| | | | | |
|---|---|---|---|---|
| Blood sample for | X | | | |
| archiving^{x} | | | | |
| Immunophenoty | | X | X | |
| ping/RN A | | | | |
| sequencing | | | | |
| (ToleDYNAMIC/ | | | | |
| optional | | | | |
| substudy at | | | | |
| selected sites)^{aa} | | | | |
| Lymphocyte | | X | | |
| phenotyping by | | | | |
| flow cytometry | | | | |
| in whole blood | | | | |
| (subset of | | | | |
| participants)^{bb} | | | | |
| Plasma samples | | X | X | X |
| (NfL), serum | | | | |
| samples | | | | |
| (Chi3L1)^{v} | | | | |
| Serum samples | | X | | X |
| (Ig levels)^{v} | | | | |

| | Procedure | | | | | | | | | | |
|---|---|---|---|---|---|---|---|---|---|---|---|
| | | | | | | | | | | | Only for |
| | | | | | | | | | Only for | | participants who |
| | | | | | | | | | participants who | | completed treatment |
| | | | | | | | | | prematurely | For all | to EOS but do not |
| | To Year 2 (M 24)^{b} | | | | | | From M 27 to EOS^{b} | | discontinue IMP | participants | enter LTS^{c} |
| | Visit (a window of ±7 days is allowed for all visits after D 1) | | | | | | | | | | |
| | | | | | | | Quarterly visits | Semi-annual | | EOS | |
| | | | | | | | (M 27, M 30, M 33, | visits | | “Common | Follow-up |
| | | | | | | | M 36, M 39, M 42, | (M 30, M 36, | | study end | visit |
| | M 10 M 11 | M 12 | M 15 | M 18 | M 21 | M 24 | M 45, M 48 . . .) | M 42, M48 . . .) | pEOT^{e} | date” visit^{f} | (4 to 8 weeks) |
| | Visit Number | | | | | | | | | | |
| | | | | | | | V15, V16, V17, | | | | |
| | | | | | | | V18, V19, V20, | V16, V18, | | | |
| | | V10 | V11 | V12 | V13 | V14 | V21, V22 | V20, V22 | pEOT^{e} | EOS | FUV |
| Informed | | | | | | | | | | | |
| consent | | | | | | | | | | | |
| Demography | | | | | | | | | | | |
| Inclusion/ | | | | | | | | | | | |
| exclusion criteria | | | | | | | | | | | |
| Medical/surgical | | | | | | | | | | | |
| history | | | | | | | | | | | |
| Prior/concomitant | <====================================================================================================> | | | | | | | | | | |
| medications^{g} | | | | | | | | | | | |
| Randomization | | | | | | | | | | | |
| IRT contact | | X | X | X | X | X | X | X | X | X | X |
| Study treatment administration | | | | | | | | | | | |
| IMP | | X | X | X | X | X | X | X | | | |
| dispensation | | | | | | | | | | | |
| IMP Compliance | | X | X | X | X | X | X | X | X^{e} | X | |
| Paper diary | | X | X | X | X | X | X | X | X | X | |
| dispensation/ | | | | | | | | | | | |
| collection | | | | | | | | | | | |
| Safety^{y} | | | | | | | | | | | |
| Physical | | X | X | X | X | X | X | X | X | X | X |
| examination^{h} | | | | | | | | | | | |
| and vital signs | | | | | | | | | | | |
| Height | | | | | | | | | | | |
| Body weight | | X | | X | | X | | X | X | X | X |
| Serology tests | | | | | | | | | | | |
| for hepatitis B | | | | | | | | | | | |
| and C | | | | | | | | | | | |
| HIV and other | | | | | | | | | | | |
| infectious | | | | | | | | | | | |
| diseases, if | | | | | | | | | | | |
| required locally | | | | | | | | | | | |
| TB/QuantiFERON ® | | | | | | | | | | | |
| TB Gold | | | | | | | | | | | |
| test or | | | | | | | | | | | |
| equivalentⁱ | | | | | | | | | | | |
| Body | | X | X | X | X | X | X | X | X | X | X |
| temperature | | | | | | | | | | | |
| 12-lead ECG^{j} | | X | | | | X | | yearly | X | X | |
| Hematology, | | X | X | X | X | X | X | X | X | X | X |
| biochemistry^{k} | | | | | | | | | | | |
| Liver function | X | | | | | | | | | | |
| tests^{l} | | | | | | | | | | | |
| Iron panel | | | | | | | | | | | |
| (serum): iron, | | | | | | | | | | | |
| ferritin, transferrin | | | | | | | | | | | |
| saturation, TIBC; | | | | | | | | | | | |
| to be repeated | | | | | | | | | | | |
| during the study if | | | | | | | | | | | |
| needed | | | | | | | | | | | |
| Coagulation: | | | | | | | | | | | |
| PT/INR, aPTT | | | | | | | | | | | |
| (to be repeated | | | | | | | | | | | |
| during the study, | | | | | | | | | | | |
| if needed) | | | | | | | | | | | |
| Urinalysis | | X | | X | | X | | X | X | X | |
| Pregnancy test | | X | X | X | X | X | X | X | X | X | X |
| (if applicable)^{m} | | | | | | | | | | | |
| Serum FSHⁿ | | | | | | | | | | | |
| Suicidality | | X | X | X | X | X | X | X | X | X | X |
| assessment by | | | | | | | | | | | |
| C-SSRS | | | | | | | | | | | |
| Adverse event | <====================================================================================================> | | | | | | | | | | |
| collection | | | | | | | | | | | |
| Efficacy | | | | | | | | | | | |
| EDSS | | X | X | X | X | X | X | X | X | X | |
| Timed 25-foot | | X | X | X | X | X | X | X | X | X | |
| walk test | | | | | | | | | | | |
| 9-hole peg test | | X | X | X | X | X | X | X | X | X | |
| SDMT and | | X | X | X | X | X | X | X | X | X | |
| CVLT-II, where | | | | | | | | | | | |
| available^{o} | | | | | | | | | | | |
| Basic or | | X | | X | | X | | yearly | X | X | |
| expanded MRI^{p} | | | | | | | | | | | |
| Actigraphy | X | | | | | | | | | | |
| (optional for | | | | | | | | | | | |
| subset of | | | | | | | | | | | |
| participants)^{r} | | | | | | | | | | | |
| Clinical outcome assessments^{s} | | | | | | | | | | | |
| MSQoL-54 | | X | | X | | X | | X | X | X | |
| EQ-5D-5L | | X | | X | | X | | X | X | X | |
| Pharmacokinetics^{s} | | | | | | | | | | | |
| Tolebrutinib and | | X^{u} | | | | | | | X^{e} | | |
| relevant | | | | | | | | | | | |
| metabolite(s) | | | | | | | | | | | |
| pharmacokinetic | | | | | | | | | | | |
| plasma samples^{t} | | | | | | | | | | | |
| Pharmacogenetics^{s} | | | | | | | | | | | |
| DNA sample | | | | | | | | | | | |
| (optional)^{w} | | | | | | | | | | | |
| Pharmacodynamics/biomarkers^{s} | | | | | | | | | | | |
| Blood sample for | | | | | | | | | | | |
| archiving^{x} | | | | | | | | | | | |
| Immunophenoty | | X | | | | | | | | | |
| ping/RN A | | | | | | | | | | | |
| sequencing | | | | | | | | | | | |
| (ToleDYNAMIC/ | | | | | | | | | | | |
| optional | | | | | | | | | | | |
| substudy at | | | | | | | | | | | |
| selected sites)^{aa} | | | | | | | | | | | |
| Lymphocyte | | | | | | | | | X | X | |
| phenotyping by | | | | | | | | | | | |
| flow cytometry | | | | | | | | | | | |
| in whole blood | | | | | | | | | | | |
| (subset of | | | | | | | | | | | |
| participants)^{bb} | | | | | | | | | | | |
| Plasma samples | | X | | | | X | | yearly | X | X | |
| (NfL), serum | | | | | | | | | | | |
| samples | | | | | | | | | | | |
| (Chi3L1)^{v} | | | | | | | | | | | |
| Serum samples | | X | | | | X | | yearly | X | X | |
| (Ig levels)^{v} | | | | | | | | | | | |

aPTT, activated partial thromboplastin time; β-HCG, β-human chorionic gonadotropin; ALP, alkaline phosphatase; ALT, alanine aminotransferase; AST, aspartate aminotransferase; Chi3L1, chitinase-3-like protein 1; C-SSRS, Columbia Suicide Severity Rating Scale; D, day; DNA, deoxyribonucleic acid; ECG, electrocardiogram; EDSS, Expanded Disability Status Scale; EOS, end of study; EOT, end of treatment; EQ-5D-5L, EuroQol 5-dimension 5-level instrument; FSH, follicle stimulation hormone; FUV, follow-up visit; ICF, informed consent form; Ig, immunoglobulin; IRT, interactive response technology; HIV, human immunodeficiency virus; IMP, investigational medicinal product; INR, international normalized ratio; LTS, long term safety study; M, month; MCV, mean corpuscular volume; MCH, mean corpuscular hemoglobin; MRI: magnetic resonance imaging; MS: multiple sclerosis; SDMT, Symbol Digit Modalities Test; CVLT-II, California Verbal Learning Test-II; MSQoL-54: Multiple Sclerosis Quality of Life-54; NfL: neurofilament light chain; pEOT: premature end of treatment; PK: pharmacokinetic; PT: prothrombin time; RBC, red blood cell; SWI, susceptibility weighted imaging; TB, tuberculosis; TIBC: total iron-binding capacity; V: visit; WBC, white blood cell.
Note:
All assessments should be done as designated in this SoA unless not permitted according to local regulations. All visit assessments should be performed during the visit window unless otherwise specified in this protocol.
^{a}Screening period can range from D −28 to D −1; Randomization visit can be performed only once IMPs are available at site. The interval between screening and randomization visits can range from 11 days (minimum) to 28 days (maximum). However, if required, the randomization visit can be performed earlier than 11 days upon IMP receipt at the site, assuming the participant is eligible for randomization. In case of any delay to screening (MRI rescheduling, lab retests, etc), an additional period of up to 2 weeks is allowed.
^{b}From D 1 to EOS, unscheduled visits may be performed at any time by the Investigator (eg, for evaluation of an adverse event). Assessments may be done on as needed basis to evaluate the participant in accordance with the Investigator's best judgement and in-line with the study protocol. At a minimum, a physical examination should be performed, and body temperature and vital signs should be measured.
^{c}At the EOS, the participants who have completed treatment with IMP (double blind or open label if meeting CDP) may be offered participation in LTS study. Follow-up visit assessment only performed for those participants who completed treatment and are not willing to take part in the LTS study. For other situations where a follow-up visit is needed, please see Section 7.1 and the Study Manual.
^{d}These visits may be done as home health visits (where applicable) or onsite visits (it is preferable that tests are performed at the central laboratory). In any situations where this is not possible (to be documented in source documents), the tests for these visits may be performed at a local laboratory.
^{e}If a participant prematurely permanently discontinues treatment with IMP, the participant will undergo pEOT visit as soon as possible. A PK sample should also be collected if the pEOT visit can be scheduled within a maximum 24 hours after the last IMP dose. Participants will then be asked to continue with the study visits as scheduled until the common EOS Visit is reached. During these visits, all study procedures/assessments will be performed except IMP administration and blood sampling for PK and biomarkers (NfL, Chi3L1, and Ig levels). MRI scans for these participants will only be performed annually (using the next annual visit as the starting point). Additional information is provided in the Study Manual.
^{f}For participants continuing in the study, the common EOS visit will be done when the prespecified number of events for 6-month CDP is expected to be reached. The timing and window of this visit will be communicated to sites.
^{g}Any disease-modifying therapy for MS taken at any time prior to signing the informed consent needs to be reported in the eCRF; other prior medications will be reported for the period of 6 months prior to signing the ICF.
^{h}Complete physical examination due at screening, baseline, yearly (M 12, M 24, M 36, M 48) and at EOS; brief physical examination is sufficient for the rest of the visits (complete and brief physical examinations will include neurological examination and collection of the following vital signs: arterial blood pressure, heart rate, temperature).
ⁱTo be performed at screening for all participants. Tuberculosis examination will be repeated based on clinical judgment, borderline results, or clinical suspicion of TB infection. For further details, refer to E 01.
^{j}ECG and 30 second rhythm strips will be obtained locally.
^{k}Hematology (platelet count, RBC count, hemoglobin, hematocrit, MCV, MCH, reticulocytes, WBC count with differential: neutrophils, lymphocytes, monocytes, eosinophils, basophils).
Biochemistry (blood urea nitrogen [BUN], creatinine, glucose, sodium, potassium, bicarbonate, calcium, liver function tests [AST, ALT, ALP, albumin, total and direct bilirubin], total protein; creatine phosphokinase. Lipase will be tested at the Screening Visit, then quarterly. Monthly visits (M 1, M 2, M 4, and M 5) will include hematology and full liver panel only. Additional safety assessments can be performed if required by local regulations. Such testing shall be performed at local laboratories.
Note:
a one-time retest at screening may be performed if laboratory test abnormality is considered temporary. Additional safety assessments can be performed if required by local regulations; such testing shall be performed locally whenever possible. Additional visits may be added if required by local regulations.
^{l}At intermediate timepoints (W 2, W 3, W 5, W 6, W 7, W 9, W 10, W 11, M 7, M 8, M 10, and M 11), only liver function tests will be collected (AST, ALT, albumin, alkaline phosphatase, total and direct bilirubin, total protein) and creatine phosphokinase); these can be performed at central laboratory (preferred, as on-site visits or home nursing as applicable for the site) or at local laboratory; a window of ±3 days is permitted for weekly liver function test (LFT) timepoints and ±7 days for the other timepoints. Additional information is provided in the Study Manual. For participants switching to open-label treatment, please refer to Section 6.6.1.
^{m}At screening, perform the serum β-hCG pregnancy test at the central laboratory. At randomization and other scheduled visits during the study, urine pregnancy tests should be performed. At randomization, a pregnancy test should be performed prior to the first dose of IMP. Additional serum or urine pregnancy tests may be performed, as determined necessary by the Investigator or required by local regulation, to establish the absence of pregnancy at any time during the participant's participation in the study. Communication by phone of the result of a pregnancy test performed at home to the site is allowed.
ⁿOnly in female participants, if needed to establish menopausal status.
^{o}The SDMT and CVLT-II will be performed in all participants. If for some reason, CVLT-II is not available at a given site due to reasons such as lack of translation, local certification, etc., then only the SDMT will be assessed for that participant.
^{p}A subset of sites that have 3T MRI capacity will perform additional sequences (eg, SWI). Further details will be defined in a central MRI manual.
^{q}A visit window of ±21 days is acceptable for MRIs performed after D 1. For systemic corticosteroids and adrenocorticotropic hormone, 1-month wash-out required prior to the MRI scans. The screening MRI scan should be performed as close as possible before the start of IMP. As much as possible, the MRI scan should be performed during the screening period only after it has been established that the participant meets all inclusion and no exclusion criteria.
^{r}A noninvasive activity monitor (actigraphy) may be optionally implemented by the Sponsor in a subset of participants during the course of the study if results from pilot assessment demonstrate feasibility.
^{s}When available, clinical outcome assessments are to be completed by the participant prior to discussing their health status and prior to study treatment administration or other study related procedures where available per local regulations.
^{t}On days of PK sampling, the IMP needs to be taken at the study site after a “regular meal”. In case a participant forgot IMP at home or took IMP prior to arriving at the site on the day of visits with PK sampling, he/she will be asked to have a repeat assessment within 3 days of the missed PK sampling. A PK assessment shall be done as soon as possible after an overdose or if otherwise specified per protocol
^{u}(eg, investigation of abnormal laboratory test values).
^{v}M 6 and M 12: Two samples: one sample between 30 to 90 minutes and one sample between 2.5 to 5 hours after IMP administration. M 9: one sample 30-90 minutes after IMP.
^{w}Pharmacodynamics and biomarkers samples will be collected only if permitted per local regulations.
^{x}DNA testing will be allowed at any time after signature of consent (in case it could not be done for some reason at Day 1). Participation is optional. Participants who do not wish to participate in the genetic research may still participate in the study. A separate consent is required for the genetic analysis component of the study.
^{y}This sample will be collected and stored for use if any unexpected safety issue occurs to ensure that a pre-dose baseline value is available for previously not assessed parameters (eg, serology) and for biomarkers research, if agreed.
^{z}Additional safety assessments can be performed if required by local regulations; such testing shall be performed locally whenever possible. Additional visits may be added if required by local regulations. Samples for hematology and biochemistry tests on Day 1 (randomization) must be collected prior to administration of the first IMP dose.
^{aa}Samples must be shipped within 24 hours.
^{bb}Blood sample collection for lymphocyte phenotyping by flow cytometry will be performed in a subset of randomized participants. Participants who did not have a baseline sample collected, will no longer have this test performed; participants who had a baseline sample collected will have a second sample collected at EOT/pEOT.

Objectives and endpoints for the treatment are shown in Table 1B.

TABLE 1B
Objectives and endpoints

| Objectives | Endpoints | |
|---|---|---|
| Primary | | |
| To determine the efficacy of tolebrutinib | Time to onset of 6-month CDP defined as | |
| compared to placebo in delaying disability | follows: | |
| progression in nrSPMS | - | Increase of ≥1.0 point from the |
| | | baseline EDSS score when the |
| | | baseline score is ≤5.0, OR |
| | - | Increase of ≥0.5 points when the |
| | | baseline EDSS score is >5.0 |
| Secondary | | |
| To evaluate efficacy of tolebrutinib | Time to onset of sustained 20% | |
| compared to placebo on clinical endpoints, | increase in the 9-hole peg test (9-HPT) | |
| MRI lesions, cognitive performance, | confirmed over at least 3 months | |
| physical function, and quality of life | Time to onset of sustained 20% | |
| | increase in the timed 25-foot walk | |
| | (T25-FW) test confirmed over at least 3 | |
| | months | |
| | Time to onset of 3-month CDP as | |
| | assessed by the EDSS score | |
| | Total number of new and/or enlarging | |
| | T2-hyperintense lesions as detected by | |
| | MRI, defined as the sum of the | |
| | individual number of new and/or | |
| | enlarging T2 lesions at all scheduled | |
| | visits starting after baseline up to and | |
| | including the end of study (EOS) visit | |
| | Time to onset of CDI defined as ≥1.0 | |
| | point decrease on the EDSS score from | |
| | baseline confirmed over at least 6 | |
| | months | |
| | Percent change in brain volume loss | |
| | (BVL) as detected by brain MRI scans | |
| | at EOS compared to Month 6 | |
| | Change in cognitive function at the | |
| | EOS compared to baseline as assessed | |
| | by SDMT | |
| | Change in cognitive function at EOS | |
| | compared to baseline as assessed by | |
| | CVLT-II, where available | |
| | Change in MSQoL-54 questionnaire | |
| | score from baseline through the EOS | |
| To evaluate safety and tolerability of | Adverse events (AEs), serious AEs, | |
| tolebrutinib | AEs leading to permanent study | |
| | intervention discontinuation, AEs of | |
| | special interest, and potentially | |
| | clinically significant abnormalities in | |
| | laboratory tests, safety scales, ECG, | |
| | and vital signs during the study period | |
| To evaluate population PK of tolebrutinib | Plasma concentration of tolebrutinib | |
| and relevant metabolite(s) in nrSPMS and | (population PK assessment) at Months | |
| its relationship to efficacy and safety | 6, 9, and 12 | |
| To evaluate pharmacodynamics of | Change in plasma NfL levels at the | |
| tolebrutinib | EOS compared to baseline | |
| | Change in lymphocyte phenotype | |
| | subsets in whole blood at the | |
| | EOS compared to baseline in a subset | |
| | of participants | |
| | Change in serum immunoglobulin level | |
| | at the EOS compared to baseline | |
| | Change in serum Chi3L1 levels at the | |
| | EOS compared to baseline | |
| Tertiary/exploratory | | |
| To evaluate efficacy of tolebrutinib on | Time to onset of sustained 20% increase | |
| disease progression and activity in | in the 9-HPT for at least 6 months | |
| nrSPMS, assessed by other clinical and | Time to onset of sustained 20% increase | |
| imaging measures and by self reported | in the T25-FW for at least 6 months | |
| assessment | Time to onset of a 4-point decrease in the | |
| | SDMT, confirmed over at least 3 and 6 | |
| | months | |
| | The proportion of participants with CDI | |
| | confirmed over at least 6 months | |
| | The proportion of participants with CDI | |
| | confirmed over at least 6 months and | |
| | maintained until the EOS | |
| | Change from baseline to Months 12, 18, | |
| | and 24 and to the EOS in the EDSS score, | |
| | T25-FW test, 9-HPT, SDMT, and CVLT- | |
| | II | |
| | Change from baseline to Months 12, 18, | |
| | and 24 and to the EOS in modified | |
| | MSFC-3, assessed as the composite of the | |
| | T25-FW test, 9-HPT, and SDMT | |
| | Proportion of participants with NEDA-3 | |
| | at Months 18, 24, 30, 36, and the EOS | |
| | The annualized adjudicated relapse rate | |
| | (ARR) | |
| | Actigraphic analysis of activity counts and | |
| | indices of change from baseline to the | |
| | EOS summarized over time (in a subset of | |
| | participants) | |
| | Change from baseline of total volume of | |
| | T2-hyperintense lesions as detected by | |
| | brain MRI at Months 18, 24, and the EOS | |
| | Total number of new Gd-enhancing T1 | |
| | hyperintense lesions as detected by MRI, | |
| | defined as the sum of the individual | |
| | number of new Gd-enhancing T1- | |
| | hyperintense lesions at all scheduled visits | |
| | starting after baseline up to and including | |
| | the EOS visit | |
| | Change from baseline by visit in the | |
| | volume of T1-hypointense lesions and | |
| | cumulative number of new T1 | |
| | hypointense lesions | |
| | MTR recovery at EOS in new MTR | |
| | lesions detected at months 6 and 12 | |
| | Change in number of phase rim lesions in | |
| | SWI MRI from baseline through the EOS | |
| | (subset of centers with capacity of 3T | |
| | MRI) | |
| | Number and volume of slowly evolving | |
| | lesions (SELs) Normalized T1 (nT1) | |
| | intensity evolution in SELs | |
| To evaluate the treatment effect of | Change in EQ-5D-5L from baseline by | |
| tolebrutinib via changes in participants' | visit over time | |
| health-related quality of life (HRQoL), and | | |
| working capacity | | |

Abbreviations: 9-HPT, 9-hole peg test; AE, adverse event; AESI, adverse event of special interest; CDI, confirmed disability improvement; CDP, confirmed disability progression; Chi3L1, chitinase-3-like protein 1; ECG, electrocardiogram; EDSS, Expanded Disability Status Scale; EOS, end of study; MRI, magnetic resonance imaging; MSQoL-54, Multiple Sclerosis Quality of Life-54 Questionnaire; NfL, neurofilament light chain; PD, pharmacodynamic; PK, pharmacokinetic; nrSPMS, non-relapsing secondary progressive multiple sclerosis; T25-FW, timed 25-foot walk.

General Statistical Analyses Considerations. The baseline values of efficacy parameters are generally defined as the last available value prior to the first dose of study medication unless otherwise specified. For the EDSS, the baseline value will be taken as the average of the screening and randomization visit values. If one of the values is missing, the non-missing value will be used as baseline. The baseline value of safety parameters is defined as the last available value prior to the first dose of IMP. Unless otherwise indicated, 2-sided p-values and 95% confidence intervals [CI(s)] will be provided for assessment of treatment differences.

### Number of Participants:

Approximately 1700 people will be screened to achieve 1290 participants randomly assigned to study intervention.

Enrolled participants will be randomly assigned at a ratio of 2:1 to 60 mg (established from dose-finding Study DRI15928) of oral, daily tolebrutinib or daily matching placebo.

Randomization will be stratified by age at screening (>40 versus ≤40 years) and geographic region (US versus non-US).

Note: “Enrolled” means a participant's, or their legally acceptable representative's, agreement to participate in a clinical study following completion of the informed consent process. Potential participants who are screened for the purpose of determining eligibility for the study, but do not participate in the study, are not considered enrolled, unless otherwise specified by the protocol.

### Intervention Groups and Duration:

### Study Intervention(s)

Investigational medicinal product(s)
Formulation: tolebrutinib film coated tablet
Route(s) of administration: oral
Dose regimen: 60 mg once daily

### Investigational Medicinal Product(s)

Formulation: placebo to match tolebrutinib film coated tablet
Route(s) of administration: oral
Dose regimen: once daily

Noninvestigational Medicinal Product(s)
Formulation: MRI contrast-enhancing preparations
Route(s) of administration: intravenous (IV)
Dose regimen: per respective label

### Temporary Investigational Medicinal Product (IMP) Interruption Due to Surgery

If surgery is needed during the study, consider the benefit-risk of withholding the IMP for at least 3 to 7 days pre- and post-surgery and the risk of bleeding

The duration of treatment will vary for individual participants, depending on the time of recruitment. With a planned recruitment period of approximately 24 months and an assumed event rate (discussed below), the duration of the study should be approximately 48 months. All recruited participants will be followed in the study until the common study end, which will be estimated and announced by the Sponsor to ensure that approximately 288 events of 6-month CDP have been observed before the study end.

### Statistical Considerations:

### Primary Endpoint:

The primary endpoint will be the treatment difference between tolebrutinib and placebo in time to onset of 6 month-CDP regardless of completion of the treatment period. This endpoint corresponds to a “treatment policy strategy”. This endpoint will be considered primary for supporting regulatory decision making.

The time to onset of 6-month CDP will be analyzed by a Cox proportional hazards model with terms for treatment, age at screening (>40, ≤40 years) and geographic region (US, non-US). A log-rank test stratified by age at screening (>40, ≤40 years) and geographic region (US, non-US) to compare tolebrutinib to placebo will also be examined.

In this primary ITT analysis:
For participants who complete the study without an initial disability progression or prematurely discontinue the study before 6-month confirmation of an onset of disability progression, the participant's event time will be censored at the date of last EDSS assessment.
For participants who have an initial onset of disability progression but reach the common study end date prior to 6-month confirmation, the event status of the participant will be determined by an imputation approach. Since in this setting, the partial missing data can reasonably be assumed to be missing at random, this approach leverages the partial information and follows the ITT principle. A logistic model with terms for age at screening (>40, ≤40 years) and geographic region (US, non-US) will be used to determine the event status as the imputation model within each treatment. A multiple imputation approach will be used to summarize the results.

Only EDSS assessments measured more than 90 days after the onset of an adjudicated relapse will be used to determine onset of disability progression. In addition, for the purpose of confirmation, only EDSS scores measured more than 90 days after the onset of an adjudicated relapse will be used. In case of such MS relapse, the next quarterly EDSS assessment will be used for CDP confirmation. The minimum increase in score required for progression must also be maintained for any non-confirmatory (ie, intervening) EDSS assessment(s) between the initial (onset) and confirmation EDSS scores.

### Main Secondary Endpoints:

For other time-to-event endpoints (time to onset of sustained 20% increase in the 9-HPT, of sustained 20% increase in the T25-FW, of 3-month CDP, and of CDI), similar analysis as for the primary analysis of the primary efficacy endpoint will be performed in the ITT population.

Continuous endpoints (percent change in brain volume loss, change in cognitive function, change in physical function, and change in MSQoL-54 at EOS) will be analyzed using a mixed-effect model with repeated measures (MMRM) approach. The model will include change/percent change values for the respective endpoint at each scheduled visit as response variables, and treatment, age at screening (>40, ≤40 years), geographic region (US, non-US), visit, treatment by-visit interaction, baseline value for the endpoint being assessed and baseline value-by-visit interaction as covariates.

Categorical efficacy endpoints with count data (new and/or enlarging T2 hyperintense over the study period after baseline) will be analyzed using a negative binomial regression model. The model will include the total count occurring during the observation period as the response variable, with treatment group, age at screening (>40, ≤40 years), and geographic region (US, non-US) as covariates. Log transformed number of scans will be the offset variable.

### Analysis of Safety Data:

All safety summaries will be descriptive; no statistical significance tests will be performed on safety data. This includes treatment-emergent adverse events (TEAEs) and other safety information (eg, clinical laboratory evaluations, electrocardiograms [ECGs], and vital signs). TEAEs are defined as adverse events (AEs) that developed or worsened or became serious during the treatment period. These analyses will be based on the safety population, defined as all participants randomly assigned and exposed to study intervention, regardless of the amount of exposure, analyzed according to the treatment actually received.
There was no death or treatment-emergent adverse event (TEAE) leading to permanent treatment discontinuation during the study. One treatment-emergent serious adverse event (SAE) (MS relapse) was reported in a participant treated with 60 mg tolebrutinib; the remainder of the reported TEAEs were of mild or moderate intensity.
There was no direct correlation between the doses of tolebrutinib administered and number or intensity of TEAEs. The most common events reported in participants in the tolebrutinib treatment arms were headache, upper respiratory tract infection, and nasopharyngitis.
Two participants had treatment-emergent transient alanine aminotransferase (ALT) increase >3×ULN, 1 during the 30 mg tolebrutinib treatment period (at Week 8, 105 U/L [normal range 6 to 34 U/L]) that returned to normal range within 4 days and 1 during the 60 mg tolebrutinib treatment period (at Week 4, 107 U/L [normal range 6 to 34 U/L]). The participant in the 60 mg group had slightly elevated ALT at screening (48 U/L) and at baseline (50 U/L); ALT levels returned to the normal range in 8 weeks. Both participants continued study treatment during this period. All other liver enzyme levels for both participants were within normal ranges during the treatment period; one event was assessed as related and one as unrelated to the study drug by the Investigators. Both participants completed the DRI15928 study and successfully rolled over to the LTS follow-up study.
One event of mild petechia in a female participant (at Week 8 in the tolebrutinib 30 mg group) and 2 events of mild microscopic hematuria in 2 male participants (1 event at Week 16 in the tolebrutinib 30 mg group and 1 event on Day 1 in the tolebrutinib 60 mg group, with occult blood noted in urine) were reported during the treatment period in the tolebrutinib Phase 2b trial. The hematology results were clinically insignificant for all 3 participants from the onset of the events. The participant with mild petechia had benign pigmentary lesions noted during screening, and the event was assessed as related to the study drug by the Investigator. The 2 events of mild microscopic hematuria were assessed as unrelated to the study drug. All 3 events resolved spontaneously.
No severe infections occurred. The most frequently reported (≥3 events total) in the tolebrutinib treatment period were upper respiratory tract infection, nasopharyngitis, gastroenteritis, and respiratory tract infection.
No clinically significant cytopenia, including thrombocytopenia and neutropenia, were reported or detected based on hematologic laboratory results, and no clinically significant cardiac arrhythmia was observed via ECG monitoring during the study. An identified risk for tolebrutinib has been identified as follows:
Treatment-emergent SAEs of drug-induced liver injury (DILI) were reported in the ongoing Phase 3 trials; however, all cases occurred between Months 2 to 3 and appear reversible after treatment discontinuation, with potential confounders identified for some of the cases.

Drug-induced liver injury has been identified in the ongoing Phase 3 trials. The reported events occurred Months 1 to 3 after the start of the IMP, and the elevation of liver enzymes appears reversible after IMP discontinuation. Exclusion criteria and monitoring frequency have been updated in all actively recruiting protocols to mitigate risk of hepatic injury.

This is a Phase 3, randomized, double-blind, 2-arm, placebo-controlled, parallel group, multicenter, event-driven (6-month CDP) trial with a variable treatment duration ranging from approximately 24 to 48 months in participants with nrSPMS.

The study will consist of the following study periods:

Screening period: Day −28 to Day −1.

Randomization/start of IMP: Eligible participants will be randomly assigned at a 2:1 ratio to receive oral tolebrutinib (60 mg) daily or matching placebo daily.

Intervention period: Double-blind treatment period for assessment of efficacy and safety up to the EOS as described in the End of Study Definition.

A month is defined as a period of 28 days by convention.

Safety follow-up period/EOS: 4 to 8 weeks after the last dose of study treatment (for participants completing IMP treatment [double blind or open-label, if rescued after 6-month CDP] and not entering the LTS study) to collect safety data.

EOS: A participant is considered to have completed the study if he/she has completed all periods of the study including the EOS Visit, whether remaining on IMP or not.

Participants with 6-month CDP are eligible for open-label active treatment as rescue (tolebrutinib) (under heading Rescue medicine).

The duration of the treatment period will vary for individual participants, depending on the time of recruitment and study end as described below. All recruited participants will be followed in the study until approximately 288 events of 6-month CDP are observed. With a planned recruitment period of approximately 24 months and an assumed event rate, the duration of the study should be approximately 48 months, with estimated mean treatment duration of 33 to 36 months.

Participants will be encouraged to remain in the study and comply with all study visits until the EOS in the case that they discontinue the study intervention early.

To minimize possible biases in the study outcome, the study is double blinded. The blind of initial treatment will be kept from participants, any Investigator site staff, and the Sponsor until the study end.

### Study Population

Prospective approval of protocol deviations to recruitment and enrollment criteria, also known as protocol waivers or exemptions, is not permitted.

### Inclusion Criteria

Participants are eligible to be included in the study only if all of the following criteria apply as shown in Table 1C:

TABLE 1C
Inclusion Criteria

| Category | Criteria | |
|---|---|---|
| Age | I 01. | Participant must be 18 to 60 years of age inclusive, at the |
| | | time of signing the informed consent. |
| Type of | I 02. | The participant must have a previous diagnosis of RRMS |
| participant | | in accordance with the 2017 revised McDonald criteria |
| and disease | | (Thompson et al. Lancet Neurol. 2018, 17, 162-173). |
| characteristics | I 03. | The participant must have a current diagnosis of SPMS in |
| | | accordance with the clinical course criteria (Lublin et al. |
| | | Neurology 1996 46, 907-911) revised in 2013 (Lublin et |
| | | al. Neurology 2014, 83, 278-286) and endorsed by an |
| | | Adjudication Committee. |
| | I 04. | The participant must have documented evidence of |
| | | disability progression observed during the 12 months |
| | | before screening. Eligibility will be analyzed by an |
| | | Adjudication Committee (to evaluate source data for |
| | | disability confirmation). |
| | I 05. | Absence of clinical relapses for at least 24 months. |
| | I 06. | The participant must have an EDSS score at screening |
| | from 3.0 to 6.5 points, inclusive. | |
| Weight | I 08. | Not applicable. |
| Sex | I 09. | Male and/or female |
| | Contraceptive use should be consistent with local regulations | |
| | regarding the methods of contraception for those | |
| | participating in clinical studies. | |
| | A) | Male participants Not applicable. |
| | B) | Female participants |
| | • | A female participant is eligible to participate if she is not |
| | | pregnant or breastfeeding, and at least one of the |
| | | following conditions applies: |

| | |
|---|---|
| - | Is not a woman of childbearing potential (WOCBP) |
| | OR |
| - | Is a WOCBP and agrees to use an acceptable |
| | contraceptive method during the intervention period. |
| | WOCBP must use reliable means of contraception at |
| | a minimum. |
| - | A WOCBP must have a negative highly sensitive |
| | pregnancy test (urine or serum as required by local |
| | regulations) at screening and before the first dose of |
| | study intervention. |
| - | If a urine test cannot be confirmed as negative (eg, an |
| | ambiguous result), a serum pregnancy test is required. |
| | In such cases, the participant must be excluded from |
| | participation if the serum pregnancy result is positive. |
| - | Requirements for pregnancy testing during |
| | and after study intervention are located in the |
| | SoA (Table 1A). |

| | |
|---|---|
| • | Additional requirements for pregnancy |
| | testing during and after study intervention are |
| | located in Table 1E. |
| • | The Investigator is responsible for review of medical |
| | history, menstrual history, and recent sexual activity to |
| | decrease the risk for inclusion of a woman with an early |
| | undetected pregnancy, if allowed by local regulations. |

| | |
|---|---|
| Informed | I 10. The participant is capable of giving signed informed |
| Consent | consent which includes compliance with the requirements and |
| | restrictions listed in the informed consent form (ICF). A specific |
| | ICF for legally minor participants must also be signed by the |
| | participant's legally authorized representative. |

### Exclusion Criteria

Participants are excluded from the study if any of the following criteria apply as shown in Table 1D:

TABLE 1D
Exclusion Criteria

| Category | Criteria | |
|---|---|---|
| Medical | E 01. | The participant has a history of infection or may be at risk |
| conditions | for infection: | |
| | • | A history of T-lymphocyte or T-lymphocyte-receptor |
| | | vaccination, transplantation (including solid organ, stem |
| | | cell, and bone marrow transplantation) and/or antirejection |
| | | therapy. |
| | • | The participant has received any live (attenuated) vaccine |
| | | (including but not limited to varicella zoster, oral polio, and |
| | | nasal influenza) within 2 months before the first treatment |
| | | visit. |
| | • | The participant has a lymphocyte count less than the lower |
| | | limit of normal (LLN) at the Screening Visit. |
| | • | A history of diagnosis of progressive multifocal |
| | | leukoencephalopathy (PML) or evidence of findings |
| | | suggestive of PML on the screening MRI. |
| | • | A history of infection with human immunodeficiency virus |
| | | (HIV). |
| | • | A history of active or latent tuberculosis (TB); TB testing |
| | | should be performed at screening and again during the |
| | | study, if clinically indicated and maybe repeated based on |
| | | clinical judgment, borderline results, or clinical suspicion |
| | | of TB infection. In case of confirmed active or latent TB |
| | | the patient can be re-screened after full completion of anti- |
| | | tuberculosis treatment. |
| | NOTE: The Investigator may consult with an infectious disease | |
| | expert if required, eg, test results are unclear or there is a | |
| | suspicion of false-positive test results. If the infectious disease | |
| | expert considers the test results as false-positive and not | |
| | clinically relevant and confirms that the participant can be | |
| | enrolled in the trial, the Investigator must document this in the | |
| | source data and may then randomize the participant. | |
| | • | Persistent chronic or active or recurring system infection |
| | | that may adversely affect participation or IMP |
| | | administration in this study, as judged by the Investigator. |
| | • | Fever within 4 weeks of the Screening Visit (≥38° C.; |
| | | however, if due to brief and mild ear, nose, throat viral |
| | | infection participant may be included based on the |
| | | Investigator's judgment). |
| | • | Participants at risk of developing or having reactivation of |
| | | hepatitis: results at screening for serological markers for |
| | | hepatitis B and C indicating acute or chronic infection. See |
| | | the Study Manual for further details. |
| | E 02. | The presence of psychiatric disturbance or substance abuse |
| | as evidenced by: | |
| | • | A history of any psychiatric disease, behavioral condition, or |
| | | depression requiring hospitalization within 2 years prior to |
| | | the Screening Visit. |
| | • | A documented history of attempted suicide or suicidal |
| | | ideation of category 4 or |
| | | 5 according to the Columbia Suicide Severity Rating Scale |
| | | (C-SSRS) baseline/screening version over the 6 months |
| | | prior to the Screening Visit, OR if in the Investigator's |
| | | judgment, the participant is at risk for a suicide attempt. |
| | • | Active alcohol use disorder or a history of alcohol or drug |
| | | abuse within 1 year prior to the Screening Visit. |
| | • | Current alcohol intake >2 drinks per day for men and >1 |
| | | drink per day for women |
| | | (1 drink = approximately 14 grams of alcohol = 350 mL |
| | | beer = 140 mL wine = 40 mL of spirits). |
| | E 03. | The following findings obtained during the Screening Visit |
| | | considered in the Investigator's judgment to be clinically |
| | | significant in the context of this trial: |
| | • | Any screening laboratory values outside normal limits. |
| | • | Abnormal ECG. |
| | E 04. | Conditions that may predispose the participant to excessive |
| | bleeding: | |
| | • | A bleeding disorder or known platelet dysfunction at any |
| | | time prior to the Screening Visit. |
| | • | A platelet count <150 000/μL at the Screening Visit. |
| | • | The participant has had major surgery within 4 weeks prior |
| | | to the Screening Visit, which could affect the participant's |
| | | safety or affect immune response (as judged by the |
| | | Investigator) or has planned any elective major surgery |
| | | during the study. |
| | • | A history of significant bleeding event within 6 months |
| | | prior to screening, according to the Investigator's judgment |
| | | such as, but not limited to cerebral or gastrointestinal |
| | | bleeding. |
| | E 05. | Conditions that would adversely affect participation in the |
| | | study or make the primary efficacy endpoint non-evaluable: |
| | • | A short life expectancy due to pre-existing health |
| | | condition(s) as determined by their treating neurologist. |
| | • | A history or presence of significant other concomitant |
| | | illness according to the Investigator's judgment such as, but |
| | | not limited to cardiovascular (including Stage III or IV |
| | | cardiac failure according to New York Heart Association |
| | | [NYHA] classification), or renal (ie, undergoing dialysis), |
| | | neurological, endocrine, gastrointestinal, metabolic, |
| | | pulmonary, or lymphatic disease that would adversely |
| | | affect participation in this study. |
| | • | Acute liver disease, cirrhosis, chronic liver disease (unless |
| | | considered stable for >6 months). |
| | • | Confirmed screening ALT >1.5 × upper limit of normal |
| | | (ULN) OR AST >1.5 × ULN OR alkaline phosphatase >2 × |
| | | ULN (unless caused by non-liver-related disorder or |
| | | explained by a stable chronic liver disorder) OR total |
| | | bilirubin >1.5 × ULN (unless due to Gilbert syndrome or |
| | | non-liver-related disorder). |
| | • | At screening, elevated transferrin saturation (>50% in |
| | | males and >40% in females) and/or with elevated ferritin |
| | | levels >500 μg/L. |
| | • | Any malignancy within 5 years prior to the Screening Visit |
| | | (except for effectively treated carcinoma in situ of the |
| | | cervix or adequately treated non-metastatic squamous or |
| | | basal cell carcinoma of the skin) will also be exclusionary. |
| | • | Any other medical condition(s) or concomitant disease(s) |
| | | making them nonevaluable for the primary efficacy |
| | | endpoint or that would adversely affect participation in this |
| | | study, as judged by the Investigator. |
| Prior/ | E 06. | A requirement for concomitant treatment that could bias |
| concomitant | | the primary evaluation, such as any of the following |
| therapy | | medications/treatments within the specified time frame |
| | | before any randomization assessment (no wash out is |
| | | required for dimethyl fumarate, interferon beta or |
| | | glatiramer acetate treatments although use is not permitted |
| | | on or after Day 1): |

| | Exclusionary if used/used within |
|---|---|
| Medication | required wash-out period |
| Systemic corticosteroids, | 1 month prior to |
| adrenocorticotropic hormone | screening MRI scan |
| Siponimod, ponesimod | 1 week before |
| | randomization |
| | with MRI and |
| | clinical assessment |
| | for PML prior to |
| | randomization |
| Plasma exchange | 1 month prior to |
| | randomization |
| IV immunoglobulin | 2 months prior to |
| | randomization |
| Fingolimod, ozanimod | 6 weeks before |
| | randomization |
| | with MRI and |
| | clinical assessment |
| | for PML |
| Teriflunomide^{a} mildly to moderately | 3 months prior to |
| immunosuppressive/chemotherapeutic | randomization |
| medications such azathioprine, | |
| mycophenolate mofetil, and | |
| methotrexate | |
| Natalizumab | 2 months before |
| | randomization |
| | with MRI and |
| | clinical |
| | assessments for |
| | PML |
| B-cell depleting therapies such as | 6 months prior to |
| ocrelizumab and rituximab | randomization |
| Ofatumumab | 4 months |
| Highly | 2 years prior to |
| immunosuppressive/chemotherapeutic | randomization |
| medications: mitoxantrone up to 120 | |
| mg/m² body surface area, | |
| cyclophosphamide, cladribine, | |
| cyclosporine | |
| Alemtuzumab | 4 years prior to |
| | randomization |
| Other MS-disease-modifying | 5 half-lives or until |
| therapies | end of |
| | pharmacodynamics |
| | activity, whichever |
| | is longer |
| Lymphoid irradiation, bone marrow | No patient who has |
| transplantation, mitoxantrone (with | received any of |
| evidence of cardiotoxicity following | these treatments at |
| treatment, or cumulative lifetime | any time is |
| dose >120 mg/m²), other strongly | eligible. |
| immunosuppressive treatments with | |
| very long-lasting effects | |

| Prior/ | E 07. | The participant is receiving potent and moderate inducers of |
|---|---|---|
| concurrent | | CYP3A or potent inhibitors of CYP2C8 hepatic enzymes as |
| clinical study | | listed in Table 1I. |
| experience | E 08. | The participant is receiving anticoagulant/antiplatelet |
| | | therapies; those that are not permitted to be taken |
| | | concomitantly with the IMP, include the following: |
| | • | Acetylsalicylic acid (aspirin) >81 mg/day. |
| | • | Antiplatelet drugs (eg, clopidogrel). |
| | • | Warfarin (vitamin K antagonist). |
| | • | Heparin, including low molecular weight heparin |
| | | (antithrombin agents). |
| | • | Dabigatran (direct thrombin inhibitor). |
| | • | Apixaban, edoxaban, rivaroxaban (direct factor Xa |
| | | inhibitors). |
| | | Note: All the above-mentioned drugs must be stopped at |
| | | least 5 half-lives before study drug administration except |
| | | for aspirin, which must be stopped at least 8 days before. |
| | | These washout periods are only applicable in the case that |
| | | the Investigator deems it clinically appropriate to |
| | | discontinue the listed medications or there is a recent |
| | | history of use of these medications (as in the case when |
| | | short-term treatment with anticoagulants is clinically |
| | | recommended for certain thrombotic events), and |
| | | therefore these washout periods will need to be followed |
| | | prior to randomization. |
| | | If however the participant has a chronic underlying medical |
| | | condition (stroke, coronary or carotid artery disease, |
| | | valvular heart disease etc) requiring continued use of these |
| | | medications, the participant cannot be enrolled in the study. |
| | E 09. | The participant has sensitivity to any of the study |
| | | interventions, or components thereof, or has a drug or other |
| | | allergy that, in the opinion of the Investigator, |
| | | contraindicates participation in the study. |
| | E 10. | The participant was previously exposed to any BTK inhibitor, |
| | including tolebrutinib. | |
| | E 11. | The participant has taken other investigational drugs within |
| | 3 months or 5 half-lives, whichever is longer, before the Screening | |
| | Visit. | |
| Diagnostic | E 12. | The participant has a contraindication for MRI, ie, presence |
| assessments | | of pacemaker, metallic implants in high risk areas (ie, |
| | | artificial heart valves, aneurysm/vessel clips), presence of |
| | | metallic material (eg, shrapnel) in high risk areas, known |
| | | history of allergy to any contrast medium, or history of |
| | | claustrophobia that would prevent completion of all |
| | | protocol scheduled MRI. |
| | | Note: People with a contraindication to Gd can be enrolled |
| | | into the study but cannot receive Gd contrast dyes during |
| | | their MRI scan. |
| Other | E 13. | Individuals accommodated in an institution because of |
| exclusions | | regulatory or legal order; prisoners or participants who are |
| | | legally institutionalized. |
| | E 14. | Any country-related specific regulation that would prevent |
| | | the participant from entering the study. |
| | E 15. | The participant is not suitable for participation, whatever the |
| | | reason, as judged by the Investigator, including medical or |
| | | clinical conditions, or participants potentially at risk of |
| | | noncompliance to study procedures or not able to follow the |
| | | schedule of protocol assessments due to other reasons. |
| | E 16. | Participants are employees of the clinical study site or other |
| | | individuals directly involved in the conduct of the study, or |
| | | immediate family members of such individuals (in |
| | | conjunction with Section 1.61 of the International Council |
| | | for Harmonisation [ICH] - Good Clinical Practice [GCP] |
| | | Ordinance E6). |
| | E 17. | Any other situation during study implementation/course that |
| | | may raise ethics considerations. |
| | | Note: a one-time retest at screening may be |
| | | performed if an abnormal laboratory test value is |
| | | considered temporary. |

IV: intravenous,
MRI: magnetic resonance imaging,
MS: multiple sclerosis,
PML: progressive multifocal leukoencephalopathy
^{a}No time restriction if accelerated elimination procedure is done.

### Lifestyle Considerations

Meals and dietary restrictions: tolebrutinib shall be taken with a regular meal. When possible, the meal with which tolebrutinib is taken (eg, breakfast, lunch, or dinner) should be consistent throughout the study. The typical meal with which the IMP is taken will be recorded at each visit. In case the mealtime needs to be changed for IMP administration, a gap of a minimum of 12 hours between 2 doses should be maintained.

Caffeine, alcohol, and tobacco: For each visit with PK/PD assessment (refer to Table 1A), participants will abstain from ingesting caffeine- or xanthine-containing products (eg, coffee, tea, cola drinks, and chocolate) for 2 hours before the start of treatment until after collection of the final PK and/or PD sample later that day.

For each visit with PK/PD assessment (refer to Table 1A), participants will abstain from alcohol for 24 hours before the start of treatment until after collection of the final PK and/or PD sample later that day.

During the entire study, participants should be warned not to consume substantial quantities of alcohol, defined as >14 grams (1 standard drink) per day in female participants or >28 grams (2 standard drinks) per day in male participants on a regular basis.

### Study Intervention

Study intervention is defined as any investigational intervention(s), marketed product(s), or placebo intended to be administered to a study participant according to the study protocol.

### Criteria for Temporarily Delaying Enrollment and Administration of Study Intervention

During a regional or national emergency declared by a governmental agency, if the site is unable to adequately follow protocol mandated procedures, contingency measures should be considered for screening, enrollment, randomization, and administration of the study intervention.

TABLE 1E
Overview of study interventions administered

| | | |
|---|---|---|
| ARM name | tolebrutinib | Placebo |
| Intervention name | tolebrutinib | Placebo |
| Type | Drug | Drug |
| Dose formulation | Film coated tablet | Film coated tablet |
| Unit dose | 60 mg | 0 mg |
| strength(s) | | |
| Dosage level(s) | Once daily | Once daily |
| Route of | Oral | Oral |
| administration | | |
| Use | Experimental | Placebo |
| IMP and NIMP | IMP | IMP |
| Packaging and | Study intervention will be | Study intervention will be provided in |
| labeling | provided in wallet blister | wallet blister packaging. Each wallet |
| | packaging. Each wallet blister | blister packaging will be labeled as per |
| | packaging will be labeled as | country requirements. |
| | per country requirements. | |

IMP: investigational medicinal product; NIMP: noninvestigational medicinal product.

Between the protocol-scheduled on-site visits, interim visits may be required for IMP dispensing. As an alternative to these visits or to replace on-site IMP dispensation, if needed, tolebrutinib may be supplied from the site to the participant via a Sponsor-approved courier company (direct-to-patient [DTP]) where allowed by local regulations and approved by the Sponsor.

### Noninvestigational Medicinal Product

### MRI Contrast-Enhancing Preparations

Route(s) of administration: IV
Dose regimen: as per respective label

### Concomitant Therapy

Any medication or vaccine (including over-the-counter or prescription medicines, vitamins, and/or herbal supplements) that the participant is receiving at the time of enrollment or receives during the study must be recorded along with:
Reason for use.
Dates of administration including start and end dates.
Dosage information including dose and frequency.

Any live (attenuated) vaccine within 2 months before the first treatment visit and during the intervention period is prohibited.

Therapies for MS noted in the exclusion criterion E 06 are not permitted after randomization while the participant is on study treatment. Short-term use (3 to 5 days) of glucocorticoids (eg, for MS relapse treatment or an acute illness) and local corticosteroids (eg, topical, nasal, ocular, otic, intra-articular) are allowed.

The Medical Monitor should be contacted if there are any questions regarding concomitant or prior therapy.

Participants must abstain from taking prescription or nonprescription drugs (including vitamins and dietary or herbal supplements) within 7 days (or 14 days if the drug is a potential enzyme inducer) or 5 half-lives (whichever is longer) before the start of study intervention until completion of the follow-up visit, unless, in the opinion of the Investigator and Sponsor, the medication will not interfere with the study.

Medications for treatment of MS symptoms (eg, walking impairment, fatigue, spasticity, incontinence, pain) should be maintained at a stable dose prior to screening and for the duration of the treatment period, if clinically feasible.

Anticoagulant/antiplatelet therapies are not permitted to be taken concomitantly with the IMP, including the following:
Acetylsalicylic acid (aspirin) >81 mg/day.
Antiplatelet drugs (eg, clopidogrel).
Warfarin (vitamin K antagonist).
Heparin, including low molecular weight heparin (antithrombin agents).
Dabigatran (direct thrombin inhibitor).
Apixaban, edoxaban, rivaroxaban (direct factor Xa inhibitors).

Paracetamol/acetaminophen, at doses of ≤3 grams/day, is permitted for use at any time during the study. Short courses (up to 5 days) of nonsteroidal anti-inflammatory drugs (NSAIDs) (other than acetylsalicylic acid), preferably selective cyclooxygenase-2 inhibitors at the lowest effective dose, may be given during the study if clinically necessary for the treatment of an existing medical condition or a new event. The Investigator must record the use of NSAIDs (and any other comedication) in the eCRF.

### CYP Inhibitors and Inducers:

Potent and moderate inducers of CYP3A or potent inhibitors of CYP2C8 hepatic enzymes are not permitted throughout the study (Table 1I).

Based on nonclinical drug metabolism studies, tolebrutinib is a substrate of the CYP3A and CYP2C8 isoenzymes. In healthy participants, potent CYP3A4 inhibitor (itraconazole 200 mg once daily for 4 days) increased tolebrutinib area under the curve AUC exposure 1.8-fold (Study INT16385) and potent CYP2C8 inhibitor (gemfibrozil 600 mg twice daily for 6 days) increased tolebrutinib AUC exposure 8.4-fold (Study INT16726). Based on a satisfactory safety and tolerability profile and on the observed exposure in healthy participants who received tolebrutinib at a dose of up to 240 mg tolebrutinib once daily for 14 days under fed conditions (Study TDR16862), drugs that strongly inhibit CYP3A4 are allowed and drugs that strongly inhibit CYP2C8 are not permitted. In healthy participants, potent CYP3A4 and moderate CYP2C8 induction by rifampicin (600 mg once daily for 8 days) decreased tolebrutinib exposure 6-fold (Study INT16726). Therefore, potent and also moderate (based on prediction) CYP3A inducers are not permitted due to their potential to decrease tolebrutinib exposure and efficacy. See Table 1I for the list of drugs to be avoided.

The Sponsor should be contacted if there are any questions regarding concomitant or prior therapy.

### Open-Label Treatment

If a participant achieves the primary endpoint (6-month CDP), they may, in conjunction with the Treating Investigator, choose to receive one of the following:
1. To switch to open-label tolebrutinib treatment and continue regularly planned study visits;
2. To switch to a non-study treatment approved for nrSPMS in their respective country. The participant will be encouraged to remain in the study for planned clinical visits until common study end.

In case the participant switches to open-label tolebrutinib treatment, he/she will need to be monitored for liver function tests after the first open-label dose at Weeks 2, 3, 4, 5, 6, 7, 8, 9, 10, 11, and 12, and then monthly for the next 9 months. After that, the schedule visits timepoints per SoA will be resumed (ie, every 3 months), until the common study end. Whenever a timepoint will coincide with a scheduled visit as per SoA (Table 1A), the full scheduled visit assessments will be performed instead of the liver monitoring testing alone.

For participants achieving 6-month CDP, the Investigator must ensure that the participant does not meet any of the following criteria prior to the switch to open-label tolebrutinib treatment:
Current alcohol intake >2 drinks per day for men and >1 drink per day for women (1 drink=approximately 14 grams of alcohol=350 mL beer=140 mL wine=40 mL of spirits).
Acute liver disease, cirrhosis, chronic liver disease (unless considered stable for >6 months).
Confirmed ALT>1.5×ULN OR AST>1.5×ULN OR ALP>2×ULN (unless caused by non-liver-related disorder or explained by a stable chronic liver disorder) OR total bilirubin >1.5×ULN (unless due to Gilbert syndrome or non-liver-related disorder).

Liver function tests will be performed for all participants prior to the start of open-label treatment.

Should the participant and Investigator opt for the provision of open-label medicine, they will remain blinded to the original treatment assignment.

All individual blinded data will be reviewed and all queries resolved, if possible, before the switch to the open-label treatment to ensure data integrity for primary assessment. Prior to initiation of open-label treatment, the Investigator shall confirm that there has been no adjudicated relapse within 90 days prior to the onset or confirmation of 6-month CDP. Based on individual symptoms and assessed risk of further progression, the Investigator and participant may choose for the participant to remain on the initial double-blind treatment after achieving 6-month CDP.

The initial treatment assignment will be kept blinded from participants, any Investigator site staff, and the Sponsor until the study end.

The supply of tolebrutinib as open-label medication will be specified at the country level.

Multiple sclerosis relapses are not frequent in the nrSPMS population, but their occurrence during the study cannot be ruled out completely. In the case of MS relapse, treatments are allowed as per local routine practice (eg, high dose IV methylprednisolone for 3 to 5 days). The date and time of relapse treatment administration as well as the name and dosage regimen of the medication must be recorded.

### Dose Modification

Dose modification is not foreseen in this study. Treatment might need to be interrupted or permanently discontinued if deemed necessary due to an AE.

### Discontinuation of Study Intervention and Participant Discontinuation/Withdrawal

### Discontinuation of Study Intervention

### Definitive Discontinuation

The study intervention should be continued whenever possible.

Permanent intervention discontinuation is any treatment discontinuation associated with the definitive decision by the Investigator or the participant not to re-expose the participant to the study intervention at any time.

In rare instances, it may be necessary for a participant to permanently discontinue the study intervention. If study intervention is permanently discontinued, the participant will be asked to remain in the study to be evaluated until the EOS visit. For this set of participants (participants who discontinued the IMP and/or switched to other DMTs), no PK or biomarker samples will be collected after the pEOT visit, and MRI assessments will be performed only annually using the next annual visit as the starting point.

This will be important to continue to evaluate for safety and efficacy. See the SoA (Table 1A) for data to be collected at the time of discontinuation of the study intervention. In the case that the study intervention is permanently discontinued, the participant should be treated for MS according to local clinical practice and the best judgment of the Investigator.

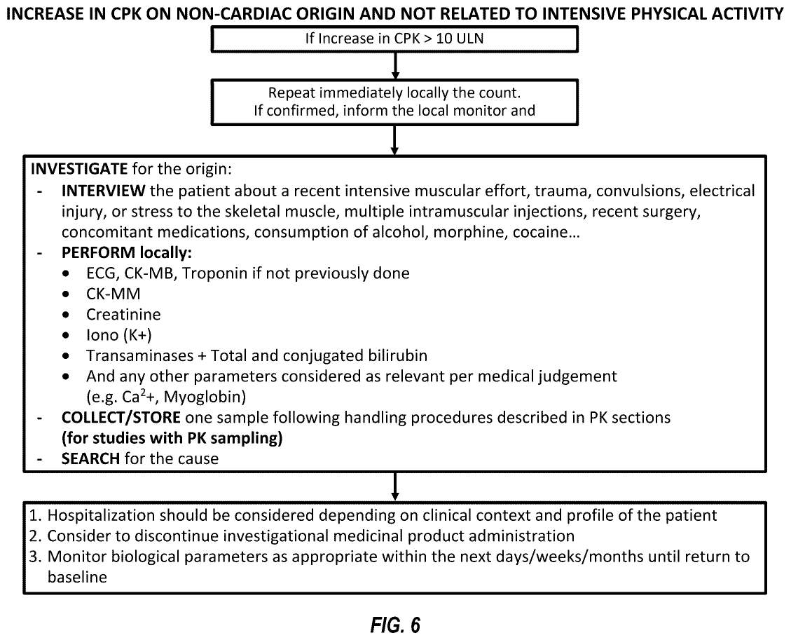

The following may be justifiable reasons for the Investigator or Sponsor to discontinue a participant from treatment:
Adverse events that endanger the safety of the participant, or if discontinuation of study intervention is desired or considered necessary by the Investigator and/or participant.
If IMP discontinuation criteria are met as per the guidance for the follow up of laboratory abnormalities (Table 1F).
The participant is no longer deriving a therapeutic/clinical benefit in the opinion of the Investigator.
If a female participant becomes pregnant or wishes to become pregnant during the study.
At participant's request, ie, withdrawal of the consent for treatment.
Any serious opportunistic infections (eg, PML [see FIG. 6], HIV)
Continued need for/chronic use of prohibited a concomitant medication (see Table 1I).
Use of open-label tolebrutinib or non-study disease modifying therapy approved for nrSPMS in their respective countries (eg, after 6-month CDP).
A brief physical examination will include, at a minimum, assessments of the skin, lungs, cardiovascular system, neurological examination, and abdomen (liver and spleen).
Investigators should pay special attention to clinical signs related to previous serious illnesses.
Any clinically significant new finding or worsening of previous finding should be reported as an AE, per Investigator's judgement.

### Safety Assessments

### Physical Examinations

A physical examination will be performed at the time points specified in the SoA (Table 1A).
A complete physical examination will include, at a minimum, assessments of the general appearance, head and neck, abdomen, lymph nodes, skin, cardiovascular system, respiratory system, musculoskeletal system, and neurological examination by the Treating Investigator. Height (at screening) and weight will also be measured and recorded. Further details will be provided in the Study Manual.
A brief physical examination will include, at a minimum, assessments of the skin, lungs, cardiovascular system, neurological examination, and abdomen (liver and spleen).
Investigators should pay special attention to clinical signs related to previous serious illnesses.
Any clinically significant new finding or worsening of previous finding should be reported as an AE, per Investigator's judgement.

The extent of the physical examination can be broadened at the discretion of the Treating Investigator in order to evaluate AEs or abnormal clinical laboratory test values.

### Vital Signs

Body temperature, heart rate, and blood pressure will be assessed.
Blood pressure and pulse measurements will be assessed with the participant in a supine or sitting position with a completely automated device. Manual techniques will be used only if an automated device is not available.
Blood pressure and pulse measurements should be preceded by at least 5 minutes of rest for the participant in a quiet setting without distractions (eg, television, cell phones).
Vital signs (to be taken before blood collection for laboratory tests) will consist of 1 heart rate and 3 blood pressure measurements (3 consecutive blood pressure readings will be recorded at intervals of at least 1 minute).

### Electrocardiograms

12-lead ECG will be obtained as outlined in the SoA (see Table 1A) using an ECG machine that automatically calculates the heart rate and measures PR, QRS, QT, and QTcF intervals. In case the ECG machine does not automatically calculate QTcF, manual calculation using nomogram or automatic website calculator (eg, https://reference.medscape.com/calculator/48/ecg-corrected-qt) is acceptable
ECG and 30 second rhythm strips will be obtained locally. Further details will be included in the Study Manual.

### Clinical Safety Laboratory Assessments

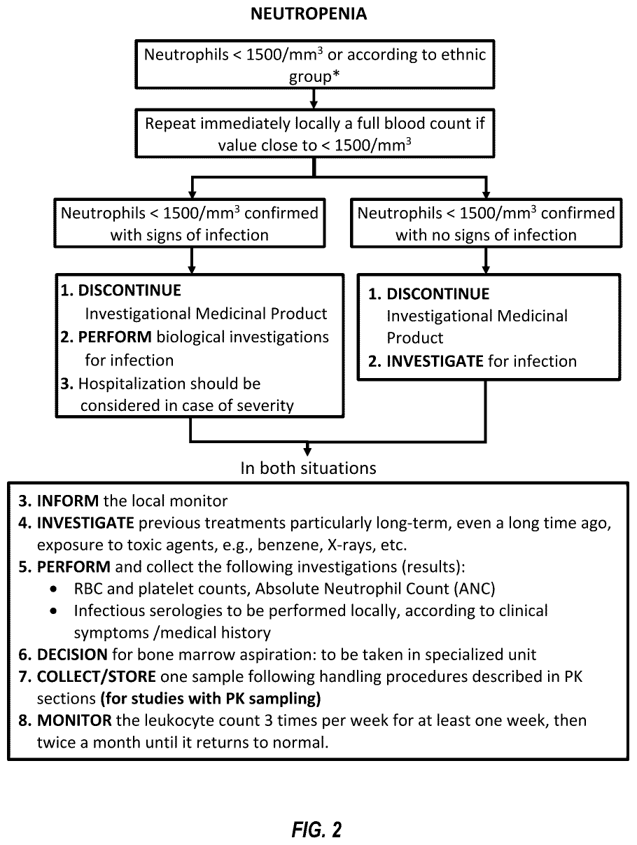

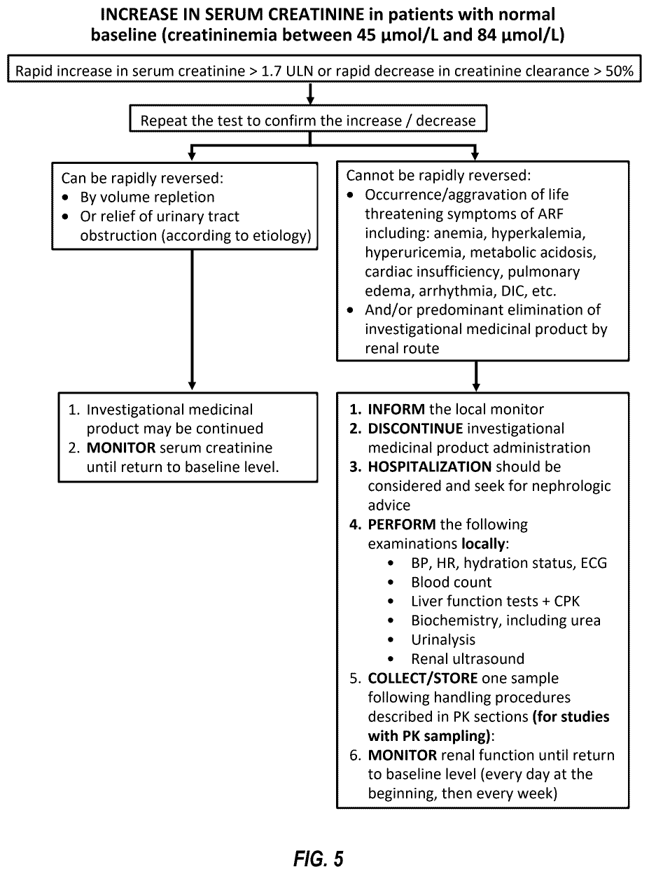

See Table 1I for the list of clinical laboratory tests to be performed. Per the SoA, serology tests for hepatitis B and C will be performed during screening; testing for other infectious disease should be performed during screening if required locally.
The Treating Investigator may solicit emergency local laboratory data in case of emergent safety events to allow for appropriate treatment decisions. All clinically relevant solicited emergency local laboratory data will be recorded in the eCRF.
The Treating Investigator must review the laboratory report, document this review, and record any clinically relevant changes occurring during the study in the AE section of the eCRF. The laboratory reports must be filed with the source documents. Clinically significant abnormal laboratory findings are those which are not associated with the underlying disease, unless judged by the Investigator to be more severe than expected for the participant's condition.
All laboratory tests with values considered clinically significantly abnormal during participation in the study or within 30 days after the last dose of study intervention should be repeated until the values return to normal or baseline or are no longer considered clinically significant by the Investigator.
   If abnormal laboratory values do not return to normal/baseline within a period of time judged reasonable by the Investigator, the etiology should be identified and the Sponsor notified.
All protocol-required laboratory assessments, as defined in Table 11, must be conducted in accordance with the laboratory manual and the SoA (see Table 1A). In the event the laboratory assessments in FIGS. 2-8 indicate discontinuation of IMP, temporary discontinuation should be considered unless otherwise specified.
   If laboratory values from non-protocol specified laboratory assessments performed at the institution's local laboratory require a change in participant management or are considered clinically significant by the Investigator (eg, SAE or AE or dose modification), then the results must be recorded in the eCRF.

### Suicidal Ideation and Behavior Risk Monitoring

Tolebrutinib crosses the blood-brain barrier. Assessment of suicidal ideation and behavior as well as treatment-emergent suicidal ideation and behavior will be monitored during Study EFC16645 using the C-SSRS. For safety reasons, C-SSRS will be administered throughout the study by the Treating Investigator or delegated to an individual who is certified to administer the scale.

Study drug administration must be interrupted if a participant scores “yes” on items 4 or 5 of the Suicidal Ideation Section of the C-SSRS, or “yes” on any item of the Suicidal Behavior Section. A psychiatrist will be consulted and decide whether the study drug can be restarted and if any additional risk mitigation strategies are required (eg, increased monitoring, antidepressant administration).

### Adverse Event of Special Interest

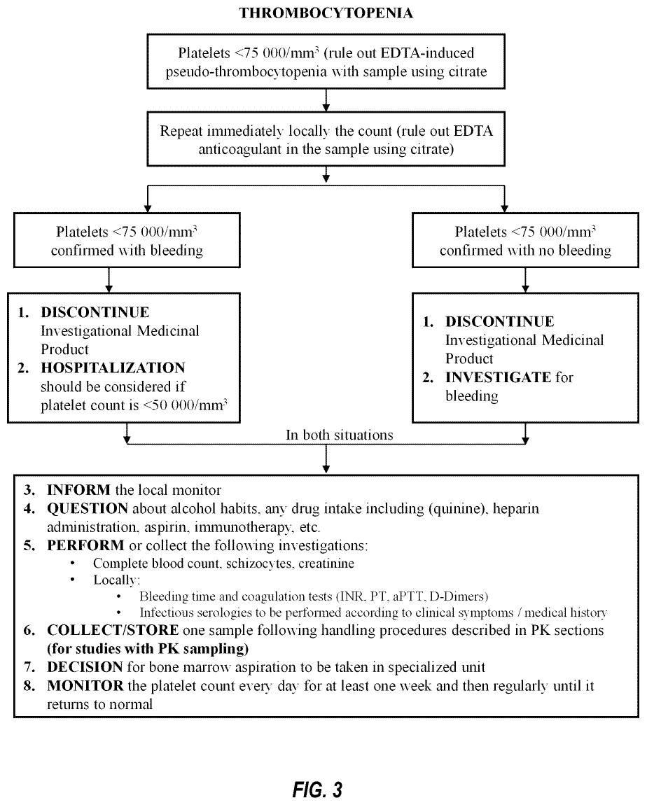

An AESI is an AE (serious or nonserious) of scientific and medical concern specific to the Sponsor's product or program, for which ongoing monitoring and immediate notification by the Investigator to the Sponsor is required. Such events may require further investigation in order to characterize and understand them. Adverse events of special interest may be added, modified, or removed during a study by protocol amendment.
Pregnancy of a female participant entered in a study as well as pregnancy occurring in a female partner of a male participant entered in a study with IMP/NIMP
   It will be qualified as an SAE only if it fulfills one of the seriousness criteria,
   In the event of pregnancy in a female participant, IMP should be discontinued,
   Follow-up of the pregnancy in a female participant or in a female partner of a male participant is mandatory until the outcome has been determined.
Symptomatic overdose (serious or nonserious) with IMP
   An overdose (accidental or intentional) with the IMP is an event suspected by the Investigator or spontaneously notified by the participant (not based on systematic pills count) and defined as at least twice the intended dose within the intended therapeutic interval (eg, >2 tablets of the IMP within a 12-hour interval).
Increase in alanine transaminase (ALT)>3×ULN
   ALT increase >3×ULN confirmed by retest within 72 hours or in the absence of a retest within 72 hours.
Other project specific AESI(s)
   ECG observation of atrial fibrillation or atrial flutter,
   Severe infection (NCI CTCAE Grade 3 or above) infection, that may or may not meet seriousness criteria (eg, a Grade 3 opportunistic infection),
   Moderate or severe hemorrhagic events (NCI CTCAE Grade 2 or above), including but not limited to symptomatic bleeding, bleeding in a critical area or organ such as the CNS, or intraocular bleeding,
   Thrombocytopenia, platelet count <75 000/mm³ (see FIG. 3 for management flow chart).

### Clinical Laboratory Tests

The tests detailed in Table 1F will be performed by the central laboratory when feasible.

Local laboratory results are only required in the event that the central laboratory results are not available in time for either study intervention administration and/or response evaluation.

Additionally, if the local laboratory results are used to make either a study intervention decision or response evaluation, the results must be entered into the eCRF.

Protocol-specific requirements for inclusion or exclusion of participants are detailed in Tables 1C and 1D.

Additional tests may be performed at any time during the study as determined necessary by the Investigator or required by local regulations. Additional serum or urine pregnancy tests may be performed, as determined necessary by the Investigator or required by local regulation, to establish the absence of pregnancy at any time during the participant's participation in the study.

TABLE 1F
Protocol-required laboratory assessments

| Laboratory | | | |
|---|---|---|---|
| assessments | Parameters | | |
| Hematology | Platelet count | RBC indices: | WBC count with |
| | | | differential: |
| | RBC count | MCV | Neutrophils |
| | Hemoglobin | MCH | Lymphocytes |
| | Hematocrit | % Reticulocytes | Monocytes |
| | | | Eosinophils |
| | | | Basophils |
| Clinical | BUN | Sodium | AST/SGOT |
| chemistry^{a} | Creatinine | Calcium | ALT/SGPT |
| | Glucose | Total and direct bilirubin | Alkaline phosphatase |
| | Potassium | Total protein | Albumin |
| | | Chloride | Creatine phosphokinase |
| | | Bicarbonate | Lipase |

| | |
|---|---|
| Routine | Specific gravity |
| urinalysis | pH, glucose, protein, blood, ketones, bilirubin, urobilinogen, nitrite, leukocyte |
| | esterase by dipstick |
| | Microscopic examination (if blood or protein is abnormal and for signs of |
| | infection) |
| Other | FSH (if needed, only in female participants to confirm postmenopausal |
| screening | state) |
| tests | Highly sensitive serum or urine β-hCG pregnancy test (as needed for |
| | women of childbearing potential) ^{b} |
| | Serology tests for hepatitis B (HBs Ag, anti-HBC IGM and total, anti-HBs) |
| | and C virus (anti-HCV); in case these results are inconclusive (eg anti-HBs |
| | negative and anti-HBc positive or anti-HC IgG positive), HBV-DNA or |
| | HCV-RNA testing, respectively, should be performed for confirmation. HIV |
| | and other infectious diseases, if locally required. |
| | Coagulation: PT/ INR, aPTT |
| | Tuberculosis test: Blood testing (eg, QuantiFERON ® TB Gold test) is |
| | preferred; skin testing (eg, tuberculin skin test) with ancillary testing will be |
| | allowed if blood testing is not available. T-SPOT can also be performed, if |
| | available ^{c}. |
| | Iron panel (serum): iron, ferritin, trnasferrin saturation TIBC. |

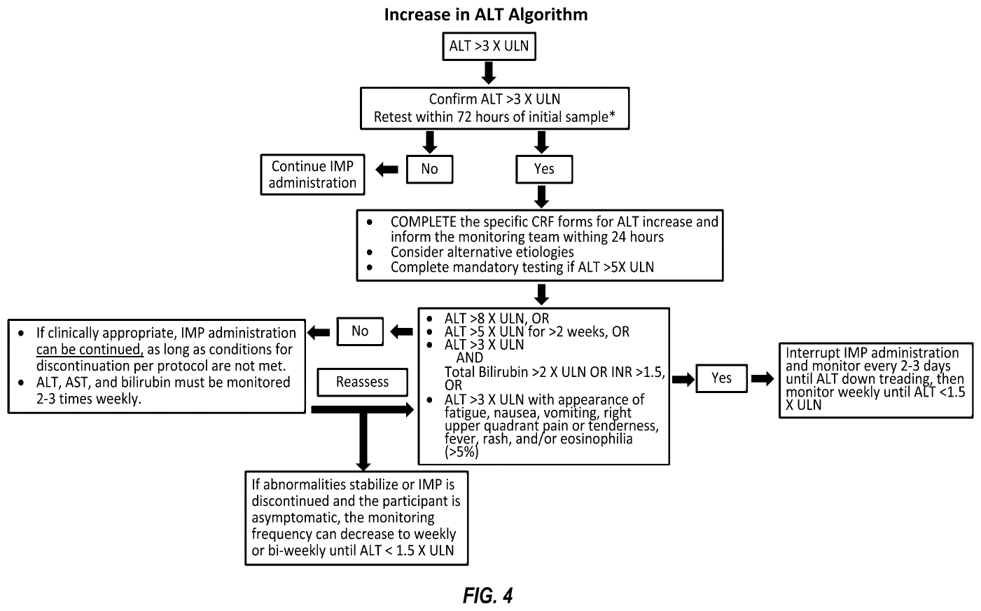

ALT: alanine aminotransferase;
anti-HBc; antibody to hepatitis B core antigen;
anti-HBs: hepatitis B surface antibody;
aPTT: activated partial thromboplastin time;
AST: aspartate aminotransferase;
BUN: blood urea nitrogen;
β-hCG: human chorionic gonadotropin;
FSH; follicle-stimulating hormone;
IEC: independent ethics committee;
INR: international normalized ratio;
HBsAg: hepatitis B surface antigen;
HBV: hepatitis B virus;
HCV: hepatitis C virus;
HIV: human immunodeficiency virus;
Ig: immunoglobulin;
IRB: institutional review board;
MCH: mean corpuscular hemoglobin;
MCV: mean corpuscular volume;
PT: prothrombin time;
RBC: red blood cell;
SGOT: serum glutamic-oxaloacetic transaminase;
SGPT: serum glutamic-pyruvic transaminase;
TB: tuberculosis;
ULN: upper limit of normal;
WBC: white blood cell
^{a}Details of liver chemistry stopping criteria and required actions and follow-up assessments after observations of ALT >3 × ULN are given in FIG. 4. Clinical laboratory findings of ALT >3 × ULN and bilirubin ≥2 × ULN (>35% direct bilirubin), or ALT >3 × ULN and international normalized ratio (INR) >1.5, if INR measured, that may suggest severe liver injury and must be reported as an SAE.
^{b} Local urine testing for pregnancy will be standard for the protocol (except for the Screening Visit, when a serum pregnancy test is required) unless serum testing is required by local regulation or IRB/IEC.
^{c} Further details are given in E 01

Investigators must document their review of each laboratory safety report.

Laboratory/analyte results that could unblind the study will not be reported to investigative sites or other blinded personnel until the study has been unblinded. This includes PK assessments and any post-baseline biomarker or PD assessments.

### Liver and Other Safety: Actions and Follow-Up Assessments

These actions described in Table 1G and FIGS. 3-4 are required for ALT increase and thrombocytopenia events ONLY. For all other safety events described, these are suggested per the Investigator's medical judgement.

Neutropenia is to be recorded as an AE only if at least 1 of the criteria listed in the general guidelines for reporting AEs in is met.

Thrombocytopenia is to be recorded as an AE only if at least 1 of the criteria listed in the general guidelines for reporting AEs in is met.

Note: “Baseline” refers to ALT sampled at baseline visit; or if baseline value unavailable, to the latest ALT sampled before the baseline visit. The algorithm does not apply to the instances of increase in ALT during Screening. Normalization is defined as ≤ULN or baseline value if baseline value is >ULN.

TABLE 1G
Actions for cases of confirmed ALT elevation
In ANY CONFIRMED CASE of ALT >5 × ULN, or ALT >3 × ULN with
bilirubin >2 × ULN, the following steps are REQUIRED (recommended
for ALT >3 × ULN but ALT <5 × ULN, as clinically indicated):

INFORM the Site Monitor, who will forward the information to the Study Manager.
INVESTIGATE specifically for malaise with or without loss of consciousness,
dizziness, and/or hypotension and/or episode of arrhythmia in the previous 72
hours; rule out muscular injury.
PERFORM the following tests:
LFTs: AST, ALT, alkaline phosphatase, GGT, total and conjugated bilirubin,
and prothrombin time/INR (mandatory assessments for ALT >3 × ULN);
CPK, serum creatinine, complete blood count;
Anti-HAV IgM, anti-HBc IgM, (HBV-DNA if clinically indicated), hepatitis B
surface antigen (HBsAg), anti-HCV and HCV RNA, anti-CMV IgM, and anti-
HEV IgM antibodies;
Iron, ferritin, transferrin saturation;
Auto-antibodies: serum IgG levels, antinuclear, anti-DNA, anti-smooth muscle,
anti-LKM, anti-mitochondrial;
Evaluate recent infection with EBV, herpes viruses. Depending on the clinical
context, consider testing for toxoplasma;
Collect and freeze serum sample (5 mL × 2);
Collect and store one PK sample following the instructions in the central
laboratory manual;
Perform hepatobiliary imaging (ultrasonography or other imaging
investigations is required);
Consider DNA test for Gilbert's disease if clinically indicated;
Recommend consulting a hepatologist (mandatory if ALT >8 × ULN or is
associated with elevated bilirubin);
Discuss with the hepatologist the clinical indication for potential liver
biopsy (strongly recommended if the participant meets Hy's law criteria
or has ALT >20 × ULN) and/or initiation of treatment with steroids;
Consider patient hospitalization if INR >2 (or PT <50%) and/or central nervous
system disturbances suggesting hepatic encephalopathy.
MONITOR LFTs after discontinuation of IMP:
Monitor closely (every 2-3 days) until ALT is down-trending,
then weekly until <1.5 × ULN, and then at every scheduled
visit;
This frequent LFT monitoring may be done through central or
local lab, or via home visit (depending on the Investigator's
assessment and/or local regulatory requirements).
Rechallenge Re-initiation of the study drug can only be considered after
discussion with the Sponsor's Medical Monitor once the ALT/AST decreases
to <1.5 × ULN, and there is no clinical contraindication. Rechallenge is not
permitted for the following participants unless a clear non-DILI etiology is
identified:
ALT >8 × ULN
ALT >5 × ULN for greater than two weeks
ALT >3 × ULN and total bilirubin >1 × ULN
In case it is agreed to re-start the study drug, it is recommended that
ALT/AST be assessed per protocol schedule of assessments for the
first 6 months of the treatment period.
The occurrence of new elevation to >3 × ULN for the ALT/AST values
will lead to permanent discontinuation of the study drug
ALT, alanine aminotransferase; AST, aspartate aminotransferase; CMV, cytomegalovirus; CPK, creatine phosphokinase; CRF, case report form; EBV, Epstein-Barr virus; GGT, gamma glutamyl transferase; HAV, hepatitis A virus; HBV, hepatitis B virus; HCV, hepatitis C virus; HEV, hepatitis E virus; IgG, immunoglobulin G; IgM, immunoglobulin M; IMP, investigational medicinal product; INR, international normalized ratio; LFT, liver function test; LKM, liver-kidney microsomal antibody; PT, prothrombin time; ULN, upper limit of normal.

Increase in serum creatinine is to be recorded as an AE only if at least 1 of the criteria listed in the general guidelines for reporting adverse events is met.

Increase in CPK is to be recorded as an AE only if at least 1 of the criteria in the general guidelines for reporting adverse events is met.

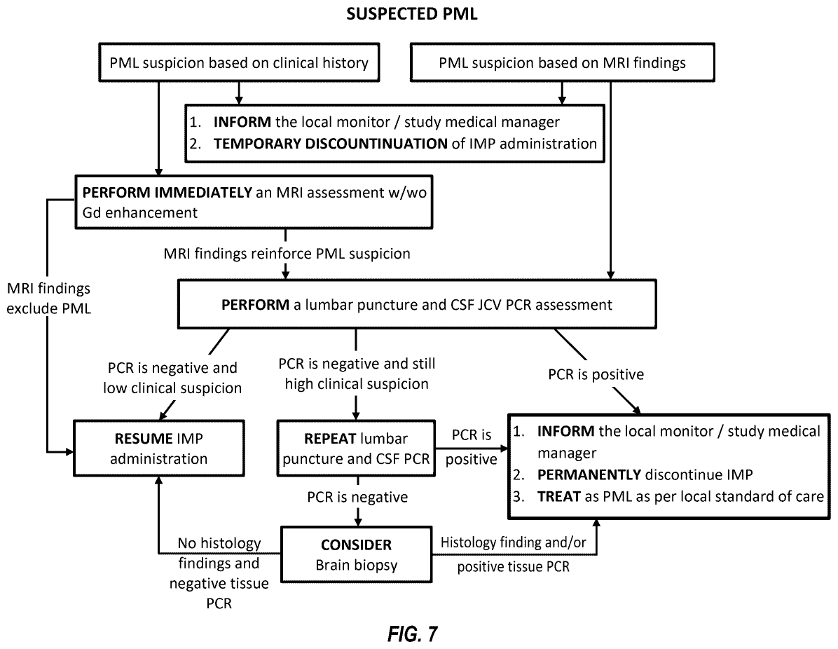

SUSPECTED PML: If either the clinical presentation or MIRI features of a participant are suggestive of PML, the diagnostic and action algorithm described in FIG. 7 is recommended.

Clinical manifestations or MRI lesions features suspicious for PML are proposed in Table 1H (based on Berger et al. Neurology 2013, 80, 1430-1438 and Kappos et al. Lancet Neurol. 2007, 6, 431-441).

TABLE 1H
Clinical and MRI features suggestive of PML

| | |
|---|---|
| Clinical history | Subacute onset of weakness, sensory deficits, cognitive |
| | or behavioral abnormalities, gait dysfunction, speech/language |
| | difficulties or any other signs of cortical dysfunction, |
| | retrochiasmal visual defects or seizure |
| Brain MRI | ≥1 T2/FLAIR hyperintense and T1 hypointense lesions |
| | involving the subcortical and juxtacortical white matter, sparing |
| | the cortex, with no mass effect, with a continuous progression; |
| | new lesions with no enhancement (even when large) or with faint |
| | rim enhancement |

In the event that PML is suspected based on imaging results, the local radiologist will directly inform the Investigator and a central review of the MRI will not be required. The Investigator will obtain additional plasma, urine, and CSF samples for John Cunningham virus (JCV) analysis. Samples will be analyzed upon receipt and the results will be provided directly to the investigational site and to the Sponsor. Further management will be deferred to the Treating Investigator. However, next steps will include discontinuation of study treatment. Additional imaging will be at the discretion of the Investigator depending on the diagnostic workup and treatment plan.
The detection of John Cunningham virus (JCV) DNA in the cerebrospinal fluid of a patient with clinical and MRI features suggestive of PML establishes the diagnosis of PML.
If JCV DNA is not detected in cerebrospinal fluid and if clinical suspicion of PML remains high, another lumbar puncture should be performed.
If diagnosis remains uncertain and suspicion of PML remains high, a brain biopsy may be considered to establish a definitive diagnosis

Clinical or MRI features suggestive of PML should be recorded as an AE/AESI/SAE.

Example of Drugs with a Potential to Change Tolebrutinib Metabolism or Absorption

The following drugs should not be taken during the study concomitantly with IMP due to their potential to change tolebrutinib kinetics due to interaction with P450-mediated metabolism, being potent and moderate inducers of CYP3A or potent inhibitors of CYP2C8 liver enzymes (per the lists of the Drug Interaction Database Program of the University of Washington) (see also Table 1I).

Additionally, participants in the US, Israel, and any other sites following FDA partial clinical hold conditions must not take medications that are mild, moderate, or potent inhibitors of CYP3A or CYP2C8 (Table 1J).

Please note that the lists provided in Table 1I and 1J are not exhaustive and that the product information of drugs intended for concomitant use should be consulted.

TABLE 1I
CYP3A Inducers and CYP2C8 Inhibitors

| | |
|---|---|
| Potent CYP3A Inducers: | |
| Rifampin | Carbamazepine |
| St John's Wort extract | Phenobarbital |
| Avasimibe | Lumacaftor |
| Rifapentine | Rifabutin |
| Phenytoin | |
| Potent CYP2C8 Inhibitors: | |
| Gemfibrozil | Clopidogrel |
| Moderate CYP3A Inducers: | |
| Semagacestat | Asunaprevir/beclabuvir/daclatasvir |
| Cenobamate | Nafcillin |
| Lesinurad | Telotristat ethyl |
| Bosentan | Elagolix |
| Thioridazine | |
| Rifabutin | |

TABLE 1J
Mild, moderate, and potent inhibitors of CYP3A and
CYP2C8, and moderate and potent inducers of CYP3A

| | Potent | Moderate | Mild |
|---|---|---|---|
| CYP2C8 inhibitors | Clopidogrel | Trimethoprim | Sulfamethoxazole |
| | Gemfibrozil | | trimethoprim |
| | | | Fluvoxamine |

| | Potent | Moderate |
|---|---|---|
| CYP3A inducers | Avasimibe | Elagolix |
| | Rifampin | Cenobamate |
| | Carbamazepine | Nafcillin |
| | Lumacaftor | Asunaprevir/beclabuvir/daclatasvir |
| | Phenobarbital | Lesinurad |
| | Phenytoin | Bosentan |
| | Rifapentine | Thioridazine |
| | St. John's Wort | Rifabutin |

| | Potent | Moderate | Mild |
|---|---|---|---|
| CYP3A | Clarithromycin | Ciprofloxacin | Alprazolam |
| inhibitors | Itraconazole | Diltiazem | Atorvastatin |
| | Ketoconazole | Erythromycin | Amlodipine |
| | Nirmatrelvir and ritonavir | Fluconazole | Cimetidine |
| | Fluoxetine | Verapamil | Ranitidine |
| | Grapefruit juice | Sertraline | Roxithromycin |
| | | | Ginkgo biloba |
| | | | Isoniazid |

TABLE 1L
Demographic and disease characteristics of study patients

| | nrSPMS |
|---|---|
| | Hercules |
| | (EFC16645) |
| Baseline Characteristics | |
| Age (mean) years | 49 |
| Female | 62% |
| White | 93% |
| Asian | 5% |
| Black/African American | 1% |
| US | 10% |
| Eastern Europe | 36% |
| Western Europe | 37% |
| North America | 14% |
| Rest of World | 13% |
| Disease Characteristics | |
| EDSS (mean) | 5.5 |
| Symptom onset (median) years | 16^{#} |
| Diagnosis (median) years | 5.5 |
| Treatment naive | 26% |
| ≥2 prior DMT | 47% |
| Gd+ T1 lesion free | 87% |
| Relapse in prior year | No relapse in |
| | prior 2 years |

^{#}onset of RMS

TABLE 1M
Baseline characteristics of study patients (intention-to-treat population)^{a}

| | Placebo | | Tolebrutinib | |
|---|---|---|---|---|
| Characteristic | (N = 377) | | (N = 754) | |
| Age, years | 48.9 | (8.0) | 48.9 | (8.0) |
| Female-no. (%) | 242 | (64.2) | 454 | (60.2) |
| Race-no. (%) | | | | |
| White | 348 | (92.3) | 703 | (93.2) |
| Black or African American | 4 | (1.1) | 6 | (0.8) |
| Asian | 19 | (5.0) | 36 | (4.8) |
| Other/unknown/not reported | 6 | (1.6) | 9 | (1.2) |
| Region-no. (%) | | | | |
| US | 37 | (9.8) | 74 | (9.8) |
| Non-US | 340 | (90.2) | 680 | (90.2) |
| EDSS score^{b} | | | | |
| Mean (SD) | 5.6 | (0.9) | 5.5 | (1.0) |
| Median (IQR) | 6.0 | (5.0-6.3) | 6.0 | (4.8-6.3) |
| Time since onset of symptoms of relapsing-remitting multiple | 17.6 | (8.4) | 17.1 | (8.3) |
| sclerosis-years | | | | |
| Time since diagnosis of secondary progressive multiple sclerosis- | 8.4 | (7.8) | 7.9 | (7.3) |
| years | | | | |
| Time since most recent clinical relapse-years | 7.6 | (5.5) | 7.4 | (5.3) |
| Number of previous disease-modifying therapies received-no. (%) | | | | |
| 0 | 89 | (23.6) | 205 | (27.2) |
| 1 | 102 | (27.1) | 200 | (26.5) |
| ≥2 | 186 | (49.3) | 349 | (46.3) |
| Previous disease-modifying therapies received^{c, d}-no. (%) | | | | |
| Interferons^{e} | 177 | (46.9) | 354 | (46.9) |
| Glatiramer acetate | 99 | (26.3) | 176 | (23.3) |
| Fingolimod | 66 | (17.5) | 113 | (15.0) |
| Dimethyl fumarate | 61 | (16.2) | 93 | (12.3) |
| Ocrelizumab | 48 | (12.7) | 89 | (11.8) |
| Teriflunomide | 49 | (13.0) | 82 | (10.9) |
| Natalizumab | 42 | (11.1) | 72 | (9.5) |
| Rituximab | 23 | (6.1) | 47 | (6.2) |
| Other | 66 | (17.5) | 115 | (15.3) |
| Participants with ≥1 gadolinium-enhancing | 49 | (13.1) | 93 | (12.5) |
| T1-weighted lesions-no. (%) | | | | |
| Number of gadolinium-enhancing T1-weighted lesions | | | | |
| Mean (SD) | 0.6 | (3.5) | 0.4 | (2.0) |
| Median (IQR) | 0 | (0-0) | 0 | (0-0) |
| Number of T2-weighted lesions | | | | |
| Mean (SD) | 56.5 | (33.4) | 57.2 | (31.8) |
| Median (IQR) | 49 | (33-75) | 50 | (35-73) |
| T2-weighted lesion volume-cm³ | | | | |
| Mean (SD) | 19.2 | (14.7) | 18.7 | (14.5) |
| Median (IQR) | 14.9 | (7.6-28.3) | 15.3 | (7.2-25.8) |

| | | |
|---|---|---|
| Normalized brain volume-cm³, median (IQR) | 1462.0 | 1467.8 |
| | (1401.5-1515.0) | (1404.2-1529.3) |

^{a}Values are mean (SD) unless otherwise indicated. Note variations in subpopulation numbers due to missing data: time since most recent clinical relapse, tolebrutinib N = 752; participants with ≥1 gadolinium-enhancing T1-weighted lesions and number of gadolinium-enhancing T1-weighted lesions, placebo N = 373, tolebrutinib N = 742; T2-weighted lesion volume, tolebrutinib N = 747; normalized brain volume, tolebrutinib N = 746, teriflunomide N = 375.
^{b}EDSS scores range from 0 to 10.0, with higher scores indicating worse disability. Baseline values represent the average of screening and randomization assessments.
^{c}A participant can be counted in more than 1 category. Disease-modifying therapies used by <5% of participants in either group are not shown.
^{d}A washout period was required for participants who received certain prior disease-modifying therapies.
^{e}Interferon therapies include interferon-beta-1a, interferon-beta-1b, and peginterferon beta-1a.
EDSS, Expanded Disability Status Scale; Gd, gadolinium; IQR, interquartile range; SD, standard deviation.

About 50% of study participants were age 50 or under. About 40% of study participants had EDSS of 5.5 or under.

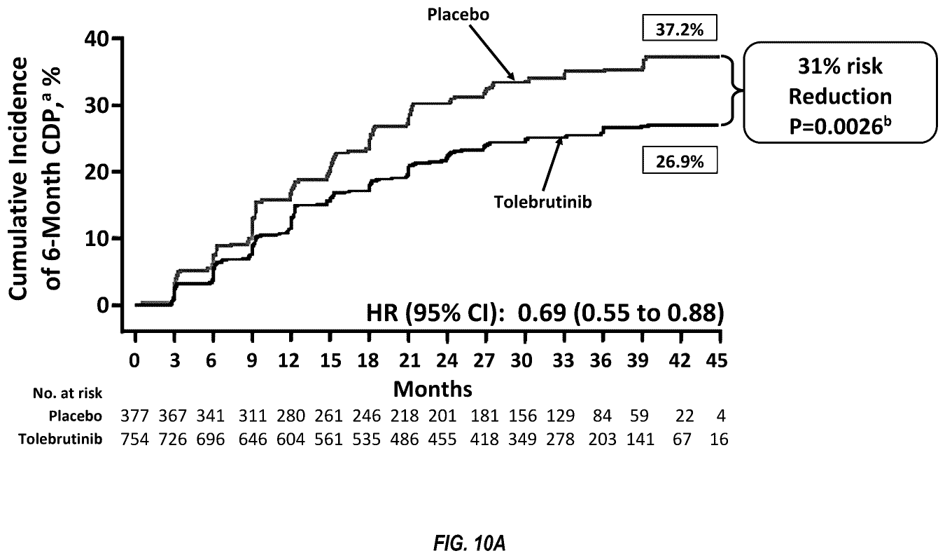

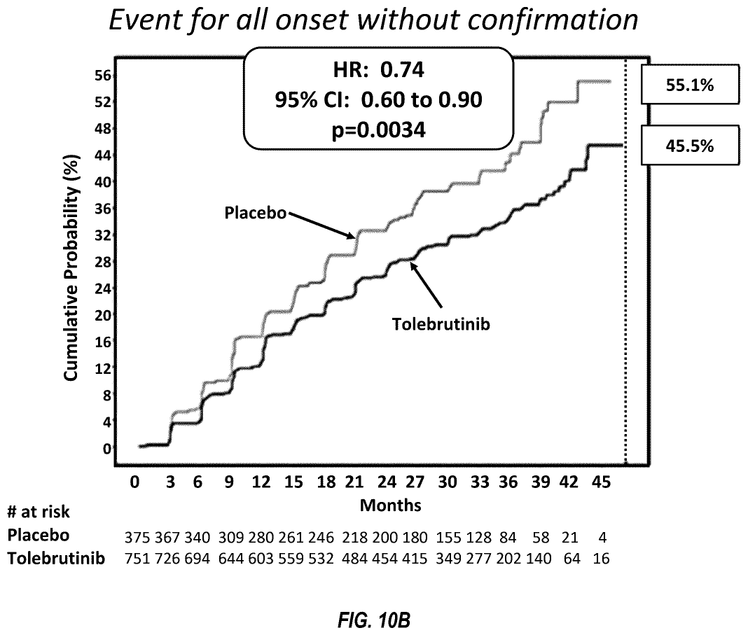

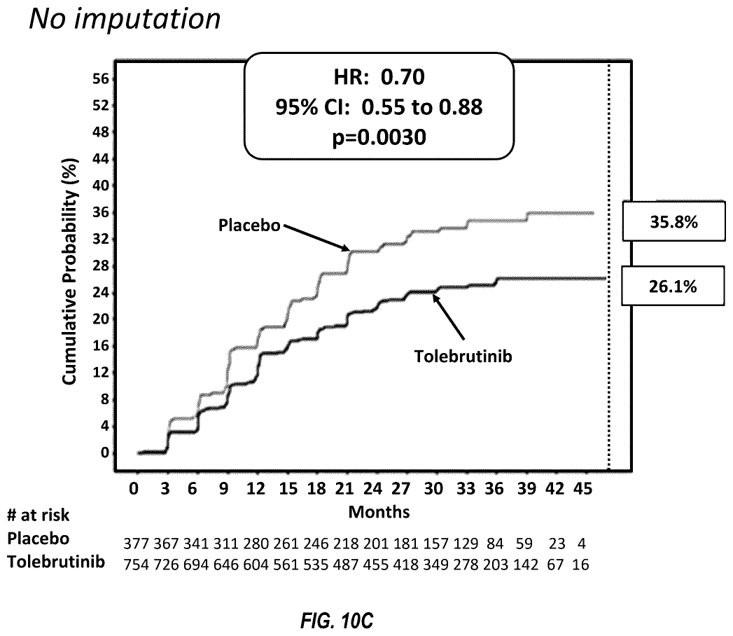

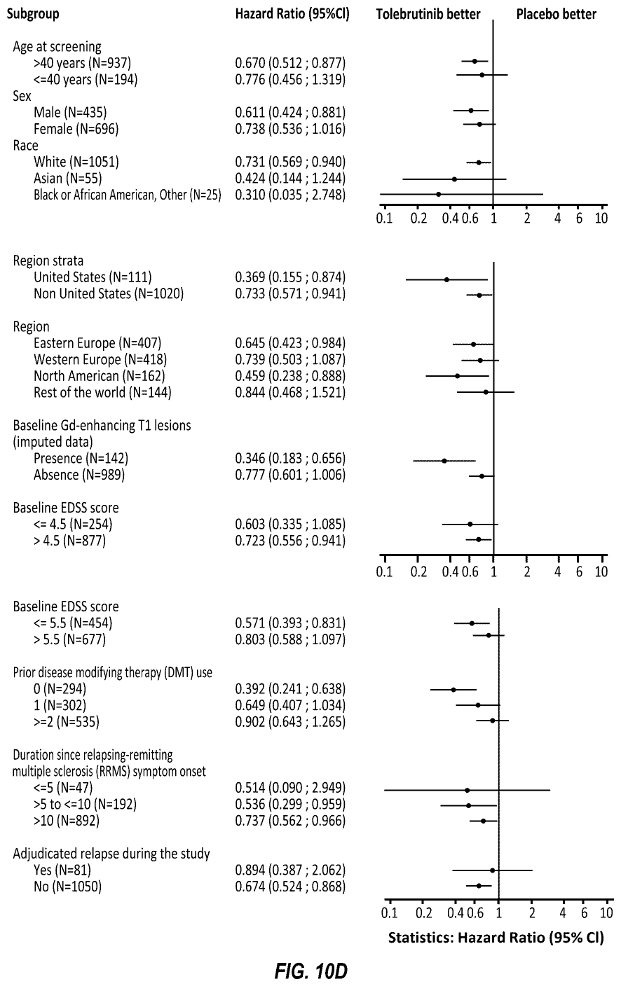

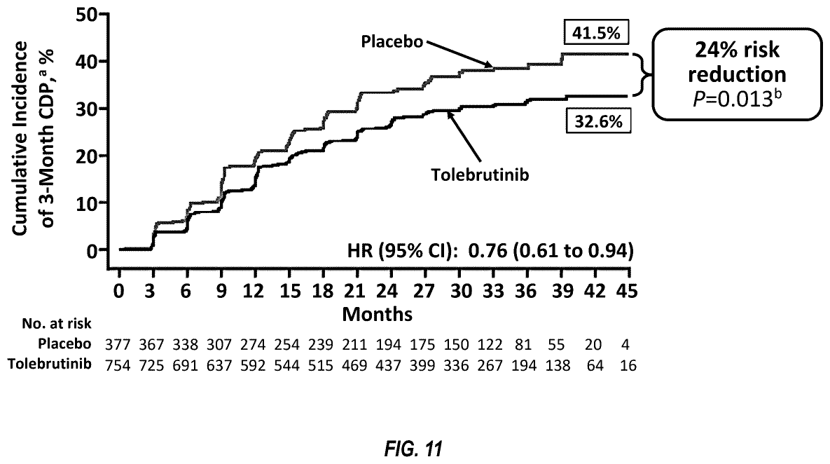

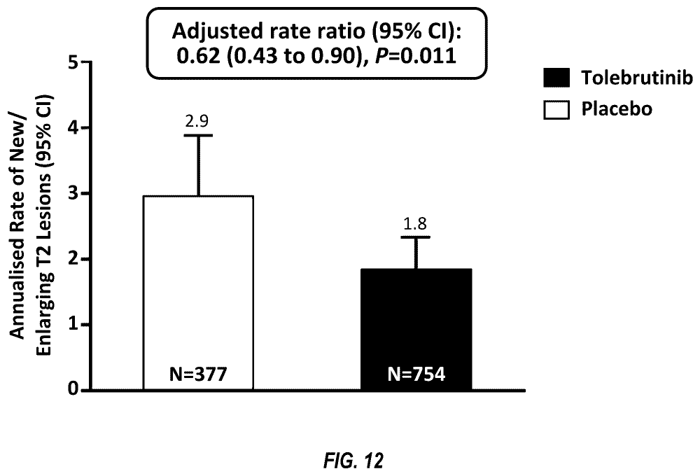

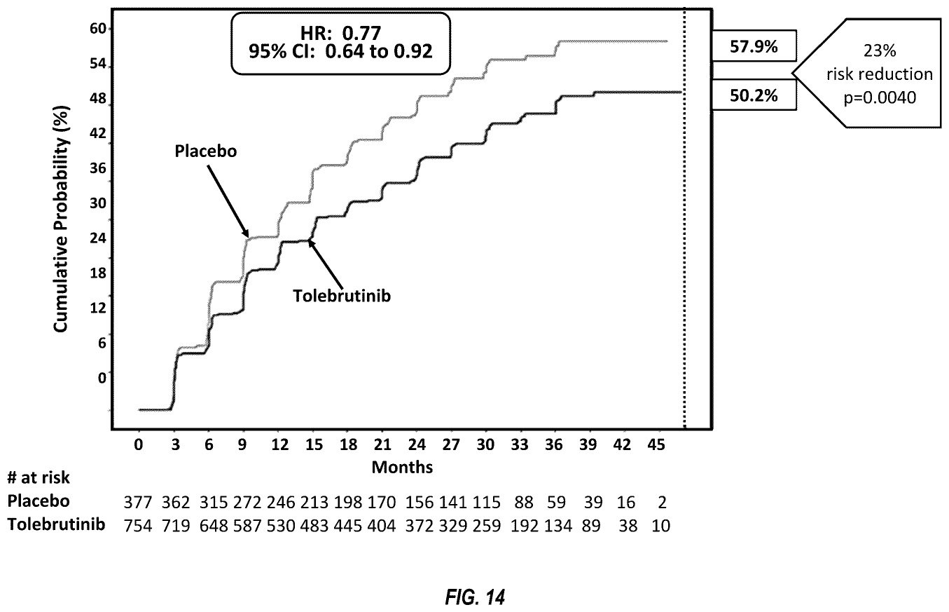

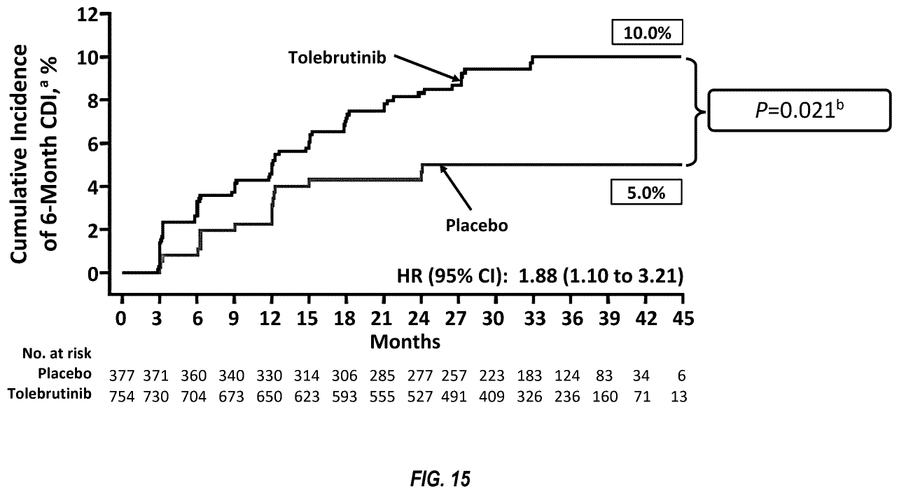

The study results showed treatment with tolebrutinib reduced disability accumulation in patients with nrSPMS compared to placebo (e.g., FIGS. 10A-10D, 11-12, 14, and 15). Specifically, the primary endpoint of time to onset of 6-month CDP measured by EDSS scores was met. Tolebrutinib delayed the time to onset of 6-month confirmed disability progression (CDP) by 31% compared to placebo (HR 0.69; 95% CI 0.55-0.88; p=0.0026) (FIG. 10A). Tolebrutinib also delayed time to onset of 3-month confirmed disability progression (CDP) by 24% compared to placebo (HR 0.76; 95% CI 0.61-0.94; p=0.013) (FIG. 11). Tolebrutinib also significantly reduced the annualized rate of the total number of new and/or enlarging T2-hyperintense lesions as detected by MRI by 1.1 compared to placebo (HR 0.62; 95% CI 0.43-0.90; p=0.011) (FIG. 12). Tolebrutinib also delayed time to onset of sustained 20% increase in the timed 25-foot walk (T25-FW) test confirmed over at least 3 months by 23% compared to placebo (HR 0.77; 95% CI 0.64-0.92; p=0.0040) (FIG. 14). In further analysis of secondary endpoints, tolebrutinib also demonstrated that the number of participants who experienced confirmed disability improvement (CDI) increased by nearly two-fold, 10% with tolebrutinib compared to 5% with placebo (HR 1.88; 95% CI 1.10 to 3.21; p=0.0206) (FIG. 15).

These results demonstrate tolebrutinib's significant slowing of disability progression in people with nrSPMS. In the GEMINI 1 and 2 phase 3 trials (Example 2), the rate of 6-month confirmed disability worsening was 8.3% with tolebrutinib and 11.3% with teriflunomide (hazard ratio 0.71; 95% CI, 0.53 to 0.95). The totality of results from the HERCULES and GEMINI 1 and 2 trials indicates that tolebrutinib has an impact on disability, that may be driven by effects on chronic inflammation within the CNS.

The totality of the data from the HERCULES study (Example 1) and GEMINI studies (Example 2) indicate that tolebrutinib has a consistent impact on disability accumulation that may be largely driven by effects on smoldering neuroinflammation.

A summary of the results collected from the Hercules (EFC16645) study described in this example are listed in Table 1N.

TABLE 1N
Summary of the multiplicity adjustment for the primary and other
secondary efficacy endpoints in Hercules study (EFC16645)

| | | | Comparison between placebo |
|---|---|---|---|
| Category | Endpoint | | and tolebrutinib 60 mg |
| Primary Endpoint | 6-month CDP | Hazard Ratio (95% CI) | 0.693 (0.546, 0.880) |
| | | p-value | 0.0020 |
| | | Adjusted significance level | 0.05 |
| Secondary | 3-month CDP | Hazard Ratio (95% CI) | 0.757 (0.607, 0.944) |
| Endpoints | | | |
| | | p-value | 0.0106 |
| | | Adjusted significance level | 0.05 |
| | New and/or enlarging T2- | Relative risk (95% CI) | 0.622 (0.432, 0.897) |
| | hyperintense lesions | | |
| | | p-value^{a} | 0.0110 |
| | | Adjusted significance level | 0.05 |
| | Sustained 20% increase in | Hazard Ratio (95% CI) | 0.972 (0.735, 1.286) |
| | 9-HPT for at least 3 months | | |
| | | p-value | 0.6614 |
| | | Adjusted significance level | 0.05 |
| | Sustained 20% increase in | Hazard Ratio (95% CI) | 0.767 (0.640, 0.919) |
| | T25-FW for at least 3 | | |
| | months | | |
| | | p-value | 0.0036 |
| | | Adjusted significance level | NA |
| | 6-month CDI | Hazard Ratio (95% CI) | 1.882 (1.102, 3.214) |
| | | p-value | 0.0120 |
| | | Adjusted significance level | NA |
| | Brain Volume | LS Mean difference (95% CI) | 0.082 (−0.034, 0.197) |
| | | p-value for the difference | 0.1646 |
| | | between groups | |
| | | Adjusted significance level | NA |

^{a}Chi-square test for relative risk.
All values in bold font are statistically significant according to the hierarchical testing procedure

TABLE 1O
Adverse events and ALT elevations (safety population)^{a}

| | Placebo | | Tolebrutinib | |
|---|---|---|---|---|
| Event,^{b} n (%) | (N = 375) | | (N = 752) | |
| Any adverse event | 293 | (78.1) | 613 | (81.5) |
| Adverse events leading to treatment discontinuation | 11 | (2.9) | 29 | (3.9) |
| Any serious adverse event | 39 | (10.4) | 113 | (15.0) |
| Serious infection | 13 | (3.5) | 39 | (5.2) |
| Deaths^{c} | 1 | (0.3) | 2 | (0.3) |
| Incidence of increase ALT levels >3 × ULN^{d, e} | 6 | (1.6) | 30 | (4.0) |
| 3-5x ULN | 3 | (0.8) | 15 | (2.0) |
| 5-10x ULN | 2 | (0.5) | 8 | (1.1) |
| 10-20x ULN | 1 | (0.3) | 3 | (0.4) |
| >20x ULN | 0 | | 4 | (0.5) |
| ALT >3 × ULN and total bilirubin >2 × ULN | 0 | | 3 | (0.4)^{e} |
| Most common adverse events (≥2% in either group) | | | | |
| Blood and lymphatic system disorders | 19 | (5.1) | 64 | (8.5) |
| Anemia | 2 | (0.5) | 16 | (2.1) |
| Neutropenia | 8 | (2.1) | 12 | (1.6) |
| Gastrointestinal disorders | 59 | (15.7) | 150 | (19.9) |
| Diarrhea | 14 | (3.7) | 33 | (4.4) |
| Constipation | 12 | (3.2) | 22 | (2.9) |
| Abdominal pain upper | 2 | (0.5) | 19 | (2.5) |
| Nausea | 8 | (2.1) | 17 | (2.3) |
| Dyspepsia | 7 | (1.9) | 15 | (2.0) |
| General disorders and administration site conditions | 54 | (14.4) | 128 | (17.0) |
| Fatigue | 11 | (2.9) | 35 | (4.7) |
| Oedema peripheral | 12 | (3.2) | 27 | (3.6) |
| Pyrexia | 17 | (4.5) | 25 | (3.3) |
| Infections and infestations | 185 | (49.3) | 409 | (54.4) |
| COVID-19 | 85 | (22.7) | 192 | (25.5) |
| Urinary tract infection | 49 | (13.1) | 85 | (11.3) |
| Nasopharyngitis | 26 | (6.9) | 70 | (9.3) |
| Influenza | 13 | (3.5) | 42 | (5.6) |
| Cystitis bacterial | 15 | (4.0) | 31 | (4.1) |
| Upper respiratory tract infection | 18 | (4.8) | 31 | (4.1) |
| Cystitis | 14 | (3.7) | 29 | (3.9) |
| Viral upper respiratory tract infection | 12 | (3.2) | 28 | (3.7) |
| Bronchitis | 5 | (1.3) | 19 | (2.5) |
| Pharyngitis | 4 | (1.1) | 17 | (2.3) |
| Injury, poisoning and procedural complications | 88 | (23.5) | 179 | (23.8) |
| Fall | 41 | (10.9) | 72 | (9.6) |
| Contusion | 4 | (1.1) | 29 | (3.9) |
| Accidental overdose | 19 | (5.1) | 25 | (3.3) |
| Ligament sprain | 7 | (1.9) | 15 | (2.0) |
| Rib fracture | 9 | (2.4) | 7 | (0.9) |
| Investigations | 36 | (9.6) | 108 | (14.4) |
| Alanine aminotransferase increased^{f} | 6 | (1.6) | 33 | (4.4) |
| Musculoskeletal and connective tissue disorders | 94 | (25.1) | 186 | (24.7) |
| Arthralgia | 19 | (5.1) | 49 | (6.5) |
| Back pain | 24 | (6.4) | 47 | (6.3) |
| Muscular weakness | 10 | (2.7) | 18 | (2.4) |
| Pain in extremity | 9 | (2.4) | 18 | (2.4) |
| Neck pain | 9 | (2.4) | 9 | (1.2) |
| Nervous system disorders | 77 | (20.5) | 176 | (23.4) |
| Headache | 27 | (7.2) | 54 | (7.2) |
| Dizziness | 7 | (1.9) | 17 | (2.3) |
| Muscle spasticity | 6 | (1.6) | 17 | (2.3) |
| Psychiatric disorders | 30 | (8.0) | 65 | (8.6) |
| Depression | 2 | (0.5) | 17 | (2.3) |
| Insomnia | 11 | (2.9) | 16 | (2.1) |
| Respiratory, thoracic and mediastinal disorders | 22 | (5.9) | 55 | (7.3) |
| Cough | 9 | (2.4) | 16 | (2.1) |
| Oropharyngeal pain | 5 | (1.3) | 15 | (2.0) |
| Skin and subcutaneous tissue disorders | 36 | (9.6) | 125 | (16.6) |
| Petechiae | 1 | (0.3) | 20 | (2.7) |
| Alopecia | 6 | (1.6) | 18 | (2.4) |
| Vascular disorders | 16 | (4.3) | 57 | (7.6) |
| Hypertension | 11 | (2.9) | 38 | (5.1) |

^{a}The safety population included all participants exposed to tolebrutinib or placebo. Data were collected from the first dose to 10 days after the last dose, the date of death, or last contact date, whichever occurred first.
^{b}Adverse events were grouped by System Organ Class and Preferred Term according to the Medical Dictionary for Regulatory Activities 27.0. System Organ Class is sorted alphabetically and Preferred Terms within each System Organ Class are sorted by descending incidence in the tolebrutinib column.
^{c}The deaths that occurred in the tolebrutinib arm were due to post-operative complications of a liver transplant (assessed as related to tolebrutinib) and assisted suicide (assessed as unrelated to tolebrutinib). The death in the placebo arm was due to cerebral edema and hemorrhage from a fall.
^{d}Tolebrutinib: N = 741; placebo: N = 372.
^{e}One case of ALT >3 × ULN and total bilirubin >2 × ULN occurred secondary to herpes simplex virus infection.
^{f}Increased alanine aminotransferase was collected as an adverse event by investigators (reported in this row) and was also determined from clinical laboratory data (rows 6-10). Small differences in these values are due to the fact that investigator reporting may not conform with the definition of >3x the upper limit of normal.
TEAE, treatment-emergent adverse event.

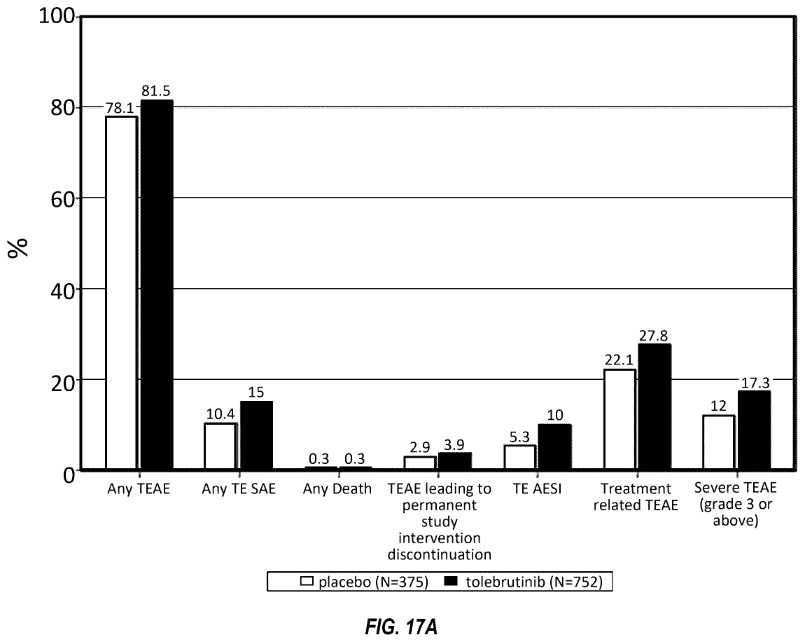

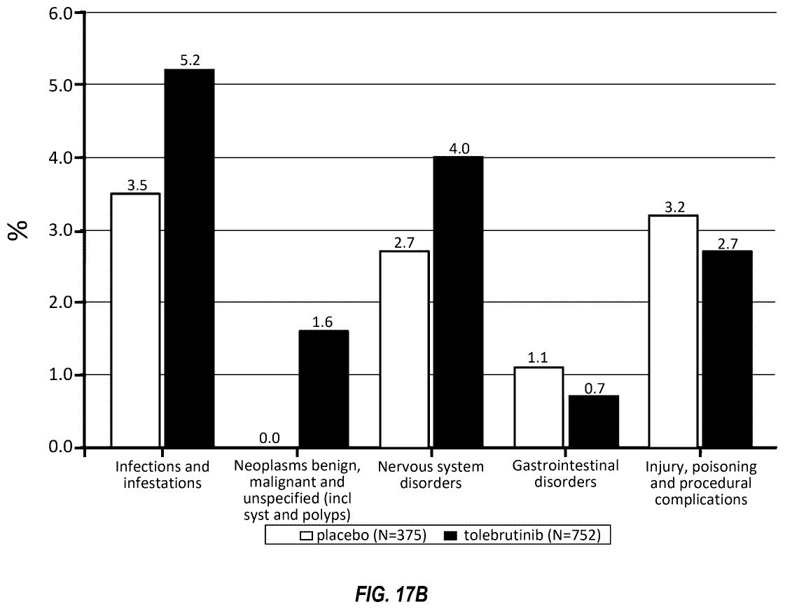

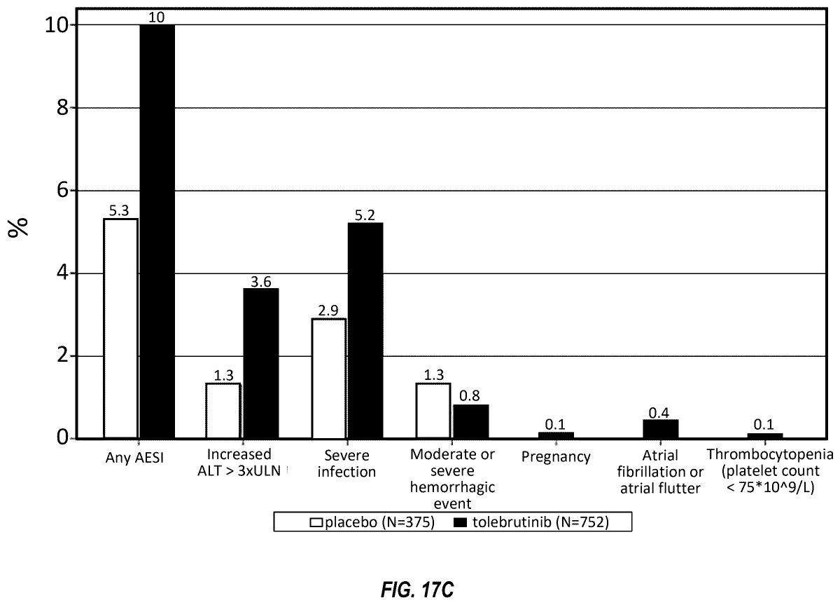

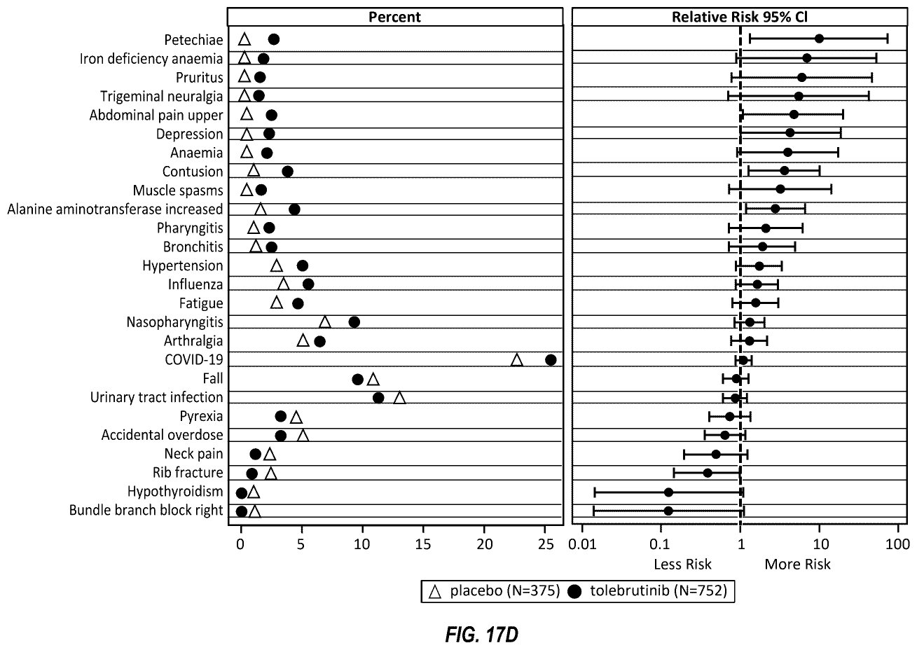

Further, the study results demonstrated the safety profile of tolebrutinib was generally consistent with that reported previously (FIGS. 17A-17D). Based on preliminary analysis of the Hercules study, there was a slight increase in the tolebrutinib arm in some adverse events from previously reported studies (Table 10). The most common adverse events (≥10%) for participants receiving tolebrutinib were COVID-19 infection (n=192 [25.5%]) and urinary tract infection (n=85 [11.3%%]). The most common adverse events (≥10%) for participants on placebo were COVID-19 infection (n=85 [22.7%]) and urinary tract infections (n=49 [13.1%]).

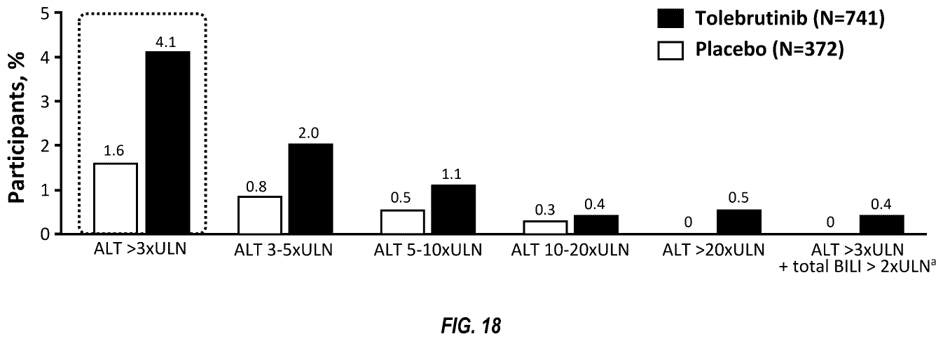

Preliminary analysis of the liver safety of tolebrutinib was generally consistent with that reported previously (FIG. 18). Liver enzyme elevations (>3×ULN) were observed in 4.1% of participants receiving tolebrutinib, compared with 1.6% in the placebo group, a side effect also reported with other BTK inhibitors in MS. A small (0.5%) proportion of participants in the tolebrutinib group experienced peak ALT increases of >20×ULN, all occurring within the first 90 days of treatment. All but one case of liver enzyme elevations resolved without further medical intervention. Prior to the implementation of the revised study protocol with more stringent monitoring, one participant in the tolebrutinib arm received a liver transplant and died to due post-operative complications. Other deaths in the trial were assessed as unrelated to treatment by investigator; deaths were even across the placebo and tolebrutinib arms at 0.3%.

### Participants

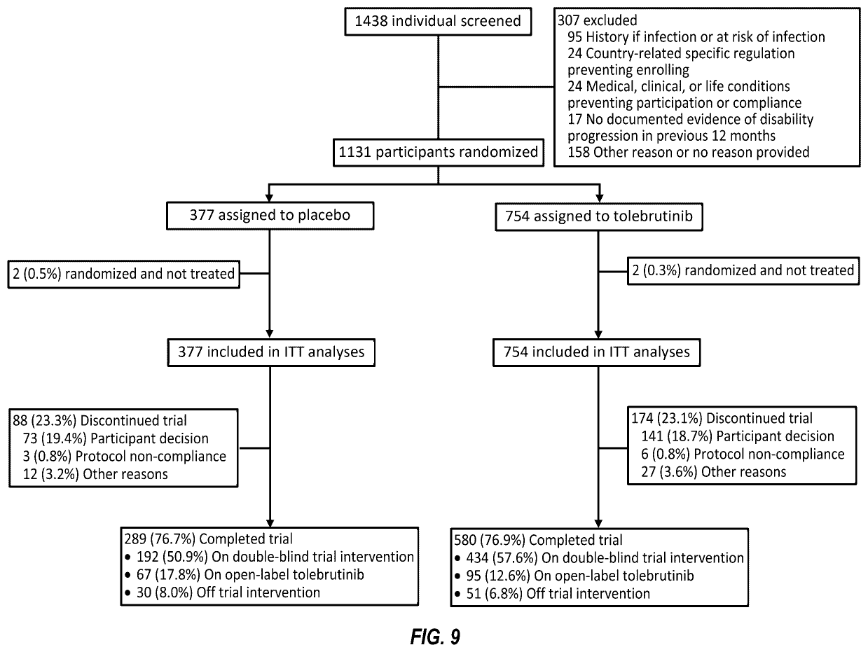

Of 1456 screened participants, 1131 were randomized between Oct. 23, 2020, and Jan. 12, 2023, 754 to tolebrutinib and 377 to placebo. Of these, 76.9% and 76.7%, respectively, completed the trial (see FIG. 9). The primary reason for early trial discontinuation in both groups was participant decision (18.7% of all participants randomized to tolebrutinib and 19.4% of all participants randomized to placebo). Median follow-up was 133.1 weeks (IQR 108.3-156.4) for the tolebrutinib group and 133.6 weeks (108.4-156.9) for the placebo group).

Demographic and disease characteristics were similar across treatment groups. The mean (standard deviation) time since secondary progressive multiple sclerosis diagnosis and most recent clinical relapse were 8.1 (7.5) years and 7.5 (5.4) years, respectively (see Table 1M). During the trial, the adjusted annualized adjudicated relapse rate was 0.033 (95% CI, 0.024 to 0.045) for tolebrutinib and 0.032 (0.021 to 0.049) for placebo. Most participants (74.0%) had previously received one or more disease-modifying therapies, and 12.7% had gadolinium-enhancing T1-weighted lesions at baseline.

### Efficacy

Primary Endpoint. The percentage of participants with 6-month confirmed disability progression during the trial was 22.6% for tolebrutinib versus 30.7% for placebo (hazard ratio, 0.69; 95% CI, 0.55 to 0.88; P=0.003; see Table 1P, FIG. 10A); the corresponding Kaplan-Meier estimates at month 24 were 21.9% and 30.2%, respectively.

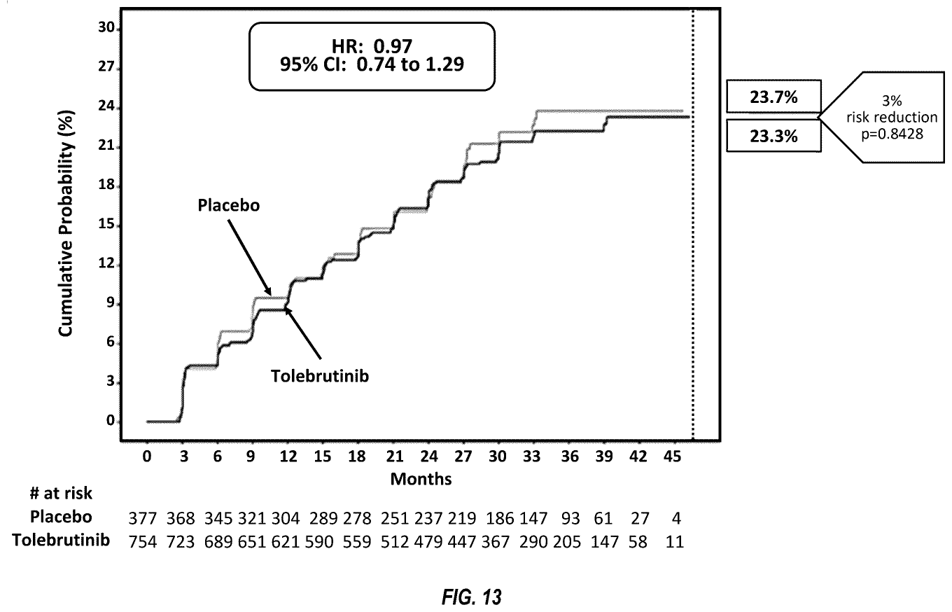

Disability-Related Secondary Endpoints. The percentage of participants with 3-month confirmed disability progression during the trial was 27.6% for tolebrutinib versus 34.2% for placebo (hazard ratio, 0.76; 95% CI, 0.61 to 0.94; P=0.01; see Table 1P, FIG. 11). The percentage of participants with sustained 20% increase in the 9-HPT for ≥3 months during the trial was 19.0% for tolebrutinib versus 19.6% for placebo (hazard ratio, 0.97; 95% CI, 0.74 to 1.29; P=0.84; see Table 1P, FIG. 13). Due to this non-significant result in the hierarchical testing, the results for all hierarchically lower secondary end points are considered nominal. The percentage of participants with sustained 20% increase in the T25-FW for ≥3 months during the trial was 41.1% for tolebrutinib versus 49.6% for placebo (hazard ratio, 0.77; 95% CI, 0.64 to 0.92; see Table 1P, FIG. 14). The percentage of participants with 6-month confirmed disability improvement during the trial was 8.6% for tolebrutinib versus 4.5% for placebo (hazard ratio, 1.88; 95% CI, 1.10 to 3.21; nominal P=0.02, see Table 1P, FIG. 15).

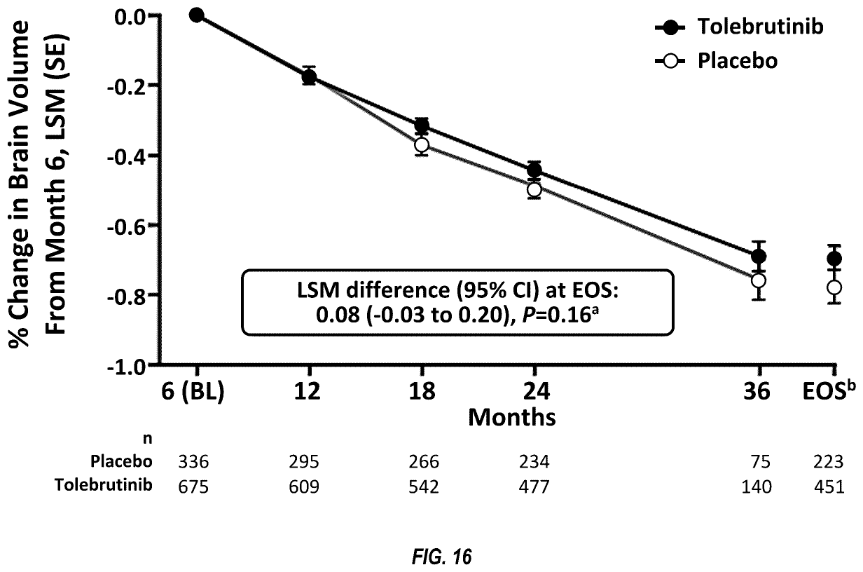

MRI-Related Secondary Endpoints. The mean annualized rate of new and/or enlarging T2-weighted lesions was 1.84 for tolebrutinib and 2.95 for placebo (relative rate, 0.62; 95% CI, 0.43 to 0.90; P=0.01; see Table 1P, FIG. 12). The least squares mean percentage change in brain volume at EOS compared to month 6 was −0.69 for tolebrutinib versus −0.78 for placebo (difference of 0.08; 95% CI, −0.03 to 0.20; see Table 1P, FIG. 16).

TABLE 1P
Primary and secondary endpoints (intention-to-treat population)

| | | |
|---|---|---|
| | Tolebrutinib | Placebo |
| End point | (N = 754) | (N = 377) |

Primary end point

| 6-month confirmed disability progression^{a} | | | | |
|---|---|---|---|---|
| No. of events/no. of participants evaluated (%) | 171/754 | (22.6) | 116/377 | (30.7) |
| Kaplan-Meier estimate at 24 months, % (95% CI) | 21.9 | (18.8 to 25.1) | 30.2 | (25.3 to 35.1) |

| | |
|---|---|
| Hazard ratio (95% CI) | 0.69 (0.55 to 0.88) |
| P value | P = 0.003 |

Secondary end points (hierarchically ordered)

| 3-month confirmed disability progression^{a} | | | | |
|---|---|---|---|---|
| No. of events/no. of participants evaluated (%) | 208/754 | (27.6) | 129/377 | (34.2) |
| Kaplan-Meier estimate at 24 months, % (95% | 26.7 | (23.5 to 30.2) | 33.3 | (28.5 to 38.7) |
| CI) | | | | |
| Hazard ratio (95% CI) | 0.76 (0.61 to 0.94) | | | |
| P value | P = 0.01 | | | |
| Annualized rate of new and/or | | | | |
| enlarging T2-weighted lesions | | | | |
| Number of participants evaluated | 719 | | 361 | |
| Mean estimate (95% CI) | 1.84 | (1.44 to 2.34) | 2.95 | (2.24 to 3.88) |
| Relative rate (95% CI) | 0.62 (0.43 to 0.90) | | | |
| P value | P = 0.01 | | | |
| Sustained 20% increase in the 9-HPT for ≥3 months | | | | |
| No. of events/no. of participants evaluated (%) | 143/754 | (19.0) | 74/377 | (19.6) |
| Kaplan-Meier estimate at 24 months, % (95% CI) | 17.1 | (14.5 to 20.2) | 16.4 | (12.9 to 20.8) |
| Hazard ratio (95% CI) | 0.97 (0.74 to 1.29) | | | |
| P value | P = 0.84 | | | |
| Sustained 20% increase in the T25-FW for ≥3 months | | | | |
| No. of events/no. of participants evaluated (%) | 310/754 | (41.1) | 187/377 | (49.6) |
| Kaplan-Meier estimate at 24 months, % (95% CI) | 36.9 | (33.4 to 40.7) | 46.9 | (41.7 to 52.4) |
| Hazard ratio (95% CI) | 0.77 (0.64 to 0.92) | | | |
| P value | P = 0.004 | | | |
| 6-month confirmed disability improvement^{b} | | | | |
| No. of events/no. of participants evaluated (%) | 65/754 | (8.6) | 17/377 | (4.5) |
| Kaplan-Meier estimate at 24 months, % (95% CI) | 8.3 | (6.5 to 10.7) | 4.3 | (2.6 to 7.1) |
| Hazard ratio (95% CI) | 1.88 (1.10 to 3.21) | | | |
| P value | P = 0.02 | | | |
| Percentage change in brain | | | | |
| volume from month 6 to EOS | | | | |
| Number of participants evaluated | 451 | | 223 | |
| LSM (SE) | −0.69 | (0.03) | −0.78 | (0.05) |

| | |
|---|---|
| LSM difference vs placebo (95% CI) | 0.08 (−0.03 to 0.20) |
| P value | P = 0.16 |

Other secondary endpoints

| Change from baseline in cognitive | | | | |
|---|---|---|---|---|
| function assessed by SDMT at EOS | | | | |
| Number of participants evaluated | 271 | | 546 | |
| LSM (SE) | 0.17 | (0.4) | 0.14 | (0.03) |
| LSM difference vs placebo (95% CI) | −0.03 (−0.12 to 0.06) | | | |
| Change from baseline in cognitive | | | | |
| function assessed by CVLT-II at EOS | | | | |
| Number of participants evaluated | 262 | | 531 | |
| LSM (SE) | 13.4 | (1.0) | 14.2 | (0.7) |
| LSM difference vs placebo (95% CI) | 0.7 (−1.6 to 3.0) | | | |
| Change from baseline in MSQoL-54 | | | | |
| Questionnaire Score at EOS | | | | |
| Physical health composite score | | | | |
| Number of participants evaluated | 262 | | 536 | |
| LSM (SE) | −4.0 | (0.8) | −3.5 | (0.6) |
| LSM difference vs placebo (95% CI) | 0.5 (−1.4 to 2.4) | | | |
| Mental health composite score | | | | |
| Number of participants evaluated | 268 | | 552 | |
| LSM (SE) | −4.6 | (1.0) | −3.9 | (0.7) |

| | |
|---|---|
| LSM difference vs placebo (95% CI) | 0.7 (−1.7 to 3.1) |

Note:
26 participants in the tolebrutinib group and 13 participants in the placebo group had missing data for 9-HPT and T25-FW and at baseline; 12 participants in the tolebrutinib group and 4 participants in the placebo group had missing data for the presence of gadolinium-enhancing lesions at baseline, which was a covariate used in the Cox proportional hazard models for the primary end point and disability-related secondary end points; 7 participants in the tolebrutinib group had missing data for the number of T2 lesions at baseline, which was a covariate in the negative binomial regression model for the annualized rate of new and/or enlarging T2-weighted lesions; 79 participants in the tolebrutinib group and 41 participants in the placebo group had missing data for brain volume at month 6, which was a covariate in the mixed effect model with repeated measures for percentage change in brain volume from month 6 to EOS.
^{a}3-month or 6-month confirmed disability progression was defined as an increase of ≥1.0 point in EDSS score from baseline for baseline scores ≤5.0, or an increase of ≥0.5 points from baseline for baseline scores >5.0, confirmed over ≥3 or ≥6 months, respectively.
^{b}6-month confirmed disability improvement was defined as a ≥1.0-point decrease in EDSS score from baseline, confirmed over ≥6 months.
9-HPT, 9-Hole Peg Test; CI, confidence interval; CVLT-II, California Verbal Learning Test - Second Edition; EOS, end of study; LSM, least squares mean; MSQoL-54: Multiple Sclerosis Quality of Life-54; SDMT, Symbol Digit Modalities Test; SE, standard error; T25-FW = timed 25-foot walk.

### Statistical Considerations

The primary end point was time to onset of 6-month confirmed disability progression, defined as an EDSS score increase from baseline of ≥1.0 point for baseline scores ≤5.0 or of ≥0.5 points for baseline scores >5.0, confirmed over ≥6 months. Only confirmed disability progression events with onset and confirmation 90 days outside of relapses were counted as events for the primary endpoint and were therefore independent of relapses.

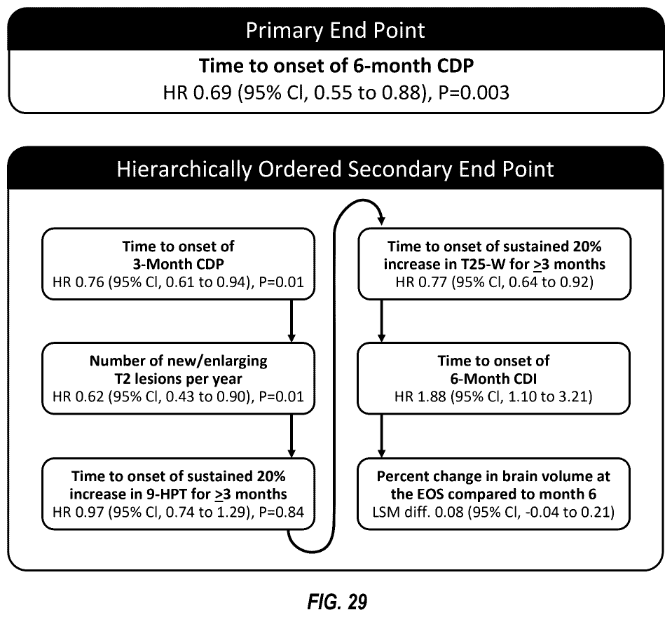

There were six hierarchically ordered secondary end points: 3-month confirmed disability progression; number of new/enlarging T2-weighted lesions per year; 3-month sustained 20% increase in the 9-Hole Peg Test (9-HIPT) (Fischer J S, et al., 1999; 5:244-50); 3-month sustained 20% increase in the timed 25-foot walk (T25-FW) (National MS Society. Timed 25-foot walk test); 6-month confirmed disability improvement (defined as ≥1.0 point decrease in EDSS score from baseline confirmed over ≥6 months); and percentage change in brain volume from month 6 to the end of study (EOS) visit (see FIG. 29).

Approximately 1700 participants were planned to be screened to facilitate randomization of up to 1290 participants. It was estimated that 288 primary end point events of 6-month confirmed disability progression would provide more than 80% power to detect a 30% relative risk reduction with tolebrutinib compared with placebo (two-sided type 1 error rate=0.05). Sample size calculations estimated a 2-year placebo event rate of 23.6% based on previous data from a similar population, 18 annual discontinuation rate of 10%, constant hazard rates using a log-rank test, and an estimated enrollment period of ˜24 months with the last randomized participant followed for an additional 24 months.

Efficacy analyses were performed per the intention-to-treat principle, with the efficacy population consisting of all randomized participants including those who prematurely discontinued treatment. The time to onset of 6-month confirmed disability progression was analyzed by a Cox proportional hazards model with terms for treatment, age at screening (>40 or ≤40 years), and geographic region (US or non-US). Data were censored at the date of last EDSS assessment for participants who completed the trial without an initial disability progression or discontinued before 6-month confirmation of disability progression. Confirmed disability progression event status was imputed for participants who completed the trial, met 3-month confirmed disability progression criteria, and continued to meet criteria for EDSS disability progression through the final trial assessment, but did not reach the 6-month confirmation visit. Since in this setting the partial missing data can reasonably be assumed to be at random, this approach leverages the partial information and follows the intention to treat principle.

Overall type 1 error rate was controlled at two-sided α=0.05 for key secondary outcomes using a predefined, sequential, hierarchical testing procedure (see FIG. 29). If statistical significance for the primary end point was met, hierarchically ordered secondary end points were tested in the order described above when all preceding end points were significant.

An imputation approach was used for the primary end point for participants who completed the trial, met 3-month confirmed disability progression criteria, and continued to meet criteria for EDSS disability progression through the final trial assessment, but did not reach 6-month confirmation. The following logistic regression model was used:

$${Y}_{i} ∼ Bernoulli ( {p}_{ij} ) , i = 1 , … , {n}_{j} ; j = 1 , 2$$

$$logit ( {p}_{ij} ) = In ( \frac{{p}_{ij}}{1 - {p}_{ij}} ) = {β}_{0 j} + {β}_{1 j} {x}_{1 ij} + {β}_{2 j} {x}_{2 ij} + {β}_{3 j} {x}_{3 ij} + {β}_{4 j} {x}_{4 ij}$$

In this model, Yᵢⱼ is the indicator for event status of 6-month confirmed disability progression for participant i from group j (1=6-month confirmed disability progression and 0=no 6-month confirmed disability progression); pᵢⱼ is the probability that participant i from group j has a 6-month CDW event; x₁ᵢⱼ is the age at screening (>40 or ≤40 years), x₂ᵢⱼ is the geographic region (US or non-US); x₃ᵢⱼ is the baseline EDSS score, x₄ᵢⱼ is the baseline gadolinium-enhancing T1-weighted lesion status (absence or presence); and βₖⱼ are the model parameters (k=0,1,2,3,4; j=1,2). The logistic model parameters βₖⱼ were estimated from participants who did not have missing value due to end of study. Separate logistic models were estimated for each treatment arm. The estimated parameters were then used to calculate the predicted probability of 6-month confirmed disability progression for participants who met the 3-month confirmed disability progression criteria and continued to meet criteria for EDSS disability progression through the final trial assessment. The missing value for those participants with an initial onset of disability worsening and missingness due to the common end of study was imputed via a randomly generated value from a Bernoulli distribution with pᵢⱼ. A total of 1000 complete data sets were generated with imputed missing data, and the results were combined using Rubin's formula.

Time to onset of 3-month confirmed disability progression, time to onset of 6-month confirmed disability improvement, time to onset of sustained 20% increase in 9-HPT for ≥3 months, and time to onset of sustained 20% increase in 9-HPT for ≥3 months were analyzed similarly to the primary analysis of 6-month confirmed disability progression, but without imputation.

The number of new and/or enlarging T2-weighted lesions was analyzed using a negative binomial regression model without imputation, with total lesion count as the response variable and treatment group, age at screening (>40, ≤40 years), geographic region (US, non-US), baseline EDSS score, and baseline number of T2 lesions as covariates, and log transformed observation duration as the offset variable.

Percentage change in brain volume from month 6 was analyzed using a mixed-effect model with repeated measures (MMRM), which included percent change values at each scheduled visit as the response variable, and treatment, age at screening (>40 or ≤40 years), geographic region (US or non-US), visit, treatment by-visit interaction, baseline value and baseline value-by-visit interaction as covariates. Difference in LS means and the corresponding 95% CI were provided for the comparison of tolebrutinib versus placebo. As the normality assumption may not hold for this end point, MMRM analysis of log transformed data were conducted for estimating treatment effects.

Definition of multiple sclerosis relapse. Multiple sclerosis relapses were confirmed by the Relapse Adjudication Committee. Multiple sclerosis relapses were defined as monophasic, acute/subacute onset of new neurological symptoms or worsening of previous neurological symptoms with an objective change on neurological examination. Symptoms had to be attributable to multiple sclerosis, last for ≥24 hours (with or without recovery), be present at normal body temperature, and be preceded by ≥30 days of clinical stability (including no previous multiple sclerosis relapse). A confirmed relapse had to be accompanied by clinically relevant changes in EDSS score (i.e., ≥0.5-point increase in EDSS score, 1-point increase on two functional scores, or a 2-point increase in any single functional score, except bowel/bladder and cerebral functional scores).

MRI Assessments and Endpoints. MRI scans were performed at baseline, every 6 months until month 24, and every 12 months until the end of study. MRI scans required 1-month washout period for systemic corticosteroids and adrenocorticotropic hormone. The MRI protocol included T2- and T1-weighted sequences before and after gadolinium contrast agent administration, except where contraindicated. MRI-related end points were assessed by central reviewers who were blinded to treatment assignments and other participant data. The total number of new/enlarging T2-weighted lesions was defined as the sum of the individual number of new or enlarging T2-weighted lesions at all scheduled visits starting after baseline up to and including the end of study visit.

9-Hole Peg Test. The 9-Hole Peg Test (9-HPT), which assesses manual dexterity and fine motor skills (Fischer J S, et al., 1999; 5:244-50), was administered at baseline and every 3 months until the end of study. In the 9-HPT, participants are instructed to insert pegs one at a time into each of the nine holes arranged in a square pattern, and then to remove these pegs one at a time (Fischer, et al.) The dominant and non-dominant hands were both tested twice. The mean time to test completion was used as a measure of the participant's hand dexterity. An increase of >20% from baseline in the 9-HPT time was considered meaningful worsening (Schwid S, et al., Neurology 2002; 58:1294-6). Data were transformed so that higher Z-scores corresponded to improvement and lower Z-scores corresponded to deterioration. The inverse of the test values was taken prior to computing the Z-score. The baseline mean and standard deviation (SD) of all participants were used to create Z-scores:

$$Z = \frac{\frac{1}{2} ( \frac{1}{trialarm , left} ) + ( \frac{1}{trialarm , right} ) - Baseline Mean}{Baseline SD}$$

The times from the two trials for each hand were averaged and converted to the reciprocals of the mean times for each hand, which were then averaged, for example,

$$\frac{1}{trialarm , left} = \frac{1}{average of the two times of the left arm trials}$$

In cases where one of the two times for the same arm was missing, the available time was used. In cases where 9-HPT was assessed for only one hand for any reason other than for a physical limitation, the Z-score was set to missing. For participants who could not complete the 9-HPT due to a physical limitation, the maximum time (300 sec) was used. Missing values for other reasons were not imputed. Values below the lower test boundary (10 sec) or above the upper test boundary outside (300 sec) were set to the respective boundary.

Timed 25-Foot Walk. The timed 25-foot walk (T25-FW) test was administered at baseline and every 3 months until the end of study. The participant was directed to one end of a clearly marked 25-foot course and instructed to walk 25 feet as quickly and as safely possible (National MS Society. Timed 25-foot walk test). Two trials were performed and the times were averaged, unless only one trial was completed. An increase of >20% from the baseline in the T25-FW test time was considered meaningful worsening (Schwid S, et al., Neurology 2002; 58:1294-6). The baseline mean and SD of all participants were used to create Z-scores so that higher Z-scores corresponded to improvement and lower Z-scores corresponded to worsening:

$$Z = ( - 1 ) \times \frac{Average s core - Baseline Mean}{Baseline SD}$$

For participants who could not complete the T25-FW due to a physical limitation, the maximum time (180 sec) was used. Missing values for other reasons were not imputed. If results from both trials were missing due to reasons other than a physical limitation, the Z-score was set to missing. Values below the lower test boundary (2.2 sec) or above the upper test boundary outside (180 sec) were set to the respective boundary.

### Safety

Adverse Events. During the double-blind treatment period, adverse events were reported for 613 of 752 (81.5%) participants who were treated with tolebrutinib and 293 of 375 (78.1%) participants who received placebo (see Table 1O). Adverse events that were reported for ≥10% of participants who received tolebrutinib were COVID-19 and urinary tract infections; adverse events reported for ≥10% of participants who received placebo were COVID-19, urinary tract infection, and falls (see Table 10). Respiratory infections were more common with tolebrutinib versus placebo, including COVID-19 (25.5% vs. 22.7%), nasopharyngitis (9.3% vs. 6.9%), and influenza (5.6% vs. 3.5%). Serious adverse events occurred in 15.0% of participants who received tolebrutinib and 10.4% of participants who received placebo (see Table 1O). The most common serious adverse events with tolebrutinib were COVID-19 pneumonia (1.1%), multiple sclerosis relapse (1.1%), COVID-19 (0.9%), and pneumonia (0.7%). The most common serious adverse events with placebo were pneumonia (0.8%) and urosepsis (0.8%). Deaths were balanced across the groups. In the tolebrutinib group, one participant one participant developed liver failure assessed as related to tolebrutinib and died due to post-operative complications of a liver transplant; and one participant died by assisted suicide, assessed as unrelated to tolebrutinib. In the placebo group, one participant died from cerebral edema and hemorrhage due to a fall.

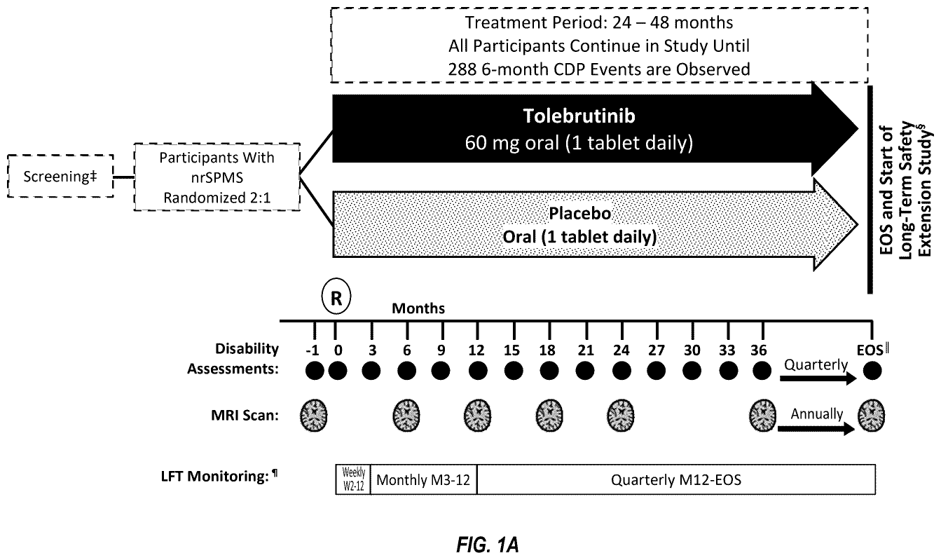

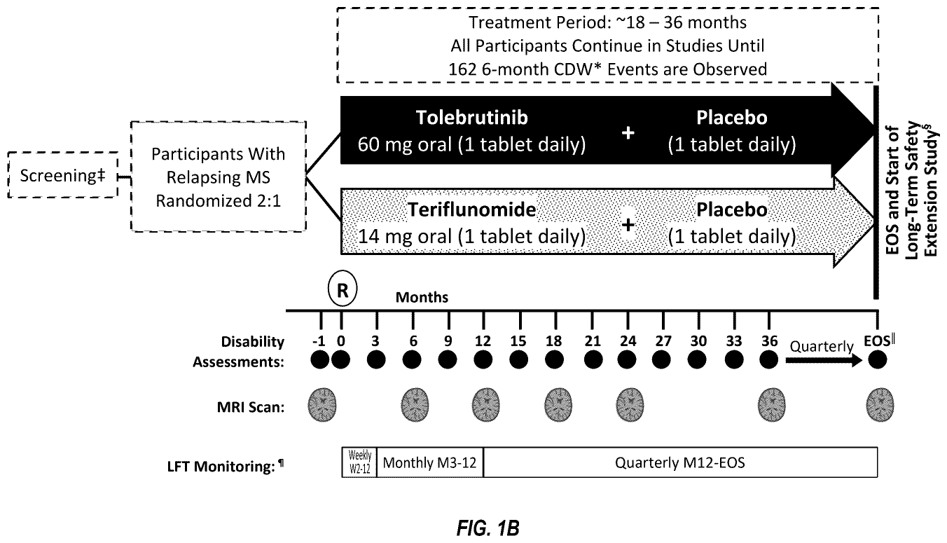

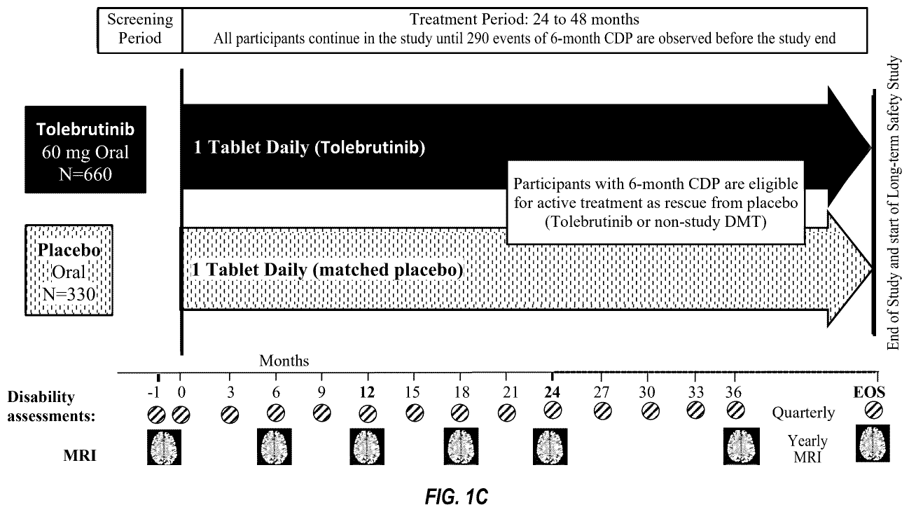

Liver Safety. Increased alanine aminotransferase levels above three-times the upper limit of normal were reported in 30 of 741 (4.0%) participants who received tolebrutinib and six of 372 (1.6%) participants who received placebo (see FIG. 18). Alanine aminotransferase level increases above 20-times the upper limit of normal were reported for 4 (0.5%) participants who received tolebrutinib and no participant who received placebo. All cases occurred within 90 days of treatment start, and all but one occurred before implementation of a revised protocol with more frequent liver monitoring. Three (0.4%) participants in the tolebrutinib group had levels of alanine aminotransferase greater than three-times the upper limit of normal and total bilirubin greater than two-times the upper limit of normal. The single participant who died due to post-operative complications of a liver transplant, with the underlying liver failure assessed as relative to tolebrutinib, showed an increased alanine aminotransferase level before implementation of the revised liver monitoring protocol (see FIG. 1). After weekly liver monitoring was implemented, all cases of elevated liver enzyme levels resolved without sequalae.

### DISCUSSION

In the HERCULES trial, tolebrutinib reduced the risk of 6-month confirmed disability progression in participants with non-relapsing secondary progressive multiple sclerosis. All disability progression events occured independent of relapse activity. Tolebrutinib treatment was also associated with fewer new and/or enlarging brain lesions on MRI than placebo, consistent with its effect on peripheral B lymphocytes (Turner, et al., Drugs in R&D 2024; 24:263-274).

Tolebrutinib effectively penetrates the CNS, reaching cerebrospinal fluid concentrations that exceed the 90% inhibitory concentration (Turner, et al., Drugs in R&D 2024; 24:263-274). This feature was not observed for other tested BTK inhibitors (Turner, et al.), including evobrutinib, which did not reduce the rate of disability worsening versus teriflunomide in two phase 3 randomized trials involving participants with relapsing multiple sclerosis (Montalban et al., Lancet Neurology 2024; 23:1119-1132; Montalban et al., New England J. Med. 2019; 380:2406-17).

Most increases in alanine aminotransferase observed in this trial were mild to moderate, and the majority resolved without sequelae. In the single participant who required a liver transplant, the initial liver enzyme abnormality occurred prior to a protocol amendment that increased the frequency of liver monitoring to weekly for the first 12 weeks of treatment.

In this trial involving participants with non-relapsing secondary progressive multiple sclerosis, tolebrutinib reduced the risk of disability progression versus placebo. These results support the role of tolebrutinib in slowing disability accrual in people with non-relapsing secondary progressive multiple sclerosis, a population with an unmet need for treatments that delay disability.

### Subgroup Analysis

In the intention-to-treat population, a potential quantitative interaction for subgroups of baseline Gd-enhancing T1-hyperintense lesions and prior disease modifying therapy (DMT) use was observed. Despite the differences in magnitude, the treatment effect in each subgroup analyzed is consistent with that of total population, which is in favor of tolebrutinib (see Table 1Q, FIG. 10D).

The time to onset of 6-month CDP in the subgroup of non-active participants, with no relapse in the 2 years prior to screening and without Gd-enhancing T6-hyperintense lesions at baseline, 152 (23.0%) participants in the tolebrutinib group had an event compared to 96 (29.2%) in the placebo group, with a relative risk reduction of 23% (HR [95% CI]: 0.767 [0.593 to 0.992]; unadjusted p=0.0433).

TABLE 1Q
Analysis of time to onset of 6-month CDP using Cox model
by all subgroups (intention-to-treat population)

| | | | | Placebo | | Tolebrutinib | |
|---|---|---|---|---|---|---|---|
| Subgroup | | | Statistic | (N = 377) | | (N = 754) | |
| Age at | >40 | years | No. | 313 | | 624 | |
| screening | | | No. with 6-month CDP, n (%) | 93 | (29.7) | 130 | (20.8) |

| | | Hazard ratio (95% CI)^{a} | 0.670 (0.512; 0.877) | |
|---|---|---|---|---|
| <=40 | years | No. | 64 | 130 |

| | | | | |
|---|---|---|---|---|
| No. with 6-month CDP, n (%) | 21 | (32.8) | 38 | (29.2) |

| | | | |
|---|---|---|---|
| | | Hazard ratio (95% CI)^{a} | 0.776 (0.456; 1.319) |
| | | p-value^{b} | 0.5827 |
| <50 | years | No. | 518 |
| | | Hazard ratio (95% CI) | 0.74 (0.54; 1.02) |
| ≥50 | years | No. | 613 |

| | | Hazard ratio (95% CI) | 0.66 (046; 0.94) | | | |
|---|---|---|---|---|---|---|
| Sex | Male | No. | 135 | | 300 | |
| | | No. with 6-month CDP, n (%) | 49 | (36.3) | 75 | (25.0) |
| | | Hazard ratio (95% CI)^{a} | 0.611 (0.424; 0.881) | | | |
| | Female | No. | 242 | | 454 | |
| | | No. with 6-month CDP, n (%) | 65 | (26.9) | 93 | (20.5) |
| | | Hazard ratio (95% CI)^{a} | 0.738 (0.536; 1.016) | | | |
| | Overall | p-value^{b} | 0.4603 | | | |
| Race | White | No. | 348 | | 703 | |
| | | No. with 6-month CDP, n (%) | 102 | (29.3) | 160 | (22.8) |
| | | Hazard ratio (95% CI)^{a} | 0.731 (0.569; 0.940) | | | |
| | Asian | No. | 19 | | 36 | |
| | | No. with 6-month CDP, n (%) | 7 | (36.8) | 6 | (16.7) |
| | | Hazard ratio (95% CI)^{a} | 0.424 (0.144; 1.244) | | | |
| | Black or | No. | 10 | | 15 | |
| | African | No. with 6-month CDP, n (%) | 5 | (50.0) | 2 | (13.3) |
| | American, | Hazard ratio (95% CI)^{a} | 0.310 (0.035; 2.748) | | | |
| | Other | | | | | |
| | Overall | p-value^{b} | 0.3038 | | | |
| Region strata | United States | No. | 37 | | 74 | |
| | | No. with 6-month CDP, n (%) | 12 | (32.4) | 10 | (13.5) |
| | | Hazard ratio (95% CI)^{a} | 0.369 (0.155; 0.874) | | | |
| | Non United | No. | 340 | | 680 | |
| | States | No. with 6-month CDP, n (%) | 102 | (30.0) | 158 | (23.2) |
| | | Hazard ratio (95% CI)^{a} | 0.733 (0.571; 0.941) | | | |
| | Overall | p-value^{b} | 0.1324 | | | |
| Region | Eastern Europe | No. | 134 | | 273 | |
| | | No. with 6-month CDP, n (%) | 38 | (28.4) | 55 | (20.1) |
| | | Hazard ratio (95% CI)^{a} | 0.645 (0.423; 0.984) | | | |
| | Western | No. | 150 | | 268 | |
| | Europe | No. with 6-month CDP, n (%) | 44 | (29.3) | 64 | (23.9) |
| | | Hazard ratio (95% CI)^{a} | 0.739 (0.503; 1.087) | | | |
| | North America | No. | 46 | | 116 | |
| | | No. with 6-month CDP, n (%) | 15 (32.6) | | 19 (16.4) | |
| | | Hazard ratio (95% CI)^{a} | 0.459 (0.238; 0.888) | | | |
| | Rest of the | No. | 47 | | 97 | |
| | world | No. with 6-month CDP, n (%) | 17 (36.2) | | 30 (30.9) | |
| | | Hazard ratio (95% CI)^{a} | 0.844 (0.468; 1.521) | | | |
| | Overall | p-value^{b} | 0.5226 | | | |
| Baseline Gd- | Presence | No. | 49 | | 93 | |
| enhancing T1 | | No. with 6-month CDP, n (%) | 20 | (40.8) | 17 | (18.3) |
| lesions | | Hazard ratio (95% CI)^{a} | 0.346 (0.183; 0.656) | | | |
| (imputed | Absence | No. | 328 | | 661 | |
| data) | | No. with 6-month CDP, n (%) | 94 | (28.7) | 151 | (22.8) |
| | | Hazard ratio (95% CI)^{a} | 0.777 (0.601; 1.006) | | | |
| | Overall | p-value^{b} | 0.0183 | | | |
| Baseline | <=4.5 | No. | 71 | | 183 | |
| EDSS score | | No. with 6-month CDP, n (%) | 19 | (26.8) | 32 | (17.5) |
| | | Hazard ratio (95% CI)^{a} | 0.603 (0.335; 1.085) | | | |
| | >4.5 | No. | 306 | | 571 | |
| | | No. with 6-month CDP, n (%) | 95 | (31.0) | 136 | (23.8) |
| | | Hazard ratio (95% CI)^{a} | 0.723 (0.556; 0.941) | | | |
| | Overall | p-value^{b} | 0.5661 | | | |
| Baseline | <=5.5 | No. | 145 | | 309 | |
| EDSS score | | No. with 6-month CDP, n (%) | 50 | (34.5) | 65 | (21.0) |
| | | Hazard ratio (95% CI)^{a} | 0.571 (0.393; 0.831) | | | |
| | >5.5 | No. | 232 | | 445 | |
| | | No. with 6-month CDP, n (%) | 64 | (27.6) | 103 | (23.1) |
| | | Hazard ratio (95% CI)^{a} | 0.803 (0.588; 1.097) | | | |
| | Overall | p-value^{b} | 0.1618 | | | |
| Prior disease | 0 | No. | 89 | | 205 | |
| modifying | | No. with 6-month CDP, n (%) | 31 | (34.8) | 33 | (16.1) |
| therapy | | Hazard ratio (95% CI)^{a} | 0.392 (0.241; 0.638) | | | |
| (DMT) use | 1 | No. | 102 | | 200 | |
| | | No. with 6-month CDP, n (%) | 29 | (28.4) | 44 | (22.0) |
| | | Hazard ratio (95% CI)^{a} | 0.649 (0.407; 1.034) | | | |
| | >=2 | No. | 186 | | 349 | |
| | | No. with 6-month CDP, n (%) | 54 | (29.0) | 91 | (26.1) |
| | | Hazard ratio (95% CI)^{a} | 0.902 (0.643; 1.265) | | | |
| | Overall | p-value^{b} | 0.0274 | | | |
| Duration | <=5 | No. | 11 | | 36 | |
| since | | No. with 6-month CDP, n (%) | 3 | (27.3) | 8 | (22.2) |
| relapsing- | | Hazard ratio (95% CI)^{a} | 0.514 (0.090; 2.949) | | | |
| remitting | >5 to <=10 | No. | 64 | | 128 | |
| multiple | | No. with 6-month CDP, n (%) | 23 | (35.9) | 27 | (21.1) |
| sclerosis | | Hazard ratio (95% CI)^{a} | 0.536 (0.299; 0.959) | | | |
| (RRMS) | >10 | No. | 302 | | 590 | |
| symptom | | No. with 6-month CDP, n (%) | 88 | (29.1) | 133 | (22.5) |
| onset | | Hazard ratio (95% CI)^{a} | 0.737 (0.562; 0.966) | | | |
| | Overall | p-value^{b} | 0.5411 | | | |
| Adjudicated | Yes | No. | 27 | | 54 | |
| relapse | | No. with 6-month CDP, n (%) | 10 | (37.0) | 21 | (38.9) |

| during the | | Hazard ratio (95% CI)^{a} | 0.894 (0.387; 2.062) | |
|---|---|---|---|---|
| study | No | No. | 350 | 700 |

| | | | | |
|---|---|---|---|---|
| No. with 6-month CDP, n (%) | 104 | (29.7) | 147 | (21.0) |

| | | |
|---|---|---|
| | Hazard ratio (95% CI)^{a} | 0.674 (0.524; 0.868) |
| Overall | p-value^{b} | 0.4526 |

^{a}Derived using Cox proportional-hazards model with treatment group, age at screening (>40, <=40 years), geographic region (US, non-US), baseline EDSS score and baseline Gd-enhancing T1 lesions (presence, absence) as covariates.
^{b}p-value for treatment by subgroup interaction, derived using Cox proportional-hazards model with treatment group, age at screening (>40, <=40 years), geographic region (US, non-US), baseline EDSS score and baseline Gd-enhancing T1 lesions (presence, absence), subgroup (if different than the aforementioned covariates) and treatment by subgroup interaction as covariates.

### Annualized Adjudicated Relapse Rate

Annualized adjudicated relapse rate (ARR) was very low in both the placebo and tolebrutinib groups, corresponding to about 1 relapse every 30 patient-years. Across both the placebo and tolebrutinib groups, 93% of participants were relapse-free during the trial. In the placebo group (N=377), the adjusted ARR was 0.032 (95% CI, 0.021 to 0.049). In the tolebrutinib group (N=754), the adjusted ARR was 0.033 (95% CI, 0.024 to 0.045). Adjusted ARR was derived using a negative binomial model with number of adjudicated relapses with onset between randomization and end of study as the response variable, treatment group, age at screening (≤40, >40 years), region (US, non-US), baseline EDSS score and baseline Gd-enhancing T1 lesions (presence, absence) as covariates, and log transformed observation duration as the offset variable.

### Effect on MSQoL-54

Multiple Sclerosis Quality of Life-54 (MSQoL-54) is a multidimensional health-related quality of life instrument combinine both generic and MS-specific items. (Vickrey, B G, et al. Qual. Life Res. 1995; 4:187-206). It includes 12 subscale scores and 2 single item measures (change in health and satisfaction with sexual function), all ranging from 0 (worst) to 100 (best). The physical composite health score is based on the subscale score for physical function, role limitation—physical, health perceptions, energy/fatigue, pain, sexual function, social function, and health distress. The mental health composite score is based on subscale scores for health distress, overall quality of life, emotional well-being, role limitation—emotional, and cognivite function.

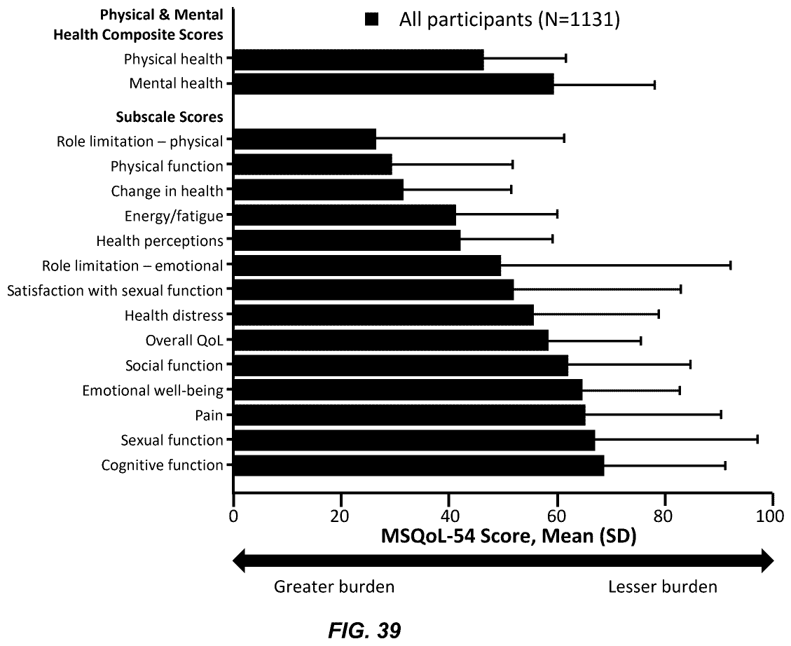

In the HERCULES study, at baseline the 3 subscales with the greatest burden (lowest score) were role limitation—physical, physical function, and change in health (FIG. 39). Composite and subscale least squares mean changes from baseline were analyzed using a mixed effect model with repeated measures with covariates for treatment group age at screening (≤40, >40 years), region (US, non-US), visit, treatment-by-visit interaction, baseline value for the endpoint being assessed and baseline value-by-visit interaction. Between group LSM difference was pre-specified for EOS and estimated post-hoc for all other timepoints.

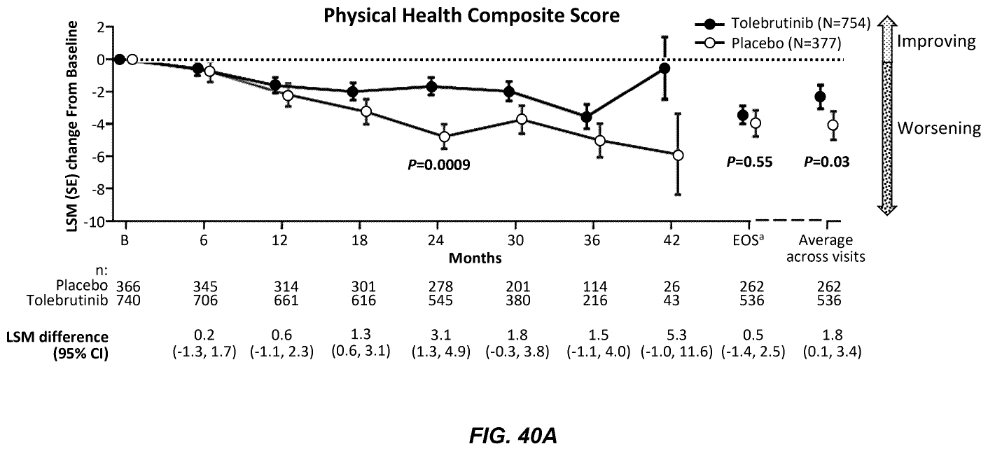

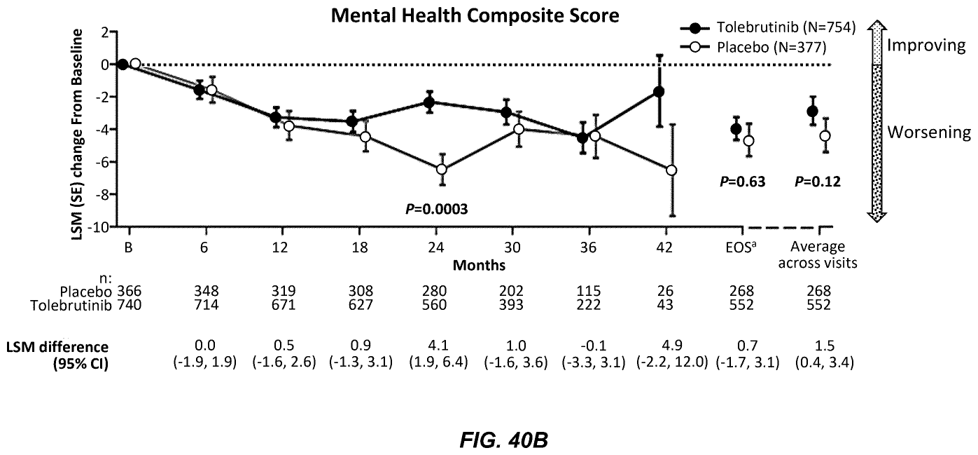

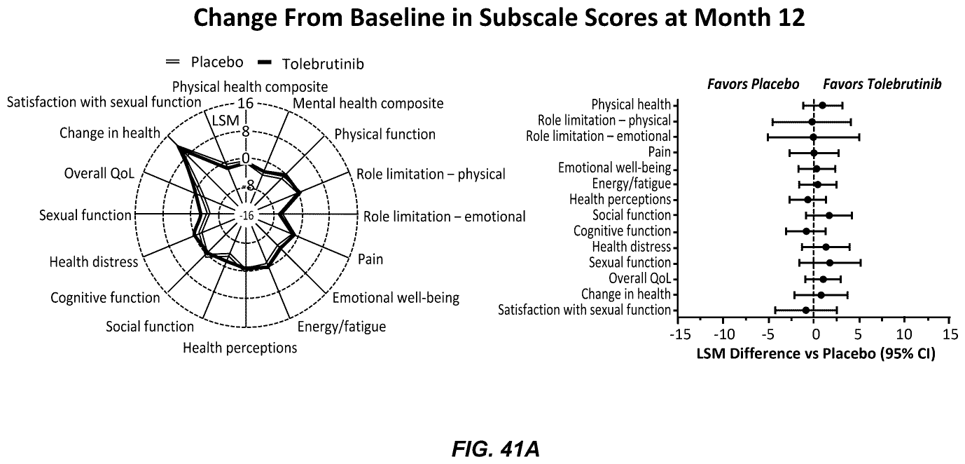

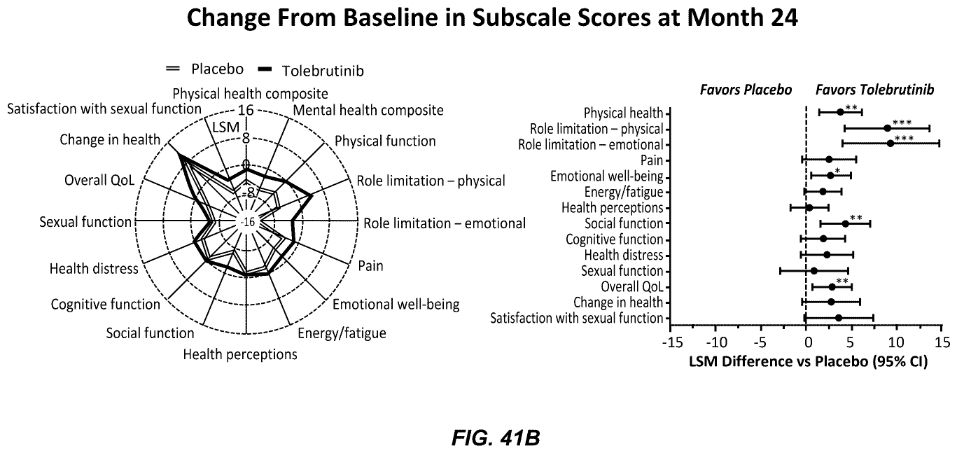

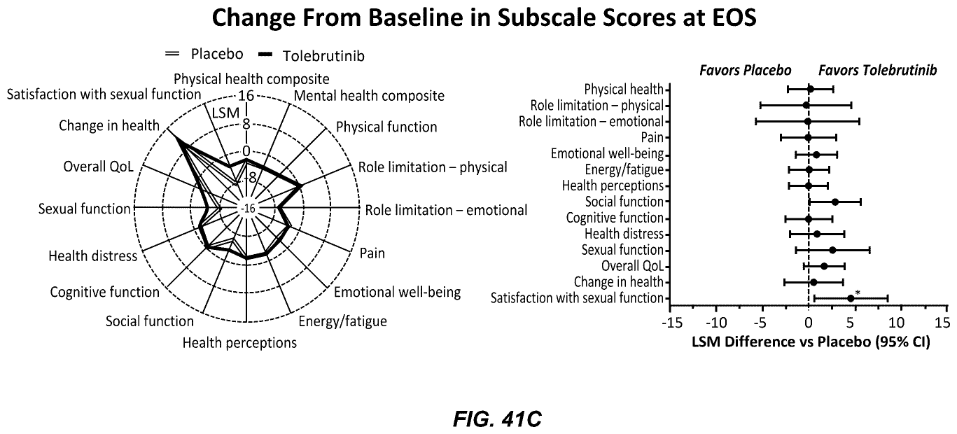

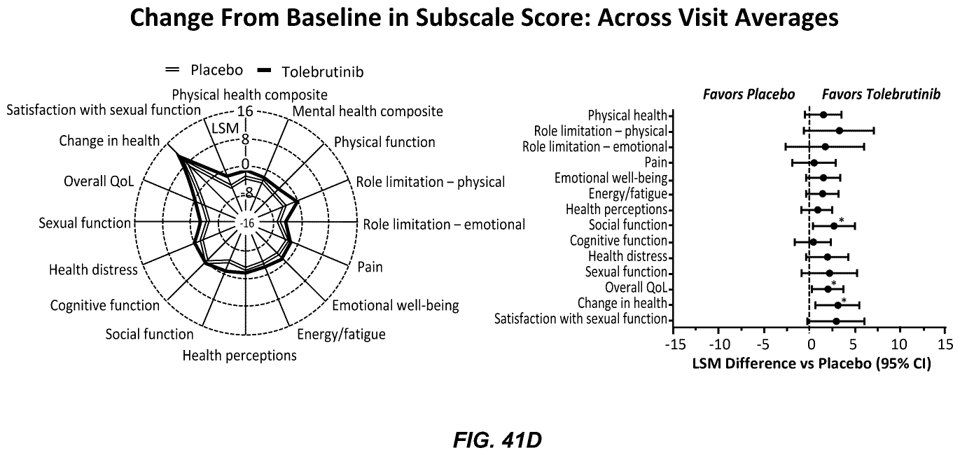

The change from baseline in physical health composite score and mental health composite score generally favored tolebrutinib, which was associated with more favorable MSQoL-54 physical and mental health composite scores as well as several of the subscale scores particularly at month 24 and when averaged across visits. (See Table 1P; FIGS. 40A-40B and 41A-41D). At month 12, no between-group differences were statistically significant for any score (FIG. 41A). At month 24, most domains favored tolebrutinib (FIG. 41B). At the end of study assessment, which occurred between 18-43 months post-baseline (median, 32 months, IQR, 28-36 months), significant between-group differences were not observed for the composite scores and most of the subscale score, potentially reflecting variability in the timing of the EOS assessment between participants (FIGS. 40A-40B and 41C). When averaged across visits, significant mean differences favored tolebrutinib for the social function, overall quality of life, and change in health domain scores (FIG. 41D).

### Pharmacokinetic Measures

Blood samples were collected at specified timepoints to assess maximum observed plasma concentration (Cₘₐₓ), time to maximum observed plasma concentration (Tₘₐₓ), and area under the plasma concentration-time curve over the last 24-hour dosing interval (AUC₀₋₂₄) of tolebrutinib and M2 metabolite using a population pharmacokinetics (PopPK) model, with three compartments for tolebrutinib and one for M2 and exposure parameters determined at steady-state for each sample and averaged within individual participants. Samples were collected 30 to 90 minutes post-dose at Months 6, 9, and 12 and 2.5 to 5 hours post-dose at Months 6 and 12. For PK visits, participants were required to administer treatment at the study site after a regular meal. Samples were evaluated for 669 participants using a validated method of liquid chromatography with tandem mass spectrometry. Mean Cₘₐₓ (SD) was 9.94 (6.18) ng/L for tolebrutinib and 27.5 (17.3) ng/mL for M2 metabolite. Mean Tₘₐₓ (SD) was 1.42 (0.674) h for tolebrutinib and 1.52 (0.667) h for M2 metabolite. Mean AUC₀₋₂₄ was 29.6 (17.8) ng*h/mL for tolebrutinib and 84.6 (53.7) ng*h/mL for M2 metabolite.

Cytochrome P450 2C8 catalyses the conversion of tolebrutinib into the active M2 metabolite, which has a similar pharmacological profile as tolebrutinib, including covalent BTK inhibition and brain penetrance (Cabanis M J, et al. Clin. Transl. Sci. 2024; 17:e13693; Nicolas O, et al., Clin. Drug. Investig. 2023; 43:653-665). M2 metabolite has the following formula:

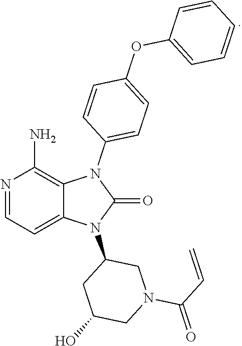 [SMILES: C=CC(=O)N1C[C@H](O)C[C@@H](n2c(=O)n(-c3ccc(Oc4ccccc4)cc3)c3c(N)nccc32)C1]

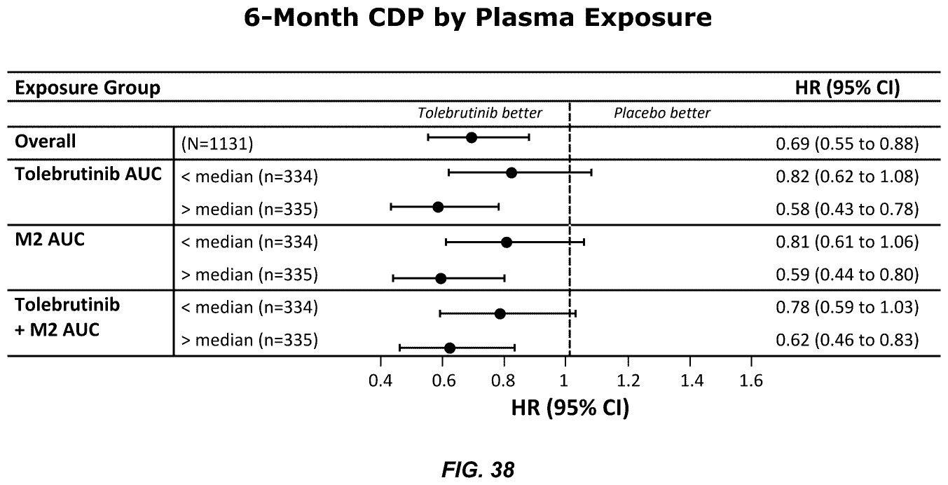

The relationship between plasma exposures and M2 and time to onset of 6-month CDP used a Cox proportional hazards model with robust variance estimation including covariates for age at screening (≤40 years, >40 years), region (US, non-US), baseline EDSS score, and presence of baseline Gd-enhancing T1 lesions and categorical variables of AUC for tolebrutinib, M2, or combined tolebrutinib/M2 divided into tertiles and placebo. Subgroup analysis for participants with tolebrutinib AUC, M2 AUC, and tolebrutinib+M2 AUC above and below median is shown in FIG. 38. Reduction in the risk of 6-month CDP with tolebrutinib treatment was greater for participants with plasma exposures of tolebrutinib and M2 greater than median. This analysis suggests that higher exposures of tolebrutinib and M2 are associated with more favorable disability outcomes. Since tolebrutinib and M2 exposures are higher under fed than fasted conditions, these findings reinforce the benefit of tolebrutinib being taken with food.

### Pharmacodynamic Measures

Change from Baseline in Plasma Neurofilament Light Chain (NfL) Levels at End of Study (EOS). Levels of NfL in plasma were measured as a potential marker of neuronal damage. Baseline was defined as the last available value prior to the first dose of study intervention. The median change (% change; IQR; number of participants evaluated) from baseline at the EOS in plasma NfL levels was 1.900 μg/mL (22.689%; −0.370 to 4.470; n=403) in the tolebrutinib group and 1.070 μg/mL (14.556%; −1.285 to 3.615; n=168) in the placebo group.

Change from Baseline in Serum Chitinase-3 Like Protein-1 Levels at End of Study (EOS). Levels of Chi3L1 in serum were measured as a potential biomarker for the progression of MS. The median change (% change; IQR; number of participants evaluated) from baseline at the EOS in serum Chi3L1 levels was 3132.250 μg/mL (13.349%; −2822.100 to 13407.100; n=410) in the tolebrutinib group and 5156.900 (15.485%; 1555.700 to 12099.200; n=171) in the placebo group.

Change from Baseline in Cluster of Differentiation CD19+ B cells at End of Study (EOS). Baseline was defined as the last available value prior to the first dose of study intervention. The median change (% change; IQR; number of participants evaluated) from baseline at the EOS in CD19+ B cells was −63.000 cells/μL (−40.694%; −105.000 to −8.000; n=58) in the tolebrutinib group and 10.000 cells/μL (3.433%; −18.000 to 77.000; n=26) in the placebo group.

Change from Baseline in Serum Immunoglobulin (Ig) Levels at End of Study (EOS). Baseline was defined as the last available value prior to the first dose of study intervention. The median change (% change; IQR; number of participants evaluated) from baseline at the EOS in serum IgG was −0.085 g/L (−0.723%; −0.845 to 0.665; n=320) in the tolebrutinib group and 0.350 g/L (3.642%; −0.280 to 1.310; n=149) in the placebo group. The median change (% change; IQR; number of participants evaluated) from baseline at the EOS in serum IgM was −0.240 g/L (−26.506%; −0.450 to 0.120; n=323) in the tolebrutinib group and 0.050 g/L (5.312%; −0.080 to 0.150; n=142) in the placebo group. Mean IgG levels remained overall stable and mean IgM levels remained above the lower limit of normal (LLN) in both the tolebrutinib and placebo groups over the duration of the trial. In the placebo group (n=375), 6.9% (n=26) of participants had IgG less than LLN during treatment, 2.1% (n=8) had normal IgG at baseline and 4.8% (n=18) had IgG less than LLN at baseline. In the tolebrutinib group (n=752), 10.1% (n=76) of participants had IgG less than LLN during treatment, 4.5% (n=34) had normal IgG at baseline and 5.6% (n=42) had IgG less than LLN at baseline. In the placebo group (n=375), 7.2% (n=27) of participants had IgM less than LLN during treatment, 1.3% (n=5) had normal IgM at baseline and 5.8% (n=22) had IgM less than LLN at baseline. In the tolebrutinib group (n=752), 18.0% (n=135) of participants had IgM less than LLN during treatment, 9.8% (n=74) had normal IgM at baseline and 8.1% (n=61) had IgM less than LLN at baseline. LLN values for IgG were 5.86 g/L for females and 6.03 for males, and for IgM were 0.26 g/L for females and 0.20 g/L for males.

Immune Cell Populations. Over 42 months, mean leukocyte, lymphocyte, neutrophil, monocyte, and platelet counts remained stable and within the normal range in the tolebrutinib and placebo groups. 0.1% of participants developed leukopenia in the tolebrutinib group and 0.8% of participants developed leukopenia in the placebo group. 0.7% of participants developed lymphopenia in the tolebrutinib group and 1.1% of participants developed lymphopenia in the placebo group. 1.6% of participants developed neutropenia in the tolebrutinib group and 2.1% of participants developed neutropenia in the placebo group. Thrombocytopenia (platelet count <75×10⁹/nL) occurred in 1 participant in the tolebrutinib group and none in the placebo group.

### Example 2—A Phase 3, Randomized, Double-Blind Efficacy and Safety Study Comparing tolebrutinib to Teriflunomide (Aubagio®) in Participants with Relapsing Forms of Multiple sclerosis (GEMINI 1 and 2)

The goal of this Phase 3 study is to assess tolebrutinib in the RMS population. Efficacy will be assessed by adjudicated relapse rate, disability progression, and MRI findings of disease activity (Gd-enhancing lesions and new/enlarging T2-hyperintense lesions). Together with evaluation of other secondary and exploratory endpoints, this study will provide a comprehensive evaluation of the efficacy and safety of tolebrutinib in the RMS population.

This Example describes Gemini 1 (EFC16033) and Gemini 2 (EFC16034), two trials investigating therapeutic intervention for patients with relapsing forms of MS, each of which follow the identical study design (except where specifically noted).

A graphical scheme of the study design is shown in FIG. 1B. Abbreviations used in FIG. 1B: EOS, end of study; MRI, magnetic resonance imaging; R, randomization. ‘Month-1 (D-28-D-1)’ refers to screening period as “Day-28 to Day-1”; ‘Month 0 (Di)’ refers to randomization on Day 1.

Table 2A shown below describe the schedule of activities during the course of study. Table 2B that follows describes the objective and endpoints of the overall study.

TABLE 2A
Schedule of Activities (SOA)

| | Procedure | | | | | | | | | | | | |
|---|---|---|---|---|---|---|---|---|---|---|---|---|---|
| | | Randomization/ | | | | | | | | | | | |
| | Screening^{a} | start of IMP | Year 1 (M 12)^{b} | | | | | | | | | | |
| | Visit (a window of ±7 days is allowed for all visits after D 1) | | | | | | | | | | | | |
| | | | | | M 1.25 (W 5) | | M 2.25 (W 9) | | | | | | |
| | D −28 | | M 0.5 (W 2) | | M 1.5 (W 6) | | M 2.5 (W 10) | | | | | | |
| | to D −1 | D 1 | M 0.75 (W 3) | M 1^{d} | M 1.75 (W 7) | M 2 (W 8)^{d} | M 2.75 (W 11) | M 3 | M 4, M 5^{d} | M 6 | M 7 M 8 | M 9 | M 10 M 11 |
| | Visit Number | | | | | | | | | | | | |
| | V1 | V2 | | V3 | | V4 | | V5 | V6, V7 | V8 | | V9 | |
| Informed consent | X | | | | | | | | | | | | |
| Demography | X | | | | | | | | | | | | |
| Inclusion/ | X | X | | | | | | | | | | | |
| exclusion criteria | | | | | | | | | | | | | |
| Medical/ | X | | | | | | | | | | | | |
| surgical history | | | | | | | | | | | | | |
| Prior/ | <================================================================================================> | | | | | | | | | | | | |
| concomitant | | | | | | | | | | | | | |
| medications^{g} | | | | | | | | | | | | | |
| Randomization | | X | | | | | | | | | | | |
| IRT contact | X | X | | | | | | X | | X | | X | |
| IMP dispensation^{e,t} | | X | | | | | | X | | X | | X | |
| IMP compliance | | | | | | | | X | | X | | X | |
| Paper diary | | X | | | | | | X | | X | | X | |
| dispensation/ | | | | | | | | | | | | | |
| collection | | | | | | | | | | | | | |

Safety

| Physical | X | X | | | | | | X | | X | | X | |
|---|---|---|---|---|---|---|---|---|---|---|---|---|---|
| examination^{h} and | | | | | | | | | | | | | |
| vital signs | | | | | | | | | | | | | |
| Height | X | | | | | | | | | | | | |
| Body weight | X | X | | | | | | X | | X | | | |
| Serology tests for | X | | | | | | | | | | | | |
| hepatitis B, C (HIV | | | | | | | | | | | | | |
| and other infectious | | | | | | | | | | | | | |
| diseases, if required | | | | | | | | | | | | | |
| locally) | | | | | | | | | | | | | |
| TB/QuantiFERON ® | X | | | | | | | | | | | | |
| TB Gold test or | | | | | | | | | | | | | |
| equivalentⁱ | | | | | | | | | | | | | |
| Body temperature | X | X | | | | | | X | | X | | X | |
| 12-lead ECG | X | | | | | | | X | | X | | X | |
| Hematology, | X | X^{y} | | X | | X | | X | X | X | | X | |
| biochemistry^{j} | | | | | | | | | | | | | |
| Liver function tests^{l} | | | X | | X | | X | | | | X | | X |
| Iron panel (serum): | X | | | | | | | | | | | | |
| iron, ferritin, | | | | | | | | | | | | | |
| transferrin | | | | | | | | | | | | | |
| saturation, TIBC; to | | | | | | | | | | | | | |
| be repeated during | | | | | | | | | | | | | |
| the study if needed | | | | | | | | | | | | | |
| Coagulation: | X | | | | | | | | | | | | |
| PT/INR, aPTT (to | | | | | | | | | | | | | |
| be repeated during | | | | | | | | | | | | | |
| the study, if needed) | | | | | | | | | | | | | |
| Urinalysis | X | | | | | | | X | | | | | |
| Pregnancy test (if | X | X | | X^{m} | | | | X | X^{m} | X | | X | |
| applicable)^{k,m} | | | | | | | | | | | | | |
| Serum FSHⁿ | X | | | | | | | | | | | | |
| Suicidality | X | X | | | | | | X | | X | | X | |
| assessment by C- | | | | | | | | | | | | | |
| SSRS | | | | | | | | | | | | | |
| Adverse event | <================================================================================================> | | | | | | | | | | | | |
| collection | | | | | | | | | | | | | |

Efficacy

| | | | | | |
|---|---|---|---|---|---|
| EDSS | X | X | X | X | X |
| Timed 25-foot walk | | X | X | X | X |
| test | | | | | |
| 9-hole peg test | | X | X | X | X |
| SDMT and CVLT- | | X | X | X | X |
| II, | | | | | |
| where available^{o} | | | | | |
| Basic or expanded | X^{q} | | | X | |
| MRI scan^{p} | | | | | |

Clinical outcome assessment^{r}

| | | |
|---|---|---|
| MSQoL-54 | X | X |
| EQ-5D-5L | X | X |

Pharmacokinetics^{s}

| | | |
|---|---|---|
| Tolebrutinib and | X^{u} | X^{v} |
| relevant | | |
| metabolite(s) | | |
| pharmacokinetic | | |
| plasma samples^{t} | | |

Pharmacogenetics^{s}

| | |
|---|---|
| DNA sample^{w} | X |

Pharmacodynamics/biomarkers^{s}

| | | | | |
|---|---|---|---|---|
| Blood sample for | X | | | |
| archiving^{x} | | | | |
| Plasma samples | | X | X | X^{v} |
| (NfL), serum | | | | |
| samples (Chi3L1) | | | | |
| Lymphocyte | X | | | |
| phenotypes by flow | | | | |
| cytometry^{z} | | | | |
| Serum samples (Ig | | X | | X^{v} |
| levels)^{t} | | | | |

| | Procedure | | | | | |
|---|---|---|---|---|---|---|
| | | | | | | Only for |
| | | | | Only for | | participants |
| | | | | participants | | who treatment to |
| | | | | who prematurely | For all | EOS completed but |
| | Year 1 (M 12)^{b} | From M 15 to EOSb | | discontinue IMP | participants | do not enter LTS^{c} |
| | Visit (a window of ±7 days is allowed for all visits after D 1) | | | | | |
| | | Quarterly Visits | Semi-annual | | EOS^{f} | |
| | | (M 15, M 18, M 21, | visits | | “Common | FU visit |
| | | M 24, M 27, M 30, | (M 18, M 24, | | study end | (4 to 8 weeks |
| | M 12 | M 33, M 36 . . .) | M 30, M 36 . . .) | pEOT^{e} | date” visit | after EOS) |
| | Visit Number | | | | | |
| | | V11, V12, V13, | | | | |
| | | V14, V15, V16, | V12, V14, | | | |
| | V10 | V17, V18 . . . | V16, V18 . . . | pEOT^{e} | EOS | FUV |
| Informed consent | | | | | | |
| Demography | | | | | | |
| Inclusion/ | | | | | | |
| exclusion criteria | | | | | | |
| Medical/ | | | | | | |
| surgical history | | | | | | |
| Prior/ | <====================================================================================================> | | | | | |
| concomitant | | | | | | |
| medications^{g} | | | | | | |
| Randomization | | | | | | |
| IRT contact | X | X | X | X | X | X |
| IMP dispensation^{e,t} | X | X | X | | | |
| IMP compliance | X | X | X | X^{e} | X | |
| Paper diary | X | X | X | X | X | |
| dispensation/ | | | | | | |
| collection | | | | | | |
| Safety | | | | | | |
| Physical | X | X | X | X | X | X |
| examination^{h} and | | | | | | |
| vital signs | | | | | | |
| Height | | | | | | |
| Body weight | | | X | X | X | X |
| Serology tests for | | | | | | |
| hepatitis B, C (HIV | | | | | | |
| and other infectious | | | | | | |
| diseases, if required | | | | | | |
| locally) | | | | | | |
| TB/QuantiFERON ® | | | | | | |
| TB Gold test or | | | | | | |
| equivalentⁱ | | | | | | |
| Body temperature | X | X | X | X | X | X |
| 12-lead ECG | X | | yearly | X | X | |
| Hematology, | X | X | X | X | X | X |
| biochemistry^{j} | | | | | | |
| Liver function tests^{l} | | | | | | |
| Iron panel (serum): | | | | | | |
| iron, ferritin, | | | | | | |
| transferrin | | | | | | |
| saturation, TIBC; to | | | | | | |
| be repeated during | | | | | | |
| the study if needed | | | | | | |
| Coagulation: | | | | | | |
| PT/INR, aPTT (to | | | | | | |
| be repeated during | | | | | | |
| the study, if needed) | | | | | | |
| Urinalysis | X | | X | X | X | |
| Pregnancy test (if | X | X | X^{m} | X | X | X |
| applicable)^{k,m} | | | | | | |
| Serum FSHⁿ | | | | | | |
| Suicidality | X | X | X | X | X | X |
| assessment by C- | | | | | | |
| SSRS | | | | | | |
| Adverse event | <====================================================================================================> | | | | | |
| collection | | | | | | |
| Efficacy | | | | | | |
| EDSS | X | X | X | X | X | |
| Timed 25-foot walk | X | X | X | X | X | |
| test | | | | | | |
| 9-hole peg test | X | X | X | X | X | |
| SDMT and CVLT- | X | X | X | X | X | |
| II, | | | | | | |
| where available^{o} | | | | | | |
| Basic or expanded | X | | M 18, M 24, | X | X | |
| MRI scan^{p} | | | M 36 | | | |
| Clinical outcome assessment^{r} | | | | | | |
| MSQoL-54 | X | | X | X | X | |
| EQ-5D-5L | X | | X | X | X | |
| Pharmacokinetics^{s} | | | | | | |
| Tolebrutinib and | X^{u} | | | X^{e} | | |
| relevant | | | | | | |
| metabolite(s) | | | | | | |
| pharmacokinetic | | | | | | |
| plasma samples^{t} | | | | | | |
| Pharmacogenetics^{s} | | | | | | |
| DNA sample^{w} | | | | | | |
| Pharmacodynamics/biomarkers^{s} | | | | | | |
| Blood sample for | | | | | | |
| archiving^{x} | | | | | | |
| Plasma samples | X^{v} | | yearly | X | X | |
| (NfL), serum | | | | | | |
| samples (Chi3L1) | | | | | | |
| Lymphocyte | | | | X | X | |
| phenotypes by flow | | | | | | |
| cytometry^{z} | | | | | | |
| Serum samples (Ig | X^{v} | | yearly | X | X | |
| levels)^{t} | | | | | | |

aPTT: activated partial thromboplastin time; β-HCG: β-human chorionic gonadotropin; AST: aspartate aminotransferase; ALT: alanine aminotransferase; Chi3L1: chitinase-3 like protein-1; CRF: case report form; C-SSRS: Columbia Suicide Severity Rating Scale; D: day; DNA: deoxyribonucleic acid; ECG: electrocardiogram; EDSS: Expanded Disability Status Scale; EOS: end of study; EOT: end of treatment; EQ-5D-5L: EuroQol 5-dimension 5-level questionnaire; FSH: follicle-stimulating hormone; FU: follow-up; Ig: immunoglobulin; HIV: human immunodeficiency virus; ICF: informed consent form; IMP: investigational medicinal product; INR: international normalized ratio; IRT: interactive response technology; LDH: lactate dehydrogenase; LTS: long-term safety study; M: month (28 days); MRI: magnetic resonance imaging; MS: multiple sclerosis; MSQoL-54: Multiple Sclerosis Quality of Life-54; NfL: neurofilament light chain; pEOT: premature end of treatment; PK: pharmacokinetic(s); PT: prothrombin time; SDMT: Symbol Digit Modalities Test; SWI: susceptibility-weighted imaging; TB: tuberculosis; TIBC: total iron-binding capacity
Note:
All assessments shall be done as designated in this SoA unless not permitted according to local regulations. All visit assessments should be performed during the visit window unless otherwise specified in this protocol.
^{a}Screening period can range from D −28 to D −1; Randomization visit can be performed only once IMPs are available at site. The interval between screening and randomization visits can range from 11 days (minimum) to 28 days (maximum). However, if required, the randomization visit can be performed earlier than 11 days upon IMP receipt at the site, assuming the participant is eligible for randomization. In case the screening MRI must be rescheduled (eg, technical issues) or repeated, an additional 1 week (7 days) is allowed.
^{b}From D 1 to EOS, unscheduled visits may be performed at any time by the Investigator (eg, for evaluation of an adverse event). Assessments may be done as needed to evaluate the participant in accordance with the Investigator's best judgment and in line with the study protocol. At a minimum, a physical examination should be performed, and, body temperature and vital signs should be measured.
^{c}At the EOS, the participants who have completed the study and remain on IMP will be offered participation in a long-term safety study. Follow-up visit assessment only performed for those participants who are not willing to take part in the long-term safety study.
^{d}These visits may be done as home health visits (where applicable) or onsite visits (preferable that tests are performed at the central laboratory). In any situations where this is not possible (to be documented in source documents), the tests for these visits may be performed at a local laboratory.
^{e}If a participant prematurely permanently discontinues treatment with IMP, the participant will undergo a premature EOT visit as soon as possible. A PK sample should also be collected if the pEOT visit can be scheduled within a maximum 24 hours after the last IMP dose. Participants will then be asked to continue with the study visits as scheduled, until global EOS visit is reached. During these visits, all study procedures/assessments will be performed except IMP administration and blood sampling for biomarkers (NfL, Chi3L1, and Ig levels). MRI scans for these participants will only be performed annually (using the next annual visit as the starting point).
^{f}Common EOS visit will be done when the prespecified number of events for 6-month CDW is expected to be reached. The timing and window of this visit will be communicated to sites.
^{g}Any disease-modifying therapy for MS taken at any time prior to signing the informed consent need to be reported in the CRF; other prior medications will be reported for the period of 6 months prior to signing the ICF.
^{h}Complete physical examination due at screening, baseline, yearly and EOS. Brief physical examination is sufficient for the rest of the visits (complete and brief physical examination will include neurological examination and collection of the following vital signs: arterial blood pressure, heart rate, and temperature).
ⁱTo be performed at screening for all participants. To be repeated based on clinical judgment, borderline results, or clinical suspicion of TB infection. Screening tests for TB are described in Table 2F.
^{j}Hematology (platelet count, red blood cell count, hemoglobin, hematocrit, mean corpuscular volume, mean corpuscular hemoglobin, reticulocytes, white blood cell count with differential: neutrophils, lymphocytes, monocytes, eosinophils, basophils).
Biochemistry (blood urea nitrogen [BUN], creatinine, glucose, potassium, sodium, chloride, bicarbonate, calcium; liver function tests [AST, ALT, albumin, alkaline phosphatase, total and direct bilirubin, total protein; creatine phosphokinase], lipase at Screening Visit, then quarterly). Monthly visits (M 1, M 2, M 4, M 5) will include hematology and full liver panel only. Additional safety assessments can be performed if required by local regulations. Such testing shall be performed at local laboratories.
Note:
a one-time retest at screening may be performed if an abnormal laboratory test value is considered temporary. Additional visits may be added if required by local regulations.
^{k}Serum β-HCG pregnancy test at central laboratory at screening and urine pregnancy tests within 24 hours before the first dose of IMP and at scheduled times during study. Additional serum or urine pregnancy tests may be performed, as determined necessary by the Investigator or required by local regulation, to establish the absence of pregnancy at any time during the participant's participation in the study. Communication by phone of the result of a pregnancy test performed at home to the site is allowed.
^{l}At intermediate timepoints (W 2, W 3, W 5, W 6, W 7, W 9, W 10, W 11, M 7, M 8, M 10, and M 11) only liver function tests will be collected (AST, ALT, albumin, alkaline phosphatase, total and direct bilirubin, total protein, and creatine phosphokinase); these can be central or local labs.
^{m}Pregnancy tests will be performed monthly in all concerned EU countries.
ⁿOnly in female participants, if needed to establish menopausal status.
^{o}SDMT and CVLT-II will be performed in all participants. If for some reason, CVLT-II is not available at a given site due to reasons such as lack of translation, local certification, etc, then only the SDMT will be assessed for that participant.
^{p}A subset of sites that have 3T MRI capacity will perform additional sequences (eg, SWI). Further details will be defined in a separate central MRI manual. For systemic corticosteroids and adrenocorticotropic hormone, 1 month washout is required prior to the MRI scans. A visit window of ±21 days is acceptable for MRIs performed after D 1.
^{q}The screening MRI scan should be performed as close as possible before the start of IMP. As much as possible, the MRI scan should be performed during the screening period after it has been established that the participant meets all inclusion and no exclusion criteria.
^{r}When available, clinical outcome assessments are to be completed by the participant prior to discussing their health status and prior to study treatment administration or other study-related procedures.
^{s}Where available per local regulations.
^{t}On days of PK sampling the IMP needs to be taken at the study site after a regular meal. In case a participant took IMP prior to arriving at the site on the day of visits with PK sampling, he/she will be asked to have a PK assessment within 3 days of the missed PK sampling. A PK assessment shall be done as soon as possible after an overdose or if otherwise specified per protocol (eg, investigation of abnormal laboratory test values).
^{u}Two samples: one sample between 30 and 90 minutes and one sample between 2.5 and 5 hours after IMP administration.
^{v}One sample between 30 and 90 minutes after IMP administration, if applicable per local regulation. Pharmacodynamics/biomarkers samples collected at other visits are not timed samples.
^{w}The DNA testing may be done, if locally applicable, at any time after signature of consent (in case it could not be done for some reason at Day 1).
^{x}This sample will be collected and stored for use if any unexpected safety issue occurs to ensure that a pre-dose baseline value is available for previously not assessed parameters (eg, serology) and for biomarkers research, if agreed.
^{y}Samples for hematology and biochemistry tests on Day 1 (randomization) must be collected prior to administration of the first IMP dose.
^{z}Blood collection for lymphocyte phenotypes by flow cytometry will be performed in a subset of participants randomized (approximately 400).

TABLE 2B
Objectives and endpoints

| Objectives | Endpoints | |
|---|---|---|
| Primary | | |
| To assess efficacy of daily tolebrutinib | ARR during the study period assessed by | |
| compared to a daily dose of 14 mg | confirmed protocol-defined adjudicated | |
| teriflunomide (Aubagio ®) measured by | relapses | |
| annualized adjudicated relapse rate (ARR) in | | |
| participants with relapsing forms of MS | | |
| Secondary | | |
| To assess efficacy of tolebrutinib compared | Time to onset of confirmed disability | |
| to teriflunomide (Aubagio ®) on disability | worsening (CDW), confirmed over at least | |
| progression, magnetic resonance imaging | 6 months, defined as follows: | |
| (MRI) lesions, cognitive performance and | - | increase of ≥1.5 points from the |
| quality of life | baseline Expanded Disability Status | |
| | Scale (EDSS) score when the baseline | |
| | score is 0, OR | |
| | - | increase of ≥1.0 point from the |
| | baseline EDSS score when the baseline | |
| | score is 0.5 to ≤5.5, OR | |
| | - | increase of ≥0.5 point from the |
| | baseline EDSS score when the baseline | |
| | score is >5.5 | |
| | Time to onset of CDW, assessed by the | |
| | EDSS score and confirmed over at least 3 | |
| | months | |
| | Total number of new and/or enlarging T2- | |
| | hyperintense lesions as detected by MRI, | |
| | defined as the sum of the individual | |
| | number of new and/or enlarging T2 | |
| | lesions at all scheduled visits starting after | |
| | baseline up to and including the End-of- | |
| | Study (EOS) visit and number of new | |
| | and/or enlarging T2-hyperintense lesions | |
| | by visit over time | |
| | Total number of new gadolinium- (Gd-) | |
| | enhancing T1-hyperintense lesions as | |
| | detected by MRI, defined as the sum of the | |
| | individual number of new Gd-enhancing T1- | |
| | hyperintense lesions at all scheduled visits | |
| | starting after baseline up to and including | |
| | the EOS visit | |
| | Time to confirmed disability improvement | |
| | (CDI), defined as a ≥1.0 point decrease on | |
| | the EDSS from the baseline EDSS score | |
| | confirmed over at least 6 months | |
| | Percent change in brain volume loss as | |
| | detected by brain MRI scans at the EOS | |
| | compared to Month 6 | |
| | Change in cognitive function at the EOS | |
| | compared to baseline as assessed by the | |
| | Symbol Digit Modalities Test (SDMT) | |
| | Change in cognitive function at the EOS | |
| | compared to baseline as assessed by the | |
| | CVLT-II, where available | |
| | Change in Multiple Sclerosis Quality of | |
| | Life 54 (MSQoL-54) questionnaire score at | |
| | the EOS compared to baseline [MSQoL-54 | |
| | was only measured for patients in the | |
| | Gemini 2 (EFC16034) study.] | |
| To evaluate the safety and tolerability of | Adverse events (AEs), serious AEs, AEs | |
| daily tolebrutinib | leading to permanent study intervention | |
| | discontinuation, AEs of special interest, and | |
| | potentially clinically significant | |
| | abnormalities in laboratory tests, safety | |
| | scales, ECG, and vital signs during the | |
| | study period | |
| To evaluate pharmacodynamics (PD) of | Change in plasma neurofilament | |
| tolebrutinib | light chain (NfL) levels at the EOS | |
| | compared to baseline, where | |
| | available | |
| | Changes in serum | |
| | immunoglobulin level at the | |
| | EOS compared to baseline | |
| | Change in serum Chi3L1 levels at the EOS | |
| | compared to baseline | |
| Tertiary/exploratory | | |
| To evaluate the efficacy of tolebrutinib on | EDSS score change from baseline at | |
| disease activity as measured by additional | scheduled visits starting after baseline and | |
| clinical, brain MRI, and composite | including the EOS visit | |
| measurements | Proportion of adjudicated relapse-free | |
| | participants from randomization until the | |
| | EOS visit | |
| | Time to onset of 20% worsening in the 9- | |
| | hole peg test (9-HPT) confirmed over at | |
| | least 3 and 6 months | |
| | Time to onset of 20% worsening in the | |
| | timed 25-foot walk (T25-FW) test | |
| | confirmed over at least 3 and 6 months | |
| | Time to onset of 4-point decrease in Symbol | |
| | Digit Modalities Test (SDMT) confirmed | |
| | over at least 3 and 6 months | |
| | Change from baseline of total volume of | |
| | T2-hyperintense lesions as detected by brain | |
| | MRI at Months 18, 24, and the EOS | |
| | Magnetization transfer ratio recovery at the | |
| | EOS in new magnetization transfer ratio | |
| | lesions detected at Months 6 and 12 | |
| | Change in number of phase rim lesions in | |
| | susceptibility weighted imaging (SWI) MRI | |
| | from baseline by visit over time (subset of | |
| | centers with capacity of 3T MRI) | |
| | Proportion of participants with no evidence | |
| | of disease activity (NEDA-3) at Months 18, | |
| | 24, and the EOS | |
| | Change from baseline to Months 12, 18, and | |
| | 24 and to the EOS in modified Multiple | |
| | Sclerosis Functional Composite 3 (MSFC- | |
| | 3), assessed as the composite of the T25- | |
| | FW test, 9-HPT, and SDMT | |
| | Change from baseline by visit over time in | |
| | volume of T1-hypointense lesions, and | |
| | cumulative number of new T1-hypointense | |
| | lesions | |
| | Number and volume of slowly evolving | |
| | lesions (SELs) | |
| | Normalized T1 intensity evolution in SELs | |
| To evaluate the treatment effect of | Change in EuroQol 5-dimension 5-level | |
| tolebrutinib via changes in participants' | questionnaire (EQ-5D-5L) from baseline by | |
| health-related quality of life (HRQoL), and | visit over time | |
| working capacity | | |

General Statistical Analyses Considerations. The baseline values of efficacy parameters are generally defined as the last available value prior to the first dose of study medication unless otherwise specified. For the EDSS, the baseline value will be taken as the average of the screening and randomization visit values. If one of the values is missing, the non-missing value will be used as baseline. The baseline value of safety parameters is defined as the last available value prior to the first dose of IMP. Unless otherwise indicated, 2-sided p-values and 95% confidence intervals [CI(s)] will be provided for assessment of treatment differences.

### Overall Design:

This is a Phase 3, randomized, double-blind, double-dummy, 2-arm, active-controlled, parallel group, multicenter, event-driven (6-month confirmed disability worsening [CDW]) trial with a variable treatment duration ranging from approximately 18 to 36 months.

### Disclosure Statement:

This is a parallel treatment study with 2 arms that is blinded/masked for participants, any Investigator, site staff, and the Sponsor.

### Number of Participants:

Approximately 1200 people will be screened to achieve approximately 900 (±10%) participants randomly assigned to the study intervention with a total sample size of at least 1800 patients across the 2 RMS studies of identical design (Gemini 1, EFC16033; and Gemini 2, EFC16034).

### Intervention Groups and Duration:

Participants will be randomly assigned at a 1:1 ratio to receive the 60 mg selected dose (established from dose-finding Study DRI15928) of oral tolebrutinib daily as well as a placebo to match the teriflunomide tablet or 14 mg oral teriflunomide as well as a placebo to match the tolebrutinib tablet daily. Randomization will be stratified by the Expanded Disability Status Scale (EDSS) score at screening (<4 versus ≥4) and geographic region (US versus non-US).

### Study Intervention(s)

### Investigational Medicinal Product

Formulation: tolebrutinib film-coated tablet
Route(s) of administration: oral
Dose regimen: 60 mg once daily

### Investigational Medicinal Product

Formulation: teriflunomide tablet
Route of administration: oral
Dose regimen: 14 mg once daily

### Investigational Medicinal Product

Formulation: placebo to match tolebrutinib film-coated tablet
Route of administration: oral
Dose regimen: once daily

### Investigational Medicinal Product

Formulation: placebo to match teriflunomide tablet
Route of administration: oral
Dose regimen: once daily

### Noninvestigational Medicinal Products

Formulation: MRI contrast-enhancing preparations
Route(s) of administration: intravenous (IV)
Dose regimen: as per respective label

### Noninvestigational Medicinal Products

Formulation: cholestyramine
Route(s) of administration: oral
Dose regimen: 8 gram 3 times daily for 11 days for accelerated elimination procedure
   (4 gram 3 times daily for 11 days in case of intolerance). The teriflunomide local label should be followed.

### Temporary Investigational Medicinal Product (IMP) Interruption Due to Surgery

If surgery is needed during the study, consider the benefit/risk of withholding the IMP for at least 3 to 7 days pre- and post-surgery depending upon the type of surgery and the risk of bleeding.

The goal of this Phase 3 study is to assess tolebrutinib in the RMS population. Efficacy will be assessed by adjudicated relapse rate, disability progression, and MRI findings of disease activity (Gd-enhancing lesions and new/enlarging T2-hyperintense lesions). Together with evaluation of other secondary and exploratory endpoints, this study will provide a comprehensive evaluation of the efficacy and safety of tolebrutinib in the RMS population.

In the ongoing Phase 3 and LTS studies, tolebrutinib has been generally well tolerated to date. An identified risk for tolebrutinib has been identified as follows:
Treatment-emergent SAEs of drug-induced liver injury (DILI) were reported in the ongoing Phase 3 trials; however, all cases occurred between Months 2 to 3 and appear reversible after treatment discontinuation, with potential confounders identified for some of the cases.

### Study Population

### Inclusion Criteria

Participants are eligible to be included in the study only if all of the following criteria apply as shown in Table 2C.

TABLE 2C
Inclusion Criteria

| Category | Criteria | |
|---|---|---|
| Age | I 01. | The participant must be 18 to 85 years of age inclusive, at the time |
| | | of signing the informed consent. |
| Type of | I 02. | The participant must have been diagnosed with RMS according to |
| participant | | the 2017 revision of the McDonald diagnostic criteria (Thompson |
| and disease | | et al. Lancet Neurol. 2018, 17, 162). |
| characteristics | I 03. | The participant has an EDSS score ≤5.5 at the first visit (Screening |
| | | Visit) |
| | I 04. | The participant must have at least 1 of the following prior to |
| | | screening: |

| | |
|---|---|
| • | ≥1 documented relapse within the previous year OR |
| • | ≥2 documented relapses within the previous 2 years, |
| | OR |
| • | ≥1 documented Gd-enhancing lesion on an MRI scan |
| | within the previous year |

```
  Note: The initial clinical demyelinating episode
  of MS should be counted as a relapse for the first
  2 criteria.
```

| Weight | I 05. | Not Applicable. |
|---|---|---|
| Sex | I 06. | Male or Female. |
| | Contraceptive use by men or women should be consistent with local | |
| | regulations regarding the methods of contraception for those participating | |
| | in clinical studies. | |
| | Male participants wishing to conceive a child and female participants | |
| | becoming pregnant or wishing to become pregnant must permanently | |
| | discontinue the study intervention and follow the local teriflunomide label | |
| | recommendation. | |
| | a) | Male participants |
| | Male participants are eligible to participate if they agree to the following | |
| | during the intervention period and until the accelerated elimination | |
| | procedure is performed. | |
| | Refrain from donating sperm. Plus either: | |
| | • | Be abstinent from heterosexual intercourse as their preferred and |
| | | usual lifestyle (abstinent on a long-term and persistent basis) and |
| | | agree to remain abstinent |

OR

| | |
|---|---|
| • | Must agree to use contraception/barrier method as detailed below |

| | |
|---|---|
| - | Agree to use a male condom and should also be advised of |
| | the benefit for a female partner to use a highly effective |
| | method of contraception as a condom may break or leak |
| | when having sexual intercourse with a woman of |
| | childbearing potential (WOCBP) who is not currently |
| | pregnant |

| | |
|---|---|
| b) | Female participants |

| | |
|---|---|
| • | A female participant is eligible to participate if she is not |
| | pregnant or breastfeeding, and at least one of the following |
| | conditions applies: |
| - | Is not a woman of childbearing potential (WOCBP); or |
| - | Is a WOCBP and agrees to use a contraceptive method that is |
| | highly effective (with a failure rate of <1% per year), |
| | preferably with low user dependency, during the intervention |
| | period and until the accelerated elimination procedure is |
| | completed after the last dose of study intervention |
| - | A WOCBP must have a negative highly sensitive pregnancy |
| | test at screening and within 24 hours before the first dose of |
| | study intervention. If a urine test cannot be confirmed as |
| | negative (eg, an ambiguous result), a serum pregnancy test is |
| | required. In such cases, the participant must be excluded from |
| | participation if the serum pregnancy result is positive. |
| • | Additional requirements for pregnancy testing during and after |
| | study intervention are located in the schedule of activities |
| | (SoA); Table 2A). |
| • | The Investigator is responsible for review of medical history, |
| | menstrual history, and recent sexual activity to decrease the |
| | risk for inclusion of a woman with an early undetected |
| | pregnancy, if allowed by local regulations. |

### Exclusion Criteria

Participants are excluded from the study if any of the following criteria apply as shown in Table 2D.

TABLE 2D
Exclusion Criteria

| Category | Criteria | |
|---|---|---|
| Medical | E 01. | The participant has been diagnosed with PPMS according to |
| conditions | | the 2017 revision of the McDonald diagnostic criteria |
| | | (Thompson et al. Lancet Neurol. 2018, 17, 162) or with non- |
| | | relapsing SPMS (Lublin et al. Neurology 2014, 83, 278-286). |
| | E 02. | The participant has a history of infection or may be at risk for |
| | | infection: |

| | |
|---|---|
| • | A history of T-lymphocyte or T-lymphocyte-receptor |
| | vaccination, transplantation (including solid organ, |
| | stem cell, and bone marrow transplantation) and/or |
| | antirejection therapy. |
| • | The participant has received any live (attenuated) |
| | vaccine (including but not limited to varicella zoster, |
| | oral polio, and nasal influenza) within 2 months |
| | before the first treatment visit. |
| • | The participant has a lymphocyte count less than the |
| | lower limit of normal (LLN) at the Screening Visit. |
| • | A history of diagnosis of progressive multifocal |
| | leukoencephalopathy (PML) or evidence of findings |
| | suggestive of PML on the screening MRI. |
| • | A history of infection with human immunodeficiency |
| | virus (HIV) (eg, any known positive HIV test or |
| | information from participant interview). |
| • | A history of active or latent tuberculosis (TB); TB |
| | testing should be performed at screening and again |
| | during the study, if clinically indicated, and may be |
| | repeated based on clinical judgment, borderline |
| | results, or clinical suspicion of TB infection. |
| | Screening tests for TB are described in Table 2F. |
| • | NOTE: The Investigator may consult with an |
| | infectious disease expert if required, eg, test results |
| | are unclear or there is a suspicion of false positive test |
| | results. If the infectious disease expert considers the |
| | test results as false positive and not clinically relevant |
| | and confirms that the participant can be enrolled in |
| | the trial, the Investigator must document this in |
| | source data and may then randomize the participant. |
| • | Persistent chronic or active recurring infection |
| | requiring treatment with antibiotics, antivirals, or |
| | antifungals. |
| • | Fever within 4 weeks of the Screening Visit (≥38° C.; |
| | however, if due to brief and mild ear, nose, throat |
| | viral infection participant may be included based on |
| | the Investigator's judgment). |
| • | Participants at risk of developing or having |
| | reactivation of hepatitis, ie, results at screening for |
| | serological markers for hepatitis B and C viruses |
| | indicating acute or chronic infection. See the Study |
| | Manual for further details. |
| • | Any other active infections that would adversely |
| | affect participation or IMP administration in this |
| | study, as judged by the Investigator. |

| | |
|---|---|
| E 03. | The presence of psychiatric disturbance or substance abuse as |
| | evidenced by: |

| | |
|---|---|
| • | A history of any psychiatric disease, behavioral |
| | condition, or depression requiring hospitalization |
| | within 2 years prior to the Screening Visit. |
| • | A documented history of attempted suicide or suicidal |
| | ideation of category 4 or 5 according to the Columbia |
| | Suicide Severity Rating Scale (C-SSRS) |
| | baseline/screening version over the 6 months prior to |
| | the Screening Visit, OR if in the Investigator's |
| | judgment, the participant is at risk for a suicide |
| | attempt. |
| • | Active alcohol use disorder or a history of alcohol or |
| | drug abuse within 1 year prior to the Screening Visit. |
| • | Current alcohol intake >2 drinks per day for men and >1 |
| | drink per day for women (1 drink = approximately |
| | 14 grams of alcohol = 350 mL beer = 140 mL |
| | wine = 40 mL of spirits). |

| | |
|---|---|
| E 04. | The following findings obtained during the Screening Visit |
| | considered in the Investigator's judgment to be clinically |
| | significant in the context of this clinical trial: |

| | |
|---|---|
| • | Any screening laboratory values outside normal |
| | limits. |
| • | Abnormal ECG. |

| | |
|---|---|
| E 05. | Conditions that may predispose the participant to excessive |
| | bleeding: |

| | |
|---|---|
| • | A bleeding disorder or known platelet dysfunction at |
| | any time prior to the screening visit. |
| • | A platelet count <150 000/μL at the screening visit. |
| • | The participant has had major surgery within 4 weeks |
| | prior to the screening visit, which could affect the |
| | participant's safety (as judged by the Investigator) or |
| | has planned any elective major surgery during the |
| | study. |
| • | A history of significant bleeding event within 6 |
| | months prior to screening, according to the |
| | Investigator's judgment such as, but not limited to |
| | cerebral or gastrointestinal bleeding. |

| | |
|---|---|
| E 06. | Conditions that would adversely affect participation in the |
| | study or make the primary efficacy endpoint non-evaluable: |

| | |
|---|---|
| • | Sensitivity to any of the study interventions, |
| | or components thereof, or has a drug or other |
| | allergy that, in the opinion of the |
| | Investigator, contraindicates participation in |
| | the study. |
| • | A short life expectancy due to pre-existing |
| | health condition(s) as determined by their |
| | treating neurologist. |
| • | A history or presence of significant other |
| | concomitant illness according to the |
| | Investigator's judgment such as, but not |
| | limited to cardiovascular (including Stage III |
| | or IV cardiac failure according to New York |
| | Heart Association [NYHA] classification), or |
| | renal (ie, undergoing dialysis), neurological, |
| | endocrine, gastrointestinal, metabolic, |
| | pulmonary (eg, interstitial pneumonia or |
| | pulmonary fibrosis), or lymphatic disease |
| | that would adversely affect participation in |
| | this study. |
| • | Acute liver disease, cirrhosis, chronic liver |
| | disease (unless considered stable |
| | for >6 months). |
| • | Confirmed screening ALT >1.5 × ULN OR |
| | AST >1.5 × ULN OR alkaline phosphatase >2 × ULN |
| | (unless caused by non-liver-related disorder or |
| | explained by a stable chronic liver disorder) OR total |
| | bilirubin >1.5 × ULN (unless due to Gilbert syndrome |
| | or non-liver-related disorder). |
| • | At screening, elevated transferrin saturation (>50% in |
| | males and >40% in females) and/or with elevated |
| | ferritin levels >500 μg/L. |
| • | Any malignancy within 5 years prior to the |
| | Screening Visit (except for effectively |
| | treated carcinoma in situ of the cervix or |
| | adequately treated non-metastatic squamous |
| | or basal cell carcinoma of the skin) will also |
| | be exclusionary. |
| • | Any other medical condition(s) or |
| | concomitant disease(s) making them non- |
| | evaluable for the primary efficacy endpoint |
| | or that would adversely affect participation |
| | in this study, as judged by the Investigator. |

| | | |
|---|---|---|
| Prior/concomitant | E 07. | The participant has received any of the following |
| therapy | | medications/treatments within the specified time frame before |
| | | any baseline assessment (no washout is required for dimethyl |
| | | fumarate, interferon beta, or glatiramer acetate treatments): |

| | Exclusionary if used/used within |
|---|---|
| Medication | required wash-out period |
| Systemic corticosteroids, | 1 month prior to |
| adrenocorticotropic hormone | screening MRI |
| | scan |
| Siponimod, ponesimod | 1 week before |
| | randomization with |
| | MRI and clinical |
| | assessment for |
| | PML prior to |
| | randomization |
| Plasma exchange | 1 month prior to |
| | randomization |
| IV immunoglobulin | 2 months prior to |
| | randomization |
| Fingolimod, ozanimod | 6 weeks before |
| | randomization |
| | with MRI and |
| | clinical |
| | assessment for |
| | PML |
| Mildly to moderately | 3 months prior to |
| immunosuppressive/chemotherapeutic | randomization |
| medications such as azathioprine and | |
| methotrexate, mycophenolate | |
| Lymphoid irradiation, bone marrow | A participant who |
| transplantation, mitoxantrone (with | has received any |
| evidence of cardiotoxicity following | of these |
| treatment, or cumulative lifetime | treatments at any |
| dose >120 mg/m²), other strongly | time is not |
| immunosuppressive treatments with | eligible. |
| very long-lasting effects | |
| Teriflunomide | 3 months prior to |
| <3 months treatment | randomization* |
| ≥3 months treatment | A participant who |
| | has received this |
| | treatment at any |
| | time is not |
| | eligible. |
| Natalizumab | 2 months before |
| | randomization |
| | with MRI and |
| | clinical |
| | assessments for |
| | PML |
| B-cell-depleting therapies such as | 6 months prior |
| ocrelizumab and rituximab | |
| Ofatumumab | 4 months prior |
| Highly | 2 years prior to |
| immunosuppressive/chemotherapeutic | randomization |
| medications: mitoxantrone up to 120 | |
| mg/m² body surface area, | |
| cyclophosphamide, cladribine, | |
| cyclosporine | |
| Alemtuzumab | 4 years prior to |
| | randomization |
| Other MS-disease modifying | 5 half-lives or until |
| treatments | end of |
| | pharmacodynamics |
| | activity, whichever |
| | is longer |

| | |
|---|---|
| E 08. | The participant is receiving potent and moderate inducers of |
| | cytochrome P450 3A (CYP3A) or potent inhibitors of |
| | CYP2C8 hepatic enzymes. |
| E 09. | The participant is receiving anticoagulant/antiplatelet |
| | therapies, including: |

| | |
|---|---|
| • | Acetylsalicylic acid (aspirin) >81 mg/day, |
| • | Antiplatelet drugs (eg, clopidogrel), |
| • | Warfarin (vitamin K antagonist), |
| • | Heparin, including low molecular weight heparin |
| | (antithrombin agents), |
| • | Dabigatran (direct thrombin inhibitor), |
| • | Apixaban, edoxaban, rivaroxaban (direct factor Xa |
| | inhibitors). |

| | Note: All the above-mentioned drugs must be stopped at least 5 half- | |
|---|---|---|
| | lives before study drug administration except for aspirin, which must | |
| | be stopped at least 8 days before. If this is not clinically appropriate, | |
| | the participant cannot be included. These washout periods are only | |
| | applicable in the case that the Investigator deems it clinically | |
| | appropriate to discontinue the listed medications or there is a recent | |
| | history of use of these medications (as in the case when short term | |
| | treatment with anticoagulants is clinically recommended for certain | |
| | thrombotic events), and therefore these washout periods will need to | |
| | be followed prior to randomization. | |
| | If the participant has a chronic underlying medical condition (stroke, | |
| | coronary or carotid artery disease, heart valvular disease etc.) | |
| | requiring continued use of these medications, the participant cannot | |
| | be enrolled in the study. | |
| | E 10. | A history of a hypersensitivity reaction to teriflunomide, |
| | | leflunomide, or to any of the inactive ingredients in Aubagio |
| | | (this includes anaphylaxis, angioedema, and serious skin |
| | | reactions). |
| Prior/concurrent | E 11. | The participant was previously exposed to any BTK inhibitor, |
| clinical study | | including tolebrutinib. |
| experience | E 12. | The participant has taken other investigational drugs within 3 |
| | | months or 5 half-lives, whichever is longer, before the |
| | | screening visit. |
| Diagnostic | E 13. | The participant has had a relapse in the 30 days prior to |
| assessments | | randomization. |
| | E 14. | The participant has a contraindication for MRI, ie, presence of |
| | | pacemaker, metallic implants in high-risk areas (ie, artificial |
| | | heart valves, aneurysm/vessel clips), presence of metallic |
| | | material (eg, shrapnel) in high risk areas, known history of |
| | | allergy to any contrast medium, or history of claustrophobia |
| | | that would prevent completion of all protocol scheduled MRI. |
| | | Note: People with a contraindication to Gd can be enrolled |
| | | into the study but cannot receive Gd contrast dyes during their |
| | | MRI scan. |
| Other exclusions | E 15. | Individuals accommodated in an institution because of |
| | | regulatory or legal order; prisoners or participants who are |
| | | legally institutionalized. |
| | E 16. | Participant not suitable for participation, whatever the reason, |
| | | as judged by the Investigator, including medical or clinical |
| | | conditions, or participants potentially at risk of noncompliance |
| | | to study procedures. |
| | E 17. | Participants are employees of the clinical study site or other |
| | | individuals directly involved in the conduct of the study, or |
| | | immediate family members of such individuals. |
| | E 18. | Any other situation during study implementation/course that |
| | | may raise ethics considerations |
| | | Note: a one-time retest at screening may be performed if an |
| | | abnormal laboratory test value is considered temporary |
| | Additional Exclusion Criteria for Japan | |
| | E 19. | A chest X-ray is required at screening. |
| | E 20. | People with a history of interstitial pneumonia or pulmonary |
| | | fibrosis are excluded from this study. |
| | E 21. | Monthly hematology and ALT/AST monitoring will be |
| | | continued until to 6 months, will then be repeated every 1.5 |
| | | months for another 6 months, and then will be repeated on a |
| | | quarterly basis. Monitoring will also include KL-6, CRP, and |
| | | lactate dehydrogenase (LDH) at each visit. |
| | E 22. | Temporary IMP interruption will occur for all ALT >5 × ULN |
| | | events regardless of the duration of ALT elevation above 5 × |
| | | ULN. |
| | E 23. | Temporary IMP interruption will occur if ALT >3 × ULN and |
| | | INR >1.3 or bilirubin >2 × ULN. Monitoring should then |
| | | occur every 2 to 3 days until ALT and INR/bilirubin start |
| | | down trending, then monitoring can be decreased to weekly |
| | | until ALT <1.5 × ULN. |
| | E 24. | In any confirmed case of ALT >5 × ULN and recommended |
| | | for ALT >3 × ULN but ALT <5 × ULN, testing of anti-HEV |
| | | IgA antibody will be performed as part of the workup. The |
| | | remaining components of the ALT algorithm apply for |
| | | participants in Japan. |
| | E 25. | It is strongly recommended to avoid initiating and continuing |
| | | use of hepatotoxic drugs and hepatotoxic herbs/supplements |
| | | while receiving IMP, due to the risk of hepatotoxicity |
| | | associated with the administration of tolebrutinib. |
| | E 26. | Large, publicly available resources such as the DILIrank |
| | | reference drug list (42, 43) can be a useful guide in identifying |
| | | hepatotoxic medications. Examples of hepatotoxic |
| | | herbals/supplements determined by the European Association |
| | | for the Study of Liver |

Abbreviations:
MRI: magnetic resonance imaging,
MS: multiple sclerosis,
PML: progressive multifocal leukoencephalopathy
*No time restriction if accelerated elimination procedure is done

### Lifestyle Considerations

Meals and dietary restrictions: tolebrutinib (IMP) shall be taken with a regular meal. When possible, the meal with which IMP is taken (eg, breakfast, lunch, or dinner) should be consistent throughout the study. The typical meal with which the IMP is taken will be recorded at each visit. In case the mealtime for IMP administration needs to be changed, a gap of a minimum of 12 hours between 2 doses should be maintained.

Caffeine, alcohol, and tobacco [for patients in Gemini 1 (EFC16033) study only]:
For each visit with PK/PD assessment, participants will abstain from ingesting caffeine- or xanthine-containing products (eg, coffee, tea, cola drinks, and chocolate) for 2 hours before the start of treatment until after collection of the final PK and/or PD sample later that day.
For each visit with PK/PD assessment, participants will abstain from alcohol for 24 hours before the start of treatment until after collection of the final PK and/or PD sample later that day.
During the entire study, participants should be warned not to consume substantial quantities of alcohol, defined as >14 grams (1 standard drink) per day in female participants or >28 grams (2 standard drinks) per day in male participants on a regular basis.

### Study Intervention

Study intervention is defined as any investigational intervention(s), marketed product(s), or placebo intended to be administered to a study participant according to the study protocol.

TABLE 2E
Overview of study interventions administered

| | | |
|---|---|---|
| ARM name | tolebrutinib | Teriflunomide |
| Intervention | tolebrutinib 60 mg | Teriflunomide 14 mg |
| name | Placebo matched to | Placebo matched to |
| | teriflunomide | tolebrutinib |
| Type | Drug | Drug |
| Dose | tolebrutinib: Film- | Teriflunomide: |
| formulation | coated tablet | Tablet |
| | Placebo matched to | Placebo matched to |
| | teriflunomide: | tolebrutinib: |
| | Tablet | Film-coated |
| | | tablet |
| Unit dose strength(s) | 60 mg | 14 mg |
| Dosage level(s) | Once daily | Once daily |
| Route of | Oral | Oral |
| administration | | |
| IMP and NIMP | IMP | IMP |
| Packaging | Study intervention will | Study intervention will |
| and labeling | be provided in wallet | be provided in wallet |
| | blister packaging. The | blister packaging. The |
| | content of the labeling | content of the labeling |
| | is in accordance with | is in accordance with |
| | the local regulatory | the local regulatory |
| | specifications and | specifications and |
| | requirements. | requirements. |
| Current/Former name(s) | Not applicable | Aubagio |
| or alias(es) | | |

IMP: investigational medicinal product,
NIMP: noninvestigational medicinal product

Between the protocol-scheduled, onsite visits, interim visits may be required for IMP dispensing. As an alternative to these visits or to replace onsite IMP dispensation, if needed, IMP may be supplied from the site to the participant via a Sponsor-approved courier company (direct-to-patient shipment) where allowed by local regulations and approved by the Sponsor.

### Noninvestigational Medicinal Product

### MRI Contrast-Enhancing Preparations

Route(s) of administration: IV
Dose regimen: as per respective label

### Cholestyramine

Route(s) of administration: oral
Dose regimen: 8 gram 3 times daily for 11 days for accelerated elimination procedure (4 gram 3 times daily for 11 days in case of intolerance). The teriflunomide local label should be followed.

### Concomitant Therapy

Any medication or vaccine (including over-the-counter or prescription medicines, vitamins, and/or herbal supplements) that the participant is receiving at the time of enrollment or receives during the study must be recorded along with:
Reason for use
Dates of administration including start and end dates
Dosage information including dose and frequency

Participants must abstain from taking prescription or nonprescription drugs (including vitamins and dietary or herbal supplements) within 7 days (or 14 days if the drug is a potential enzyme inducer) or 5 half-lives (whichever is longer) before the start of study intervention until completion of the follow-up visit, unless, in the opinion of the Investigator, the medication will not interfere with the study.

Live (attenuated) vaccines should not be administered during the intervention period.

Therapies for MS noted in the exclusion criterion E07 are not permitted after randomization while the participant is on study treatment. Short term use (3 to 5 days) of glucocorticoids (eg, for MS relapse treatment or an acute illness) and local corticosteroids (eg, topical, nasal, ocular, otic, intra-articular) are allowed.

The Medical Monitor should be contacted if there are any questions regarding concomitant or prior therapy.

For some prohibited concomitant medications (eg, aspirin >81 mg/day for headache), if use is not chronic, temporary discontinuation of IMP can be considered prior to a decision to permanently stop the IMP.

Medications for treatment of MS symptoms (eg, walking impairment, fatigue, spasticity, incontinence, pain) should be maintained at a stable dose prior to screening and for the duration of the treatment period, if clinically feasible.

Anticoagulant/antiplatelet therapies are not permitted to be taken concomitantly with the IMP, including:
Acetylsalicylic acid (aspirin) >81 mg/day
Antiplatelet drugs (eg, clopidogrel)
Warfarin (vitamin K antagonist)
Heparin, including low molecular weight heparin (antithrombin agents)
Dabigatran (direct thrombin inhibitor)
Apixaban, edoxaban, rivaroxaban (direct factor Xa inhibitors)

Paracetamol/acetaminophen, at doses of ≤3 grams/day, is permitted for use at any time during the study. A short course (up to 5 days) of nonsteroidal anti-inflammatory drugs (NSAIDs) (other than acetylsalicylic acid), preferably selective cyclooxygenase-2 inhibitors at the lowest effective dose, may be given during the study if clinically necessary for the treatment of an existing medical condition or a new event. The Investigator must record the use of NSAIDs (and any other comedication) in the eCRF.

### CYP Inhibitor/Inducer:

Potent and moderate inducers of CYP3A or potent inhibitors of CYP2C8 hepatic enzymes are not permitted throughout the study.
Tolebrutinib: Tolebrutinib is a substrate of the CYP3A4 and CYP2C8 isoenzymes. In healthy participants, potent CYP3A4 inhibitor (itraconazole 200 mg once daily for 4 days) increased tolebrutinib area under the curve (AUC) exposure 1.8-fold (INT16385 study) and potent CYP2C8 inhibitor (gemfibrozil 600 mg twice daily for 6 days) increased tolebrutinib (AUC) exposure 8.4-fold (INT16726 study). Based on a satisfactory safety and tolerability profile and on the observed exposure in healthy participants who received tolebrutinib at a dose of up to 240 mg once daily for 14 days under fed conditions (TDR16862 study), drugs that strongly inhibit CYP3A4 are allowed and drugs that strongly inhibit CYP2C8 are not permitted. In healthy participants, potent CYP3A4 and moderate CYP2C8 induction by rifampicin (600 mg once daily for 8 days) decreased tolebrutinib exposure 6-fold (INT16726 study). Therefore, potent and also moderate (based on prediction) CYP3A inducers are not permitted due to their potential to decrease tolebrutinib exposure and efficacy. See Tables 2H-A and 2H-B for the list of drugs not to be used.
Teriflunomide: Other potent CYP and transporter inducers should be avoided due to their potential to decrease teriflunomide exposure. Rifampicin and other known potent CYP and transporter inducers such as carbamazepine, phenobarbital, phenytoin, avasimibe, lumacaftor, rifapentine, rifabutin, and St John's wort, should be avoided. Leflunomide is prohibited, because teriflunomide is a metabolite of leflunomide.

Cholestyramine (and other bile acid sequestrants) and activated charcoal use should be avoided because these may lead to significant decrease in plasma concentration of teriflunomide.

Cholestyramine can be used only when an accelerated elimination procedure is needed. In exceptional situations (eg, when cholestyramine is not tolerated, or cholestyramine is not available) use of activated charcoal (as per the current Aubagio SmPC) can be considered.

Breast cancer resistance protein (BCRP) inhibitors (eg, eltrombopag and gefitinib) should be avoided. Based on in vitro studies, teriflunomide is a substrate of the efflux transporter BCRP, so these drugs may increase exposure of teriflunomide.

A number of drugs need to be used with caution as their exposure may be altered by teriflunomide. Adequate arrangements need to be made to monitor their effects and to ensure timely decision for change of medication.
Medicinal products metabolized by CYP2C8 (eg, repaglinide, pioglitazone, and rosiglitazone) should be used with caution during treatment with the IMP due to potential of increase of concentrations of these drugs following CYP2C8 inhibition by teriflunomide.
Increase in ethinylestradiol and levonorgestrel exposure following repeated doses of teriflunomide has been observed (Aubagio (teriflunomide) [package insert]. Genzyme Corporation. Cambridge, MA 02142; 2021).
While this interaction of teriflunomide is not expected to adversely impact the efficacy of oral contraceptives, it should be considered when selecting or adjusting oral contraceptive treatment used during this trial.
Medicinal products metabolized by CYP1A2 (eg, duloxetine, alosetron, theophylline, and tizanidine) should be used with caution during treatment with IMP due to the capacity of teriflunomide to induce CYP1A2 and to reduce efficacy of these products.
Administration of substrates of organic anion transporter 3 (OAT3) (eg, cefaclor, benzylpenicillin, ciprofloxacin, indomethacin, ketoprofen, furosemide, and cimetidine) should be performed with caution during the study due to the potential of teriflunomide to increase exposure to these medications through inhibition of OAT3.
For substrates of BCRP (eg, sulfasalazine) and the organic anion transporter polypeptide (OATP) family (eg, rosuvastatin, simvastatin, atorvastatin, pravastatin, nateglinide, repaglinide, and rifampicin), concomitant administration with teriflunomide should be used with caution during the study due to the potential of teriflunomide to increase exposure to these products. Participants should be closely monitored for signs and symptoms of excessive exposure to the medicinal products, and reduction of the dose should be considered. If used together with the IMP, the dose of rosuvastatin should not exceed 10 mg once daily.

### Rescue Medicine

There is no rescue medication planned for this study.

Multiple sclerosis relapse treatments are allowed as per local routine practice (eg, high dose IV methylprednisolone for 3 to 5 days). These and all other concomitant medications must be reported in the eCRF.

### Dose Modification

Dose reduction is not foreseen in this study. Treatment may need to be interrupted or permanently discontinued if deemed necessary due to an AE.

Intervention after the End of the Study

After the end of this study, participants who successfully complete the trial and are taking the IMP until the end of the trial will be offered the option to participate in the LTS study for an additional 2 years, or until the drug is approved in their respective country, whichever comes first. Details of the LTS study will be described in a separate protocol. Post-trial access for more than 2 years may be considered if mandated by local regulations.

### Discontinuation of Study Intervention

### Definitive Discontinuation

The study intervention should be continued whenever possible.

Permanent intervention discontinuation is any treatment discontinuation associated with the definitive decision from the Investigator or the participant not to re-expose the participant to the study intervention at any time.

In rare instances, it may be necessary for a participant to permanently discontinue the study intervention. If study intervention is permanently discontinued, the participant shall be asked to remain in the study to be evaluated until the EOS visit. This will be important to continue to evaluate for safety. See the SoA (Table 2A) for data to be collected at the time of discontinuation of the study intervention. In the case that the study intervention is permanently discontinued, the participant should be treated for MS according to local clinical practice and the best judgment of the Investigator.

The following may be justifiable reasons for the Investigator or Sponsor to discontinue a participant from study treatment:
Adverse events that endanger the safety of the participant, or if discontinuation of study intervention is desired or considered necessary by the Investigator and/or participant.
If IMP discontinuation criteria are met as per guidance for the follow up of laboratory abnormalities in FIGS. 2-8.
The participant is no longer deriving a therapeutic/clinical benefit in the opinion of the Investigator.
At participant's request, ie, withdrawal of the consent for treatment.
If a female participant becomes pregnant or wishes to become pregnant during the study.
If a male participant wishes to conceive a child during the study.
Any serious opportunistic infections (eg, PML [see FIG. 7], HIV).
Continued need for/chronic use of a prohibited concomitant medication (see CONCOMITANT THERAPY in this example and Table 2H-A and 2H-B).

Discontinuation of the study intervention for abnormal liver function should be considered by the Investigator when a participant meets one of the conditions outlined in the algorithm (FIG. 4) or if the Investigator believes that it is in the best interest of the participant.

Any clinically significant abnormal laboratory value or ECG parameter will be immediately rechecked for confirmation after 24 hours before making a decision of definitive discontinuation of the IMP for the concerned participant.

If a clinically significant finding is identified in the ECG (including, but not limited to changes from baseline in QT interval corrected (QTc) using Fridericia's formula [QTcF] after enrollment), the Investigator or a qualified designee will determine if the participant can continue in the study and if any change in participant management is needed. Review of ECG findings by a cardiologist may be considered for a decision of a definitive discontinuation of study intervention because of ECG changes. This review of the ECG findings recorded at the time of collection must be documented. Any new clinically relevant ECG finding should be reported as an AE.

See the SoA (Table 2A) for data to be collected at the time of intervention discontinuation and follow up and for any further evaluations that need to be completed.

Additional tests may be performed at any time during the study as determined necessary by the Investigator or required by local regulations.

TABLE 2F
Protocol-required laboratory assessments

Laboratory

| assessments | Parameters | | |
|---|---|---|---|
| Hematology | Platelet count | RBC indices: | WBC count with |
| | | | differential: |
| | RBC count | MCV | Neutrophils |
| | Hemoglobin | MCH | Lymphocytes |
| | Hematocrit | % Reticulocytes | Monocytes |
| | | | Eosinophils |
| | | | Basophils |
| Clinical | BUN | Sodium | AST |
| chemistry^{a} | Creatinine^{b} | Calcium | ALT |
| | Glucose | Total and direct bilirubin | Alkaline phosphatase |
| | Potassium | Total protein | Albumin |
| | | Chloride | Creatine phosphokinase |
| | | Bicarbonate | Lipase (screening only) |

| | |
|---|---|
| Routine | - Specific gravity |
| urinalysis | - pH, glucose, protein, blood, ketones, bilirubin, urobilinogen, nitrite, |
| | leukocyte esterase by dipstick |
| | - Microscopic examination (if blood or protein is abnormal and for signs of |
| | infection) |
| Other | - FSH and estradiol (if needed, only in female participants to confirm |
| screening | postmenopausal state) |
| tests | - Highly sensitive serum or urine β-hCG pregnancy test (as needed for |
| | women of childbearing potential)^{c} |
| | - Coagulation: PT/INR, aPTT |
| | - Serology tests for hepatitis B (HBs Ag, anti-HBc IGM and total, anti-HBs) |
| | and C virus (anti-HCV); in case these results are inconclusive (eg anti-HBS |
| | negative and anti-HBc positive or anti-HC IgG positive), HBV-DNA or |
| | HCV-RNA testing, respectively, should be performed for confirmation. HIV |
| | and other infectious diseases, if locally required. |
| | - Tuberculosis test: Blood testing (eg, QuantiFERON ® TB Gold test) is |
| | preferred; skin testing (eg, tuberculin skin test) with ancillary testing will be |
| | allowed if blood testing is not available. T-SPOT can also be performed, if |
| | available. |
| | - Iron panel (serum): iron, ferritin, transferrin saturation TIBC. |

ALT: alanine aminotransferase;
anti-HBc; antibody to hepatitis B core antigen;
anti-HBs: hepatitis B surface antibody;
aPTT: activated partial thromboplastin time;
AST: aspartate aminotransferase;
BUN: blood urea nitrogen;
β-hCG: human chorionic gonadotropin;
FSH; follicle-stimulating hormone;
IEC: independent ethics committee;
INR: international normalized ratio;
HBsAg: hepatitis B surface antigen;
HBV: hepatitis B virus;
HCV: hepatitis C virus;
HIV: human immunodeficiency virus;
Ig: immunoglobulin;
IRB: institutional review board;
MCH: mean corpuscular hemoglobin;
MCV: mean corpuscular volume;
PT: prothrombin time;
RBC: red blood cell;
SGOT: serum glutamic-oxaloacetic transaminase;
SGPT: serum glutamic-pyruvic transaminase;
TB: tuberculosis;
ULN: upper limit of normal;
WBC: white blood cell
^{a}Details of liver chemistry stopping criteria and required actions and follow-up assessments after observations of ALT >3 × ULN are given in FIG. 4. Clinical laboratory findings of ALT >3 × ULN and bilirubin ≥2 × ULN (>35% direct bilirubin), or ALT >3 × ULN and international normalized ratio (INR) >1.5, if INR measured, that may suggest severe liver injury and must be reported as an SAE.
^{b}Other renal function parameters, creatinine clearance (CrCl) will be calculated.
^{c}Local urine testing for pregnancy will be standard for the protocol (except for the Screening Visit, when a serum pregnancy test is required) unless serum testing is required by local regulation or IRB/IEC.

### Liver and Other Safety: Actions and Follow-Up Assessments

These actions described in Table 2G-A and FIGS. 3-4 are required for ALT increase and thrombocytopenia events ONLY. For all other safety events described, these are suggested per the Investigator's medical judgement.

Neutropenia is to be recorded as an AE only if at least 1 of the criteria listed in the general guidelines for reporting AEs is met.

Thrombocytopenia is to be recorded as an AE only if at least 1 of the criteria listed in the general guidelines for reporting AEs is met.

Note: “Baseline” refers to ALT sampled at baseline visit; or if baseline value unavailable, to the latest ALT sampled before the baseline visit. The algorithm does not apply to the instances of increase in ALT during Screening.

TABLE 2G-A
Actions for cases of confirmed ALT elevation

In ANY CONFIRMED CASE of ALT >5 × ULN, or ALT >3 × ULN with bilirubin >2 ×
ULN, the following steps are REQUIRED (recommended for ALT >3 × ULN but ALT <5 ×
ULN, as clinically indicated):
INFORM the Site Monitor, who will forward the information to the Study Manager.
INVESTIGATE specifically for malaise with or without loss of consciousness,
dizziness, and/or hypotension and/or episode of arrhythmia in the previous 72
hours; rule out muscular injury.
PERFORM the following tests:
LFTs: AST, ALT, alkaline phosphatase, GGT, total and conjugated bilirubin,
and prothrombin time/INR (mandatory assessments for ALT >3 × ULN);
CPK, serum creatinine, complete blood count;
Anti-HAV IgM, anti-HBc IgM, (HBV-DNA if clinically indicated), hepatitis B
surface antigen (HBsAg), anti-HCV and HCV RNA, anti-CMV IgM, and anti-
HEV IgM antibodies;
Iron, ferritin, transferrin saturation;
Auto-antibodies: serum IgG levels, antinuclear, anti-DNA, anti-smooth muscle,
anti-LKM, anti-mitochondrial;
Evaluate recent infection with EBV, herpes viruses. Depending on the clinical
context, consider testing for toxoplasma;
Collect and freeze serum sample (5 mL × 2);
Collect and store one PK sample following the instructions in the central
laboratory manual;
Perform hepatobiliary imaging (ultrasonography or other imaging
investigations is required);
Consider DNA test for Gilbert's disease if clinically indicated;
Recommend consulting a hepatologist (mandatory if ALT >8 × ULN or is
associated with elevated bilirubin);
Discuss with the hepatologist the clinical indication for potential liver
biopsy (strongly recommended if the participant meets Hy's law
criteria or has ALT >20 × ULN) and/or initiation of treatment with
steroids;
Consider patient hospitalization if INR >2 (or PT <50%) and/or central nervous
system disturbances suggesting hepatic encephalopathy.
MONITOR LFTs after discontinuation of IMP:
Monitor closely (every 2-3 days) until ALT is down-trending,
then weekly until <1.5 × ULN, and then at every scheduled
visit;
This frequent LFT monitoring may be done through central or
local lab, or via home visit (depending on the
Investigator's assessment and/or local regulatory requirements).
Rechallenge Re-initiation of the study drug can only be considered after
discussion with the Sponsor's Medical Monitor once the ALT/AST decreases
to <1.5 × ULN, and there is no clinical contraindication. Rechallenge is not
permitted for the following participants unless a clear non-DILI etiology is
identified:
ALT >8 × ULN
ALT >5 × ULN for greater than two weeks
ALT >3 × ULN and total bilirubin >1 × ULN
In case it is agreed to re-start the study drug, it is recommended that
ALT/AST be assessed per protocol schedule of assessments for the
first 6 months of the treatment period.
The occurrence of new elevation to >3 × ULN for the ALT/AST values
will lead to permanent discontinuation of the study drug
ALT, alanine aminotransferase; AST, aspartate aminotransferase; CMV, cytomegalovirus; CPK, creatine phosphokinase; CRF, case report form; EBV, Epstein-Barr virus; GGT, gamma glutamyl transferase; HAV, hepatitis A virus; HBV, hepatitis B virus; HCV, hepatitis C virus; HEV, hepatitis E virus; IgG, immunoglobulin G; IgM, immunoglobulin M; IMP, investigational medicinal product; INR, international normalized ratio; LFT, liver function test; LKM, liver-kidney microsomal antibody; PT, prothrombin time; ULN, upper limit of normal.

Increase in serum creatinine is to be recorded as an AE only if at least 1 of the criteria listed in the general guidelines for reporting AEs is met.

Increase in CPK is to be recorded as an AE only if at least 1 of the criteria in the general guidelines for reporting AEs is met.

SUSPECTED PML: If either the clinical presentation or MRI features of a participant are suggestive of PML, the diagnostic and action algorithm described in FIG. 7 is recommended.

Clinical manifestations or MRI lesions features suspicious for PML are proposed in Table 2G-B (based on Berger et al. Neurology 2013, 80, 1430-1438 and Kappos et al. Lancet Neurol. 2007, 6, 431-441).

TABLE 2G-B
Clinical and MRI features suggestive of PML

| | |
|---|---|
| Clinical | Subacute onset of weakness, sensory deficits, cognitive |
| history | or behavioral abnormalities, gait dysfunction, speech/language |
| | difficulties or any other signs of cortical dysfunction, |
| | retrochiasmal visual defects or seizure |
| Brain MRI | ≥1 T2/FLAIR hyperintense and T1 hypointense lesions involving |
| | the subcortical and juxtacortical white matter, sparing the cortex, |
| | with no mass effect, with a continuous progression; new lesions |
| | with no enhancement (even when large) or with faint rim |
| | enhancement |

The detection of John Cunningham virus (JCV) DNA in the cerebrospinal fluid of a patient with clinical and MRI features suggestive of PML establishes the diagnosis of PML.
If JCV DNA is not detected in cerebrospinal fluid and if clinical suspicion of PML remains high, another lumbar puncture should be performed.
If diagnosis remains uncertain and suspicion of PML remains high, a brain biopsy may be considered to establish a definitive diagnosis

Clinical or MRI features suggestive of PML should be recorded as an AE/AESI/SAE.

Examples of Drugs with a Potential to Change Tolebrutinib Metabolism

The following drugs should not be taken during the study concomitantly with the IMP due to their potential to change tolebrutinib kinetics due to interaction with P450-mediated metabolism, being potent/moderate inducers of CYP3A or potent inhibitors of CYP2C8 liver enzymes (per the lists of the Drug Interaction Database Program of the University of Washington; Table 2H-A).

Additionally, participants in the US must not take medications that are mild, moderate, or potent inhibitors of CYP3A or CYP2C8 (Table 2H-B).

Please note that the lists provided in Tables 2H-A and 2H-B are not exhaustive and that the product information of drugs intended for concomitant use should be consulted.

TABLE 2H-A
CYP3A Inducers and CYP2C8 Inhibitors
Potent CYP3A Inducers:

| | |
|---|---|
| Rifampin | Carbamazepine |
| Phenobarbital | St John's wort extract |
| Avasimibe | Lumacaftor |
| Rifapentine | Rifabutin |
| Phenytoin | |

Potent CYP2C8 Inhibitors:

| | |
|---|---|
| Gemfibrozil | Deferasirox |
| Clopidogrel | |

Moderate CYP3A Inducers

| | |
|---|---|
| Semagacestat | Asunaprevir/beclabuvir/daclatasvir |
| Cenobamate | Nafcillin |
| Lesinurad | |
| Bosentan | Telotristat ethyl |
| Thioridazine | Elagolix |
| Rifabutin | |

TABLE 2H-B
Mild, moderate, and potent inhibitors of CYP3A and
CYP2C8, and moderate and potent inducers of CYP3A

| | Potent | Moderate | Mild |
|---|---|---|---|
| CYP2C8 | Clopidogrel | Trimethoprim | Sulfamethoxazole |
| inhibitors | Gemfibrozil | | trimethoprim |
| | | | Fluvoxamine |

| | Potent | Moderate |
|---|---|---|
| CYP3A | Avasimibe | Elagolix |
| inducers | Rifampin | Cenobamate |
| | Carbamazepine | Nafcillin |
| | Lumacaftor | Asunaprevir/beclabuvir/daclatasvir |
| | Phenobarbital | Lesinurad |
| | Phenytoin | Bosentan |
| | Rifapentine | Thioridazine |
| | St. John's Wort | Rifabutin |

| | Potent | Moderate | Mild |
|---|---|---|---|
| CYP3A | Clarithromycin | Ciprofloxacin | Alprazolam |
| inhibitors | Itraconazole | Diltiazem | Atorvastatin |
| | Ketoconazole | Erythromycin | Amlodipine |
| | Nirmatrelvir and ritonavir | Fluconazole | Cimetidine |
| | Fluoxetine | Verapamil | Ranitidine |
| | Grapefruit juice | Sertraline | Roxithromycin |
| | | | Ginkgo biloba |
| | | | Isoniazid |

Examples of Drugs with a Potential to Change Teriflunomide Disposition

### BCRP Inhibitors

Cyclosporine, Eltronbopag, Gefitinib
   Examples of Drugs which can be Potentially Affected by Teriflunomide

Please note that the lists provided are not exhaustive and that the local product information of drugs intended for concomitant use should be consulted. If coadministration with the IMP is needed, caution needs to be exercised to detect decreased or increased exposure of these drugs and participants need to be monitored to detect this in a timely fashion.

Coadministration of teriflunomide and warfarin may lead to decreased exposure of warfarin. Therefore, when warfarin is coadministered with IMP, close INR follow up and monitoring are recommended.

Exposure of drug substances listed below may be increased by teriflunomide:

CYP2C8 substrates

| | |
|---|---|
| Repaglinide | Pioglitazone |
| | Rosiglitazone |

Oral contraceptives

| | |
|---|---|
| Ethinvlestradiol | Levonorgestrel |

Organic anion transporter 3 (OAT3) substrates

Cefaclor
Penicillin G
Ciprofloxacin
Indomethacin
Ketoprofen
Furosemide
Cimetidine

BCRP substrates

Rosuvastatin
Sulfasalazine

Organic anion transporting polypeptide B1 and B3 (OATP1B1/B3) substrates

Simvastatin
Atorvastatin
Pravastatin
Repaglinide
Rifampin
Nateglinide

NOTE:
exposure may be increased by teriflunomide

Exposure of drug substances listed below may be decreased by teriflunomide.

CYP1A2 substrates

| | |
|---|---|
| Duloxetine | Theophylline |
| Alosetron | Tizanidine |
| Caffeine | |

TABLE 2J
Baseline and disease characteristics of study patients

RMS†

| | Gemini 1 | Gemini 2 |
|---|---|---|
| | (EFC16033) | (EFC16034) |
| Baseline Characteristics | | |
| Age (mean) years | 37 | 36 |
| Female | 68% | 66% |
| White | 82% | 92% |
| Asian | 15% | 5% |
| Black/African American | 1.4% | 2% |
| US | 9% | 12% |
| Eastern Europe | 55% | 44% |
| Western Europe | 14% | 20% |
| North America | 10% | 13% |
| Rest of World | 21% | 24% |
| Disease Characteristics | | |
| EDSS (mean) | 2.4 | 2.4 |
| Symptom onset (median) years | 5 | 3.3 |
| Diagnosis (median) years | 1.6 | 1 |
| Treatment naive | 62% | 67% |
| ≥2 prior DMT | 13% | 8% |
| Gd + T1 lesion free | 63% | 67% |
| Relapse in prior year | 94% | 94% |

TABLE 2K
Baseline characteristics of study patients (intention-to-treat population)^{a}

| | GEMINI 1 | | | | GEMINI 2 | | | |
|---|---|---|---|---|---|---|---|---|
| | Teriflunomide | | Tolebrutinib | | Teriflunomide | | Tolebrutinib | |
| Characteristic | (N = 488) | | (N = 486) | | (N = 452) | | (N = 447) | |
| Age, years | 36.6 | (9.4) | 36.8 | (9.0) | 36.1 | (9.3) | 36.6 | (9.3) |
| Female, n (%) | 325 | (66.6) | 334 | (68.7) | 293 | (64.8) | 300 | (67.1) |
| Race-no. (%) | | | | | | | | |
| White | 406 | (83.2) | 395 | (81.3) | 417 | (92.3) | 411 | (91.9) |
| Black or African American | 10 | (2.0) | 4 | (0.8) | 11 | (2.4) | 7 | (1.6) |
| Asian | 67 | (13.7) | 78 | (16.0) | 19 | (4.2) | 23 | (5.1) |
| Other/Unknown/Not reported | 5 | (1.0) | 9 | (1.9) | 5 | (1.1) | 6 | (1.3) |
| Region-no. (%) | | | | | | | | |
| US | 43 | (8.8) | 43 | (8.8) | 54 | (11.9) | 51 | (11.4) |
| Non-US | 445 | (91.2) | 443 | (91.2) | 398 | (88.1) | 396 | (88.6) |
| Multiple sclerosis subtype-no. (%) | | | | | | | | |
| Relapsing-remitting | 483 | (99.0) | 480 | (98.8) | 450 | (99.6) | 443 | (99.3) |
| Secondary progressive | 5 | (1.0) | 6 | (1.2) | 2 | (0.4) | 3 | (0.7) |
| Time since diagnosis-years | 4.6 | (6.0) | 4.8 | (6.2) | 3.8 | (5.4) | 3.8 | (5.4) |
| Time since most recent relapse- | 7.4 | (17.3) | 6.2 | (13.5) | 7.2 | (15.5) | 8.5 | (25.9) |
| months | | | | | | | | |
| Participants who were treatment- | 291 | (59.6) | 315 | (64.8) | 300 | (66.4) | 301 | (67.3) |
| naïve, n (%) | | | | | | | | |
| Participants with a previous | 197 | (40.4) | 171 | (35.2) | 152 | (33.6) | 146 | (32.7) |
| disease-modifying therapy^{b}-no. (%) | | | | | | | | |
| Interferons^{c} | 100 | (20.5) | 104 | (21.4) | 85 | (18.8) | 85 | (19.0) |
| Glatiramer acetate | 61 | (12.5) | 59 | (12.1) | 40 | (8.8) | 40 | (8.9) |
| Dimethyl fumarate | 34 | (7.0) | 25 | (5.1) | 31 | (6.9) | 21 | (4.7) |
| Fingolimod | 24 | (4.9) | 15 | (3.1) | 8 | (1.8) | 15 | (3.4) |
| Ocrelizumab | 10 | (2.0) | 11 | (2.3) | 5 | (1.1) | 1 | (0.2) |
| Diroximel fumarate | 15 | (3.1) | 9 | (1.9) | 1 | (0.2) | 2 | (0.4) |
| Teriflunomide | 9 | (1.8) | 9 | (1.9) | 11 | (2.4) | 7 | (1.6) |
| Natalizumab | 9 | (1.8) | 7 | (1.4) | 2 | (0.4) | 5 | (1.1) |
| Other | 22 | (4.5) | 23 | (4.7) | 18 | (4.0) | 14 | (3.1) |
| Number of relapses in the past 1 year | 1.2 | (0.6) | 1.2 | (0.6) | 1.2 | (0.6) | 1.1 | (0.5) |
| Number of relapses in the past 2 years | 1.5 | (0.8) | 1.6 | (1.0) | 1.5 | (0.9) | 1.5 | (0.8) |
| EDSS score^{d} | | | | | | | | |
| Mean (SD) | 2.37 | (1.20) | 2.42 | (1.19) | 2.32 | (1.19) | 2.42 | (1.17) |
| Median (IQR) | 2.0 | (1.5-3.0) | 2.0 | (1.5-3.0) | 2.0 | (1.5-3.0) | 2.3 | (1.5-3.3) |
| Participants with ≥1 gadolinium- | 186 | (38.4) | 168 | (34.7) | 146 | (32.6) | 145 | (32.4) |
| enhancing | | | | | | | | |
| T1-weighted lesion-no. (%) | | | | | | | | |
| Number of gadolinium-enhancing | 1.5 | (4.2) | 1.3 | (3.7) | 1.0 | (3.2) | 1.0 | (2.4) |
| T1-weighted lesions | | | | | | | | |
| T2-weighted lesion volume-cm³ | | | | | | | | |
| Mean (SD) | 14.0 | (12.0) | 14.6 | (13.0) | 10.8 | (9.9) | 11.5 | (10.6) |
| Median (IQR) | 10.7 | (4.9-19.4) | 11.6 | (4.8-19.5) | 7.7 | (3.5-15.4) | 8.3 | (3.8-15.5) |
| Normalised brain volume-cm³ | 1538.1 | (92.4) | 1533.0 | (103.7) | 1551.4 | (81.5) | 1547.1 | (81.1) |
| SDMT, number of correct | 0.015 | (0.989) | −0.015 | (1.012) | 0.027 | (0.976) | −0.027 | (1.024) |
| substitutions Z-score^{e} | | | | | | | | |
| CVLT-II, total correct | 51.0 | (14.6) | 49.5 | (14.0) | 51.6 | (13.0) | 50.7 | (14.0) |
| standardized score^{f} | | | | | | | | |

^{a}Data are mean (SD) except where specified. Note variations in the number of participants for the following variables: Time since diagnosis, tolebrutinib (GEMINI 1) N = 485; time since most recent relapse, tolebrutinib (GEMINI 1) N = 485, teriflunomide (GEMINI 1) N = 486, tolebrutinib (GEMINI 2) N = 445, teriflunomide (GEMINI 2) N = 449; number of relapses in the past 1 year, tolebrutinib (GEMINI 1) N = 484, teriflunomide (GEMINI 1) N = 487, teriflunomide (GEMINI 2) N = 451; number of relapses in the past 2 years, tolebrutinib (GEMINI 1) N = 483, teriflunomide (GEMINI 2) N = 451; participants with ≥1 gadolinium-enhancing T1-weighted lesion and mean number of gadolinium-enhancing T1-weighted lesions, tolebrutinib (GEMINI 1) N = 484, teriflunomide (GEMINI 1) N = 485, teriflunomide (GEMINI 2) N = 448; T2-weighted lesion volume, tolebrutinib (GEMINI 1) N = 484, teriflunomide (GEMINI 2) N = 450; normalized brain volume, tolebrutinib (GEMINI 1) N = 484, teriflunomide (GEMINI 2) N = 449; SDMT number of correct substitutions Z-score, tolebrutinib (GEMINI 1) N = 480, teriflunomide (GEMINI 1) N = 477, tolebrutinib (GEMINI 2) N = 437, teriflunomide (GEMINI 2) N = 439; CVLT-II total correct standardized score, tolebrutinib (GEMINI 1) N = 475, teriflunomide (GEMINI 1) N = 471, tolebrutinib (GEMINI 2) N = 424, teriflunomide (GEMINI 2) N = 427.
^{b}Participants can be counted in more than one category.
^{c}Interferon therapies include interferon beta-1a, interferon beta-1b, and peginterferon beta-1a.
^{d}EDSS scores range from 0 to 10.0, with higher scores indicating worse disability. Baseline values represent the average of screening and randomization assessments.
^{e}Scores on SDMT range from 0 to 110, with lower scores indicating worse cognitive function. Z scores were calculated as (score − baseline mean)/baseline SD.
^{f}Scores on CVLT-II are standardized by sex and age and have a mean of 50 and SD of 10, with lower scores indicating worse cognitive function.
CVLT-II, California Verbal Learning Test - Second Edition; EDSS, Expanded Disability Status Scale; Gd, gadolinium; IQR, interquartile range; SD, standard deviation; SDMT, Symbol Digit Modalities Test.

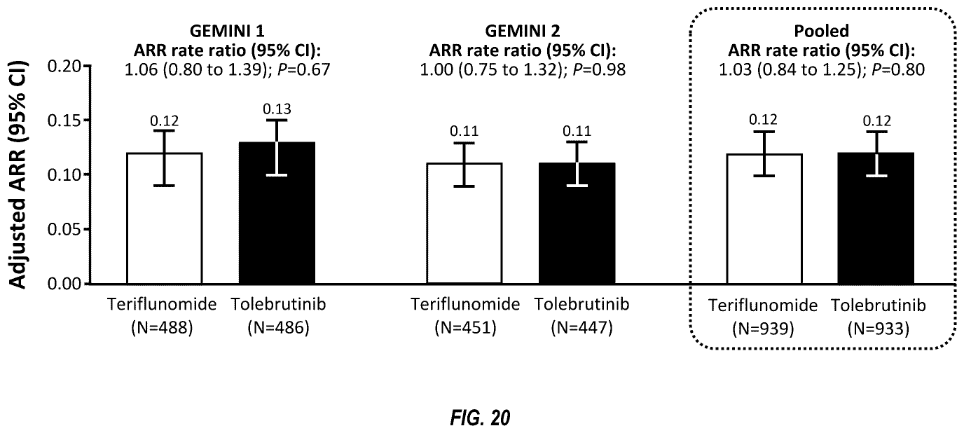

The primary endpoint results, annualized (adjudicated) relapse rates (ARR), were low in the teriflunomide arm in both GEMINI 1 and 2, and no statistically significant improvement in ARR was observed for tolebrutinib compared to teriflunomide. In GEMINI 1, tolebrutinib demonstrated an ARR of 0.13, and teriflunomide demonstrated an ARR of 0.12 (adjusted rate ratio 1.06; 95% CI: 0.81 to 1.39; p=0.67) (FIG. 20). In GEMINI 2, tolebrutinib demonstrated an ARR of 0.11, and teriflunomide demonstrated an ARR of 0.11 (adjusted rate ratio 1.00; 95% CI: 0.75 to 1.32; p=0.98). These relapse rates amount to approximately 1 relapse every 8 years. In a prespecified pooled analysis of GEMINI 1 and 2, the ARR was 0.12 (adjusted rate ratio 1.03; 95% CI: 0.84 to 1.25; p=0.80) for both tolebrutinib and teriflunomide.

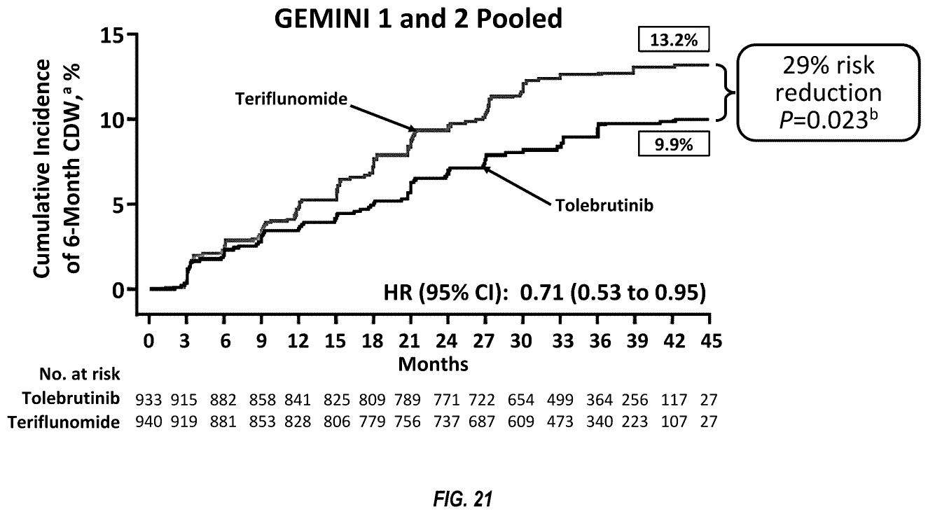

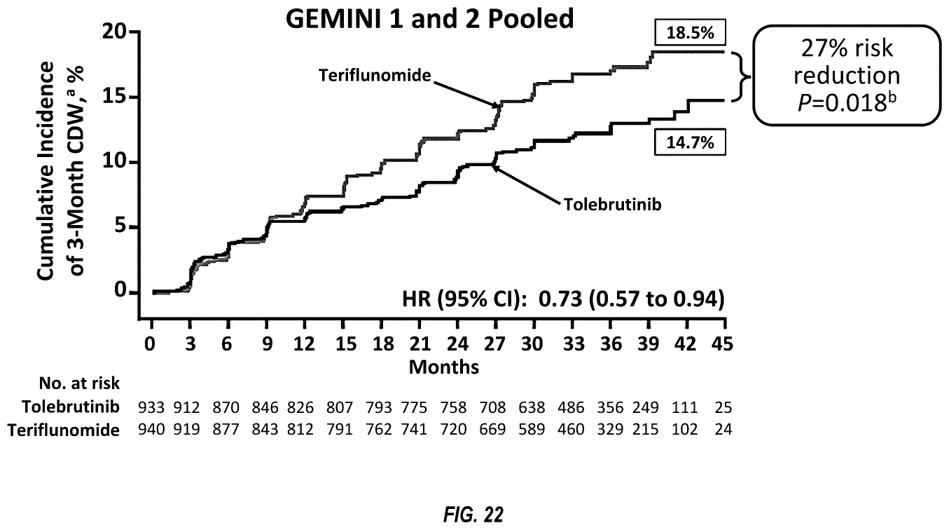

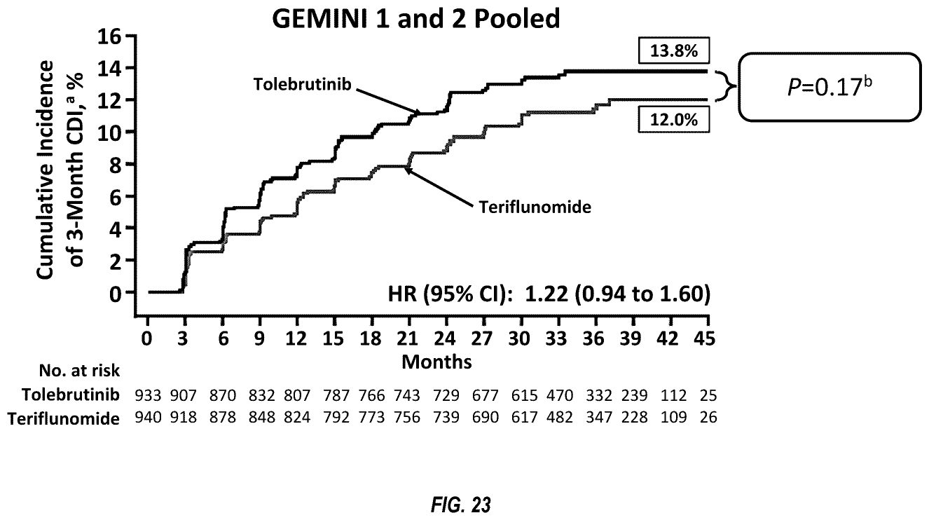

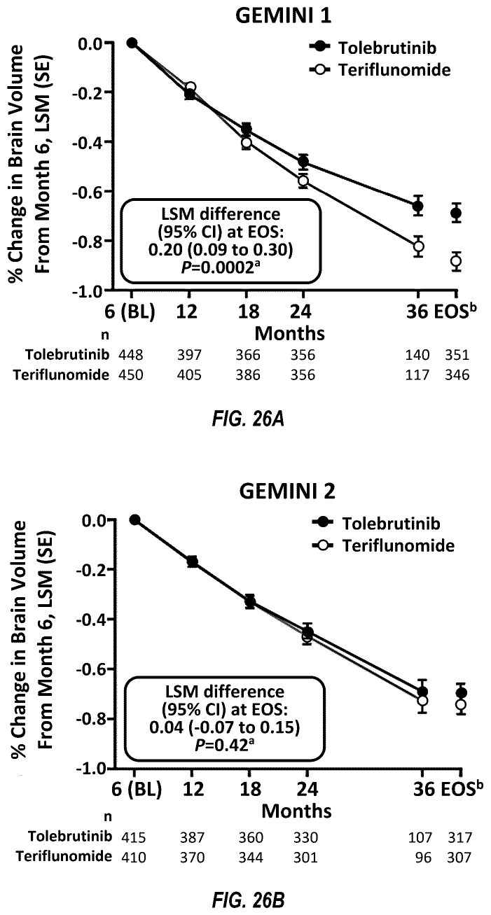

However, the study results showed treatment with tolebrutinib reduced disability accumulation in patients with RMS compared to teriflunomide (e.g., FIGS. 21, 22, and 26A-26B). Specifically, in a pooled analysis from GEMINI 1 and 2 of the key secondary endpoint, tolebrutinib delayed the time to onset of 6-month confirmed disability worsening (CDW) by 29% compared to teriflunomide (HR 0.71; 95% CI: 0.53-0.95; p=0.023) (FIG. 21). This delay in disability accumulation in RMS patients with very low relapse activity (delay in time to onset of 6-month CDW by 29%) was similar to that observed in the Hercules study for nrSPMS patients (delay in time to onset of 6-month CDP by 31%), described in Example 1. The very low relapse rates in participants with RMS suggest that the majority of CDW events were due to progression independent of relapse activity (PIRA). Tolebrutinib also delayed the time to onset of 3-month confirmed disability worsening (CDW) by 27% compared to teriflunomide (FIG. 22). In the Gemini 1 study, tolebrutinib also improved the percent change in brain volume loss (BVL) as detected by brain MRI (FIG. 26A). The changes in BVL for patients on tolebrutinib approach what is observed in the natural progression of healthy adults.

These results, and in particular, the clear reduction in disability accumulation despite no significant differences in relapses versus teriflunomide, are consistent with the hypothesis that acute focal inflammation and smoldering neuroinflammation are two distinct biological processes. Furthermore, the significant impact of tolebrutinib on disability accumulation versus teriflunomide, in the absence of statistically significant relapses, suggests that tolebrutinib may address smoldering neuroinflammation, which manifests as progression independent of relapse activity. This disability result is consistent with the findings of the HERCULES trial (Example 1) where tolebrutinib demonstrated a significantly reduced risk of confirmed disability progression versus placebo in participants with non-relapsing secondary progressive multiple sclerosis.

A summary of the results collected from the Gemini 1 and 2 (EFC16033 and EFC16034) studies described in this example are listed in Table 2L.

TABLE 2L
Summary of the multiplicity adjustment for the primary, key secondary and other
secondary efficacy endpoints in Gemini 1 and 2 studies (EFC16033 and EFC16034)

| | | | Comparison between teriflunomide | | |
|---|---|---|---|---|---|
| | | | 14 mg and tolebrutinib 60 mg | | |
| | | | Gemini 1 | Gemini 2 | EFC16033 + |
| Category | Endpoint | | (EFC16033) | (EFC16034) | EFC16034 |
| Primary Endpoint | ARR | Relative risk | 1.061 (0.808, | 0.996 (0.754, | |
| | | (95% CI) | 1.393) | 1.315) | |
| | | p-value^{a} | 0.6691 | 0.9758 | |
| | | Adjusted | 0.05 | 0.05 | |
| | | significance level | | | |
| Secondary | 6-month CDW | Hazard Ratio | | | 0.710 (0.529, |
| Endpoints | | (95% CI) | | | 0.953) |
| | | Stratified Log-Rank | | | 0.0225 |
| | | test p-value | | | |
| | | Adjusted | | | NA |
| | | significance level | | | |
| | 3-month CDW | Hazard Ratio | | | 0.732 (0.571, |
| | | (95% CI) | | | 0.939) |
| | | Stratified Log-Rank | | | 0.0175 |
| | | test p-value | | | |
| | | Adjusted | | | NA |
| | | significance level | | | |
| | 6-month CDI | Hazard Ratio | | | 1.222 (0.935, |
| | | (95% CI) | | | 1.598) |
| | | Stratified Log-Rank | | | 0.1678 |
| | | test p-value | | | |
| | | Adjusted | | | NA |
| | | significance level | | | |
| | New Gd-enhancing | Relative risk | 1.860 (1.358, | 2.118 (1.502, | |
| | T1-hyperintense | (95% CI) | 2.548) | 2.987) | |
| | lesions | | | | |
| | | p-value^{a} | 0.0001 | <0.0001 | |
| | | Adjusted | NA | NA | |
| | | significance level | | | |
| | New and/or | Relative risk | 1.084 (0.876, | 1.165 (0.905, | |
| | enlarging T2- | (95% CI) | 1.342) | 1.502) | |
| | hyperintense lesions | | | | |
| | | p-value^{a} | 0.4575 | 0.2362 | |
| | | Adjusted | NA | NA | |
| | | significance level | | | |
| | Change from | LS Mean difference | 0.035 (−0.053, | −0.053 (−0.156, | |
| | baseline in SDMT Z- | (95% CI) | 0.124) | 0.050) | |
| | score for number of | | | | |
| | correct substitutions | | | | |
| | at EOS | | | | |
| | | p-value for the | 0.4320 | 0.3100 | |
| | | difference between | | | |
| | | groups | | | |
| | | Adjusted | NA | NA | |
| | | significance level | | | |
| | Change from | LS Mean difference | 1.873 (−0.135, | −0.612 (−2.493, | |
| | baseline in | (95% CI) | 3.880) | 1.269) | |
| | standardized score | | | | |
| | of CVLT-II total | | | | |
| | correct at EOS | | | | |
| | | p-value for the | 0.0675 | 0.5235 | |
| | | difference between | | | |
| | | groups | | | |
| | | Adjusted | NA | NA | |
| | | significance level | | | |
| | Percent change in | LS Mean difference | 0.196 (0.093, | 0.044 (−0.065, | |
| | brain volume at EOS | (95% CI) | 0.298) | 0.153) | |
| | compared to Month 6 | | | | |
| | | p-value for the | 0.0002 | 0.4266 | |
| | | difference between | | | |
| | | groups | | | |
| | | Adjusted | NA | NA | |
| | | significance level | | | |

^{a}Chi-square test for relative risk.
All values in bold font are statistically significant according to the hierarchical testing procedure

TABLE 2N
Adverse events and ALT elevations (safety population)^{a}

| | GEMINI 1 and 2 | | | |
|---|---|---|---|---|
| | Teriflunomide | | Tolebrutinib | |
| Event, n (%) | (N = 939) | | (N = 933) | |
| Any adverse event | 810 | (86.3%) | 792 | (84.9%) |
| Adverse event leading to treatment discontinuation | 41 | (4.4%) | 42 | (4.5%) |
| Any serious adverse event | 77 | (8.2) | 91 | (9.8) |
| Serious infection | 21 | (2.2) | 23 | (2.5) |
| Deaths^{b} | 2 | (0.2%) | 1 | (0.1%) |
| Incidence of increase ALT levels >3x ULN^{d} | 58 | (6.3) | 52 | (5.6) |
| 3-5x ULN | 28 | (3.0) | 20 | (2.2) |
| 5-10x ULN | 21 | (2.3) | 19 | (2.0) |
| 10-20x ULN | 8 | (0.9) | 8 | (0.9) |
| >20x ULN | 1 | (0.1) | 5 | (0.5) |
| ALT >3x ULN and total bilirubin >2x ULN | 1 | (0.1) | 4 | (0.4) |
| Most common adverse events (≥2% | | | | |
| in either treatment group)^{c} | | | | |
| Blood and lymphatic system disorders | 142 | (15.1) | 122 | (13.1) |
| Anemia | 16 | (1.7) | 37 | (4.0) |
| Iron deficiency anemia | 12 | (1.3) | 29 | (3.1) |
| Neutropenia | 92 | (9.8) | 24 | (2.6) |
| Increased tendency to bruise | 3 | (0.3) | 20 | (2.1) |
| Gastrointestinal disorders | 261 | (27.8) | 208 | (22.3) |
| Diarrhea | 85 | (9.1) | 44 | (4.7) |
| Nausea | 37 | (3.9) | 30 | (3.2) |
| Abdominal pain upper | 24 | (2.6) | 26 | (2.8) |
| Constipation | 12 | (1.3) | 19 | (2.0) |
| Dyspepsia | 19 | (2.0) | 19 | (2.0) |
| Vomiting | 24 | (2.6) | 15 | (1.6) |
| Abdominal pain | 25 | (2.7) | 14 | (1.5) |
| Toothache | 19 | (2.0) | 13 | (1.4) |
| General disorders and administration site conditions | 140 | (14.9) | 143 | (15.3) |
| Fatigue | 36 | (3.8) | 46 | (4.9) |
| Pyrexia | 34 | (3.6) | 34 | (3.6) |
| Influenza like illness | 21 | (2.2) | 20 | (2.1) |
| Infections and infestations | 546 | (58.1) | 554 | (59.4) |
| COVID-19 | 252 | (26.8) | 225 | (24.1) |
| Nasopharyngitis | 105 | (11.2) | 119 | (12.8) |
| Upper respiratory tract infection | 82 | (8.7) | 77 | (8.3) |
| Urinary tract infection | 57 | (6.1) | 59 | (6.3) |
| Viral upper respiratory tract infection | 59 | (6.3) | 50 | (5.4) |
| Influenza | 52 | (5.5) | 46 | (4.9) |
| Tonsillitis | 19 | (2.0) | 31 | (3.3) |
| Sinusitis | 22 | (2.3) | 30 | (3.2) |
| Cystitis | 20 | (2.1) | 28 | (3.0) |
| Pharyngitis | 19 | (2.0) | 26 | (2.8) |
| Oral herpes | 18 | (1.9) | 24 | (2.6) |
| Bronchitis | 14 | (1.5) | 22 | (2.4) |
| Gastroenteritis | 26 | (2.8) | 17 | (1.8) |
| Rhinitis | 21 | (2.2) | 12 | (1.3) |
| Injury, poisoning and procedural complications | 146 | (15.5) | 176 | (18.9) |
| Accidental overdose | 27 | (2.9) | 34 | (3.6) |
| Fall | 25 | (2.7) | 18 | (1.9) |
| Investigations | 189 | (20.1) | 172 | (18.4) |
| Alanine aminotransferase increased^{e} | 64 | (6.8) | 44 | (4.7) |
| Blood creatine phosphokinase increased | 33 | (3.5) | 22 | (2.4) |
| Neutrophil count decreased | 20 | (2.1) | 7 | (0.8) |
| Neoplasms benign, malignant and unspecified | 25 | (2.7) | 54 | (5.8) |
| (incl cysts and polyps) | | | | |
| Uterine leiomyoma | 3 | (0.3) | 19 | (2.0) |
| Nervous system disorders | 262 | (27.9) | 247 | (26.5) |
| Headache | 98 | (10.4) | 117 | (12.5) |
| Paranesthesia | 37 | (3.9) | 31 | (3.3) |
| Dizziness | 22 | (2.3) | 26 | (2.8) |
| Hypoesthesia | 22 | (2.3) | 21 | (2.3) |
| Musculoskeletal and connective tissue disorders | 210 | (22.4) | 199 | (21.3) |
| Back pain | 55 | (5.9) | 58 | (6.2) |
| Arthralgia | 44 | (4.7) | 41 | (4.4) |
| Pain in extremity | 44 | (4.7) | 29 | (3.1) |
| Neck pain | 13 | (1.4) | 21 | (2.3) |
| Muscular weakness | 11 | (1.2) | 19 | (2.0) |
| Psychiatric disorders | 154 | (16.4) | 130 | (13.9) |
| Anxiety | 35 | (3.7) | 36 | (3.9) |
| Insomnia | 34 | (3.6) | 34 | (3.6) |
| Depression | 41 | (4.4) | 26 | (2.8) |
| Reproductive system and breast disorders | 64 | (6.8) | 87 | (9.3) |
| Heavy menstrual bleeding | 9 | (1.0) | 24 | (2.6) |
| Respiratory, thoracic and mediastinal disorders | 106 | (11.3) | 108 | (11.6) |
| Cough | 29 | (3.1) | 31 | (3.3) |
| Oropharyngeal pain | 8 | (0.9) | 22 | (2.4) |
| Vascular disorders | 77 | (8.2) | 62 | (6.6) |
| Hypertension | 62 | (6.6) | 29 | (3.1) |
| Skin and subcutaneous tissue disorders | 261 | (27.8) | 248 | (26.6) |
| Alopecia | 146 | (15.5) | 73 | (7.8) |
| Petechiae | 3 | (0.3) | 42 | (4.5) |
| Dermatitis allergic | 20 | (2.1) | 24 | (2.6) |
| Pruritus | 23 | (2.4) | 13 | (1.4) |

^{a}The safety population included all participants randomized and exposed to tolebrutinib or teriflunomide, regardless of the duration of exposure. Data were collected from the first dose to 10 days after the last dose, the date of death, or last contact date, whichever occurred first.
^{b}The death that occurred in the tolebrutinib arm was due to homicidal gunshot wound (assessed as unrelated to the investigational drug). The deaths in the teriflunomide arm were due to suicide by firearm and brain edema from traumatic subarachnoid hemorrhage (both assessed as unrelated to the investigational drug).
^{c}Adverse events were grouped by system organ class and preferred term according to the Medical Dictionary for Regulatory Activities 27.0.
^{d}Tolebrutinib: N = 929; teriflunomide: N = 926. ALT, alanine aminotransferase; ULN, upper limit of normal
^{e}Increased alanine aminotransferase was collected as an adverse event by investigators (reported in this row) and was also determined from clinical laboratory data (rows 6-10). Small differences in these values are due to the fact that investigator reporting may not conform with the definition of >3x the upper limit of normal.

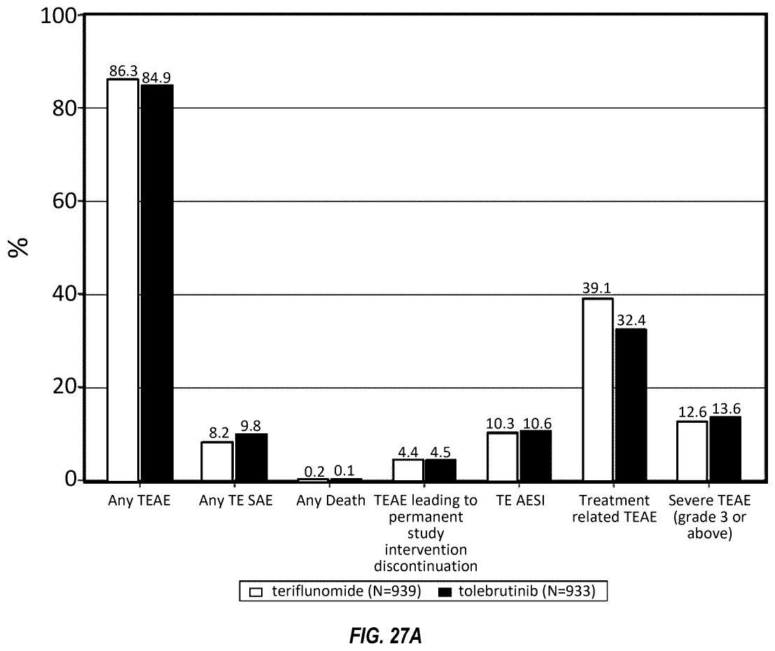

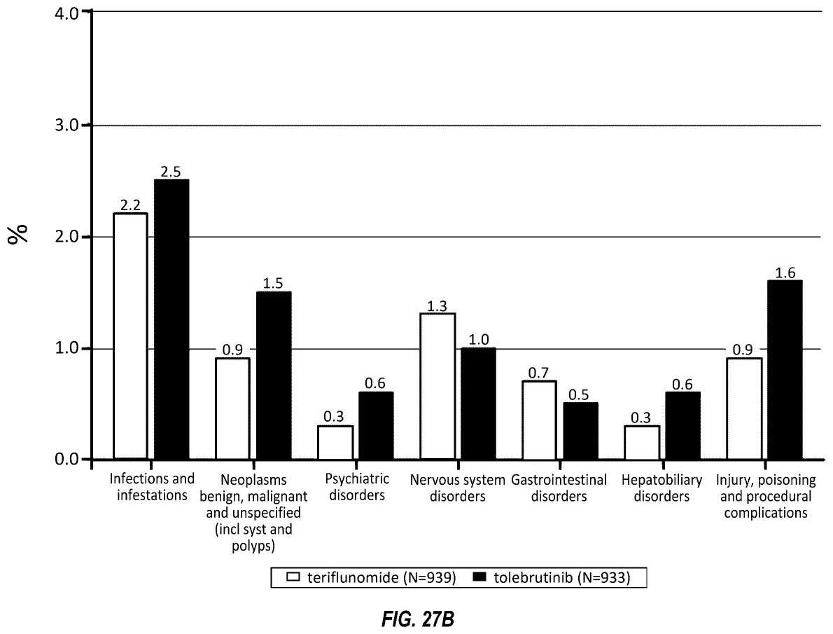

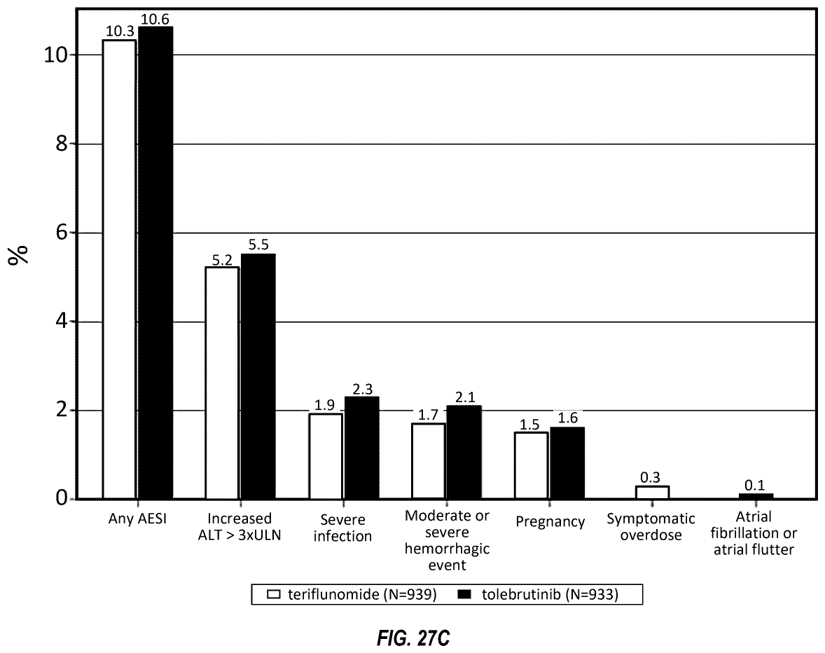

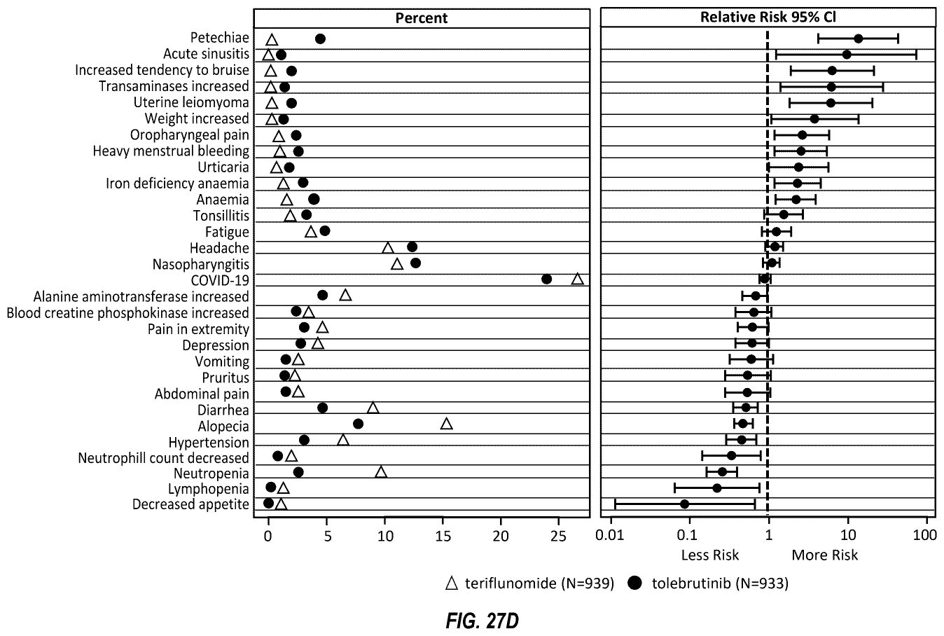

Further, the study results demonstrated the safety profile of tolebrutinib was consistent with that reported previously (FIGS. 27A-27D). In preliminary analysis of the Gemini 1 and 2 pooled safety data, adverse events observed between the tolebrutinib and teriflunomide arms were generally balanced (Table 2N). The most common adverse events (≥10%) for participants receiving tolebrutinib were COVID-19 infection (n=225 [24.1%]), nasopharyngitis (n=119 [12.8%]) and headache (n=117 [12.5%]). The most common adverse events (≥10%) for participants receiving teriflunomide were COVID-19 infection (n=252 [26.8%]), nasopharyngitis (n=105 [11.2%]) and headache (n=98 [10.4%]).

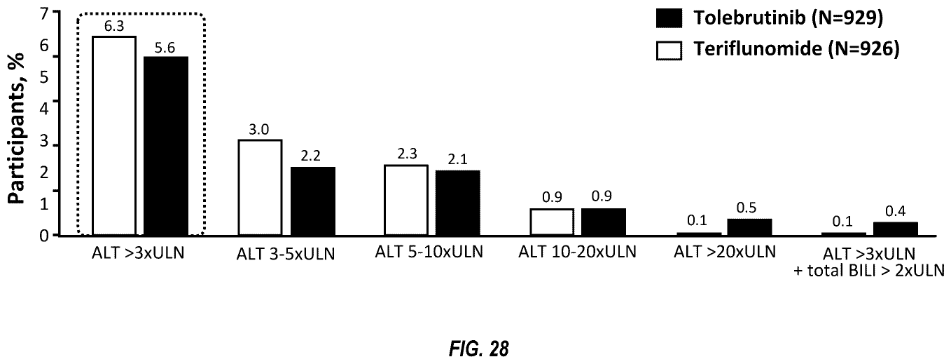

Preliminary analysis of the liver safety of tolebrutinib was consistent with that reported previously (FIG. 28). Liver enzyme elevations (>3×ULN) were observed in 5.6% of participants receiving tolebrutinib, compared with 6.3% of participants receiving teriflunomide, a side effect reported with other BTK inhibitors in MS and resolved without further medical information. A small (0.5%) proportion of participants in the tolebrutinib group experienced peak ALT increases of >20×ULN, all occurring within the first 90 days of treatment. Deaths were balanced across the teriflunomide and tolebrutinib arms, at 0.2% and 0.1% respectively, and were assessed as unrelated to treatment by investigator.

### Participants

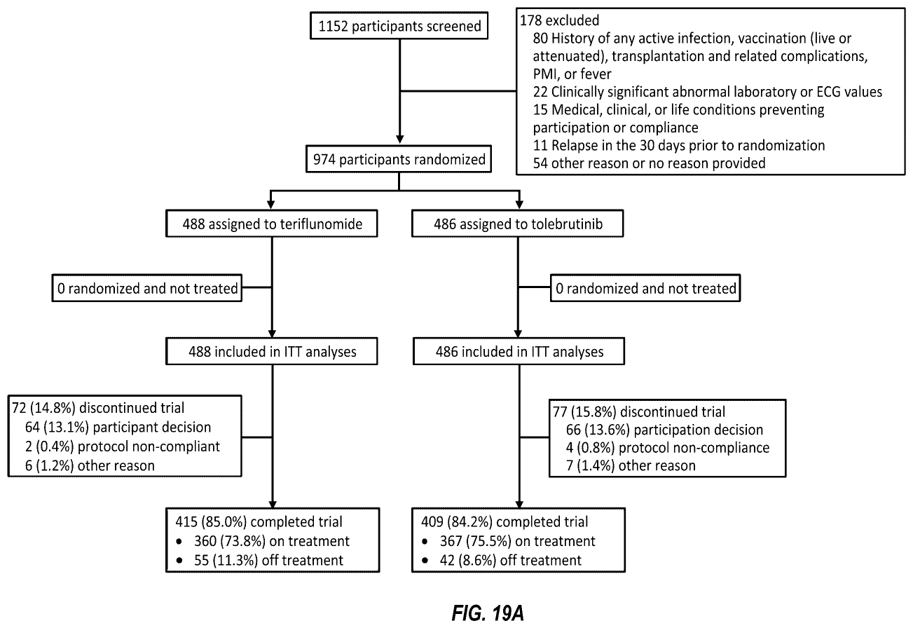

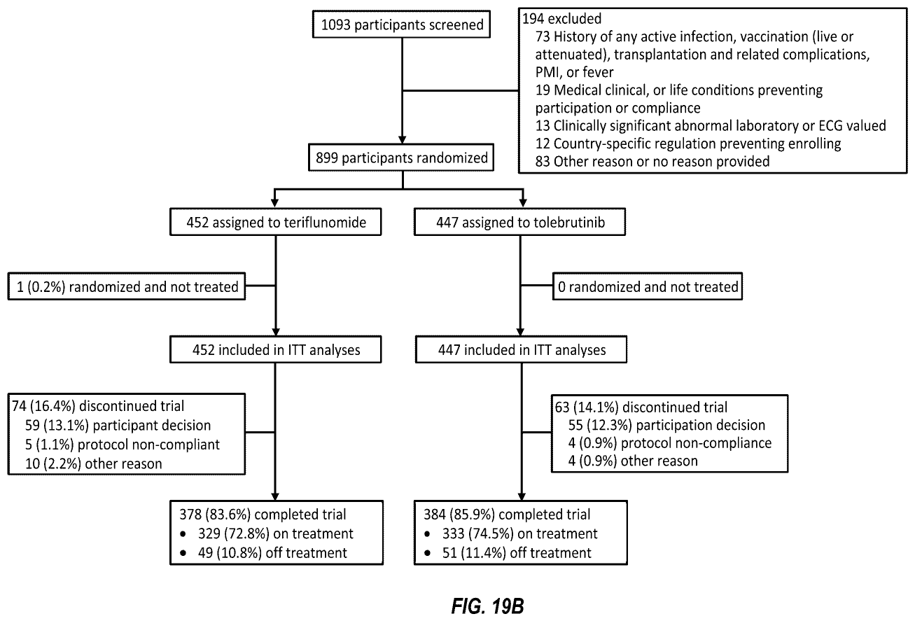

A total of 1873 participants were randomized between Jun. 25, 2020 through Aug. 8, 2022: 974 in GEMINI 1 (486 assigned to tolebrutinib and 488 to teriflunomide) and 899 in GEMINI 2 (447 assigned to tolebrutinib and 452 to teriflunomide). In GEMINI 1, 84.2% of participants randomized to tolebrutinib and 85.0% randomized to teriflunomide completed the trial (FIG. 19A). The corresponding percentages for GEMINI 2 were 85.9% and 83.6% (FIG. 19B). Median follow-up was 139 weeks. Demographic and disease characteristics were similar for the treatment groups in both trials (see Table 2K).

### Efficacy

Primary Endpoint. The adjusted ARR during the trial period was 0.13 and 0.12 for tolebrutinib and teriflunomide, respectively, in GEMINI 1 (rate ratio, 1.06; 95% confidence interval [CI], 0.81 to 1.39; P=0.67), and 0.11 for both arms in GEMINI 2 (rate ratio, 1.00; 95% CI, 0.75 to 1.32; P=0.98; see Table 2O, FIG. 20). Due to this non-significant result in the hierarchical testing, the results for all hierarchically lower end points are considered nominal.

Disability-Related Endpoints. In the prespecified pooled analyses of both trials, the percentage of participants with 6-month confirmed disability worsening during the trial was 8.3% for tolebrutinib and 11.3% for teriflunomide (hazard ratio, 0.71; 95% CI, 0.53 to 0.95; nominal P=0.02; see Table 2O, FIG. 21). The percentage of participants with 3-month confirmed disability worsening during the trial was 11.7% for tolebrutinib and 15.3% for teriflunomide (hazard ratio, 0.73; 95% CI, 0.57 to 0.94; nominal P=0.02; see Table 2O, FIG. 22). These and all subsequent results were considered nominal because of the hierarchical testing approach. The percentage of participants with 6-month confirmed disability improvement during the trial was 12.6% for tolebrutinib and 10.4% for teriflunomide (hazard ratio, 1.22; 95% CI, 0.94 to 1.60; nominal P=0.17; see Table 2O, FIG. 22).

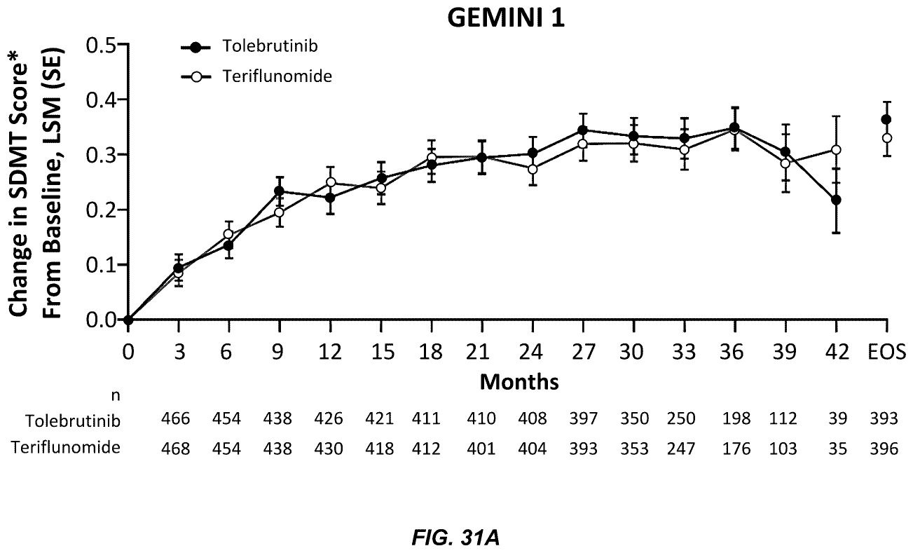

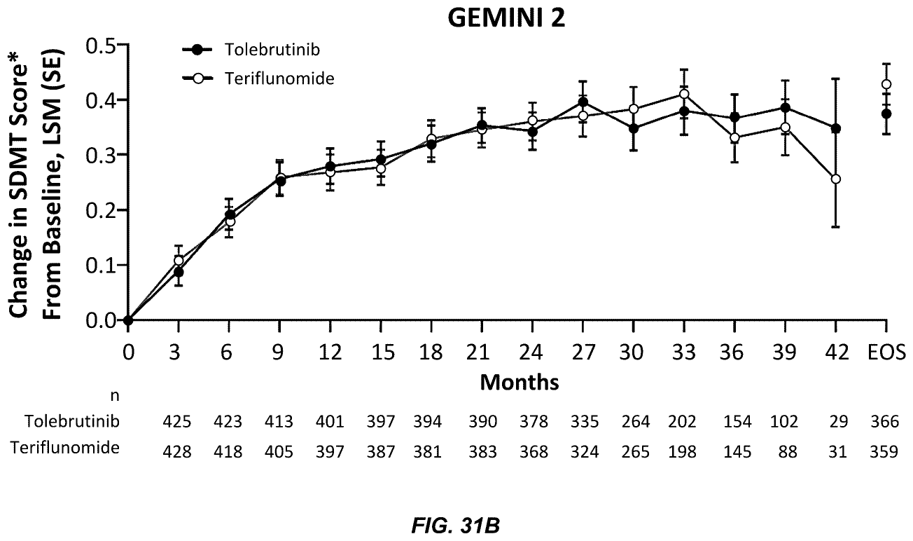

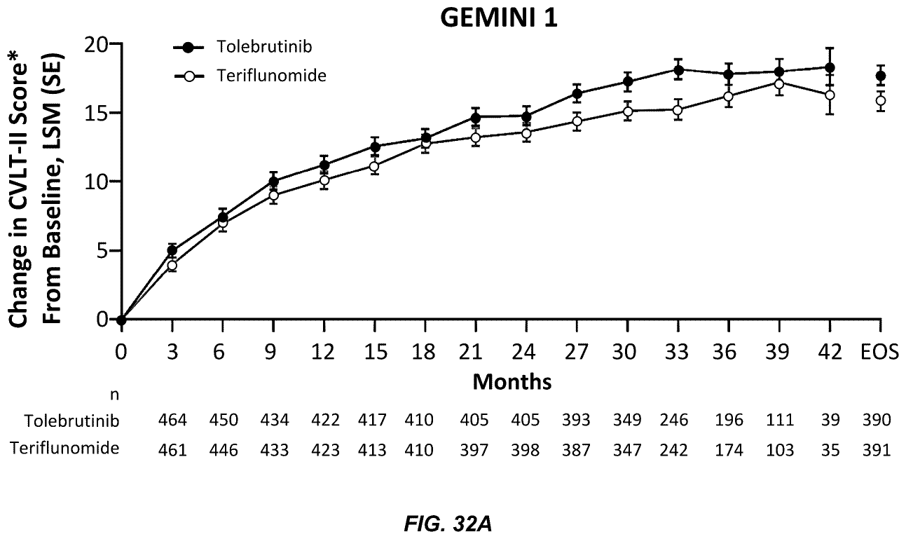

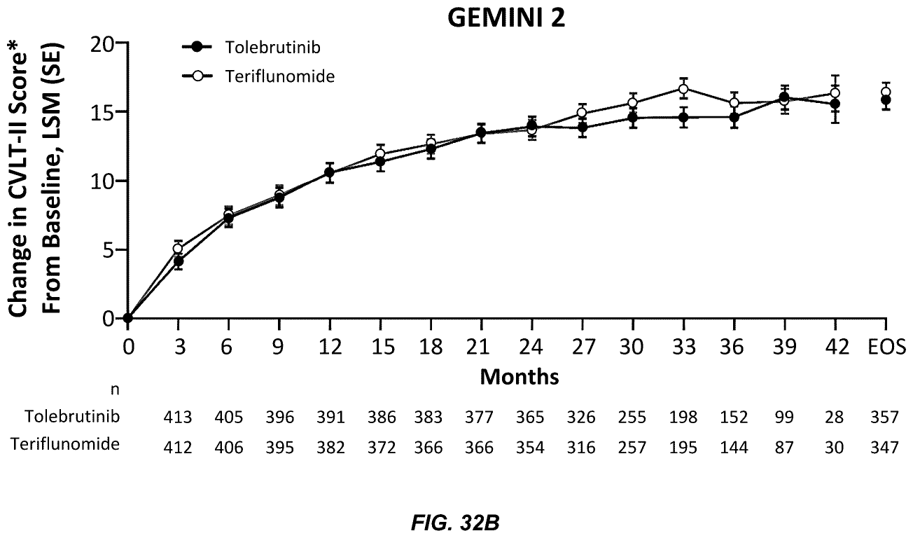

Least squares mean changes in SDMT number of correct substitutions z-score from baseline to the EOS were 0.364 and 0.329 for tolebrutinib and teriflunomide in GEMINI 1 (difference of 0.035; 95% CI, −0.053 to 0.124) (FIG. 31A), and 0.374 and 0.428 in GEMINI 2 (difference of −0.053; 95% CI, −0.156 to 0.050) (FIG. 31B); see Table 2O. Least squares mean changes in CVLT-II total correct standardized score from baseline to the EOS were 17.700 and 15.827 for tolebrutinib and teriflunomide in GEMINI 1 (difference of 1.873; 95% CI, −0.135 to 3.880) (FIG. 32A), and 15.819 and 16.431 in GEMINI 2 (difference of −0.612; 95% CI, −2.493 to 1.269) (FIG. 32B); see Table 2O.

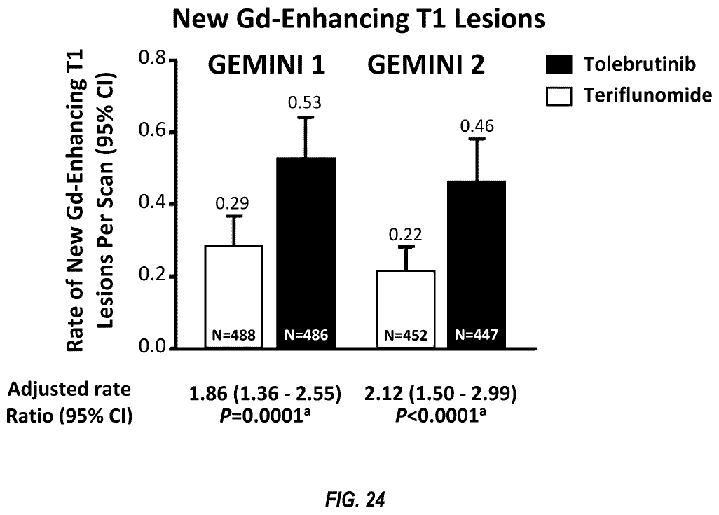

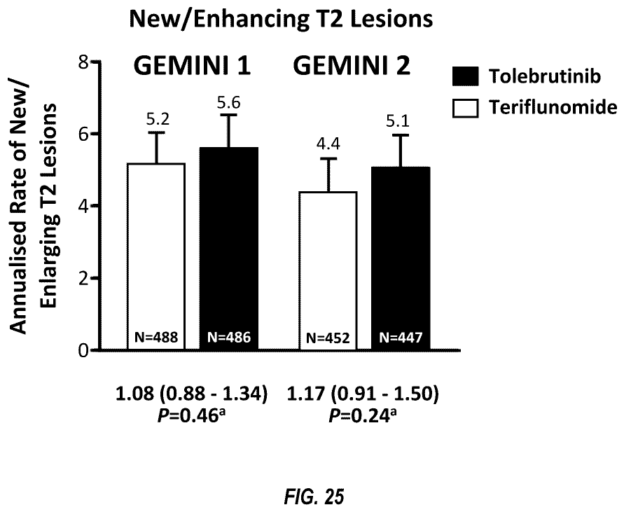

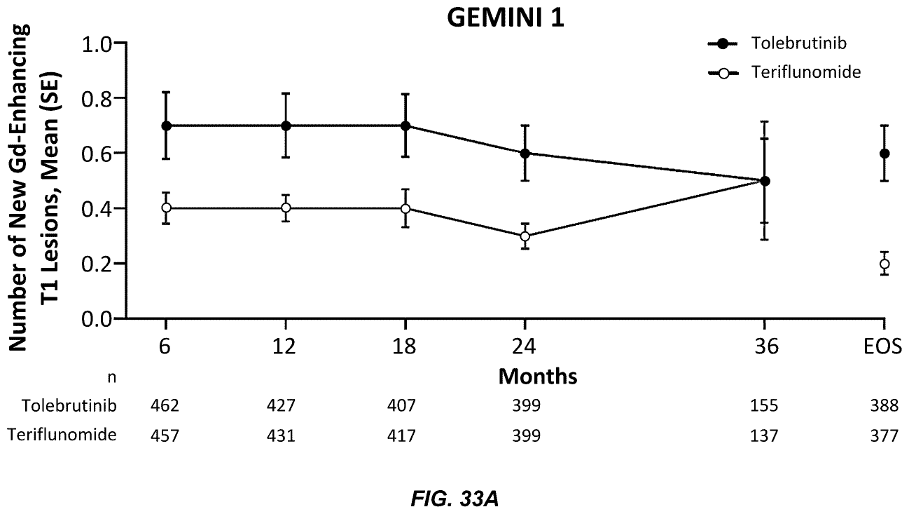

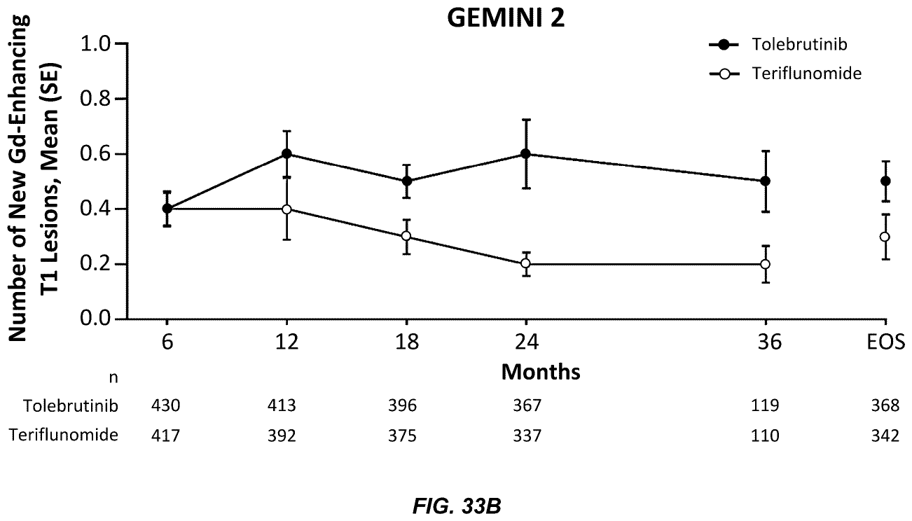

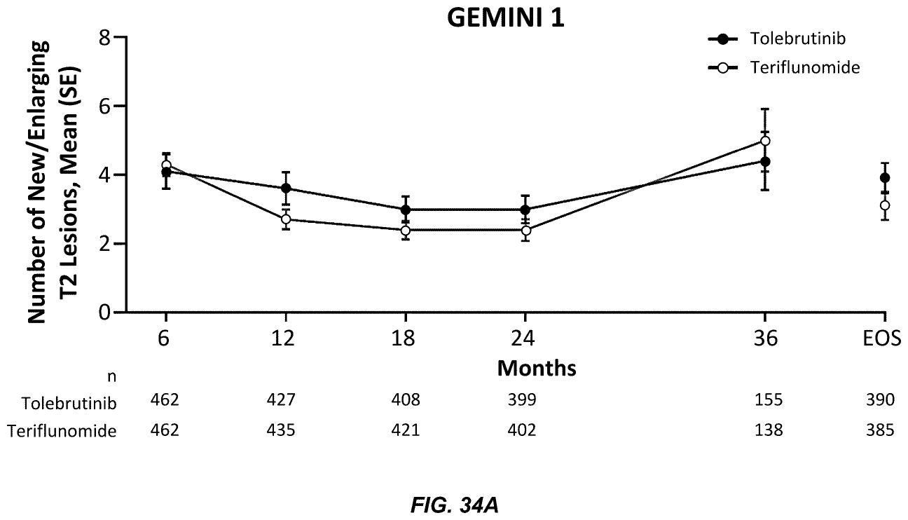

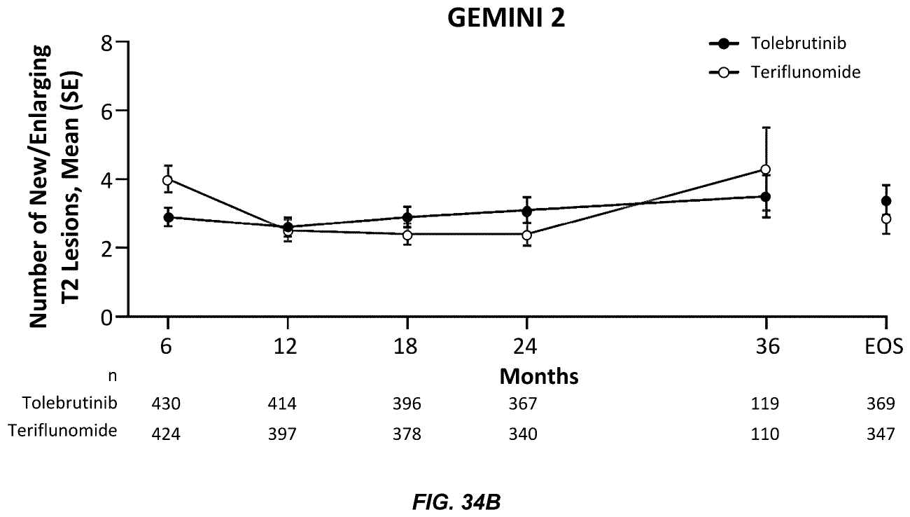

MRI-Related Endpoints. The adjusted mean number of new gadolinium-enhancing T1-weighted lesions per scan was 0.53 and 0.29 for tolebrutinib and teriflunomide, respectively, in GEMINI 1 (adjusted rate ratio 1.86; 95% CI, 1.36 to 2.55) (FIG. 33A), and 0.46 and 0.22 in GEMINI 2 (adjusted rate ratio, 2.12, 95% CI, 1.50 to 2.99) (FIG. 33B); see Table 2O, FIG. 24. The adjusted mean number of new/enlarging T2-weighted lesions per year was 5.61 and 5.18 for tolebrutinib and teriflunomide, respectively, in GEMINI 1 (adjusted rate ratio 1.08; 95% CI, 0.88 to 1.34) (FIG. 34A), and 5.09 and 4.37 in GEMINI 2 (adjusted rate ratio, 1.08; 85% CI, 0.88 to 1.34) (FIG. 34B); see Table 2O, FIG. 25.

The least squares mean percentage change in brain volume at EOS compared to month 6 was −0.69% and −0.88% for tolebrutinib and teriflunomide, respectively, in GEMINI 1 (difference of 0.20%; 9500 CI, 0.0900 to 0.30%) (FIG. 26A), and −0.70% and −0.74% in GEMINI 2 (difference of 0.040%; 95% CI, −0.07% to 0.15%) (FIG. 26B); see Table 2O.

TABLE 2O
Primary and secondary endpoints (intention-to-treat population)

| | GEMINI 1 | | GEMINI 2 | | Pooled Analysis | |
|---|---|---|---|---|---|---|
| | Tolebrutinib | Teriflunomide | Tolebrutinib | Teriflunomide | Tolebrutinib | Teriflunomide |
| End point | (N = 486) | (N = 488) | (N = 447) | (N = 452) | (N = 933) | (N = 940) |

Primary end point

| | | | | | | |
|---|---|---|---|---|---|---|
| Adjusted ARR | 0.13 (0.11 to | 0.12 (0.10 to | 0.11 (0.09 to | 0.11 (0.09 to | 0.12 (0.11 to | 0.12 (0.10 to |
| (95% CI) | 0.16) | 0.15) | 0.13) | 0.13) | 0.14) | 0.14) |

| | | | |
|---|---|---|---|
| Rate ratio (95% | 1.06 (0.81 to | 1.00 (0.75 to | 1.03 (0.85 to |
| CI) | 1.39) | 1.32) | 1.25) |
| P value | 0.67 | 0.98 | 0.80 |

Secondary end points involving prespecified pooled analysis (hierarchically ordered)

| 6-month | | |
|---|---|---|
| confirmed | | |
| disability | | |
| worsening^{a} | | |
| No. of events | 78 | 106 |
| (%)^{b} | (8.3) | (11.3) |
| Kaplan-Meier | 6.7 (5.2 to | 9.3 (7.5 to |
| estimate at 24 | 8.5) | 11.4) |
| months, % | | |
| (95% CI) | | |
| Hazard ratio | 0.71 (0.53 to 0.95), | |
| (95% CI), | nominal P = 0.02 | |
| P-value^{c} | | |
| 3-month | | |
| confirmed | | |
| disability | | |
| worsening^{d} | | |
| No. of events | 109 | 144 |
| (%) | (11.7) | (15.3) |
| Kaplan-Meier | 8.8 (7.1 to | 11.8 (9.8 to |
| estimate at 24 | 10.9) | 14.1) |
| months, % | | |
| (95% CI) | | |
| Hazard ratio | 0.73 (0.57 to 0.94), | |
| (95% CI), | nominal P = 0.02 | |
| P-value^{c} | | |
| 6-month | | |
| confirmed | | |
| disability | | |
| improvement^{e} | | |
| No. of events | 118 | 98 |
| (%) | (12.6) | (10.4) |
| Kaplan-Meier | 11.3 (9.4 to | 8.8 (7.1 to |
| estimate at 24 | 13.6) | 10.9) |
| months, % | | |
| (95% CI) | | |
| Hazard ratio | 1.22 (0.94 to 1.60), | |
| (95% CI), | nominal P = 0.17 | |
| P-value^{c} | | |

Secondary end points analyzed in each trial (hierarchically ordered)

| Adjusted | | | | |
|---|---|---|---|---|
| number of new | | | | |
| gadolinium- | | | | |
| enhancing T1- | | | | |
| weighted | | | | |
| lesions | | | | |
| per scan | | | | |
| Number of | 473 | 469 | 436 | 432 |
| participants | | | | |
| with non- | | | | |
| missing data | | | | |
| Number of | 218 | 189 | 205 | 151 |
| participants | | | | |
| with lesions | | | | |
| Mean estimate | 0.53 (0.44 to | 0.29 (0.22 to | 0.46 (0.37 to | 0.22 (0.17 to |
| (95% CI) | 0.64) | 0.37) | 0.58) | 0.28) |
| Rate ratio (95% | 1.86 (1.36 to | | 2.12 (1.50 to | |
| CI) | 2.55) | | 2.99) | |
| Adjusted | | | | |
| number of | | | | |
| new/enlarging | | | | |
| T2-weighted | | | | |
| lesions | | | | |
| per year | | | | |
| Number of | 473 | 474 | 436 | 435 |
| participants | | | | |
| with non- | | | | |
| missing data | | | | |
| Number of | 363 | 363 | 331 | 329 |
| participants | | | | |
| with lesions | | | | |
| Mean estimate | 5.61 (4.83 to | 5.18 (4.45 to | 5.09 (4.34 to | 4.37 (3.59 to |
| (95% CI) | 6.52) | 6.02) | 5.98) | 5.32) |
| Rate ratio (95% | 1.08 (0.88 to | | 1.17 (0.91 to | |
| CI) | 1.34) | | 1.50) | |
| Change in | | | | |
| SDMT number | | | | |
| of correct | | | | |
| substitutions | | | | |
| z-score from | | | | |
| baseline | | | | |
| to EOS | | | | |
| Number of | 393 | 396 | 366 | 359 |
| participants | | | | |
| LSM (SE) | 0.364 | 0.329 | 0.374 | 0.428 |
| | (0.032) | (0.032) | (0.037) | (0.037) |
| LSM | 0.035 (−0.053 to | | −0.053 (−0.156 to | |
| difference vs. | 0.124) | | 0.050) | |
| teriflunomide | | | | |
| (95% CI) | | | | |
| Change in | | | | |
| CVLT-II | | | | |
| total correct | | | | |
| standardized | | | | |
| T-score from | | | | |
| baseline | | | | |
| to EOS | | | | |
| Number of | 390 | 391 | 357 | 347 |
| participants | | | | |
| evaluated | | | | |
| LSM (SE) | 17.700 | 15.827 | 15.819 | 16.431 |
| | (0.724) | (0.724) | (0.674) | (0.683) |
| LSM | 1.873 (−0.135 to | | −0.612 (−2.493 to | |
| difference vs. | 3.880) | | 1.269) | |
| teriflunomide | | | | |
| (95% CI) | | | | |
| Percentage | | | | |
| change in | | | | |
| brain volume | | | | |
| from month | | | | |
| 6 to EOS | | | | |
| Number of | 351 | 346 | 317 | 307 |
| participants | | | | |
| evaluated | | | | |
| LSM (SE) | −0.69 | −0.88 | −0.70 | −0.74 |
| | (0.04) | (0.04) | (0.04) | (0.04) |
| LSM | 0.20 (0.09 to | | 0.04 (−0.07 to | |
| difference vs. | 0.30) | | 0.15) | |
| teriflunomide | | | | |
| (95% CI) | | | | |

Other secondary end points analyzed in each trial

| Change from | | | | |
|---|---|---|---|---|
| baseline in | | | | |
| MSQoL-54 | | | | |
| Questionnaire | | | | |
| Score at EOS | | | | |
| Physical health | | | | |
| composite | | | | |
| score | | | | |
| Number of | 394 | 393 | 367 | 353 |
| participants | | | | |
| evaluated | | | | |
| LSM (SE) | −0.46 | −2.47 | −1.20 | −1.04 |
| | (0.70) | (0.70) | (0.73) | (0.74) |
| LSM | 2.01 (0.06 to | | −0.16 (−2.20 to | |
| difference vs | 3.96) | | 1.88) | |
| teriflunomide | | | | |
| (95% CI) | | | | |
| Mental health | | | | |
| composite | | | | |
| score | | | | |
| Number of | 399 | 400 | 370 | 357 |
| participants | | | | |
| evaluated | | | | |
| LSM (SE) | −0.73 | −2.07 | −1.39 | −1.66 |
| | (0.84) | (0.83) | (0.86) | (0.87) |
| LSM | 1.34 (−0.98 to | | 0.27 (−2.13 to | |
| difference vs | 3.66) | | 2.67) | |
| teriflunomide | | | | |
| (95% CI) | | | | |

^{a}Confirmed, sustained increase in EDSS score of ≥1.5 points when baseline score is 0, ≥1.0 points when baseline score is 0.5 to ≤5.5 points, or ≥0.5 points when baseline score is >5.5 points over 6 months or longer and is not attributable to another etiology.
^{b}The number and percentage of 6-month confirmed disability worsening events is based on multiple imputation.
^{c}Derived from log-rank test with stratification of EDSS strata (<4, >=4), geographic region (US, non-US), and trial (GEMINI 1, GEMINI 2).
^{d}Confirmed, sustained increase in EDSS score of ≥1.5 points when baseline score is 0, ≥1.0 points when baseline score is 0.5 to ≤5.5 points, or ≥0.5 points when baseline score is >5.5 points over 3 months or longer and is not attributable to another etiology.
^{e}Confirmed, sustained decrease in EDSS score of ≥1.0 points lasting 6 months or longer.
ARR, annualized (adjudicated) relapse rate; CVLT-II, California Verbal Learning Test - Second Edition; CI, confidence interval; EDSS, Expanded Disability Status Scale; EOS, end of study; LSM, least squares mean; MSQoL-54: Multiple Sclerosis Quality of Life-54; SDMT, Symbol Digit Modalities Test; SE, standard error.

### Statistical Considerations

The primary end point for both trials was annualized (adjudicated) relapse rate (ARR), defined as number of confirmed (adjudicated) relapses per participant-year, according to prespecified criteria. Confirmed relapses were defined as new neurological symptoms or worsening of previous neurological symptoms accompanied by a clinically relevant change in EDSS score and confirmed by an independent adjudication committee. EDSS was scored by a certified rater who was blinded to treatment assignment and had no other role in the participant's care.

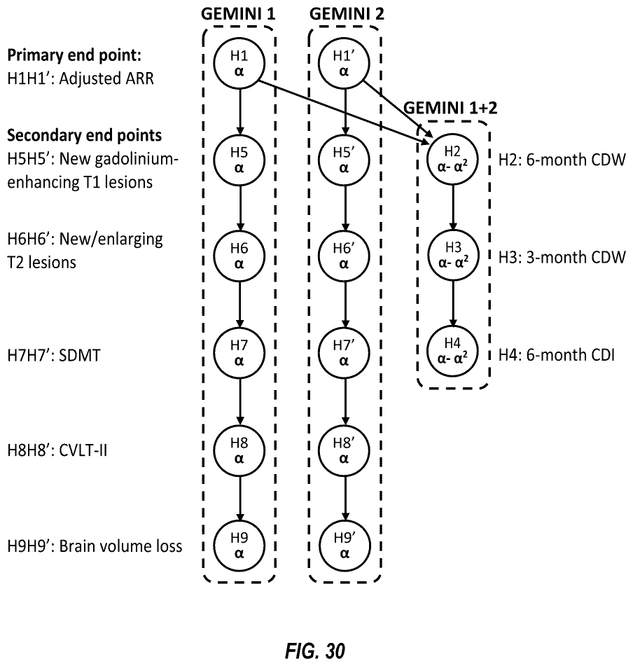

There were eight hierarchically ordered secondary end points: 6-month confirmed disability worsening (pooled across trials); 3-month confirmed disability worsening (pooled across trials); 6-month confirmed disability improvement (pooled across trials); cumulative number of new gadolinium-enhancing T1-weighted brain lesions from baseline to the end of study (EOS) visit; cumulative number of new/enlarging T2 brain lesions from baseline to the EOS visit; change from baseline to EOS in Symbol Digit Modalities Test (SDMT) score; change from baseline to EOS in California Verbal Learning Test-Second Edition (CVLT-II) score; and percentage change in brain volume from month 6 to EOS (see FIG. 30). Confirmed disability worsening events were based on EDSS score and were permitted to have an onset during or outside of relapses, but were required to be confirmed outside of relapses.

Each trial was planned to randomize approximately 900 participants (1:1 allocation ratio; approximately 450 per treatment group), which was estimated to provide more than 90% power (with a two-sided alpha of 0.05) to detect a 45% relative reduction of ARR with tolebrutinib compared to teriflunomide. Sample size calculations assumed an ARR of 0.29 for teriflunomide based on previous data (Confavreux, et al., Lancet Neurol 2014; 13:247-56; O'Connor, et al., N Engl J Med 2011; 365:1293-303), a variance inflation factor of 1.5 for each treatment group, and that 20% of randomized participants would discontinue the trial by month 24. A total of 162 events of 6-month confirmed disability worsening pooled across both trials was projected to provide approximately 90% power (with a two-sided alpha of 0.05) to detect a 40% risk reduction in 6-month confirmed disability worsening with tolebrutinib compared to teriflunomide, assuming a 24-month event rate of 12% in the teriflunomide group.

Efficacy analyses were performed in accordance with the intention-to-treat principle, with the intention-to-treat population comprising all randomized participants. Analysis of the primary end point of ARR included all adjudicated relapses from randomization to the EOS or last contact date for participants who withdrew from the trial prior to the common EOS. A negative binomial regression model with robust variance estimation was used, with treatment group, gadolinium-enhancing T1-weighted lesions at baseline (presence or absence), EDSS strata (<4 or ≥4), and geographic region (US or non-US) as covariates, and log transformed observation duration as the offset variable.

Overall type 1 error rate was controlled at two-sided α=0.05 for the primary and secondary end points in individual trials. If statistical significance for the primary end point was met at α=0.05 in both trials, then the secondary disability-related end points that were based on pooled data were tested at two-sided α=0.05-0.05²=0.0475 using a sequential, hierarchical testing procedures (see FIG. 30). In the hierarchical testing procedure, secondary end points were tested only if all preceding end points were statistically significant.

In accordance with the intention-to-treat principle, the analysis of annualized (adjudicated) relapse rate (ARR) compared participants randomized to tolebrutinib versus teriflunomide, regardless of what treatment participants received. Participants who permanently discontinued the trial intervention were encouraged to return to the clinic for all remaining visits, and all events during the planned treatment period for these participants were included up to the end of study. If a participant withdrew from the trial prior to the common end-of-study date, all observed events up to the last contact date were included in the analysis, and the observation duration was from randomization to the last contact date. No imputation was performed for the unobserved events that may have occurred after trial discontinuation. ARR was analyzed using a negative binomial regression model with robust variance estimation. The model included treatment group, gadolinium-enhancing T1-weighted lesions at baseline (presence or absence), EDSS strata (<4 or ≥4), and geographic region (US or non-US) as covariates, and log transformed observation duration as the offset variable. No interim analyses were conducted.

Analysis of the key secondary end point, time to onset of 6-month confirmed disability worsening, was performed on the pooled intention-to-treat population from GEMINI 1 and 2. Participants were censored at the date of last EDSS assessment if they completed the study without an initial onset of disability worsening or had an initial onset of disability worsening but completed the trial at the common study end without 3-month confirmation. For participants who completed the trial, met the 3-month confirmed disability worsening criteria, and continued to meet the criteria for EDSS disability worsening through the final trial assessment, but did not reach 6-month confirmation, the 6-month confirmed disability worsening event status (i.e., confirmed or not confirmed) of the participant was imputed using the following logistic regression model:

$${Y}_{i} ∼ Bernoulli ( {p}_{ij} ) , i = 1 , … , {n}_{j} ; j = 1 , 2$$

$$logit ( {p}_{ij} ) = In ( \frac{{p}_{ij}}{1 - {p}_{ij}} ) = {β}_{0 j} + {β}_{1 j} {x}_{1 ij} + {β}_{2 j} {x}_{2 ij} + {β}_{3 j} {x}_{3 ij} + {β}_{4 j} {x}_{4 ij}$$

In this model, Yᵢⱼ is the indicator for event status of 6-month confirmed disability worsening for participant i from group j (1=6-month confirmed disability worsening and 0=no 6-month confirmed disability worsening); pᵢⱼ is the probability that participant i from group j has a 6-month confirmed disability worsening event; x₁ᵢⱼ is the EDSS strata (<4 or ≥4), x₂ᵢⱼ is the geographic region (US or non-US); x₃ᵢⱼ is the gadolinium-enhancing T1-weighted lesion status at baseline (absence or presence); and βₖⱼ are the model parameters (k=0,1,2,3; j=1,2). The logistic model parameters βₖⱼ were estimated from participants who did not have missing value due to common end of study. Separate logistic models were estimated for each treatment arm. The estimated parameters were then used to calculate the predicted probability of 6-month confirmed disability worsening for participants who met the 3-month confirmed disability worsening criteria and continued to meet criteria for EDSS disability worsening through the final trial assessment prior to the common end of study. The missing value for those participants with an initial onset of disability worsening and missingness due to the common end of study was imputed via a randomly generated value from a Bernoulli distribution with pᵢⱼ. A total of 1000 complete data sets were generated with imputed missing data, and the results were combined using Rubin's formula.

Deaths due to multiple sclerosis were considered confirmed disability worsening regardless of baseline EDSS score or the change in EDSS score. The time to onset of confirmed disability worsening was calculated as date of EDSS assessment at a tentative onset of the event−date of randomization+1, or for instances without a tentative onset date, as date of death−date of randomization+1. Death for reasons other than multiple sclerosis were not considered to be disability worsening.

The time to onset of 6-month confirmed disability worsening, 3-month confirmed disability worsening, and 6-month confirmed disability improvement for tolebrutinib versus teriflunomide were compared via log-rank tests stratified by EDSS strata (<4 or ≥4), geographic region (US or non-US) and trial (GEMINI 1 or 2). The hazard ratios were estimated by Cox proportional hazards models with robust variance estimation. The covariates were treatment group, gadolinium-enhancing T1-weighted lesions at baseline (presence or absence), EDSS strata (<4 or ≥4), geographic region (US or non-US) and trial (GEMINI 1 or 2).

Categorical secondary end points with count data (average number of new gadolinium-enhancing T1-weighted lesions per scan and annualized rate of new/enlarging T2-weighted lesions) were analyzed using negative binomial regression models without imputation. The model for average number of new gadolinium-enhancing T1-weighted lesions per scan included treatment group, EDSS strata (<4 or ≥4), geographic region (US or non-US), and gadolinium-enhancing T1-weighted lesions at baseline (presence or absence) as covariates, the log transformed number of MRI scans as the offset variable, and the total number of new gadolinium-enhancing T1-weighted lesions across all scheduled MRI scans as the response variable. The model for annualized rate of new/enlarging T2-weighted lesions included treatment group, EDSS strata (<4 or ≥4), geographic region (US or non-US), and number of T2-weighted lesions at baseline as covariates, log transformed trial duration as the offset variable, and total number of new/enlarging T2-weighted lesions across all scheduled MRI scans as the response variable.

Continuous secondary end points (percentage change in brain volume and changes in SDMT and CVLT-II scores) were analyzed using mixed-effect models with repeated measures (MMRM) for estimating the treatment effect with appropriate transformation if necessary (e.g., log-transformation), with percentage change or change values at each visit as the response variable, and treatment, EDSS strata (<4 or ≥4), geographic region (US or non-US), visit, treatment-by-visit interaction, baseline (SDMT and CLVT-II scores) or Month 6 (brain volume) value for the end point, and baseline (SDMT and CLVT-II scores) or Month 6 (brain volume) value-by-visit interaction as covariates. Estimates derived from the MMRM approach were weighted by the overall proportion of participants in each stratum or categorical factor.

Definition of multiple sclerosis relapse. Multiple sclerosis relapses were defined as monophasic, acute/subacute onset of new neurological symptoms or worsening of previous neurological symptoms with an objective change on neurological examination. Symptoms had to be attributable to multiple sclerosis, last for ≥24 hours (with or without recovery), be present at normal body temperature, and be preceded by ≥30 days of clinical stability (including no previous multiple sclerosis relapse). Relapses had to be accompanied by clinically relevant changes in EDSS score (i.e., ≥0.5-point increase in EDSS score, 1-point increase on two functional scores, or a 2-point increase in any single functional score, except bowel/bladder and cerebral functional scores).

Definition of secondary disability endpoints. Confirmed disability worsening at 3 or 6 months was defined as a confirmed, sustained increase from baseline in EDSS score (of ≥1.5 points for baseline score of 0, of ≥1.0 point for baseline score of 0.5 to ≤5.5, and of ≥0.5 points for baseline EDSS score of >5.5) over at least the specified number of months that is not attributable to another etiology (e.g., fever, concurrent illness, or concomitant medication). The initial onset increase from baseline EDSS score could have been from a scheduled or unscheduled assessment and could have occurred during or outside of a relapse. All intermediate EDSS scores (i.e., EDSS scores obtained after onset of disability and before the confirmatory assessment), if any, were maintained for at least the minimum increase. Confirmatory EDSS assessments were obtained over at least the specified number of months (3 or 6 months) after onset, at a routine quarterly visit, at least 30 days after any confirmed clinical relapse, and could not be associated with an ongoing relapse. Confirmed disability improvement at 6 months was defined as a ≥1-point decrease in EDSS score from baseline lasting at least 6 months.

MRI assessments and endpoints. MRI scans were performed at baseline, every 6 months until Month 24, and every 12 months until the end of study. MRI scans required one-month washout period for systemic corticosteroids and adrenocorticotropic hormone. The MRI protocol included T2- and T1-weighted sequences before and after gadolinium contrast agent administration, except where contraindicated. These MRI sequences were used to evaluate MRI-related secondary end points. MRI-related end points were assessed by central reviewers who were blinded to treatment assignments and other participant data. The total number of new/enlarging T2-weighted lesions was defined as the sum of the individual number of new or enlarging T2-weighted lesions at all scheduled visits starting after baseline up to and including the end of study visit.

Cognitive Assessments. The oral version of the Symbol Digit Modalities Test (SDMT) was administered at baseline and every 3 months until the end of study. The SDMT measures information processing speed and involves a simple substitution task using a reference key.25 The number of correct pairings made in 90 seconds was recorded. A decrease of 4 points from baseline on the SDMT is considered meaningful worsening.26 The baseline mean and standard deviation of all participants were used to create the Z-score for SDMT number of correct substitutions: Z=(SDMT score−baseline mean SDMT)/baseline standard deviation SDMT.

The California Verbal Learning Test-Second Edition (CVLT-II) was administered at baseline and every 3 months until the end of study. The CVLT-II measures verbal learning and memory. Participants were instructed to listen a 16-item word list read by the examiner and report as many of the items as possible. Five successive trials were scored. The total correct recall across the 5 trials was converted to a standardized score by sex within each of 7 age groups. The total correct recall from the five trials was scaled to a normalized T-score metric that had a mean of 50 and standard deviation of 10, with higher values indicating better performance.

### Safety

Adverse Events. Adverse events were reported in 792 of 933 (84.9%) participants who received tolebrutinib and 810 of 939 (86.3%) participants who received teriflunomide in GEMINI 1 and 2 (see Table 2N). Adverse events that were reported for ≥10% of participants who received tolebrutinib included COVID-19, nasopharyngitis, and headache; adverse events reported for ≥10% of participants who received teriflunomide were COVID-19, alopecia, nasopharyngitis, and headache (see Table 2N). Serious adverse events occurred in 9.8% of participants who received tolebrutinib and 8.2% of participants who received teriflunomide. Deaths were balanced between the groups. One death (0.1%) occurred in the tolebrutinib group, which was due to homicidal gunshot wound, assessed as unrelated to tolebrutinib. Two deaths (0.2%) occurred in the teriflunomide group, which were due to suicide by firearm and brain edema from traumatic subarachnoid hemorrhage, assessed as unrelated to teriflunomide.

Adverse Events of Special Interest. Adverse events of special interest, based on observations from other BTK inhibitors in oncology, included increase in alanine aminotransferase levels, atrial fibrillation/atrial flutter, severe infection, moderate to severe hemorrhagic events, and thrombocytopenia. Increase in alanine aminotransferase levels was an identified risk with tolebrutinib and is described in the liver safety section. Severe infections were seen at similar rates between tolebrutinib (2.3%) and teriflunomide (1.9%). No differences were found in moderate to severe hemorrhagic events (2.1% tolebrutinib; 1.7% teriflunomide). While minor bleeding events were not an adverse event of special interest, such as petechia and heavy menses were identified with higher frequency in tolebrutinib (4.5% and 2.6%, respectively) compared to teriflunomide (0.3% and 1.0%, respectively). No differences in cardiac arrhythmias or thrombocytopenia were reported between study treatment arms.

Liver Safety. Increased alanine aminotransferase levels above three-times the upper limit of normal were reported in 52 of 929 (5.6%) of participants who received tolebrutinib and 58 of 926 (6.3%) of participants who received teriflunomide (see Table 2N, FIG. 28). Peak alanine aminotransferase level increases greater than 20-times the upper limit of normal occurred in 0.5% of participants who received tolebrutinib and 0.1% of participants who received teriflunomide. All cases of increased alanine aminotransferase levels above 20-times the upper limit of normal occurred within the first 90 days of treatment. Four (0.4%) participants in the tolebrutinib group and one (0.1%) in the teriflunomide group had levels of alanine aminotransferase greater than three times the upper limit of normal and total bilirubin greater than two-times the upper limit of normal. All cases of elevated liver enzyme levels resolved without sequelae.

### Discussion

In the GEMINI 1 and 2 trials, tolebrutinib treatment did not result in a lower annualized (adjudicated) relapse rate or fewer acute inflammatory brain lesions on MRI in comparison to the active comparator, teriflunomide. The percentage of participants with confirmed disability worsening over 6 and 3 months appeared lower with tolebrutinib than with teriflunomide, although this was non-confirmatory due to termination of the hierarchical testing procedure based on non-significance of the primary end point.

The BTK inhibitor evobrutinib similary did not lower the annualized (adjudicated) relapse rate versus teriflunomide in relapsing multiple sclerosis, (Montalban, et al., Lancet Neurolog 2024; 23:1119-1132), and showed no significant evidence of reducing the risk of disability worsening versus teriflunomide. Lack of improvement in the disability outcomes with evobrutinib is speculated to be attributed to the extent of CNS penetrance, with evidence that cerebrospinal fluid concentrations exceeding the 90% inhibitory concentration were reached by tolebrutinib but not evobrutinib (Turner, et al., Drugs in R&D 2024; 24:263-74). Conclusions about the relative efficacy between these agents cannot be inferred without direct comparison in comparative effectiveness trials.

Although the results for disability outcomes were non-confirmatory, tolebrutinib is the first treatment to show differing results on relapses and disability, which is meaningful considering that these clinical outcomes are thought to be underpinned by distinct pathological processes (Scalfari et al., Ann Neurol 2024; 96:826-45). Existing disease-modifying therapies have limited impact on disability, likely because they act predominantly on acute focal inflammatory activity in the periphery, while tolebrutinib is thought to modulate immunological drivers of chronic inflammation behind the blood-brain barrier (Gruber et al., Nat. Commun. 2024; 15:10116).

Increased alanine aminotransferase levels occurred at a similar frequency with tolebrutinib (5.6%) and teriflunomide (6.3%) and all resolved without sequelae. Most alanine aminotransferase elevations were mild to moderate. Elevations greater than 20 times the upper limit of normal were uncommon, and all occurred within the first 90 days of treatment.

In the GEMINI 1 and 2 trials, tolebrutinib did not reduce relapse rate or acute inflammatory MRI activity compared to teriflunomide, but was associated with a numerically lower risk of disability worsening and rare events of high liver enzyme elevations.

### Subgroup Analysis

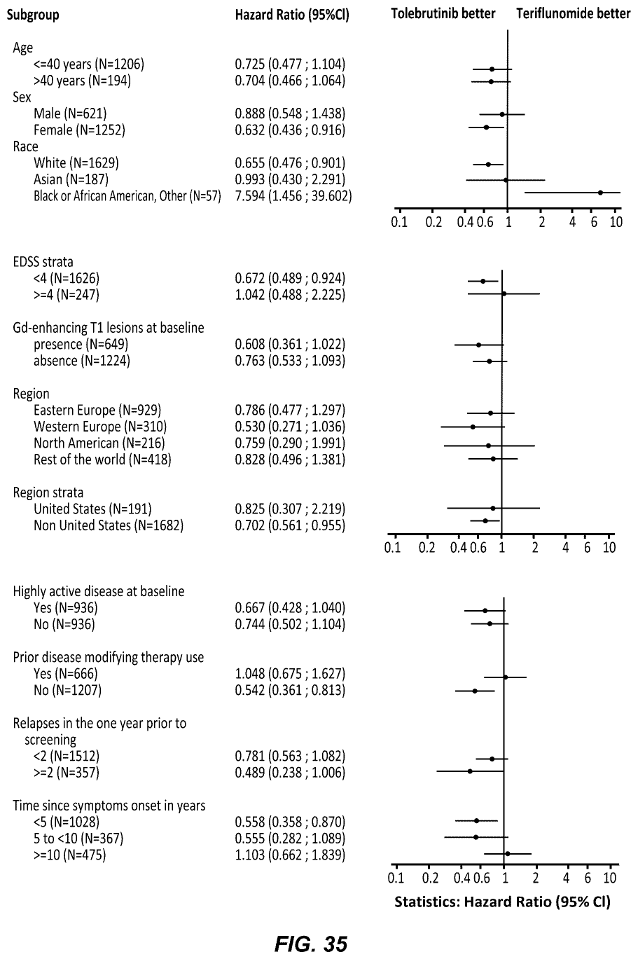

A consistent reduction in the risk of 6-month CDW with tolebrutinib versus teriflunomide was generally observed across all subgroups, with potentially higher efficacy in participants with no prior use of DMTs (see Table 1Q, FIG. 35).

TABLE 2P
Analysis of time to onset of 6-month CDW using Cox model
by all subgroups (intention-to-treat population, pooled)

| | | | Teriflunomide | | Tolebrutinib | |
|---|---|---|---|---|---|---|
| Subgroup | | Statistic | (N = 940) | | (N = 933) | |
| Age | <=40 years | No. | 608 | | 598 | |
| | | No. with 6 month CDW, n (%) | 52 | (8.6) | 37 | (6.2) |
| | | Hazard ratio (95% CI) ^{a} | 0.725 (0.477, 1.104) | | | |
| | >40 years | No. | 332 | | 335 | |
| | | No. with 6 month CDW, n (%) | 53 | (16.0) | 40 | (11.9) |
| | | Hazard ratio (95% CI) ^{a} | 0.704 (0.466, 1.064) | | | |
| | Overall | p-value^{b} | 0.9103 | | | |
| Sex | Male | No. | 322 | | 299 | |
| | | No. with 6 month CDW, n (%) | 37 | (11.5) | 30 | (10.0) |
| | | Hazard ratio (95% CI) ^{a} | 0.888 (0.548, 1.438) | | | |
| | Female | No. | 618 | | 634 | |
| | | No. with 6 month CDW, n (%) | 68 | (11.0) | 47 | (7.4) |
| | | Hazard ratio (95% CI) ^{a} | 0.632 (0.436, 0.916) | | | |
| | Overall | p-value^{b} | 0.2927 | | | |
| Race | White | No. | 823 | | 806 | |
| | | No. with 6 month CDW, n (%) | 94 | (11.4) | 63 | (7.8) |
| | | Hazard ratio (95% CI) ^{a} | 0.655 (0.476, 0.901) | | | |
| | Asian | No. | 86 | | 101 | |
| | | No. with 6 month CDW, n (%) | 10 | (11.6) | 12 | (11.9) |
| | | Hazard ratio (95% CI) ^{a} | 0.993 (0.430, 2.291) | | | |
| | Black or | No. | 31 | | 26 | |
| | African | No. with 6 month CDW, n (%) | 1 | (3.2) | 2 | (7.7) |
| | American, | Hazard ratio (95% CI) ^{a} | 7.594 (1.456, 39.602) | | | |
| | Other | | | | | |
| | Overall | p-value^{b} | 0.3545 | | | |
| EDSS strata | <4 | No. | 815 | | 811 | |
| | | No. with 6 month CDW, n (%) | 92 | (11.3) | 64 | (7.9) |
| | | Hazard ratio (95% CI) ^{a} | 0.672 (0.489, 0.924) | | | |
| | >=4 | No. | 125 | | 122 | |
| | | No. with 6 month CDW, n (%) | 13 | (10.4) | 13 | (10.7) |
| | | Hazard ratio (95% CI) ^{a} | 1.042 (0.488, 2.225) | | | |
| | Overall | p-value^{b} | 0.3156 | | | |
| Gd-enhancing | presence | No. | 336 | | 313 | |
| T1 lesions | | No. with 6 month CDW, n (%) | 39 | (11.6) | 23 | (7.3) |
| at baseline | | Hazard ratio (95% CI) ^{a} | 0.608 (0.361, 1.022) | | | |
| | absence | No. | 604 | | 620 | |
| | | No. with 6 month CDW, n (%) | 66 | (10.9) | 54 | (8.7) |
| | | Hazard ratio (95% CI) ^{a} | 0.763 (0.533, 1.093) | | | |
| | Overall | p-value^{b} | 0.5288 | | | |
| Region | Eastern | No. | 449 | | 480 | |
| | Europe | No. with 6 month CDW, n (%) | 33 | (7.3) | 29 | (6.0) |
| | | Hazard ratio (95% CI) ^{a} | 0.786 (0.477, 1.297) | | | |
| | Western | No. | 173 | | 137 | |
| | Europe | No. with 6 month CDW, n (%) | 31 | (17.9) | 14 | (10.2) |
| | | Hazard ratio (95% CI) ^{a} | 0.530 (0.271, 1.036) | | | |
| | North | No. | 113 | | 103 | |
| | America | No. with 6 month CDW, n (%) | 9 | (8.0) | 7 | (6.8) |
| | | Hazard ratio (95% CI) ^{a} | 0.759 (0.290, 1.991) | | | |
| | Rest of the | No. | 205 | | 213 | |
| | world | No. with 6 month CDW, n (%) | 32 | (15.6) | 27 | (12.7) |
| | | Hazard ratio (95% CI) ^{a} | 0.828 (0.496, 1.381) | | | |
| | Overall | p-value^{b} | 0.7658 | | | |
| Region | United States | No. | 97 | | 94 | |
| strata | | No. with 6 month CDW, n (%) | 8 | (8.2) | 7 | (7.4) |
| | | Hazard ratio (95% CI) ^{a} | 0.825 (0.307, 2.219) | | | |
| | Non United | No. | 843 | | 839 | |
| | States | No. with 6 month CDW, n (%) | 97 | (11.5) | 70 | (8.3) |
| | | Hazard ratio (95% CI) ^{a} | 0.702 (0.516, 0.955) | | | |
| | Overall | p-value^{b} | 0.7787 | | | |
| Highly | Yes | No. | 478 | | 458 | |
| active | | No. with 6 month CDW, n (%) | 49 | (10.3) | 32 | (7.0) |
| disease at | | Hazard ratio (95% CI) ^{a} | 0.667 (0.428, 1.040) | | | |
| baseline | No | No. | 462 | | 474 | |
| | | No. with 6 month CDW, n (%) | 56 | (12.1) | 45 | (9.5) |
| | | Hazard ratio (95% CI) ^{a} | 0.744 (0.502, 1.104) | | | |
| | Overall | p-value^{b} | 0.7233 | | | |
| Prior | Yes | No. | 349 | | 317 | |
| disease | | No. with 6 month CDW, n (%) | 42 | (12.0) | 40 | (12.6) |
| modifying | | Hazard ratio (95% CI) ^{a} | 1.048 (0.675, 1.627) | | | |
| therapy use | No | No. | 591 | | 616 | |
| | | No. with 6 month CDW, n (%) | 63 | (10.7) | 37 | (6.0) |
| | | Hazard ratio (95% CI) ^{a} | 0.542 (0.361, 0.813) | | | |
| | Overall | p-value^{b} | 0.0315 | | | |
| Relapses in | <2 | No. | 755 | | 757 | |
| the one year | | No. with 6 month CDW, n (%) | 81 | (10.7) | 66 | (8.7) |
| prior | | Hazard ratio (95% CI) ^{a} | 0.781 (0.563, 1.082) | | | |
| to screening | >=2 | No. | 183 | | 174 | |
| | | No. with 6 month CDW, n (%) | 24 | (13.1) | 11 | (6.3) |
| | | Hazard ratio (95% CI) ^{a} | 0.489 (0.238, 1.006) | | | |
| | Overall | p-value^{b} | 0.2209 | | | |
| Time since | <5 | No. | 522 | | 506 | |
| symptom | | No. with 6 month CDW, n (%) | 54 | (10.3) | 30 | (5.9) |

| onset in | | Hazard ratio (95% CI) ^{a} | 0.558 (0.358, 0.870) | |
|---|---|---|---|---|
| years | 5 to <10 | No. | 186 | 181 |

| | | | | |
|---|---|---|---|---|
| No. with 6 month CDW, n (%) | 23 | (12.4) | 14 | (7.7) |

| | Hazard ratio (95% CI) ^{a} | 0.555 (0.282, 1.089) | |
|---|---|---|---|
| >=10 | No. | 232 | 243 |

| | | | | |
|---|---|---|---|---|
| No. with 6 month CDW, n (%) | 28 | (12.1) | 33 | (13.6) |

| | | |
|---|---|---|
| | Hazard ratio (95% CI) ^{a} | 1.103 (0.662, 1.839) |
| Overall | p-value^{b} | 0.1047 |

^{a} Derived using Cox proportional-hazards model with treatment group, age at screening (>40, <=40 years), geographic region (US, non-US), baseline EDSS score and baseline Gd-enhancing T1 lesions (presence, absence) as covariates.
^{b}p-value for treatment by subgroup interaction, derived using Cox proportional-hazards model with treatment group, age at screening (>40, <=40 years), geographic region (US, non-US), baseline EDSS score and baseline Gd-enhancing T1 lesions (presence, absence), subgroup (if different than the aforementioned covariates) and treatment by subgroup interaction as covariates.

### Effects on Progression Independent of Relapse Activity (PIRA)

A post hoc analysis evaluated the effect of tolebrutinib vs. teriflunomide on PTRA in the GEMINI 1 & 2 relapsing MS trials. PTRA was defined as a 6-month CDW event (a sustained increase from baseline in EDSS score of ≥1.5 points when baseline score is 0, ≥1.0 points when baseline score is 0.5 to ≤5.5 or ≥0.5 points when baseline score is >5.5, confirmed over ≥6 months) with no adjudicated relapse onset 90 days prior to or after disability worsening and no adjudicated relapse onset in the 30 days prior to confirmation of disability worsening. 6-month CDW events not meeting the PIRA definition were classified as relapse-associated worsening (RAW). Initial onset of disability worsening could have occurred at a scheduled or unscheduled visit but was required to be confirmed at a scheduled quarterly visit.

Baseline characteristics for patients grouped by on-trial PIRA status are presented in Table 2Q. Participants with PIRA tended to have longer disease duration and fewer were treatment-naïve compared to participants without PIRA.

TABLE 2Q
Baseline Characteristics by On-Trial PIRA Status in GEMINI 1 & 2 (pooled)

| | Teriflunomide (N = 940) | | | | Tolebrutinib (N = 933) | | | |
|---|---|---|---|---|---|---|---|---|
| | | | Without | | | | Without | |
| | With PIRA | | PIRA | | With PIRA | | PIRA | |
| Characteristic | (n = 80) | | (n = 860) | | (n = 60) | | (n = 873) | |
| Age, years (SD) | 39.9 | (9.8) | 36.0 | (9.3) | 40.6 | (8.6) | 36.4 | (9.1) |
| Female, n (%) | 50 | (62.5) | 568 | (66.0) | 37 | (61.7) | 597 | (68.4) |
| MS subtype, n (%) | | | | | | | | |
| Relapsing remitting | 78 | (97.5) | 855 | (99.4) | 59 | (98.3) | 865 | (99.1) |
| Secondary progressive | 2 | (2.5) | 5 | (0.6) | 1 | (1.7) | 8 | (0.9) |
| EDSS score^{a} | | | | | | | | |
| Mean (SD) | 2.3 | (1.4) | 2.4 | (1.2) | 2.5 | (1.3) | 2.4 | (1.2) |
| Median (IQR) | 2.1 | (1.1-3.1) | 2.1 | (1.5-3.0) | 2.1 | (1.5-3.5) | 2.0 | (1.5-3.3) |
| Time since relapsing MS | 7.8 | (8.5) | 6.4 | (6.9) | 9.9 | (9.1) | 6.6 | (6.9) |
| symptom onset, years (SD) | | | | | | | | |
| Number of relapses within | 1.2 | (0.7) | 1.2 | (0.6) | 1.1 | 0.6 | 1.2 | (0.6) |
| previous year (SD) | | | | | | | | |
| Participants with ≥1 Gd- | 28 | (35.4) | 304 | (35.6) | 17 | (28.3) | 296 | (34.0) |
| enhancing T1 lesion, n (%) | | | | | | | | |
| Number of Gd-enhancing T1 | 1.5 | (3.7) | 1.2 | (3.8) | 0.5 | (1.0) | 1.2 | (3.3) |
| lesions (SD) | | | | | | | | |
| Participants who were | 46 | (57.5) | 545 | (63.4) | 29 | (48.3) | 587 | (67.2) |
| treatment-naïve, n (%) | | | | | | | | |

^{a}Average of screening and randomization EDSS scores

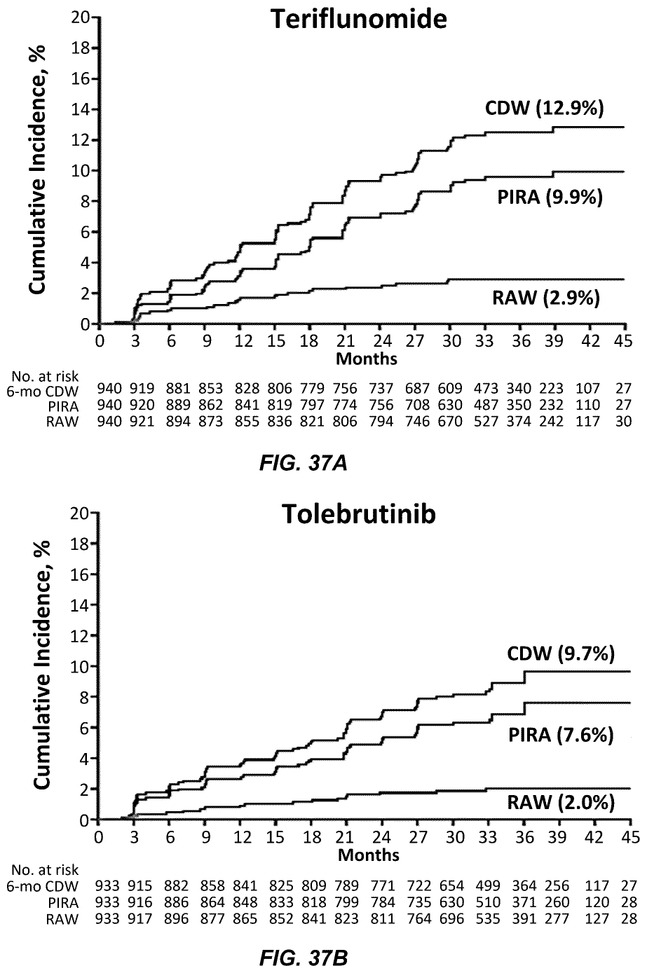

The majority (nearly 80%) of 6-month CDW events were PIRA in both the tolebrutinib and teriflunomide treatment groups. In the tolebrutinib group (N=933), 8.3% of patients had a 6-month CDW event of any kind, 6.4% classified as PIRA (77.9% of CDW events) and 1.8% classified as RAW. In the teriflunomide group (N=940), 11.2% of patients had a 6-month CDW event of any kind, 8.5% classified as PIRA (76.2% of CDW events) and 2.7% classified as RAW. Kaplan-Meier estimates of the time to onset of 6-month CDW, PIRA, and RAW are shown in FIGS. 37A-37B for teriflunomide and tolebrutinib, respectively. Tolebrutinib achieved a 27% risk reduction of PIRA vs. teriflunomide (HR [95% CI]: 0.73 [0.52-1.02]).

### Pharmacokinetic Measures

Blood samples were collected at specified timepoints to assess maximum observed plasma concentration (Cₘₐₓ), time to maximum observed plasma concentration (Tₘₐₓ), and area under the plasma concentration-time curve over the last 24-hour dosing interval (AUC₀₋₂₄) of tolebrutinib and M2 metabolite using a population pharmacokinetics (PopPK) model, with three compartments for tolebrutinib and one for M2 and exposure parameters determined at steady-state for each sample and averaged within individual participants. Samples were collected 30 to 90 minutes post-dose at Months 6, 9, and 12 and 2.5 to 5 hours post-dose at Months 6 and 12. For PK visits, participants were required to administer treatment at the study site after a regular meal. Samples were evaluated for 444 participants in the GEMINI 1 study using a validated method of liquid chromatography with tandem mass spectrometry. Mean Cₘₐₓ (SD) was 12.0 (7.75) ng/L for tolebrutinib and 28.3 (15.8) ng/mL for M2 metabolite. Mean Tₘₐₓ (SD) was 1.28 (0.513) h for tolebrutinib and 1.40 (0.508) h for M2 metabolite. Mean AUC₀₋₂₄ was 30.5 (18.2) ng*h/mL for tolebrutinib and 76.7 (44.1) ng*h/mL for M2 metabolite.

Cytochrome P450 2C8 catalyses the conversion of tolebrutinib into the active M2 metabolite, which has a similar pharmacological profile as tolebrutinib, including covalent BTK inhibition and brain penetrance (Cabanis M J, et al. Clin. Transl. Sci. 2024; 17:e13693; Nicolas O, et al., Clin. Drug. Investig. 2023; 43:653-665). M2 metabolite has the following formula:

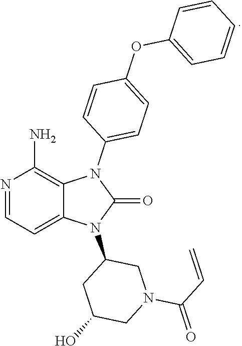 [SMILES: C=CC(=O)N1C[C@H](O)C[C@@H](n2c(=O)n(-c3ccc(Oc4ccccc4)cc3)c3c(N)nccc32)C1]

### Pharmacodynamic Measures

Change from Baseline in Plasma Neurofilament Light Chain (NfL) Levels at End of Study (EOS). Levels of NfL in plasma were measured as a potential marker of neuronal damage. Baseline was defined as the last available value prior to the first dose of study intervention. In GEMINI 1, the median change (% change; IQR; number of participants evaluated) from baseline at the EOS in plasma NfL levels was −0.325 pg/mL (−3.417%; −3.505 to 2.220; n=296) in the tolebrutinib group and −1.665 pg/mL (−18.620%; −6.100 to 0.830; n=294) in the teriflunomide group. In GEMINI 2, the median change (% change; IQR; number of participants evaluated) from baseline at the EOS in plasma NfL levels was −0.150 pg/mL (−2.160%; −3.560 to 2.750; n=314) in the tolebrutinib group and −0.900 pg/mL (−13.029%; −4.390 to 0.770; n=301) in the teriflunomide group.

Change from Baseline in Serum Chitinase-3 Like Protein-1 (Chi3L1) Levels at End of Study (EOS). Levels of Chi3L1 in serum were measured as a potential biomarker for the progression of MS. In GEMINI 1, the median change (% change; IQR; number of participants evaluated) from baseline at the EOS in serum Chi3L1 levels was 1572.250 pg/mL (7.564%; −2582.000 to 6356.600; n=350) in the tolebrutinib group and 1017.100 (5.302%; −4494.850 to 7672.450; n=344) in the teriflunomide group. In GEMINI 2, the median change (% change; IQR; number of participants evaluated) from baseline at the EOS in serum Chi3L1 levels was 1462.500 pg/mL (7.210%; −4578.600 to 7002.700; n=319) in the tolebrutinib group and −281.100 (−1.350%; −6148.400 to 5647.000; n=311) in the teriflunomide group.

Change from Baseline in Cluster of Differentiation 19 (CD19+) B cells at End of Study (EOS). Baseline was defined as the last available value prior to the first dose of study intervention. In GEMINI 1, the median change (% change; IQR; number of participants evaluated) from baseline at the EOS in CD19+ B cells was −60.500 cells/μL (−36.465%; −97.500 to −28.500; n=100) in the tolebrutinib group and −45.000 cells/μL (−24.901%; −81.000 to −5.000; n=95) in the teriflunomide group.

Change from Baseline in Serum Immunoglobulin (Ig) Levels at End of Study (EOS). Baseline was defined as the last available value prior to the first dose of study intervention. In GEMINI 1, the median change (% change; IQR; number of participants evaluated) from baseline at the EOS in serum IgG was 0.235 g/L (2.375%; −0.500 to 1.020; n=266) in the tolebrutinib group and −0.660 g/L (−6.553%; −1.460 to 0.150; n=261) in the teriflunomide group. In GEMINI 2, the median change (% change; IQR; number of participants evaluated) from baseline at the EOS in serum IgG was 0.140 g/L (1.209%; −0.720 to 1.060; n=287) in the tolebrutinib group and −0.590 g/L (−5.417%; 1.0330 to 0.230; n=281) in the teriflunomide group. In GEMINI 1, the median change (% change; IQR; number of participants evaluated) from baseline at the EOS in serum IgM was −0.340 g/L (−28.358%; −0.520 to −0.190; n=263) in the tolebrutinib group and −0.150 g/L (−14.493%; −0.330 to −0.030; n=263) in the teriflunomide group. In GEMINI 2, the median change (% change; IQR; number of participants evaluated) from baseline at the EOS in serum IgM was −0.320 g/L (−26.744%; −0.530 to −0.150; n=287) in the tolebrutinib group and −0.160 g/L (−15.328%; −0.320 to −0.020; n=281) in the teriflunomide group. Mean IgG levels remained overall stable and mean IgM levels remained above the lower limit of normal (LLN) in both the tolebrutinib and teriflunomide groups over the duration of the trials. In the GEMINI 1 teriflunomide group (n=488), 4.5% (n=22) of participants had IgG less than LLN during treatment, 2.5% (n=12) had normal IgG at baseline, 1.0% (n=5) had IgG less than LLN at baseline, and 1.0% (n=5) had a missing baseline. In the GEMINI 1 tolebrutinib group (n=486), 1.9% (n=9) of participants had IgG less than LLN during treatment, 1.0% (n=5) had normal IgG at baseline, 0.2% (n=1) had IgG less than LLN at baseline, and 0.6% (n=3) had a missing baseline. In the GEMINI 2 teriflunomide group (n=452), 3.5% (n=16) of participants had IgG less than LLN during treatment, 3.5% (n=16) had normal IgG at baseline. In the GEMINI 2 tolebrutinib group (n=447), 2.5% (n=11) of participants had IgG less than LLN during treatment, 1.1% (n=5) had normal IgG at baseline, 1.1% (n=5) had IgG less than LLN at baseline, and 0.2% (n=1) had a missing baseline. In the GEMINI 1 teriflunomide group (n=488), 0.8% (n=4) of participants had IgM less than LLN during treatment, 0.6% (n=3) had normal IgM at baseline and 0.2% (n=1) had IgM less than LLN at baseline. In the GEMINI 1 tolebrutinib group (n=486), 0.6% (n=3) of participants had IgM less than LLN during treatment, 0.2% (n=1) had normal IgM at baseline and 0.4% (n=2) had a missing baseline. In the GEMINI 2 teriflunomide group (n=452), 1.3% (n=6) of participants had IgM less than LLN during treatment, 0.9% (n=4) had normal IgM at baseline and 0.4% (n=2) had IgM less than LLN at baseline. In the GEMINI 2 tolebrutinib group (n=447), 1.3% (n=6) of participants had IgM less than LLN during treatment, 1.3% (n=6) had normal IgM at baseline. LLN values for IgG were 5.86 g/L for females and 6.03 for males, and for IgM were 0.26 g/L for females and 0.20 g/L for males.

Immune Cell Populations. Over 42 months, mean leukocyte, lymphocyte, neutrophil, monocyte, and platelet counts remained stable and within the normal range in the tolebrutinib and teriflunomide groups. The decrease in leukocyte and neutrophil counts observed in the teriflunomide groups was consistent with the 10-15% decrease reported for prior placebo-controlled studies of teriflunomide. 1.2% (GEMINI 1) and 0.2% (GEMINI 2) of participants developed leukopenia in the tolebrutinib groups and 2.5% (GEMINI 1) and 0.7% (GEMINI 2) of participants developed leukopenia in the teriflunomide groups. 0.2% (GEMINI 1) and 0.4% (GEMINI 2) of participants developed lymphopenia in the tolebrutinib groups and 1.6% (GEMINI 1) and 1.1% (GEMINI 2) of participants developed lymphopenia in the teriflunomide groups. 2.9% (GEMINI 1) and 2.2% (GEMINI 2) of participants developed neutropenia in the tolebrutinib groups and 11.9% (GEMINI 1) and 7.5% (GEMINI 2) of participants developed neutropenia in the teriflunomide groups. Thrombocytopenia (platelet count <75×10⁹/nL) was not observed in any participant in either study.

### Example 3—A Phase 3, Randomized, Double-Blind Efficacy and Safety Study Comparing Tolebrutinib to Placebo in Participants with Primary Progressive Multiple Sclerosis (PERSEUS)

The goal of this Phase 3 clinical trial is to demonstrate the efficacy and safety of tolebrutinib compared to placebo in participants with primary progressive multiple sclerosis (PPMS). The primary endpoint is time to onset of 6-month composite confirmed disability progression (6M-cCDP) assessed via the Expanded Disability Status Scale (EDSS) score. Secondary endpoints include 6-month confirmed disability progression (CDP), 3-month-cCDP, and confirmed disability improvement (CDI), each assessed via the EDSS score, which is shown in FIG. 8.

A graphical scheme of the study design is shown in FIG. 1C. Abbreviations used in FIG. 1C: CDP, confirmed disability progression; DMT, disease modifying therapy; EOS, end-of-study; MRI, magnetic resonance imaging.

Table 3A shown below describes the schedule of activities during the course of study. Table 3B that follows describes the objective and endpoints of the overall study.

TABLE 3A
Schedule of Activities (SOA)

| | | |
|---|---|---|
| | Randomization/ | |
| Screening^{a} | start of IMP | To Year 2 (M 24)^{b} |

Visit (a window of ±7 days is allowed for all visits after D 1)

| | | | | | | | | | | | |
|---|---|---|---|---|---|---|---|---|---|---|---|
| | | | | M 1.25 | | M 2.25 | | | | | |
| | | | | (W 5) | | (W 9) | | | | | |
| | | | | M 1.5 | | M 2.5 | | | | | |
| D −28 | | | | (W 6) | | (W 10) | | | | | |
| to | | M 0.5 | M 1 | M 1.75 | M 2 | M 2.75 | | M 4, | | M 7 | |
| D −1 | D 1 | (W 2) | (W 4)^{d} | (W 7) | (W 8)^{d} | (W 11) | M 3 | M 5^{d} | M 6 | M 8 | M 9 |

Visit Number

| | | | | | | V6, | | |
|---|---|---|---|---|---|---|---|---|
| Procedure | V1 | V2 | V3 | V4 | V5 | V7 | V8 | V9 |
| Informed | X | | | | | | | |
| consent | | | | | | | | |
| Demography | X | | | | | | | |
| Inclusion/ | X | X | | | | | | |
| exclusion | | | | | | | | |
| criteria | | | | | | | | |
| Lumbar | X | | | | | | | |
| puncture for | | | | | | | | |
| CSF analysis^{g} | | | | | | | | |
| Medical/surgical | X | | | | | | | |
| history | | | | | | | | |
| (including | | | | | | | | |
| substance usage) | | | | | | | | |

| | |
|---|---|
| Prior/ | <=====================================================================================================> |
| concomitant | |
| medications^{h} | |

| | | | | | |
|---|---|---|---|---|---|
| Randomization | | X | | | |
| IRT contact | X | X | X | X | X |
| IMP | | X | X | X | X |
| dispensationⁱ | | | | | |
| IMP | | | X | X | X |
| compliance | | | | | |
| Paper diary | | X | X | X | X |
| dispensation/ | | | | | |
| collection | | | | | |

Safety^{j}

| | | | | | | | | | | | | |
|---|---|---|---|---|---|---|---|---|---|---|---|---|
| Physical | X | X | | | | | | X | | X | | X |
| examination^{l} | | | | | | | | | | | | |
| Height | X | | | | | | | | | | | |
| Body weight | X | X | | | | | | X | | X | | |
| Serology tests | X | | | | | | | | | | | |
| for hepatitis B | | | | | | | | | | | | |
| and C HIV and | | | | | | | | | | | | |
| other infectious | | | | | | | | | | | | |
| diseases, if | | | | | | | | | | | | |
| required locally | | | | | | | | | | | | |
| TB/QuantiFERON ® | X | | | | | | | | | | | |
| TB | | | | | | | | | | | | |
| Gold test or | | | | | | | | | | | | |
| equivalent^{m} | | | | | | | | | | | | |
| Vital signs | X | X | | | | | | X | | X | | X |
| Body | X | X | | | | | | X | | X | | X |
| temperature | | | | | | | | | | | | |
| 12-lead ECGⁿ | X | | | | | | | X | | X | | X |
| Hematology, | X | X^{cc} | | X | | X | | X | X | X | | X |
| biochemistry^{j} | | | | | | | | | | | | |
| Liver function | | | X | | X | | X | | | | X | |
| tests^{k} | | | | | | | | | | | | |
| Iron panel | X | | | | | | | | | | | |
| (serum): iron, | | | | | | | | | | | | |
| ferritin, | | | | | | | | | | | | |
| transferrin | | | | | | | | | | | | |
| saturation, | | | | | | | | | | | | |
| TIBC; to be | | | | | | | | | | | | |
| repeated during | | | | | | | | | | | | |
| the study if | | | | | | | | | | | | |
| needed | | | | | | | | | | | | |
| Coagulation: | X | | | | | | | | | | | |
| PT/INR, aPTT | | | | | | | | | | | | |
| (to be repeated | | | | | | | | | | | | |
| during the | | | | | | | | | | | | |
| study, if | | | | | | | | | | | | |
| needed) | | | | | | | | | | | | |
| Urinalysis^{o} | X | | | | | | | X | | | | |
| Pregnancy test | X | X | | | | | | X | | X | | X |
| (if applicable)^{p} | | | | | | | | | | | | |
| Serum FSH^{q} | X | | | | | | | | | | | |
| Suicidality | X | X | | | | | | X | | X | | X |
| assessment by | | | | | | | | | | | | |
| C-SSRS | | | | | | | | | | | | |

| | |
|---|---|
| Adverse event | <=====================================================================================================> |
| collection | |

Efficacy

| | | | | | |
|---|---|---|---|---|---|
| EDSS | X | X | X | X | X |
| Timed 25-foot | | | X | X | X |
| walk test | | | | | |
| 9-hole peg test | | X | X | X | X |
| SDMT and | | X | X | X | X |
| CVLT-II, | | | | | |
| where | | | | | |
| available^{r} | | | | | |
| Basic or | X^{t} | | | X | |
| expanded MRI | | | | | |
| scan^{s} | | | | | |

| | |
|---|---|
| Actigraphy | X |
| (option for | |
| subset of | |
| participants)^{v} | |

Clinical outcome assessments^{W}

| | | |
|---|---|---|
| MSQoL-54 | X | X |
| EQ-5D-5L | X | X |

Pharmacokinetics

| | | |
|---|---|---|
| Tolebrutinib | X^{y} | X^{y} |
| pharmacokinetic | | |
| plasma | | |
| samplesⁱ | | |

Pharmacogenetics^{X}

| | |
|---|---|
| DNA | X |
| sample^{aa} | |

Pharmacodynamics/biomarkers

| | | | | |
|---|---|---|---|---|
| Blood sample | X | | | |
| for archiving^{bb} | | | | |
| Plasma | | X | X | X |
| samples | | | | |
| (NfL) serum | | | | |
| samples | | | | |
| (Chi3L1)^{z} | | | | |
| Immunophenotyping/ | | X | X | X |
| RNA | | | | |
| sequencing | | | | |
| (ToleDYNAMIC/ | | | | |
| optional | | | | |
| substudy at | | | | |
| selected | | | | |
| sites)^{ee} | | | | |

| | |
|---|---|
| OCT | X |

| | | | |
|---|---|---|---|
| substudy^{ff} | | | |
| Lymphocyte | X | | |
| phenotyping by | | | |
| flow cytometry | | | |
| in whole blood | | | |
| (subset of | | | |
| participants)^{dd} | | | |
| Serum | | X | X |
| samples (1 g | | | |
| levels)^{z} | | | |

| | | | | | | | | | | | Only for |
|---|---|---|---|---|---|---|---|---|---|---|---|
| | | | | | | | | | | | participants |
| | | | | | | | | | Only for | | who |
| | | | | | | | | | participants | | completed |
| | | | | | | | | | who | | treatment |
| | | | | | | | | | prematurely | | to EOS but |
| | | | | | | | | | discontinue | For all | do not enter |
| | To Year 2 (M 24)^{b} | | | | | | From M 27 to EOS^{b} | | IMP | participants | LTS^{c} |
| | Visit (a window of ±7 days is allowed for all visits after D 1) | | | | | | | | | | |
| | | | | | | | Quarterly | Semi- | | EOS | |
| | | | | | | | visits (M 27, | annual | | “Common | Follow-up |
| | | | | | | | M 30, M 33, | visits | | study | visit |
| | | | | | | | M 36, M 39, | (M 30, | | end | (4 to 8 |
| | M 10 | | | | | | M 42, M 45, | M 36, M 42, | | date” | weeks |
| | M 11 | M 12 | M 15 | M 18 | M 21 | M 24 | M 48 . . . ) | M 48 . . . ) | pEOT^{e} | visit^{f} | after EOS) |
| | Visit Number | | | | | | | | | | |
| | | | | | | | V15, V16, | V16, | | | |
| | | | | | | | V17, V18, | V18, | | | |
| | | | | | | | V19, V20, | V20, | | | |
| Procedure | | V10 | V11 | V12 | V13 | V14 | V21, V22 | V22 | pEOT^{e} | EOS | FUV |
| Informed | | | | | | | | | | | |
| consent | | | | | | | | | | | |
| Demography | | | | | | | | | | | |
| Inclusion/ | | | | | | | | | | | |
| exclusion | | | | | | | | | | | |
| criteria | | | | | | | | | | | |
| Lumbar | | | | | | | | | | | |
| puncture for | | | | | | | | | | | |
| CSF analysis^{g} | | | | | | | | | | | |
| Medical/surgical | | | | | | | | | | | |
| history | | | | | | | | | | | |
| (including | | | | | | | | | | | |
| substance usage) | | | | | | | | | | | |
| Prior/ | <=====================================================================================================> | | | | | | | | | | |
| concomitant | | | | | | | | | | | |
| medications^{h} | | | | | | | | | | | |
| Randomization | | | | | | | | | | | |
| IRT contact | X | X | X | X | X | | X | X | X | X | X |
| IMP | X | X | X | X | X | | X | X | | | |
| dispensationⁱ | | | | | | | | | | | |
| IMP | X | X | X | X | X | | X | X | X | X | |
| compliance | | | | | | | | | | | |
| Paper diary | X | X | X | X | X | | X | X | X | X | |
| dispensation/ | | | | | | | | | | | |
| collection | | | | | | | | | | | |
| | Safety^{j} | | | | | | | | | | |
| Physical | | X | X | X | X | X | X | X | X | X | X |
| examination^{l} | | | | | | | | | | | |
| Height | | | | | | | | | | | |
| Body weight | | X | | X | | X | | X | X | X | X |
| Serology tests | | | | | | | | | | | |
| for hepatitis B | | | | | | | | | | | |
| and C HIV and | | | | | | | | | | | |
| other infectious | | | | | | | | | | | |
| diseases, if | | | | | | | | | | | |
| required locally | | | | | | | | | | | |
| TB/QuantiFERON ® | | | | | | | | | | | |
| TB | | | | | | | | | | | |
| Gold test or | | | | | | | | | | | |
| equivalent^{m} | | | | | | | | | | | |
| Vital signs | | X | X | X | X | X | X | X | X | X | X |
| Body | | X | X | X | X | X | X | X | X | X | X |
| temperature | | | | | | | | | | | |
| 12-lead ECGⁿ | | X | | | | X | | yearly | X | X | |
| Hematology, | | X | X | X | X | X | X | X | X | X | X |
| biochemistry^{j} | | | | | | | | | | | |
| Liver function | X | | | | | | | | | | |
| tests^{k} | | | | | | | | | | | |
| Iron panel | | | | | | | | | | | |
| (serum): iron, | | | | | | | | | | | |
| ferritin, | | | | | | | | | | | |
| transferrin | | | | | | | | | | | |
| saturation, | | | | | | | | | | | |
| TIBC; to be | | | | | | | | | | | |
| repeated during | | | | | | | | | | | |
| the study if | | | | | | | | | | | |
| needed | | | | | | | | | | | |
| Coagulation: | | | | | | | | | | | |
| PT/INR, aPTT | | | | | | | | | | | |
| (to be repeated | | | | | | | | | | | |
| during the | | | | | | | | | | | |
| study, if | | | | | | | | | | | |
| needed) | | | | | | | | | | | |
| Urinalysis^{o} | | X | | X | | X | | X | X | X | |
| Pregnancy test | | X | X | X | X | X | X | X | X | X | X |
| (if applicable)^{p} | | | | | | | | | | | |
| Serum FSH^{q} | | | | | | | | | | | |
| Suicidality | | X | X | X | X | X | X | X | X | X | X |
| assessment by | | | | | | | | | | | |
| C-SSRS | | | | | | | | | | | |
| Adverse event | <=====================================================================================================> | | | | | | | | | | |
| collection | | | | | | | | | | | |
| | Efficacy | | | | | | | | | | |
| EDSS | | X | X | X | X | X | X | X | X | X | |
| Timed 25-foot | | X | X | X | X | X | X | X | X | X | |
| walk test | | | | | | | | | | | |
| 9-hole peg test | | X | X | X | X | X | X | X | X | X | |
| SDMT and | | X | X | X | X | X | X | X | X | X | |
| CVLT-II, | | | | | | | | | | | |
| where | | | | | | | | | | | |
| available^{r} | | | | | | | | | | | |
| Basic or | | X | | X | | X | | yearly | X | X | |
| expanded MRI | | | | | | | | | | | |
| scan^{s} | | | | | | | | | | | |
| Actigraphy | X | | | | | | | | | | |
| (option for | | | | | | | | | | | |
| subset of | | | | | | | | | | | |
| participants)^{v} | | | | | | | | | | | |
| | Clinical outcome assessments^{W} | | | | | | | | | | |
| MSQoL-54 | | X | | X | | X | | X | X | X | |
| EQ-5D-5L | | X | | X | | X | | X | X | X | |
| | Pharmacokinetics | | | | | | | | | | |
| Tolebrutinib | | X^{y} | | | | | | | X^{e} | | |
| pharmacokinetic | | | | | | | | | | | |
| plasma | | | | | | | | | | | |
| samplesⁱ | | | | | | | | | | | |
| | Pharmacogenetics^{X} | | | | | | | | | | |
| DNA | | | | | | | | | | | |
| sample^{aa} | | | | | | | | | | | |
| | Pharmacodynamics/biomarkers | | | | | | | | | | |
| Blood sample | | | | | | | | | | | |
| for archiving^{bb} | | | | | | | | | | | |
| Plasma | | X | | | | X | | Yearly | X | X | |
| samples | | | | | | | | | | | |
| (NfL) serum | | | | | | | | | | | |
| samples | | | | | | | | | | | |
| (Chi3L1)^{z} | | | | | | | | | | | |
| Immunophenotyping/ | | X | | | | | | | | | |
| RNA | | | | | | | | | | | |
| sequencing | | | | | | | | | | | |
| (ToleDYNAMIC/ | | | | | | | | | | | |
| optional | | | | | | | | | | | |
| substudy at | | | | | | | | | | | |
| selected | | | | | | | | | | | |
| sites)^{ee} | | | | | | | | | | | |
| OCT | X | | | | | | | | | | |
| substudy^{ff} | | | | | | | | | | | |
| Lymphocyte | | | | | | | | | X | | |
| phenotyping by | | | | | | | | | | | |
| flow cytometry | | | | | | | | | | | |
| in whole blood | | | | | | | | | | | |
| (subset of | | | | | | | | | | | |
| participants)^{dd} | | | | | | | | | | | |
| Serum | | X | | | | X | | yearly | X | X | |
| samples (1 g | | | | | | | | | | | |
| levels)^{z} | | | | | | | | | | | |

aPTT: activated partial thromboplastin time; β-HCG: β-human chorionic gonadotropin; AST: aspartate aminotransferase; ALT: alanine aminotransferase; Chi3L1: chitinase-3-like protein 1; C-SSRS: Columbia Suicide Severity Rating Scale; D: day; DNA: deoxyribonucleic acid; ECG: electrocardiogram; EDSS: Expanded Disability Status Scale; EOS: end of study; EOT: end of treatment; EQ 5D 5L: EuroQol 5-dimension 5-level questionnaire; FSH: follicle stimulating hormone; HIV: human immunodeficiency virus; Ig: immunoglobulin; IMP: investigational medicinal product; INR: international normalized ratio; IRT: interactive response technology; LTS: long term safety; M: month (28 days); MCV: mean corpuscular volume; MCH: mean corpuscular hemoglobin; MRI: magnetic resonance imaging; MS: multiple sclerosis; MSQoL-54: Multiple Sclerosis Quality of Life-54; NfL: neurofilament light chain; OCT: optical coherence tomography; pEOT: premature end of treatment; PK: pharmacokinetics; PT: prothrombin time; RBC: red blood cell; SWI: susceptibility weighted imaging; TB: tuberculosis; TIBC: total iron-binding capacity; V: visit; WBC: white blood cell.
Note:
All assessments shall be done as designated in this SoA unless not permitted according to local regulations. All visit assessments should be performed during the visit window, unless otherwise specified in this protocol.
^{a}Screening period can range from D −28 to D −1; Randomization visit can be performed only once IMPs are available at site. The interval between screening and randomization visits can range from 11 days (minimum) to 28 days (maximum). However, if required, randomization visit can be performed earlier than 11 days upon IMP receipt at the site, assuming the participant is eligible for randomization. In case of any delay to screening (MRI rescheduling, lab retests, etc), an additional period of up to 2 weeks is allowed. For other situations where a follow-up visit is needed, please see the Study Manual.
^{b}From D 1 to EOS, unscheduled visits may be performed at any time by the Investigator (eg, for evaluation of an adverse event). Assessments may be done as needed to evaluate the participant in accordance with the Investigator's best judgement and in-line with the study protocol. At a minimum, a physical examination should be performed, and body temperature and vital signs should be measured.
^{c}At the EOS, all the participants who have completed the study and remain on treatment with IMP (double blind or open label if meeting 6-month CDP) will be offered participation in a LTS study. Follow-up Visit assessment only performed for those participants who completed treatment and are not willing to take part in the LTS study. For other situations where a follow-up visit is needed, please see the Study Manual.
^{d}These visits may be done as home health visits (where applicable) or on-site visits (it is preferable that tests are performed at the central laboratory). In any situation where this is not possible (to be documented in source documents), the tests for these visits may be performed at a local laboratory.
^{e}If a participant prematurely permanently discontinues treatment with IMP, the participant will undergo a pEOT visit as soon as possible. A PK sample should also be collected if the pEOT visit can be scheduled within a maximum 24 hours after the last IMP dose. Participants will then be asked to continue with the study visits as scheduled until the global EOS Visit is reached. During these visits, all study procedures/assessments will be performed except IMP administration and blood sampling for PK and biomarkers (NfL, Chi3L1, and Ig levels). MRI scans for these participants will only be performed annually (using the next annual visit as the starting point).
^{f}Common EOS Visit will be done when the prespecified number of events for 6-month CDP is expected to be reached. The timing and window of this visit will be communicated to sites.
^{g}If no historical documentation, cerebrospinal fluid draw will be performed locally during the screening period.
^{h}Any disease modifying therapy (DMT) for MS taken at any time prior to signing the informed consent needs to be reported in the case report form (CRF); other prior medications will be reported for the period of 6 months prior to signing the informed consent form (ICF).
ⁱOn days of PK sampling the IMP needs to be taken at the study site after a “regular meal”. In case a participant forgot IMP at home or took IMP prior to arriving at the site on the day of visits with PK sampling, he/she will be asked to have a PK assessment within 3 days of the missed PK sampling. A PK assessment shall be done as soon as possible after an overdose or if otherwise specified per protocol (eg, investigation of abnormal laboratory test values).
^{j}Hematology (platelet count, RBC count, hemoglobin, hematocrit, MCV, MCH, reticulocytes, WBC count with differential: neutrophils, lymphocytes, monocytes, eosinophils, basophils).
Biochemistry (blood urea nitrogen [BUN], creatinine, glucose, potassium, sodium, chloride, bicarbonate, calcium, liver function tests [AST, ALT, albumin, alkaline phosphatase, total and direct bilirubin, total protein; creatine phosphokinase]) will be tested. Lipase will be tested at Screening only. Monthly visits (M 1, M 2, M 4, and M 5) will include hematology and full liver panel only. Additional safety assessments can be performed if required by local regulations; such testing shall be performed at local laboratories. Additional visits may be added if required by local regulations.
Note:
a one-time retest at screening may be performed if an abnormal laboratory test value is considered temporary.
^{k}At intermediate timepoints (W 2, W 5, W 6, W 7, W 9, W 10, W 11, M 7, M 8, M 10, and M 11), only liver function tests will be collected (AST, ALT, albumin, alkaline phosphatase, total and direct bilirubin, total protein) and creatine phosphokinase; these can be performed at central laboratory (preferred, as on-site visits or home nursing as applicable for the site) or at local laboratory. For participants switching to open-label treatment, please refer to Rescue medicine section.
^{l}Complete physical examination due at screening, baseline, yearly, and EOS; brief physical examination is sufficient for the rest of the visits (complete and brief physical examinations will include neurological examination and collection of the following vital signs: arterial blood pressure, heart rate, and temperature).
^{m}To be performed at screening for all participants. To be repeated based on clinical judgment, borderline results or clinical suspicion of TB infection. See E 01, Table 3D for details. Screening tests for TB are described in Table 3F.
ⁿECG and 30-second rhythm strips will be obtained locally.
^{o}Urinalysis will be done as per Table 3F.
^{p}At screening, perform the serum β-HCG pregnancy test at the central laboratory. At randomization and other scheduled visits during the study, urine pregnancy tests should be performed. At randomization, a pregnancy test should be performed prior to the first dose of IMP. Additional serum or urine pregnancy tests may be performed, as determined necessary by the Investigator or required by local regulation, to establish the absence of pregnancy at any time during participant's participation in the study.
^{q}Only in female participants, if needed to establish menopausal status.
^{r}The SDMT and CVLT-II will be performed in all participants. If for some reason, CVLT-II is not available at a given site due to reasons such as lack of translation, local certification, etc., then only the SDMT will be assessed for that participant.
^{s}A subset of sites that have 3T MRI capacity will perform additional sequences (eg, SWI). Further details will be defined in a separate manual. A visit window of ±21 days is acceptable for MRIs performed after D 1.
^{t}A visit window of ±21 days is acceptable for MRIs performed after D 1. The MRI scan should be performed as close as possible before the start of IMP. As much as possible, the screening MRI scan should be performed during the screening period only after it has been established that the participant meets all inclusion and no exclusion criteria.
^{u}MRI scan at EOS visit is mandatory to perform. To avoid unnecessary risk and reduce burden on patient and site, any MRI associated with a scheduled visit within a 3-month period until EOS may be skipped.
^{v}A noninvasive activity monitor (actigraphy) may be optionally implemented by the Sponsor in a subset of participants during the course of the study if results from pilot assessment demonstrate feasibility. Actigraphy baseline assessment will be done at screening.
^{W}When available, clinical outcome assessments are to be completed by the participant prior to discussing their health status and prior to study treatment administration or other study related procedures.
^{X}Where available per local regulations.
^{y}M 6 and M 12: Two samples: one sample between 30 to 90 minutes and one sample between 2.5 to 5 hours after IMP administration. M 9: one sample 30-90 minutes after IMP administration.
^{z}Pharmacodynamics and biomarkers untimed samples will be collected if permitted per local regulations.
^{aa}The DNA testing may be done at any time after signature of consent (in case it could not be done for some reason at Day 1). Participation is optional.
^{bb}This sample will be collected and stored for use if any unexpected safety issue occurs to ensure that a pre-dose baseline value is available for previously not assessed parameters (eg, serology) and for biomarkers research, if agreed.
^{cc}Samples for hematology and biochemistry tests on Day 1 (randomization) will be collected prior to administration of the first IMP dose.
^{dd}Blood sample collection for lymphocyte phenotyping by flow cytometry will be performed in a subset of randomized participants. Participants who did not have a baseline sample collected will no longer have this test performed. Participants who had a baseline sample collected will have a second sample collected at EOT/pEOT.
^{ee}Samples must be shipped within 24 hours.
^{ff}The substudy will be conducted at selected sites.

Objectives and endpoints for the treatment are shown in Table 3B.

TABLE 3B
Objectives and endpoints

| Objectives | Endpoints |
|---|---|
| Primary | |
| To determine the efficacy of tolebrutinib | Time to onset of 6-month cCDP defined |
| compared to placebo in delaying disability | as an increase over at least 6 months of: |
| progression in PPMS | ≥1.0 point from the baseline EDSS |
| | score when the baseline score is ≤5.5, OR |
| | ≥0.5 points when the baseline EDSS |
| | score is >5.5, OR |
| | ≥20% from the baseline timed 25- |
| | foot walk (T25-FW) test, OR |
| | ≥20% from the baseline 9-hole peg |
| | test (9-HPT) |
| Secondary | |
| To evaluate efficacy of tolebrutinib | Time to onset of 6-month CDP as |
| compared to placebo on clinical endpoints, | assessed by the EDSS score defined as an |
| MRI lesions, cognitive performance, | increase over the last 6 months of: |
| physical function, and quality of life | ≥1.0 point from the baseline EDSS |
| | score when the baseline score is ≤5.5, OR |
| | ≥0.5 points when the baseline EDSS |
| | score is >5.5 |
| | Time to onset of 3-month cCDP |
| | Total number of new and/or enlarging |
| | T2-hyperintense lesions as detected by |
| | MRI, defined as the sum of the |
| | individual number of new and/or |
| | enlarging T2 lesions at all scheduled |
| | visits starting after baseline up to and |
| | including the end of study (EOS) visit |
| | Time to onsets of CDI defined as ≥1.0 |
| | point decrease on the EDSS score from |
| | baseline confirmed over at least 6 |
| | months |
| | Percent change in brain volume loss |
| | (BVL) as detected by brain MRI scans |
| | at EOS compared to Month 6 |
| | Change in cognitive function at the |
| | EOS compared to baseline as assessed |
| | by SDMT |
| | Change in cognitive function at EOS |
| | compared to baseline as assessed by |
| | CVLT-II, where available |
| To evaluate safety and tolerability of | Change in MSQoL-54 questionnaire |
| tolebrutinib | score from baseline through the EOS |
| To evaluate population PK of tolebrutinib in | Adverse events (AEs), serious AEs, |
| PPMS and its relationship to efficacy and | AEs leading to permanent study |
| Safety | intervention discontinuation, AEs of |
| | special interest, and potentially |
| | clinically significant abnormalities in |
| | laboratory tests, safety scales, ECG, |
| | and vital signs during the study period |
| To evaluate pharmacodynamics of | Plasma concentration of tolebrutinib |
| tolebrutinib | (population PK assessment) at Months |
| | 6, 9, and 12 |
| | Change in plasma neurofilament light |
| | chain (NfL) levels at the EOS compared |
| | to baseline |
| | Change in lymphocyte phenotype |
| | subsets in whole blood at the |
| | EOS compared to baseline in a subset |
| | of participants |
| | Change in serum immunoglobulin level |
| | at the EOS compared to baseline |
| | Change in serum Chi3L1 levels at the |
| | EOS compared to baseline |
| Tertiary/exploratory | |
| To evaluate efficacy of tolebrutinib on | Time to onset of sustained 20% |
| disease progression and activity in PPMS, | increase in the 9-HPT confirmed over |
| assessed by other clinical and imaging | at least 3 months |
| measures and on physical function | Time to onset of sustained 20% |
| | increase in the T25-FW test confirmed |
| | over at least 3 months |
| | Time to onset of 4-point decrease in the |
| | SDMT confirmed over at least 3 and 6 |
| | months |
| | The proportion of participants with CDI |
| | confirmed over at least 6 months |
| | The proportion of participants with CDI |
| | confirmed over at least 6 months and |
| | maintained until the EOS |
| | Change from baseline to Months 12, |
| | 18, and 24 and to the EOS in the EDSS |
| | score, T25-FW test, 9-HPT, SDMT, |
| | and CVLT-II |
| | Change from baseline to Months 12, |
| | 18, and 24 and to the EOS in modified |
| | Multiple Sclerosis Functional |
| | Composite 3 (MSFC-3), assessed as the |
| | composite of the T25-FW test, 9-HPT, |
| | and SDMT |
| | Proportion of participants with no |
| | evidence of disease activity-3 (NEDA- |
| | 3) at Months 18, 24, 30, 36, and the |
| | EOS |
| | The annualized adjudicated relapse rate |
| | Actigraphic analysis of activity counts |
| | and indices of change from baseline to |
| | the EOS summarized over time (in a |
| | subset of participants) |
| | Change from baseline of total volume |
| | of T2-hyperintense lesions as detected |
| | by brain MRI at Months 18, 24, and the |
| | EOS |
| | Total number of new gadolinium- (Gd-) |
| | enhancing T1-hyperintense lesions as |
| | detected by MRI, defined as the sum of |
| | the individual number of new |
| | Gd-enhancing T1-hyperintense lesions |
| | at all scheduled visits starting after |
| | baseline up to and including the EOS |
| | visit |
| | Change from baseline by visit over time |
| | in volume of T1-hypointense lesions |
| | and cumulative number of new |
| | T1-hypointense lesions |
| | Magnetization transfer ratio (MTR) |
| | recovery at EOS in new MTR lesions |
| | detected at Months 6 and 12 |
| | Number and volume of slowly evolving |
| | lesions (SELs) |
| | Normalized T1 (nT1) intensity |
| | evolution in SELs |
| | Change in number of phase rim lesions |
| | in SWI MRI from baseline by visit over |
| | time (subset of centers with capacity of |
| | 3T MRI) |
| To evaluate the treatment effect of | Change in EuroQol 5-dimension 5-level |
| tolebrutinib via changes in participants' | questionnaire (EQ-5D-5L) from |
| health-related quality of life (HRQoL) | baseline by visit over time |

Abbreviations: 3M-cCDP, 3-month composite confirmed disability progression; 6M-cCDP, 6-month composite confirmed disability progression; 9-HPT, 9-hole peg test; AE, adverse event; BVL, brain volume loss; CDI, confirmed disability improvement; CDP, confirmed disability progression; Chi3L1, chitinase-3-like protein 1; CVLT-II, California Verbal Learning Test-II; ECG, electrocardiogram; EDSS, Expanded Disability Status Scale; EQ-5D-5L, EuroQol 5-dimension 5-level questionnaire; EOS, end-of-study; Gd, gadolinium; HRQoL, health related quality of life; MRI, magnetic resonance imaging; MTR, magnetization transfer ratio; MSFC-3, Multiple Sclerosis Functional Composite 3; MSQoL-54, Multiple Sclerosis Quality of Life-54 Questionnaire; NEDA-3, no evidence of disease activity-3; NfL, neurofilament light chain; PK, pharmacokinetic; PPMS, primary progressive multiple sclerosis; SDMT, Symbol Digit Modalities test; SWI, susceptibility weighted imaging; T25-FW, timed 25 foot walk.

### Dose Regimen

The choice of the dose of 60 mg of the BTK inhibitor taken with food is based on the results of a Phase 2b dose-finding trial for tolebrutinib in participants with relapsing multiple sclerosis (DRI15928). See for example, Reich, D. S., et al., Safety and efficacy of tolebrutinib, an oral brain-penetrant BTK inhibitor, in relapsing multiple sclerosis: a phase 2b, randomised, double-blind, placebo-controlled trial. Lancet Neurol, 2021. 20(9): p. 729-738.

Analysis of the PK data and effect of fed status on tolebrutinib exposure showed a positive food effect with an increase in AUC₀₋₂₄ of approximately 2-fold. Moreover, the correlation between the treatment response and the exposure to tolebrutinib showed that higher exposure was associated with low numbers of new gadolinium-enhancing T1-hyperintense lesions after 12 weeks of treatment. Taken together, these data support the recommendation to take tolebrutinib with food.

There was no correlation between the dose of tolebrutinib administered and the number of TEAEs. The most common events (preferred terms) observed in participants in the tolebrutinib treatment arms were headache, upper respiratory tract infection, and nasopharyngitis. There were low numbers of AESIs and PCSAs observed. Overall, no new risks were identified in this trial.

### Overall Design:

This is a Phase 3, randomized, double-blind, 2-arm, placebo-controlled, parallel-group, multicenter, event-driven (6M-cCDP) trial with a variable treatment duration ranging from approximately 12 to 60 months in participants with PPMS.

### Disclosure Statement:

This is a parallel treatment study with 2 arms that is blinded/masked for participants, any Investigator, site staff, and the Sponsor.

### Number of Participants:

Approximately 700 participants will be randomly assigned to study intervention (2:1 randomization ratio of tolebrutinib to placebo).

### Intervention Groups and Duration:

Participants will be randomly assigned at a 2:1 ratio to receive 60 mg of oral, daily tolebrutinib or daily matching placebo. Randomization will be stratified by age at screening (>40 versus ≤40 years), geographic region (United States [US] versus non-US) and PPMS McDonald diagnosis criteria (2005 [Polman et al. Ann Neurol. 2005, 58, 840] and 2017 version versus 2017 version [Thompson et al. Lancet Neurol. 2018, 17, 162]).

### Study Intervention(s):

### Investigational Medicinal Product(s)

Formulation: tolebrutinib film coated tablet
Route(s) of administration: oral
Dose regimen: 60 mg once daily

### Investigational Medicinal Product(s)

Formulation: placebo to match tolebrutinib film coated tablet
Route of administration: oral
Dose regimen: once daily

### Noninvestigational Medicinal Products(s)

Formulation: MRI contrast-enhancing preparations
Route(s) of administration: Intravenous (IV)
Dose regimen: per respective label

### Temporary IMP Interruption Due to Surgery

If surgery is needed during the study, consider the benefit-risk of withholding the IMP for at least 3 to 7 days pre- and post-surgery, depending upon the type of surgery and the risk of bleeding.

The goal of this Phase 3 clinical trial is to demonstrate the efficacy and safety of tolebrutinib compared to placebo in participants with PPMS. Six-month composite CDP was selected as the primary endpoint in this trial. It is being increasingly used in MS clinical trials to enhance the sensitivity of measuring disability progression (Cadavid et al. Multiple Sclerosis Journal. 2017; 23(1):94-105; A Study to Evaluate the Efficacy and Safety of Fenebrutinib Compared With Ocrelizumab in Adult Participants With Primary Progressive Multiple Sclerosis, ClinicalTrials.gov).

Six-month CDP as assessed by EDSS score, 3M-cCDP, and confirmed disability improvement (CDI) over at least 6 months measured by the EDSS score will also be used as secondary endpoints due to their clinical importance.

Magnetic resonance imaging outcomes will include change in brain volume, which is considered as a marker of the CNS degenerative process and is therefore recommended for use in progressive MS trials (European Medicines Agency (EMA). Committee for Medicinal Products for Human Use (CHMP). Guideline on clinical investigation of medicinal products for the treatment of multiple sclerosis. London, 26 Mar. 2015).

Population pharmacokinetics (PK) will be performed in order to obtain a larger and more diverse population to evaluate the PK of tolebrutinib in the PPMS population, to assess sources of PK variability (ethnicity, special populations), and to establish exposure correlation to clinical efficacy, biomarkers, and safety endpoints. Analysis of the population PK data may be performed as soon as all participants have finished the Month 12 visit (a separate unblinded team will be used to preserve blinding integrity).

Multiple sclerosis relapses may occur infrequently in the PPMS population (Montalban et al. N Engl J Med. 2017; 376(3):209-20; Kappos et al. Lancet. 2018; 391(10127):1263-73; Kappos et al. Neurology. 2017; 88 (Suppl 16):CT.002). Relapse data will be carefully collected and adjudicated, and their impact on the primary endpoint will be assessed (see also statistical considerations).

Exploratory assessments are expected to provide additional evidence for tolebrutinib activity on neuroinflammation and neurodegeneration.

In the ongoing Phase 3 and LTS studies, tolebrutinib has been generally well tolerated to date. An identified risk for tolebrutinib has been identified as follows:
Treatment-emergent SAEs of drug-induced liver injury (DILI) were reported in the ongoing Phase 3 trials; however, all cases occurred between Months 2 to 3 and appear reversible after treatment discontinuation, with potential confounders identified for some of the cases.

### End of Study Definition

A participant is considered to have completed the study if he/she has completed all periods of the study including the EOS Visit, whether remaining on IMP or not.

This study will end when approximately 360 primary endpoint events of 6M-cCDP have been observed. Considering the recruitment period of approximately 44 months and study assumptions, the duration of the study is estimated to be approximately 60 months. The event rates based on the blinded data will be regularly monitored to project a date when it is likely the required number of events will have occurred in order to operationally plan for study end.

### Study Population

### Inclusion Criteria

Participants are eligible to be included in the study only if all of the following criteria apply as shown in Table 3C.

TABLE 3C
Inclusion Criteria

| Category | Criteria | |
|---|---|---|
| Age | I 01. | The participant must be 18 to 85 years of age inclusive, at the time |
| | | of signing the informed consent. |
| Type of | I 02. | The participant must have a current diagnosis of PPMS in |
| participant | | accordance with the 2017 revision of the McDonald criteria |
| and disease | | (Thompson et al. Lancet Neurol. 2018, 17, 162). |
| characteristics | I 03. | The participant must have an EDSS score at screening from 2.0 to |
| | | 6.5 points, inclusive. |
| | I 04. | The participant must have positive CSF (isoelectric focusing |
| | | evidence of oligoclonal bands and/or elevated IgG index) either |
| | | during screening or previous historical assessment. Supportive |
| | | source documentation must be available. |
| Weight | I 05. | Not Applicable. |
| Sex | I 06. | Male or Female. |

Contraceptive use should be consistent with local regulations regarding
the methods of contraception for those participating in clinical studies.

| | |
|---|---|
| a) | Male participants |

| | |
|---|---|
| - | Not applicable |

| | |
|---|---|
| b) | Female participants |

| | | • | A female participant is eligible to participate if she is not |
|---|---|---|---|
| | | | pregnant or breastfeeding, and at least one of the following |
| | | | conditions applies: |
| | | - | Is not a woman of childbearing potential (WOCBP); or |
| | | - | Is a WOCBP and agrees to use an acceptable contraceptive |
| | | | method during the intervention period (at a minimum until |
| | | | after the last dose of study intervention). |
| | | - | A WOCBP must have a negative highly sensitive pregnancy |
| | | | test (urine or serum as required by local regulations) at |
| | | | screening before the first dose of study intervention. If a urine |
| | | | test cannot be confirmed as negative (e.g., an ambiguous |
| | | | result), a serum pregnancy test is required. In such cases, the |
| | | | participant must be excluded from participation if the serum |
| | | | pregnancy result is positive. |
| | | • | Requirements for pregnancy testing during and after study |
| | | | intervention are located in the schedule of activities (SoA; |
| | | | Table 3A). |
| | | • | The Investigator is responsible for review of medical history, |
| | | | menstrual history, and recent sexual activity to decrease the |
| | | | risk for inclusion of a woman with an early undetected |
| | | | pregnancy, if allowed by local regulations. |
| Informed | I 07. | The participant must have given written informed consent prior to | |
| Consent | | undertaking any study related procedure. This includes consent to | |
| | | comply with the requirements and restrictions listed in the | |
| | | informed consent form (ICF) and in this protocol In countries | |
| | | where the legal age of maturity is greater than 18 years, a specific | |
| | | ICF for such legally minor participants must also be signed by the | |
| | | participant's legally authorized representative. | |
| Other inclusion | I 08. | The participant must: | |
| criteria added | | • | Not have access to ocrelizumab (eg, ocrelizumab not available |

| in protocol | | on the national market or not reimbursed for the approved |
|---|---|---|
| amendments | | indication). |
| | • | Have access to and be eligible to be treated with ocrelizumab |

but: 1) does not tolerate it due to side effects or safety reasons;
and/or 2) has failed ocrelizumab treatment due to perceived
lack of efficacy; this reason must be noted in the patient record.

### Exclusion Criteria

Participants are excluded from the study if any of the following criteria apply as shown in Table 3D.

TABLE 3D
Exclusion Criteria

| Category | Criteria | |
|---|---|---|
| Medical conditions | E 01. The participant has a history of infection or may be at risk for | |
| | infection: | |
| | A history of T-lymphocyte or T-lymphocyte-receptor | |
| | vaccination, transplantation (including solid organ, | |
| | stem cell, and bone marrow transplantation) and/or | |
| | antirejection therapy. | |
| | The participant has received any live (attenuated) | |
| | vaccine (including but not limited to varicella zoster, | |
| | oral polio, and nasal influenza) within 2 months | |
| | before the first treatment visit. | |
| | The participant has a lymphocyte count less than the | |
| | lower limit of normal (LLN) at the Screening Visit. | |
| | A history of diagnosis of progressive multifocal | |
| | leukoencephalopathy (PML) or evidence of findings | |
| | suggestive of PML on the screening MRI. | |
| | A history of infection with human immunodeficiency | |
| | virus (HIV) (e.g., any known positive HIV test or | |
| | information from participant interview). | |
| | A history of active or latent tuberculosis (TB); TB | |
| | testing should be performed at screening and again | |
| | during the study, if clinically indicated, and may be | |
| | repeated based on clinical judgment, borderline | |
| | results, or clinical suspicion of TB infection. | |
| | Screening tests for TB are described in Table 3F. | |
| | NOTE: | |
| | The Investigator may consult with an | |
| | infectious disease expert if required, e.g., test results | |
| | are unclear or there is a suspicion of false positive test | |
| | results. If the infectious disease expert considers the | |
| | test results as false positive and not clinically relevant | |
| | and confirms that the participant can be enrolled in | |
| | the trial, the Investigator must document this in | |
| | source data and may then randomize the participant. | |
| | Persistent chronic or active recurring infection | |
| | requiring treatment with antibiotics, antivirals, or | |
| | antifungals. | |
| | Fever within 4 weeks of the Screening Visit (≥38° C.; | |
| | however, if due to brief and mild ear, nose, throat | |
| | viral infection participant may be included based on | |
| | the Investigator's judgment). | |
| | Participants at risk of developing or having | |
| | reactivation of hepatitis, ie, results at screening for | |
| | serological markers for hepatitis B and C viruses | |
| | indicating acute or chronic infection. See the Study | |
| | Manual for further details. | |
| | E 02. The presence of psychiatric disturbance or substance abuse as | |
| | evidenced by: | |
| | A history of any psychiatric disease, behavioral | |
| | condition, or depression requiring hospitalization | |
| | within 2 years prior to the Screening Visit. | |
| | A documented history of attempted suicide or suicidal | |
| | ideation of category 4 or 5 according to the Columbia | |
| | Suicide Severity Rating Scale (C-SSRS) | |
| | baseline/screening version over the 6 months prior to | |
| | the Screening Visit, OR if in the Investigator's | |
| | judgment, the participant is at risk for a suicide | |
| | attempt. | |
| | Active alcohol use disorder or a history of alcohol or | |
| | drug abuse within 1 year prior to the Screening Visit. | |
| | Current alcohol intake >2 drinks per day for men and | |
| | >1 drink per day for women (1 drink = approximately | |
| | 14 grams of alcohol = 350 mL beer = 140 mL wine = | |
| | 40 mL of spirits). | |
| | E 03. The following findings obtained during the Screening Visit | |
| | considered in the Investigator's judgment to be clinically | |
| | significant in the context of this clinical trial: | |
| | Any screening laboratory values outside normal | |
| | limits. | |
| | Abnormal ECG. | |
| | E 04. Conditions that may predispose the participant to excessive | |
| | bleeding: | |
| | A bleeding disorder or known platelet dysfunction at | |
| | any time prior to the screening visit. | |
| | A platelet count <150 000/μL at the screening visit. | |
| | The participant has had major surgery within 4 weeks | |
| | prior to the screening visit, which could affect the | |
| | participant's safety (as judged by the Investigator) or | |
| | has planned any elective major surgery during the | |
| | study. | |
| | A history of significant bleeding event within 6 | |
| | months prior to screening, according to the | |
| | Investigator's judgment such as, but not limited to | |
| | cerebral or gastrointestinal bleeding. | |
| | E 05. Conditions that would adversely affect participation in the | |
| | study or make the primary efficacy endpoint non-evaluable: | |
| | A short life expectancy due to pre-existing | |
| | health condition(s) as determined by their | |
| | treating neurologist. | |
| | A history or presence of significant other | |
| | concomitant illness according to the | |
| | Investigator's judgment such as, but not | |
| | limited to cardiovascular (including Stage III | |
| | or IV cardiac failure according to New York | |
| | Heart Association [NYHA] classification), or | |
| | renal (i.e., undergoing dialysis), | |
| | neurological, endocrine, gastrointestinal, | |
| | metabolic, pulmonary (e.g., interstitial | |
| | pneumonia or pulmonary fibrosis), or | |
| | lymphatic disease that would adversely | |
| | affect participation in this study. | |
| | Acute liver disease, cirrhosis, chronic liver | |
| | disease (unless considered stable for >6 months). | |
| | Confirmed screening ALT >1.5 × ULN OR | |
| | AST >1.5 × ULN OR alkaline phosphatase >2 × ULN | |
| | (unless caused by non-liver-related disorder or | |
| | explained by a stable chronic liver disorder) OR total | |
| | bilirubin >1.5 × ULN (unless due to Gilbert syndrome | |
| | or non-liver-related disorder). | |
| | At screening, elevated transferrin saturation (>50% in | |
| | males and >40% in females) and/or with elevated | |
| | ferritin levels >500 μg/L. | |
| | Any malignancy within 5 years prior to the | |
| | Screening Visit (except for effectively | |
| | treated carcinoma in situ of the cervix or | |
| | adequately treated non-metastatic squamous | |
| | or basal cell carcinoma of the skin) will also | |
| | be exclusionary. | |
| | Any other medical condition(s) or | |
| | concomitant disease(s) making them non- | |
| | evaluable for the primary efficacy endpoint | |
| | or that would adversely affect participation | |
| | in this study, as judged by the Investigator. | |
| Prior/concomitant | E 06. The participant has received any of the following | |
| therapy | medications/treatments within the specified time frame before | |
| | any randomization assessment (no washout is required for | |
| | dimethyl fumarate, interferon beta, or glatiramer acetate | |
| | treatments although use is not permitted on or after Day 1): | |
| | | Exclusionary if |
| | | used/used within |
| | | required wash- |
| | Medication | out period |
| | Systemic corticosteroids, | 1 month prior to |
| | adrenocorticotropic hormone | screening MRI |
| | | scan |
| | Siponimod, ponesimod | 1 week before |
| | | randomization with |
| | | MRI and clinical |
| | | assessment for |
| | | PML |
| | Plasma exchange | 1 month prior to |
| | | randomization |
| | IV immunoglobulin | 2 months prior to |
| | | randomization |
| | Fingolimod, ozanimod | 6 weeks before |
| | | randomization |
| | | with MRI and |
| | | clinical |
| | | assessment for |
| | | PML |
| | Natalizumab | 2 months before |
| | | randomization |
| | | with MRI and |
| | | clinical |
| | | assessments for |
| | | PML |
| | Teriflunomide^{a}, mildly to moderately | 3 months prior to |
| | immunosuppressive/chemotherapeutic | randomization |
| | medications such as azathioprine and | |
| | methotrexate, mycophenolate | |
| | Lymphoid irradiation, bone marrow | A participant who |
| | transplantation, mitoxantrone (with | has received any |
| | evidence of cardiotoxicity following | of these |
| | treatment, or cumulative lifetime | treatments at any |
| | dose >120 mg/m²), other strongly | time is not |
| | immunosuppressive treatments with | eligible. |
| | very long-lasting effects | |
| | Ofatumumab | 4 months prior |
| | B-cell-depleting therapies such as | 6 months prior to |
| | ocrelizumab and rituximab | randomization |
| | Highly | 2 years prior to |
| | immunosuppressive/chemotherapeutic | randomization |
| | medications: mitoxantrone up to 120 | |
| | mg/m2 body surface area, | |
| | cyclophosphamide, cladribine, | |
| | cyclosporine | |
| | Alemtuzumab | 4 years prior to |
| | | randomization |
| | Other MS-disease modifying | 5 half-lives or until |
| | treatments | end of |
| | | pharmacodynamics |
| | | activity, whichever |
| | | is longer |

| | |
|---|---|
| | Abbreviations: IV: intravenous MRI: magnetic resonance imaging, |
| | MS: multiple sclerosis, PML: progressive multifocal |
| | leukoencephalopathy |
| | ^{a}No time restriction if accelerated elimination procedure is |
| | done |
| | E 07. The participant is receiving potent and moderate inducers of |
| | cytochrome P450 3A (CYP3A) or potent inhibitors of |
| | CYP2C8 hepatic enzymes. |
| | E 08. The participant is receiving anticoagulant/antiplatelet |
| | therapies, including: |
| | Acetylsalicylic acid (aspirin) >81 mg/day, |
| | Antiplatelet drugs (eg, clopidogrel), |
| | Warfarin (vitamin K antagonist), |
| | Heparin, including low molecular weight heparin |
| | (antithrombin agents), |
| | Dabigatran (direct thrombin inhibitor), |
| | Apixaban, edoxaban, rivaroxaban (direct factor Xa |
| | inhibitors). |
| | Note: |
| | All the above-mentioned drugs must be stopped at least 5 half- |
| | lives before study drug administration except for aspirin, which must |
| | be stopped at least 8 days before. If this is not clinically appropriate, |
| | the participant cannot be included. These washout periods are only |
| | applicable in the case that the Investigator deems it clinically |
| | appropriate to discontinue the listed medications or there is a recent |
| | history of use of these medications (as in the case when short term |
| | treatment with anticoagulants is clinically recommended for certain |
| | thrombotic events), and therefore these washout periods will need to |
| | be followed prior to randomization. |
| | If the participant has a chronic underlying medical condition (stroke, |
| | coronary or carotid artery disease, heart valvular disease etc.) |
| | requiring continued use of these medications, the participant cannot |
| | be enrolled in the study. |
| | E 09. The participant has sensitivity to any of the study |
| | interventions, or components thereof, or has a drug or other |
| | allergy that, in the opinion of the Investigator, contraindicates |
| | participation in the study. |
| Prior/concurrent | E 10. The participant was previously exposed to any BTK inhibitor, |
| clinical study | including tolebrutinib. |
| experience | |
| | E 11. The participant has taken other investigational drugs within 3 |
| | months or 5 half-lives, whichever is longer, before the |
| | screening visit. |
| Diagnostic | E 12. The participant has a contraindication for MRI, i.e., presence |
| assessments | of pacemaker, metallic implants in high-risk areas (i.e., |
| | artificial heart valves, aneurysm/vessel clips), presence of |
| | metallic material (e.g., shrapnel) in high risk areas, known |
| | history of allergy to any contrast medium, or history of |
| | claustrophobia that would prevent completion of all protocol |
| | scheduled MRI. |
| | Note: |
| | People with a contraindication to Gd can be enrolled |
| | into the study but cannot receive Gd contrast dyes during their |
| | MRI scan. |
| Other exclusions | E 13. Individuals accommodated in an institution because of |
| | regulatory or legal order; prisoners or participants who are |
| | legally institutionalized. |
| | E 14. Participant not suitable for participation, whatever the reason, |
| | as judged by the Investigator, including medical or clinical |
| | conditions, or participants potentially at risk of noncompliance |
| | to study procedures or not able to follow the schedule of |
| | protocol assessments due to other reasons. |
| | E 15. Participants are employees of the clinical study site or other |
| | individuals directly involved in the conduct of the study, or |
| | immediate family members of such individuals. |
| | E 16. Any other situation during study implementation/course that |
| | may raise ethics considerations. |
| | Note: |
| | a one-time retest at screening may be performed if an |
| | abnormal laboratory test value is considered temporary |

### Lifestyle Considerations

Meals and dietary restrictions: tolebrutinib (IMP) shall be taken with a regular meal. When possible, the meal with which IMP is taken (e.g., breakfast, lunch, or dinner) should be consistent throughout the study. The typical meal with which the IMP is taken will be recorded at each visit. In case the mealtime for IMP administration needs to be changed, a gap of a minimum of 12 hours between 2 doses should be maintained.

### Caffeine, Alcohol, and Tobacco:

For each visit with PK/PD assessment, participants will abstain from ingesting caffeine- or xanthine-containing products (e.g., coffee, tea, cola drinks, and chocolate) for 2 hours before the start of treatment until after collection of the final PK and/or PD sample later that day.

For each visit with PK/PD assessment, participants will abstain from alcohol for 24 hours before the start of treatment until after collection of the final PK and/or PD sample later that day.

During the entire study, participants should be warned not to consume substantial quantities of alcohol, defined as >14 grams (1 standard drink) per day in female participants or >28 grams (2 standard drinks) per day in male participants on a regular basis.

### Study Intervention(s) and Concomitant Therapy

Study intervention is defined as any investigational intervention(s), marketed product(s), placebo, or medical device(s) intended to be administered to a study participant according to the study protocol. An overview of the study interventions administered is provided in Table 3E.

TABLE 3E
Overview of study interventions administered

| | | |
|---|---|---|
| ARM name | tolebrutinib | Placebo |
| Intervention name | tolebrutinib 60 mg | Placebo |
| Type | Drug | Drug |
| Dose formulation | film-coated tablet | film-coated tablet |
| Unit dose strength(s) | 60 mg | 0 mg |
| Dosage level(s) | Once daily | Once daily |
| Route of administration | Oral | Oral |
| IMP and NIMP | IMP | IMP |
| Packaging and labeling | Study intervention will be | Study intervention will be |
| | provided in wallet blister | provided in wallet blister |
| | packaging. The content of | packaging. The content of |
| | the labeling is in accordance | the labeling is in accordance |
| | with the local regulatory | with the local regulatory |
| | specifications and | specifications and |
| | requirements. | requirements. |
| Current name | Tolebrutininb | Not applicable |

### Dose Modification

Dose reduction is not foreseen in this study. Treatment may need to be interrupted or permanently discontinued if deemed necessary due to an AE.

### Concomitant Therapy

Any medication or vaccine (including over-the-counter or prescription medicines, vitamins, and/or herbal supplements) that the participant is receiving at the time of enrollment or receives during the study must be recorded along with:
Reason for use.
Dates of administration including start and end dates.
Dosage information including dose and frequency.

Live (attenuated) vaccines should not be administered during the intervention period. Therapies for MS noted in the exclusion criterion E06 are not permitted after randomization while the participant is on study treatment. Short-term use (3 to 5 days) of glucocorticoids (eg, for MS relapse treatment or an acute illness) and local corticosteroids (eg, topical, nasal, ocular, otic, intra-articular) are allowed.

The Medical Monitor should be contacted if there are any questions regarding concomitant or prior therapy.

Participants must abstain from taking prescription or nonprescription drugs (including vitamins and dietary or herbal supplements) within 7 days (or 14 days if the drug is a potential enzyme inducer) or 5 half-lives (whichever is longer) before the start of study intervention until completion of the follow-up visit, unless, in the opinion of the Investigator and Sponsor, the medication will not interfere with the study. Medications for MS treatment of MS symptoms (e.g., walking impairment, fatigue, spasticity, incontinence, pain) should be maintained at a stable dose prior to screening and for the duration of the treatment period, if clinically feasible.

Anticoagulant/antiplatelet therapies are not permitted to be taken concomitantly with the IMP, including:
Acetylsalicylic acid (aspirin) >81 mg/day.
Antiplatelet drugs (e.g., clopidogrel).
Warfarin (vitamin K antagonist).
Heparin, including low molecular weight heparin (antithrombin agents).
Dabigatran (direct thrombin inhibitor).
Apixaban, edoxaban, rivaroxaban (direct factor Xa inhibitors).

Paracetamol/acetaminophen, at doses of ≤3 grams/day, is permitted for use at any time during the study. Short courses (up to 5 days) of nonsteroidal anti-inflammatory drugs (NSAIDs) (other than acetylsalicylic acid), preferably selective cyclooxygenase-2 inhibitors at the lowest effective dose, may be given during the study if clinically necessary for the treatment of an existing medical condition or a new event. The Investigator must record the use of NSAIDs (and any other comedication) in the eCRF.

### CYP Inhibitor/Inducer:

Potent and moderate inducers of CYP3A or potent inhibitors of CYP2C8 hepatic enzymes are not allowed throughout the study (Tables 3H-A and 3H-B). Based on nonclinical drug metabolism studies, tolebrutinib is a substrate of the CYP3A4 and CYP2C8 isoenzymes. In healthy participants, potent CYP3A4 inhibitor (itraconazole 200 mg once daily for 4 days) increased tolebrutinib area under the curve (AUC) exposure 1.8-fold (INT16385 study) and potent CYP2C8 inhibitor (gemfibrozil 600 mg twice daily for 6 days) increased tolebrutinib AUC exposure 8.4-fold (INT16726 study). Based on a satisfactory safety and tolerability profile and on the observed exposure in healthy participants who received tolebrutinib at a dose of up to 240 mg tolebrutinib once daily for 14 days under fed conditions (TDR16862 study), drugs that strongly inhibit CYP3A4 are allowed and drugs that strongly inhibit CYP2C8 are not permitted. In healthy participants, potent CYP3A4 and moderate CYP2C8 induction by rifampicin (600 mg once daily for 8 days) decreased tolebrutinib exposure 6-fold (INT16726 study). Therefore, potent and also moderate (based on prediction) CYP3A inducers are not permitted due to their potential to decrease tolebrutinib exposure and efficacy. See Tables 3H-A and 3H-B for the list of drugs to be avoided.

### Open-Label Treatment

If a participant achieves the criterion of 6-month CDP as assessed by EDSS, they may, in conjunction with the Treating Investigator, choose to receive one of the following:
1. To switch to open-label tolebrutinib treatment; OR
2. To switch to a non-study treatment approved for PPMS in their respective country. In this case, the participant will permanently discontinue the IMP and will be encouraged to remain in the study for planned clinical visits until common study end.

In case the participant switches to open-label tolebrutinib treatment, he/she will need to be monitored for liver function tests after the first open-label dose at Weeks 2, 4, 5, 6, 7, 8, 9, 10, 11, and 12, and then monthly for the next 9 months. After that, the schedule visits timepoints per SoA (Table 3A) will be resumed (i.e., every 3 months), until the common study end. Whenever a timepoint will coincide with a scheduled visit as per SoA (Table 3A), the full scheduled visit assessments will be performed instead of the liver monitoring testing alone.

For participants achieving 6-month CDP as assessed by EDSS, the Investigator must ensure that the participant does not meet any of the following criteria prior to the switch to open label tolebrutinib treatment:
Current alcohol intake >2 drinks per day for men and >1 drink per day for women (1 drink=approximately 14 grams of alcohol=350 mL beer=140 mL wine=40 mL of spirits).
Acute liver disease, cirrhosis, chronic liver disease (unless considered stable for >6 months).
Confirmed ALT>1.5×ULN OR AST>1.5×ULN OR ALP>2×ULN (unless caused by non-liver-related disorder or explained by a stable chronic liver disorder) OR total bilirubin >1.5×ULN (unless due to Gilbert syndrome or non-liver-related disorder).

Liver function tests will be performed for all participants prior to the start of open-label treatment.

Should the participant and Investigator opt for the provision of open-label medicine, they will remain blinded to the original treatment assignment.

All individual blinded data will be reviewed and all queries resolved, if possible, before the switch to the chosen rescue approach to ensure data integrity for primary assessment. Prior to initiation of rescue treatment, the Investigator shall confirm that there has been no adjudicated relapse within 90 days prior to the onset or confirmation of 6-month CDP. Based on individual symptoms and assessed risk of further progression, the Investigator and participant may choose for the participant to remain on the initial double-blind treatment after achieving 6-month CDP.

### Discontinuation of Study Intervention and Participant Discontinuation/Withdrawal

### Discontinuation of Study Intervention

Definitive discontinuation

The study intervention should be continued whenever possible.

Permanent intervention discontinuation is any treatment discontinuation associated with the definitive decision by the Investigator or the participant not to re-expose the participant to study intervention at any time. In rare instances, it may be necessary for a participant to permanently discontinue study intervention. If study intervention is permanently discontinued, the participant shall be asked to remain in the study to be evaluated until the follow-up/EOS Visit. For this set of participants (participants who discontinued the IMP and/or switched to another DMT), no PK or biomarker samples will be collected after the pEOT visit, and MRI assessments will be performed once annually using the next annual visit as the starting point. This will be important to continue to evaluate for safety and efficacy. See the SoA (Table 3A) for data to be collected at the time of discontinuation of study intervention. In the case that the study intervention is permanently discontinued, the participant should be treated for MS according to local clinical practice and the best judgment of the Treating Investigator.

The following may be justifiable reasons for the Investigator or Sponsor to discontinue a participant from treatment:
Adverse events that endanger the safety of the participant, or if discontinuation of study intervention is desired or considered necessary by the Investigator and/or participant.
If IMP discontinuation criteria are met as per the guidance for the follow up of laboratory abnormalities (Table 3F).
The participant is no longer deriving a therapeutic/clinical benefit in the opinion of the Investigator.
If a female participant becomes pregnant or wishes to become pregnant during the study.
At participant's request, i.e., withdrawal of the consent for treatment.
Any serious opportunistic infections (e.g., PML [see FIG. 7], HIV)
Continued need for/chronic use of prohibited a concomitant medication (see “Concomitant Therapy”, Table 3H-A, Table 3H-B, “Examples of Drugs with a Potential to Change Teriflunomide Disposition”, and “Examples of Drugs Which Can Be Potentially Affected by Teriflunomide” of this Example).
Use of open-label tolebrutinib or non-study disease modifying therapy approved for PPMS in their respective countries (e.g., after 6-month CDP).
A brief physical examination will include, at a minimum, assessments of the skin, lungs, cardiovascular system, neurological examination, and abdomen (liver and spleen).
Investigators should pay special attention to clinical signs related to previous serious illnesses.
Any clinically significant new finding or worsening of previous finding should be reported as an AE, per Investigator's judgement.

Discontinuation of study intervention for abnormal liver function should be considered by the Investigator when a participant meets one of the conditions outlined in the algorithm (e.g., FIGS. 2-7) or if the Investigator believes that it is in the best interest of the participant.

### Clinical Laboratory Tests

The tests detailed in Table 3F will be performed by the central laboratory, when feasible. Local laboratory results are only required in the event that the central laboratory results are not available in time for either study intervention administration and/or response evaluation. Additionally, if the local laboratory results are used to make either a study intervention decision or response evaluation, the results must be entered into the eCRF.

Protocol-specific requirements for inclusion or exclusion of participants are detailed in Tables 3C and 3D of the protocol.

Additional tests may be performed at any time during the study as determined necessary by the Investigator or required by local regulations. Additional serum or urine pregnancy tests may be performed, as determined necessary by the Investigator or required by local regulation, to establish the absence of pregnancy at any time during the subject's participation in the study.

TABLE 3F
Protocol-required laboratory assessments

| Laboratory | | | |
|---|---|---|---|
| assessments | Parameters | | |
| Hematology | Platelet count | RBC indices: | WBC count with differential: |
| | RBC count | MCV | Neutrophils |
| | Hemoglobin | MCH | Lymphocytes |
| | Hematocrit | % Reticulocytes | Monocytes |
| | | | Eosinophils |
| | | | Basophils |
| Clinical | BUN | Sodium | AST |
| chemistry ^{a} | Creatinine | Calcium | ALT |
| | Glucose | Total and direct bilirubin | Alkaline phosphatase |
| | Potassium | Total protein | Albumin |
| | | Chloride | Creatine phosphokinase |
| | | Bicarbonate | Lipase |

| | |
|---|---|
| Routine | - Specific gravity |
| urinalysis | - pH, glucose, protein, blood, ketones, bilirubin, urobilinogen, nitrite, leukocyte |
| | esterase by dipstick |
| | - Microscopic examination (if blood or protein is abnormal and for signs of |
| | infection) |
| Other | - FSH and estradiol (if needed, only in female participants to confirm |
| screening | postmenopausal state) |
| tests | - Highly sensitive serum or urine β-hCG pregnancy test (as needed for women of |
| | childbearing potential) ^{b} |
| | - Coagulation: PT/INR, aPTT |
| | - Serology tests for hepatitis B (HBs Ag, anti-HBc IGM and total, anti-HBs) and C |
| | virus (anti-HCV); in case these results are inconclusive (e.g. anti-HBs negative and |
| | anti-HBc positive or anti-HC IgG positive), HBV-DNA or HCV-RNA testing, |
| | respectively, should be performed for confirmation. HIV and other infectious |
| | diseases, if locally required. |
| | - Tuberculosis test: Blood testing (e.g., QuantiFERON ® TB Gold test) is preferred; |
| | skin testing (e.g., tuberculin skin test) with ancillary testing will be allowed if |
| | blood testing is not available. T-SPOT can also be performed, if available. ^{c} |
| | - Iron panel (serum): iron, ferritin, transferrin saturation TIBC. |

ALT: alanine aminotransferase; anti-HBc; antibody to hepatitis B core antigen; anti-HBs: hepatitis B surface antibody; aPTT: activated partial thromboplastin time; AST: aspartate aminotransferase; BUN: blood urea nitrogen; β-hCG: human chorionic gonadotropin; FSH; follicle stimulating hormone; IEC: independent ethics committee; INR: international normalized ratio; HBsAg: hepatitis B surface antigen; HBV: hepatitis B virus; HCV: hepatitis C virus; HIV: human immunodeficiency virus; Ig: immunoglobulin; IRB: institutional review board; MCH: mean corpuscular hemoglobin; MCV: mean corpuscular volume; PT: prothrombin time; RBC: red blood cell; SGOT: serum glutamic-oxaloacetic transaminase; SGPT: serum glutamic-pyruvic transaminase; TB: tuberculosis; TIBC: total iron-binding capacity; ULN: upper limit of normal; WBC: white blood cell.
^{a} Details of liver chemistry stopping criteria and required actions and follow-up assessments after observations of ALT >3 × ULN are given in FIG. 4. Clinical laboratory findings of ALT >3 × ULN and bilirubin ≥2 × ULN (>35% direct bilirubin), or ALT >3 × ULN and international normalized ratio (INR) >1.5, if INR measured, that may suggest severe liver injury and must be reported as an SAE.
^{b} Local urine testing for pregnancy will be standard for the protocol (except for the Screening Visit when a serum pregnancy test is required) unless serum testing is required by local regulation or IRB/IEC.
^{c} Further details are provided in E 01, Table 3D.

Investigators must document their review of each laboratory safety report.

Laboratory/analyte results that could unblind the study will not be reported to investigative sites or other blinded personnel until the study has been unblinded. This includes PK assessments and any post-baseline biomarker or PD assessments.

### Liver and Other Safety: Actions and Follow-Up Assessments

These actions described in Table 3G-A and FIGS. 3-4 are required for ALT increase and thrombocytopenia events ONLY. For all other safety events described, these are suggested per the Investigator's medical judgement.

Neutropenia is to be recorded as an AL only if at least 1 of the criteria listed in the general guidelines for reporting AEs is met.

Thrombocytopenia is to be recorded as an AL only if at least 1 of the criteria listed in the general guidelines for reporting AEs) is met.

Note: “Baseline” refers to ALT sampled at baseline visit; or if baseline value unavailable, to the latest ALT sampled before the baseline visit. The algorithm does not apply to the instances of increase in ALT during Screening.

TABLE 3G-A
Actions for cases of confirmed ALT elevation

In ANY CONFIRMED CASE of ALT >5 × ULN or ALT >3 × ULN with bilirubin >2 ×
ULN, the following steps are REQUIRED (recommended for ALT >3 × ULN but
ALT <5 × ULN, as clinically indicated):

| • | INFORM the Site Monitor, who will forward the information to the Study Manager. | |
|---|---|---|
| • | INVESTIGATE specifically for malaise with or without loss of consciousness, | |
| | dizziness, and/or hypotension and/or episode of arrhythmia since the last visit, | |
| | particularly in the previous 72 hours; rule out muscular injury. | |
| • | PERFORM the following tests/actions: | |
| | ∘ | LFTs: AST, ALT, alkaline phosphatase, total and conjugated |
| | | bilirubin and prothrombin time/INR (mandatory assessments for |
| | | ALT >3 × ULN); |
| | ∘ | CPK, serum creatinine, complete blood count; |
| | ∘ | Anti-HAV IgM, anti-HBc IgM, (HBV-DNA if clinically indicated), |
| | | anti-HCV and HCV RNA, anti-CMV IgM and anti- HEV IgM |
| | | antibodies; |
| | ∘ | Iron, ferritin, transferrin saturation; |
| | ∘ | Auto-antibodies: antinuclear, anti-DNA, anti-smooth muscle, anti-LKM, anti- |
| | | mitochondrial; |
| | ∘ | Evaluate recent infection with EBV, herpes viruses. Depending on the clinical |
| | | context, consider testing for toxoplasma; |
| | ∘ | Collect and freeze serum sample (5 mL × 2); |
| | ∘ | Collect and store one PK sample following the instructions in the central |
| | | laboratory manual; |
| | ∘ | Consider hepatobiliary ultrasonography (or other imaging investigations if |
| | | needed); |
| | ∘ | Consider DNA test for Gilbert's disease if clinically indicated; |
| | ∘ | Consider consulting with a hepatologist (mandatory if ALT >8 × ULN or is |
| | | associated with elevated bilirubin); |

| | |
|---|---|
| - | Discuss with the hepatologist the clinical indication for potential liver |
| | biopsy (strongly recommended if the participant meets Hy's law criteria or |
| | has ALT >20 × ULN) and/or initiation of treatment with steroids; |

| | ∘ | Consider patient hospitalization if INR >2 (or PT <50%) and/or central nervous |
|---|---|---|
| | | system disturbances suggesting hepatic encephalopathy |
| • | MONITOR LFTs after discontinuation of IMP: | |
| | ∘ | Monitor closely (every 2-3 days) until ALT is down-trending, then weekly |
| | | until <1.5 × ULN, and then at every scheduled visit; |
| | ∘ | This frequent LFT monitoring may be done through central or local lab, or via |
| | | home visit (depending on the Investigator's assessment and/or local regulatory |
| | | requirements). |

| | |
|---|---|
| • | Rechallenge: Re-initiation of the study drug can only be considered after |
| | discussion with the Sponsor's Medical Monitor once the ALT/AST decreases |
| | to <1.5 × ULN, and there is no clinical contraindication. Rechallenge is not |
| | permitted for the following participants unless a clear non-DILI etiology is |
| | identified: |

```
  • ALT >8 × ULN
  • ALT >5 × ULN for greater than two weeks
  • ALT >3 × ULN and total bilirubin >1 × ULN
```

-In case it is agreed to re-start the study drug, it is recommended that

ALT/AST be assessed per protocol schedule of assessments for the
first 6 months of the treatment period.

-The occurrence of new elevation >3 × ULN for the ALT/AST values will

lead to permanent discontinuation of the study drug.
ALT, alanine aminotransferase; AST, aspartate aminotransferase; CMV, cytomegalovirus; CPK, creatine phosphokinase; CRF, case report form; EBV, Epstein-Barr virus; GGT, gamma glutamyl transferase; HAV, hepatitis A virus; HBV, hepatitis B virus; HCV, hepatitis C virus; HEV, hepatitis E virus; IgG, immunoglobulin G; IgM, immunoglobulin M; IMP, investigational medicinal product; INR, international normalized ratio; LFT, liver function test; LKM, liver-kidney microsomal antibody; PT, prothrombin time; ULN, upper limit of normal.

Increase in serum creatinine is to be recorded as an AE only if at least 1 of the criteria listed in the general guidelines for reporting AEs is met.

Increase in CPK is to be recorded as an AE only if at least 1 of the criteria in the general guidelines for reporting AEs is met.

SUSPECTED PML: If either the clinical presentation or MRI features of a participant are suggestive of PML, the diagnostic and action algorithm described in FIG. 7 is recommended.

Clinical manifestations or MRI lesions features suspicious for PML are proposed in Table 3G-B (based on Berger et al. Neurology 2013, 80, 1430-1438 and Kappos et al. Lancet Neurol. 2007, 6, 431-441).

TABLE 3G-B
Clinical and MRI features suggestive of PML

| | |
|---|---|
| Clinical | Subacute onset of weakness, sensory deficits, cognitive |
| history | or behavioral abnormalities, gait dysfunction, speech/language |
| | difficulties or any other signs of cortical dysfunction, |
| | retrochiasmal visual defects or seizure |
| Brain | ≥1 T2/FLAIR hyperintense and T1 hypointense lesions |
| MRI | involving the subcortical and juxtacortical white matter, |
| | sparing the cortex, with no mass effect, with a continuous |
| | progression; new lesions with no enhancement (even when |
| | large) or with faint rim enhancement |

In the event that PML is suspected based on imaging results, the local radiologist will directly inform the Investigator and a central review of the MRI will not be required. The Investigator will obtain additional plasma, urine, and CSF samples for John Cunningham virus (JCV) analysis. Samples will be analyzed upon receipt and the results will be provided directly to the investigational site and to the Sponsor. Further management will be deferred to the Treating Investigator. However, next steps will include discontinuation of study treatment. Additional imaging will be at the discretion of the Investigator depending on the diagnostic workup and treatment plan.
The detection of John Cunningham virus (JCV) DNA in the cerebrospinal fluid of a patient with clinical and MRI features suggestive of PML establishes the diagnosis of PML.
If JCV DNA is not detected in cerebrospinal fluid and if clinical suspicion of PML remains high, another lumbar puncture should be performed.
If diagnosis remains uncertain and suspicion of PML remains high, a brain biopsy may be considered to establish a definitive diagnosis

Clinical or MRI features suggestive of PML should be recorded as an AE/AESI/SAE following the definitions and procedures.

Example of Drugs with a Potential to Change Tolebrutinib Metabolism or Absorption

The following drugs should not be taken during the study concomitantly with IMP due to their potential to change tolebrutinib kinetics due to interaction with P450-mediated metabolism, being potent and moderate inducers of CYP3A or potent inhibitors of CYP2C8 liver enzymes (per the lists of the Drug Interaction Database Program of the University of Washington; Table 3H-A).

Additionally, participants in certain countries that may be under FDA partial clinical hold conditions must not take medications that are mild, moderate, or potent inhibitors of CYP3A or CYP2C8 (Table 3H-B).

Please note that the lists provided in Tables 3H-A and 3H-B are not exhaustive and that the product information of drugs intended for concomitant use should be consulted.

TABLE 3H-A
CYP3A Inducers and CYP2C8 Inhibitors

| | |
|---|---|
| Potent CYP3A Inducers: | |
| Rifampin | Carbamazepine |
| St John's Wort extract | Phenobarbital |
| Avasimibe | Lumacaftor |
| Rifapentine | |
| Phenytoin | Ryfabutin |
| Potent CYP2C8 Inhibitors: | |
| Gemfibrozil | |
| Moderate CYP3A Inducers: | |
| Semagacestat | Asunaprevir/beclabuvir/daclatasvir |
| Cenobamate | Nafcillin |
| Lesinurad | Telotristat ethyl |
| Bosentan | Elagolix |
| Thioridazine | Rifabutin |

TABLE 3H-B
Mild, moderate, and potent inhibitors of CYP3A and
CYP2C8, and moderate and potent inducers of CYP3A

| | Potent | Moderate | Mild |
|---|---|---|---|
| CYP2C8 inhibitors | Gemfibrozil | Clopidogrel | Sulfamethoxazole |
| | | Deferasirox | Trimethoprim |
| | | | Fluvoxamine |

| | Potent | Moderate |
|---|---|---|
| CYP3A inducers | Avasimibe | Elagolix |
| | Rifampin | Cenobamate |
| | Carbamazepine | Nafcillin |
| | Lumacaftor | Asunaprevir/beclabuvir/daclatasvir |
| | Phenobarbital | Lesinurad |
| | Phenytoin | Bosentan |
| | Rifapentine | Thioridazine |
| | St. John's Wort | Rifabutin |
| | | Semagacestat |
| | | Telotristat ethyl |

| | Potent | Moderate | Mild |
|---|---|---|---|
| CYP3A | Clarithromycin | Ciprofloxacin | Alprazolam |
| inhibitors | Itraconazole | Diltiazem | Atorvastatin |
| | Ketoconazole | Erythromycin | Amlodipine |
| | Nirmatrelvir and ritonavir | Fluconazole | Cimetidine |
| | Fluoxetine | Verapamil | Ranitidine |
| | Grapefruit juice | Sertraline | Roxithromycin |
| | | | Ginkgo biloba |
| | | | Isoniazid |

### Example 4—Paramagnetic Rim Lesions as a Prognostic and Predictive Biomarker for Disability Outcomes in the Studies of Example 1 (HERCULES) and Example 2 (GEMINI 1 and 2)

Background Paramagnetic rim lesions (PRLs) are characterized by a discrete perlesional rim on susceptibility-sensitive MRI and a T2-hyperintense core that does not enhance with gadolinium (Bagnato et al., Brain. 2024; 147:2913-2933). PRLs correspond to histopathologically-defined chronic active lesions having the following features: demyelinated core with axonal loss and inflammatory demyelinatic rim continain iron-laden microglia and macrophages (Bagnato et al.; Absinta et al., JAMA Neurology. 2019; 76:1474-1483). PRLs are correlated with disability accumulation and resistant to currently approved therapies (Absinta et al.; Cagol et al. Neurology. 2024; 102:e207768).

Objectives. PRLs observed at baseline were evaluated as a prognostic and predictive biomarker for disability accumulation and treatment response in HERCULES (Example 1) and GEMINI 1 and 2 trials (Example 2).

Methods. 437 (39%) of the 1131 HERCULES participants and 631 (34%) of the 1873 GEMINI participants were from sites with imaging capabilities allowing evaluation of PRLs (see Table 4C). The proportion of participants with PRL imaging was well balanced across the treatment groups. For HERCULES, 288 of 754 (38%) of participants assigned to tolebrutinib and 149 of 377 (40%) of participants assigned to placebo had PRL imaging. For GEMINI, 325 of 933 (35%) of participants assigned to tolebrutinib and 306 of 940 (33%) of participants assigned to teriflunomide had PRL imaging. The effect of tolebrutinib on time to onset of 6-month confirmed disability progression (6-month CDP) 6-month confirmed disability worsening (6-month CDW) was evaluated in participants with 0, 1-3, or ≥4 PRLs at baseline. PRLs were manually identified on SWI images generated from 3D-gradient echo phase images (6 echoes ranging from 4.9 to 41 ms, 0.8-mm isotropic resolution).

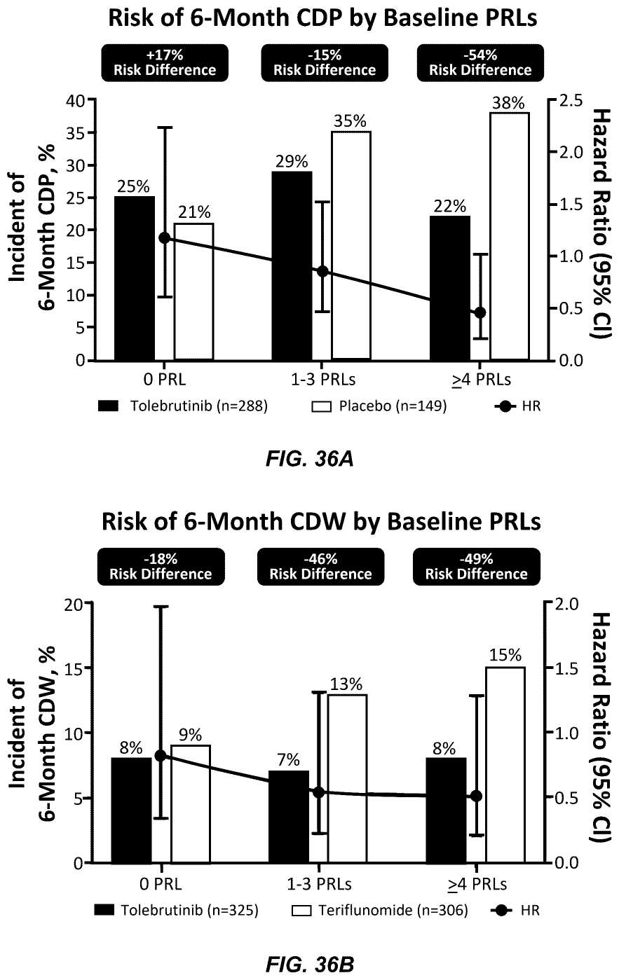

Results. Across both trials, 653 participants (61%) had PRLs, consistent with data from observational studies with high-sensitivity MRI sequences. In HERCULES, the proportion of participants with 0, 1-3, or ≥4 PRLs at baseline was 40%, 36%, and 24%, respectively. In GEMINI, the proportion of participants with 0, 1-3, or ≥4 PRLs at baseline was 38%, 35%, and 27%, respectively. In both HERCULES and GEMINI, the risk of 6-month CDP or 6-month CDW, respectively, increased as a function of baseline PRLs in the placebo and teriflunomide comparator groups, respectively. In HERCULES, tolebrutinib appeared to mitigate the risk of 6-month CDP with greater effect in participants with more baseline PRLs, reducing the risk by 54% in participants with ≥4 PRLs (see Table 4A, FIG. 36A). In GEMINI, a similar risk mitigation was observed with a 46% and 49% risk reduction for 6-month CDW in participants with 1-3 and ≥4 PRLs, respectively (see Table 4B, FIG. 36B). In tolebrutinib-treated participants with PRLs in both HERCULES and GEMINI, the risk of 6-month CDP or 6-month CDW, respectively, was numerically similar to the corresponding risk in participants without PRLs. The impact of tolebrutinib vs. placebo on 6-month CDW and tolebrutinib vs. teriflunomide on 6-month CDP was more evident in participants with PRLs at baseline.

This post hoc analysis suggests that the impact of tolebrutinib treatment may be greater in those with higher number of PRLs, consistent with the CNS bioactive mechanism of action of tolebrutinib.

TABLE 4A
Analysis of time to onset of 6-month CDP using Cox model
by baseline number of phase rim lesions (PRL) for the
study of Example 1 (intention-to-treat population)

| | | Placebo | Tolebrutinib |
|---|---|---|---|
| No. of PRL | | (N = 377) | (N = 754) |
| 0 | No. | 56 | 119 |
| | No. with 6-month CDP (%) | 12 (21.4) | 30 (25.2) |

| | |
|---|---|
| HR (95% CI)^{a} | 1.171 (0.612; 2.242) |

| | | | |
|---|---|---|---|
| 1-3 | No. | 54 | 105 |
| | No. with 6-month CDP (%) | 19 (35.2) | 30 (28.6) |

| | |
|---|---|
| HR (95% CI)^{a} | 0.846 (0.468; 1.531) |

| | | | |
|---|---|---|---|
| ≥4 | No. | 39 | 64 |
| | No. with 6-month CDP (%) | 15 (38.5) | 14 (21.9) |

| | |
|---|---|
| HR (95% CI)^{a} | 0.463 (0.210; 1.019) |

^{a}Derived using Cox proportional-hazards model with treatment group, age at screening (>40, <=40 years), geographic region (US, non-US), baseline EDSS score and baseline Gd-enhancing T1 lesions (presence, absence) as covariates.

TABLE 4B
Analysis of time to onset of 6-month CDW using Cox model
by baseline number of phase rim lesions (PRL) for the studies
of Example 2 (intention-to-treat population, pooled)

| | | Teriflunomide | Tolebrutinib |
|---|---|---|---|
| No. of PRL | | (N = 940) | (N = 937) |
| 0 | No. | 119 | 119 |
| | No. with 6-month CDW (%) | 11 (9.2) | 9 (7.6) |
| | HR | 0.815 | |
| 1-3 | No. | 114 | 108 |
| | No. with 6-month CDW (%) | 15 (13.2) | 8 (7.4) |
| | HR | 0.540 | |
| ≥4 | No. | 74 | 98 |
| | No. with 6-month CDW (%) | 11 (14.9) | 8 (8.2) |

| | |
|---|---|
| HR | 0.513 |

TABLE 4C
Baseline characteristics for total cohort and PRL imaging cohort for the studies of Example 1 and 2

| | HERCULES (Example 1) | | GEMINI 1 and 2 (Example 2) | |
|---|---|---|---|---|
| | | Cohort With | | Cohort With |
| | Total Cohort | PRL Imaging | Total Cohort | PRL Imaging |
| Characteristic | (N = 1131) | (N = 437, 39%) | (N = 1873) | (N = 631, 34%) |
| Age, years | 48.9 (8.0) | 50.4 (7.3) | 36.5 (9.3) | 37.1 (9.4) |
| Female, n (%) | 696 (61.5) | 278 (63.6) | 1252 (66.8) | 435 (68.9) |
| EDSS score^{a} | | | | |
| Mean (SD) | 5.5 (1.0) | 5.6 (1.0) | 2.4 (1.2) | 2.2 (1.2) |
| Median (IQR) | 6.0 (5.0-6.3) | 6.0 (5.0-6.3) | 2.0 (1.5-3.0) | 2.0 (1.5-3.0) |
| Time since RRMS | 17.3 (8.4) | 18.4 (8.5) | 6.6 (7.1) | 6.0 (7.1) |
| symptom onset, | | | | |
| years | | | | |
| Number of | 0 | 0 | 1.2 (0.6) | 1.2 (0.6) |
| relapses within | | | | |
| previous 1 year | | | | |
| Participants with ≥1 | 142 (12.7) | 37 (8.5) | 645 (34.6) | 196 (31.1) |
| Gd-enhancing | | | | |
| T1 lesions, n (%) | | | | |
| T2 lesion volume, | 15.2 (7.3-27.0) | 12.4 (6.1-23.4) | 9.4 (4.2-17.7) | 7.9 (3.3-15.5) |
| cm3, median (IQR) | | | | |
| Participants with ≥1 | 837 (74.0) | 331 (75.7) | 666 (35.6) | 215 (34.1) |
| prior DMTs, n (%) | | | | |

^{a}Average of screening and randomization EDSS scores.
DMT = disease-modifying therapy; EDSS = Expanded Disability status scale; Gd = gadolinium; IQR = interquartile range; SD = standard deviation (mean SD unless otherwise indicated).

### ABBREVIATIONS

ADL: activities of daily living
AE: adverse event
AESI: adverse event of special interest
ALT: alanine aminotransferase
aPTT: activated partial thromboplastin time
ARR: annualized relapse rate (annualized relapse rate and ARR refer to the annualized relapse rate for adjudicated relapses, i.e., the annualized adjudicated relapse rate)
AUC: area under the curve
BCRP: breast cancer resistance protein
BTK: Bruton's tyrosine kinase
CD: cluster of differentiation
CDW: confirmed disability worsening
CFR: Code of Federal Regulations
Chi3L1: chitinase-3 like protein-1
CNS: central nervous system
COVID-19: Coronavirus Disease 2019
CPK: creatine phosphokinase
CRF: case report form
CSF: cerebrospinal fluid
CSR: clinical study report
C-SSRS: Columbia Suicide Severity Rating Scale
CYP: cytochrome P450
DILI: drug-induced liver injury
DMC: Data Monitoring Committee
DMT: disease-modifying therapy
DNA: deoxyribonucleic acid
DTP: direct-to-patient
EC: ethics committee
ECG: electrocardiogram
eCRF: electronic case report form
EDSS: Expanded Disability Status Scale
EOS: end of study
EQ-5D-5L: EuroQol 5-dimension 5-level questionnaire
EU: European Union
FSH: follicle-stimulating hormone
GCP: Good Clinical Practice
Gd: gadolinium
GM-CSF: granulocyte-macrophage-colony stimulating factor
GrA: human granzyme A
GrB: human granzyme B
GrK: human granzyme K
GrM: human granzyme M
HIV: human immunodeficiency virus
HR: hazard ratio
HRT: hormone replacement therapy
ICF: informed consent form
ICH: International Council for Harmonisation
IEC: Independent Ethics Committee
IFNγ: Interferon γ
IL: interleukin
IMP: investigational medicinal product
INR: international normalized ratio
IRB: Institutional Review Board
IRT: interactive response technology
ITT: intent to treat
IUD: intrauterine device
IUS: intrauterine hormone-releasing system
IV: intravenous
IWRS: interactive web response system
JCV: John Cunningham virus
LTS: long-term safety
MCAM: melanoma cell adhesion molecule
MedDRA: Medical Dictionary for Regulatory Activities
MRI: magnetic resonance imaging
MS: multiple sclerosis
MSFC-3: Multiple Sclerosis Functional Composite-3
MSQol-54: Multiple Sclerosis Quality of Life-54
MTR: magnetization transfer ratio
NCI CTCAE: National Cancer Institute Common Terminology Criteria for Adverse
Events
NEDA: no evidence of disease activity-3
NfL: neurofilament light chain
NIMP: noninvestigational medicinal product
nrSPMS: nonrelapsing secondary progressive multiple sclerosis
NSAID: nonsteroidal anti-inflammatory drug
OAT3: organic anion transporter3
PD: pharmacodynamic(s)
PK: pharmacokinetic(s)
PML: progressive multifocal leukoencephalopathy
PPMS: primary progressive multiple sclerosis
pTreg: peripheral regulatory T cells
QTcF: QT interval corrected using Fridericia's formula
RMS: relapsing multiple sclerosis
RTE: Recent thymic emigrant
SAE: serious adverse event
SAP: Statistical Analysis Plan
SEL: slowly evolving lesions
SmPC: summary of product characteristics
SoA: schedule of activities
SPMS: secondary progressive multiple sclerosis
Study Manual: Study Reference Manual
SUSAR: suspected unexpected serious adverse reaction
SWI: susceptibility-weighted imaging
TB: tuberculosis
TEAE: treatment-emergent adverse event
Tfh: T follicular helper
Th 17: T helper 17 cells
Th1: T helper 1 cells
Th2: T helper 2 cells
TIGIT: T cell immunoglobulin and immunoreceptor tyrosine-based inhibitory motif (ITIM) domain
TNFα: tumor necrosis factor α
Treg: regulatory T cell
tTreg: Thymically derived Foxp3(+) regulatory T cells
ULN: upper limit of normal
US: United States
USPI United States prescribing information
WBC: white blood cells
WOCBP: woman of childbearing potential V. Enumerated Embodiments

Disclosed herein is a method of reducing disability accumulation independent of relapse activity in a patient with secondary progressive MS in need thereof comprising administering to the patient in need thereof a therapeutically effective amount of tolebrutinib or pharmaceutically acceptable salt thereof.

The present disclosure relates to methods of treating a patient with non-relapsing secondary progressive multiple sclerosis (nrSPMS) in need thereof, comprising administering to the patient in need thereof a therapeutically effective amount of tolebrutinib or a pharmaceutically acceptable salt thereof. The present disclosure also relates to methods of reducing disability accumulation independent of relapse activity in a patient with secondary progressive MS in need thereof comprising administering to the patient in need thereof a therapeutically effective amount of tolebrutinib or pharmaceutically acceptable salt thereof.

For example, embodiments of the disclosure include the following:

Embodiment 1. A method of treating a patient with non-relapsing secondary progressive multiple sclerosis (nrSPMS) in need thereof, comprising administering to the patient in need thereof a therapeutically effective amount of tolebrutinib or pharmaceutically acceptable salt thereof, wherein at least one outcome chosen from the following occurs: (a) the risk of confirmed disability progression (CDP) is reduced; (b) the total number of new and/or enlarging T2-hyperintense lesions as detected by MRI, defined as the sum of the individual number of new and/or enlarging T2 lesions, are reduced; (c) the risk of sustained 20% increase in the timed 25-foot walk (T25-FW) test confirmed over at least 3 months is reduced; and (d) the chance of confirmed disability improvement (CDI) is improved.
Embodiment 2. A method of treating a patient with non-relapsing secondary progressive multiple sclerosis (nrSPMS) in need thereof, comprising administering to the patient in need thereof a therapeutically effective amount of tolebrutinib or pharmaceutically acceptable salt thereof, wherein the risk of confirmed disability progression (CDP) is reduced.
Embodiment 3. The method of embodiment 2, wherein the CDP is measured over at least three months, such as at least six months.
Embodiment 4. The method of embodiment 2 or 3, wherein the confirmed disability progression (CDP) comprises: (a) increasing at least 1.0 point from a baseline expanded disability status scale score (EDSS) when the baseline score is less than or equal to 5.0; or (b) increasing at least 0.5 points when the baseline EDSS score is greater than 5.0.
Embodiment 5. The method of any one of embodiments 1-4, wherein the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.
Embodiment 6. A method of reducing the risk of confirmed disability progression (CDP) in a patient with non-relapsing secondary progressive multiple sclerosis (nrSPMS) in need thereof, comprising administering to the patient in need thereof a therapeutically effective amount of tolebrutinib or pharmaceutically acceptable salt thereof.
Embodiment 7. The method of embodiment 6, wherein the CDP is measured over at least three months, such as at least six months.
Embodiment 8. The method of embodiment 6 or 7, wherein confirmed disability progression (CDP) comprises: (a) increasing at least 1.0 point from a baseline expanded disability status scale score (EDSS) when the baseline score is less than or equal to 5.0; or (b) increasing at least 0.5 points when the baseline EDSS score is greater than 5.0.
Embodiment 9. The method of any one of embodiments 6-8, wherein the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.
Embodiment 10. The method of any one of embodiments 6-9, wherein the risk reduction is relative to placebo.
Embodiment 11. A method of reducing the total number of new and/or enlarging T2-hyperintense lesions as detected by MRI, in a patient with non-relapsing secondary progressive multiple sclerosis (nrSPMS) in need thereof, comprising administering to the patient in need thereof a therapeutically effective amount of tolebrutinib or pharmaceutically acceptable salt thereof.
Embodiment 12. The method of embodiment 11, wherein reducing comprises reducing the annualized rate of new and/or enlarging T2-hyperintense lesions.
Embodiment 13. The method of embodiment 11 or 12, wherein the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.
Embodiment 14. The method of embodiment 11, wherein the annualized rate is reduced relative to placebo.
Embodiment 15. A method of reducing the risk of sustained 20% increase in the timed 25-foot walk (T25-FW) test confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof.
Embodiment 16. The method of embodiment 15, wherein the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.
Embodiment 17. The method of embodiment 15 or 16, wherein the risk reduction is relative to placebo.
Embodiment 18. A method of improving the chance of confirmed disability improvement (CDI) in a patient with non-relapsing secondary progressive multiple sclerosis (nrSPMS) in need thereof, comprising administering to the patient in need thereof a therapeutically effective amount of tolebrutinib or pharmaceutically acceptable salt thereof.
Embodiment 19. The method of embodiment 18, wherein the CDI is measured over at least three months, such as at least six months.
Embodiment 20. The method of embodiment 19, wherein the confirmed disability improvement (CDI) comprises decreasing at least 1.0 point from a baseline expanded disability status scale score (EDSS).
Embodiment 21. The method of embodiment 19 or 20, wherein the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.
Embodiment 22. The method of any one of embodiments 19-21, wherein the chance is improved relative to placebo.
Embodiment 23. A method of improving time to onset of confirmed disability progression (CDP) in a patient with non-relapsing secondary progressive multiple sclerosis (nrSPMS) in need thereof, comprising administering to the patient in need thereof a therapeutically effective amount of tolebrutinib or pharmaceutically acceptable salt thereof.
Embodiment 24. The method of embodiment 23, wherein the CDP is measured over at least three months, such as at least six months.
Embodiment 25. The method of embodiment 23 or 24, wherein the confirmed disability progression (CDP) comprises: (a) increasing at least 1.0 point from a baseline expanded disability status scale score (EDSS) when the baseline score is less than or equal to 5.0; or (b) increasing at least 0.5 points when the baseline EDSS score is greater than 5.0.
Embodiment 26. The method of any one of embodiments 23-25, wherein the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.
Embodiment 27. A method of improving time to onset of sustained 20% increase in the timed 25-foot walk (T25-FW) test confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof.
Embodiment 28. The method of embodiment 27, wherein the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.
Embodiment 29. A method of improving time to onset of confirmed disability improvement (CDI) in a patient with non-relapsing secondary progressive multiple sclerosis (nrSPMS) in need thereof, comprising administering to the patient in need thereof a therapeutically effective amount of tolebrutinib or pharmaceutically acceptable salt thereof.
Embodiment 30. The method of embodiment 29, wherein the CDI is measured over at least three months, such as at least six months.
Embodiment 31. The method of embodiment 29 or 30, wherein the confirmed disability improvement (CDI) comprises decreasing at least 1.0 point from a baseline expanded disability status scale score (EDSS).
Embodiment 32. The method of any one of embodiments 29-31, wherein the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.
Embodiment 33. Tolebrutinib or a pharmaceutically acceptable salt thereof for use in treating a patient with non-relapsing secondary progressive multiple sclerosis (nrSPMS) in the method of any one of embodiments 1-5.
Embodiment 34. Tolebrutinib or a pharmaceutically acceptable salt thereof for use in reducing the risk of confirmed disability progression (CDP) in a patient with non-relapsing secondary progressive multiple sclerosis (nrSPMS) in the method of any one of embodiments 6-10.
Embodiment 35. Tolebrutinib or a pharmaceutically acceptable salt thereof for use in reducing the total number of new and/or enlarging T2-hyperintense lesions as detected by MRI in a patient with non-relapsing secondary progressive multiple sclerosis (nrSPMS) in the method of any one of embodiments 11-14.
Embodiment 36. Tolebrutinib or a pharmaceutically acceptable salt thereof for use in reducing the risk of sustained 20% increase in the timed 25-foot walk (T25-FW) test confirmed over at least 3 months in a patient with non-relapsing secondary progressive multiple sclerosis (nrSPMS) in the method of any one of embodiments 15-17.
Embodiment 37. Tolebrutinib or a pharmaceutically acceptable salt thereof for use in improving the chance of confirmed disability improvement (CDI) in a patient with non-relapsing secondary progressive multiple sclerosis (nrSPMS) in the method of any one of embodiments 18-22.
Embodiment 38. Tolebrutinib or a pharmaceutically acceptable salt thereof for use in improving time to onset of confirmed disability progression (CDP) in a patient with non-relapsing secondary progressive multiple sclerosis (nrSPMS) in the method of any one of embodiments 23-26.
Embodiment 39. Tolebrutinib or a pharmaceutically acceptable salt thereof for use in improving time to onset of sustained 20% increase in the timed 25-foot walk (T25-FW) test confirmed over at least 3 months in a patient with non-relapsing secondary progressive multiple sclerosis (nrSPMS) in the method of any one of embodiments 27-28.
Embodiment 40. Tolebrutinib or a pharmaceutically acceptable salt thereof for use in improving time to onset of confirmed disability improvement (CDI) in a patient with non-relapsing secondary progressive multiple sclerosis (nrSPMS) in the method of any one of embodiments 29-32.
Embodiment 41. The method of any one of embodiments 1-40, further comprising determining whether the patient has elevated transferrin or elevated ferritin levels and when the patient is found to not have elevated transferrin or elevated ferritin levels, administering to the patient a therapeutically effective amount of tolebrutinib or pharmaceutically acceptable salt thereof.
Embodiment 42. The method of any one of embodiments 1-40, further comprising determining the patient's iron panel, and when the patient is found to have a suitable iron panel, administering to the patient a therapeutically effective amount of tolebrutinib or pharmaceutically acceptable salt thereof.
Embodiment 43. The method of any one of embodiments 1-40, wherein the patient does not have elevated transferrin or elevated ferritin levels.
Embodiment 44. The method of any one of embodiments 1-40, wherein the patient has a suitable iron level.
Embodiment 45. The method of any one of embodiments 1-40, further comprising the steps of: (a) performing an iron panel test in a patient's blood or serum; (b) detecting levels of the iron panel test that are within normal ranges; and (c) administering a therapeutically effective amount of tolebrutinib or pharmaceutically acceptable salt thereof to the patient, wherein the iron panel test measures any one or more of levels of iron, ferritin, transferrin saturation, and total iron-binding capacity (TIBC) in a patient's blood or serum and wherein the normal ranges of the iron panel test include one or more of (i) an iron level of 60 to 170 μg/dL, (ii) a ferritin level of ≤500 g/L (iii) a transferrin saturation level ≤50% in a male patient or ≤40% in a female patient, and (iv) a TIBC of 240 to 450 μg/dL.
Embodiment 46. The method of any one of embodiments 1-40, further comprising the steps of: (a) detecting a level of transferrin saturation in a patient's blood or serum that is within normal range; and (b) administering a therapeutically effective amount of tolebrutinib or pharmaceutically acceptable salt thereof to the patient, wherein the transferrin saturation level that is within normal range in the blood or serum of a male patient is a transferrin saturation of ≤50%, and the transferrin saturation level that is within normal range in the blood or serum of a female patient is a transferrin saturation of ≤40%.
Embodiment 47. The method of any one of embodiments 1-40, further comprising the steps of: (a) detecting a level of ferritin in a patient's blood or serum that is within normal range; and (b) administering a therapeutically effective amount of tolebrutinib or pharmaceutically acceptable salt thereof to the patient, wherein the ferritin level that is within normal range in the blood or serum of the patient is ≤500 g/L.
Embodiment 48. The method of any one of embodiments 1-40, further comprising the steps of: (a) performing liver function tests in a patient; (b) detecting suitable liver function in the patient; and (c) administering a therapeutically effective amount of tolebrutinib or pharmaceutically acceptable salt thereof to the patient, wherein the liver function tests measure one or more of the levels of aspartate transaminase (AST), alanine transaminase (ALT), albumin, alkaline phosphatase, total and direct bilirubin, and total protein in a patient's blood, and wherein the patient having a suitable liver function has one or more of ALT≤1.5×upper limit of normal (ULN), AST levels of ≤1.5×ULN, alkaline phosphatase ≤2×ULN (unless caused by non-liver related disorder or explained by a stable chronic liver disorder) and total bilirubin ≤1.5×ULN (unless due to Gilbert syndrome or non-liver-related disorder).
Embodiment 49. The method of any one of embodiments 1-40, further comprising the steps of: (a) administering a therapeutically effective amount of tolebrutinib or pharmaceutically acceptable salt thereof to a patient in need thereof; (b) measuring the level of alanine aminotransferase (ALT) in the patient; (c) detecting a level of ALT of >8×upper limit of normal (ULN); (d) ceasing administration of tolebrutinib or pharmaceutically acceptable salt thereof to the patient; and optionally (e) monitoring the level of ALT in the patient; and (f) resuming administration of a therapeutically effective amount of tolebrutinib or pharmaceutically acceptable salt thereof to the patient when the patient's level of ALT is determined to be <1.5×ULN.
Embodiment 50. The method of any one of embodiments 1-40, further comprising the steps of: (a) administering a therapeutically effective amount of tolebrutinib or pharmaceutically acceptable salt thereof to a patient in need thereof; (b) measuring the level of alanine aminotransferase (ALT) in the patient; (c) detecting a level of ALT of >5×upper limit of normal (ULN) during a period of at least two weeks; (d) ceasing administration of tolebrutinib or pharmaceutically acceptable salt thereof to the patient; and optionally (e) monitoring the level of ALT in the patient; and (f) resuming administration of a therapeutically effective amount of tolebrutinib or pharmaceutically acceptable salt thereof to the patient when the patient's level of ALT is determined to be <1.5×ULN.
Embodiment 51. The method of any one of embodiments 1-40, further comprising the steps of: (a) administering a therapeutically effective amount of tolebrutinib or pharmaceutically acceptable salt thereof to a patient in need thereof; (b) measuring the level of alanine aminotransferase (ALT) in the patient; (c) detecting a level of ALT of >3×upper limit of normal (ULN); (d) measuring one or more of total bilirubin and international normalized ratio (INR) in a patient; (e) detecting one or more of total bilirubin >2×ULN and INR>1.5; (f) ceasing administration of tolebrutinib or pharmaceutically acceptable salt thereof to the patient; and optionally (g) monitoring the level of ALT in the patient; and (h) resuming administration of a therapeutically effective amount of tolebrutinib or pharmaceutically acceptable salt thereof to the patient when the patient's level of ALT is determined to be <1.5×ULN.
Embodiment 52. The method of any one of embodiments 1-40, further comprising the steps of: (a) administering a therapeutically effective amount of tolebrutinib or pharmaceutically acceptable salt thereof to a patient in need thereof; (b) measuring the level of alanine aminotransferase (ALT) in the patient; (c) detecting a level of ALT of >3×upper limit of normal (ULN); (d) ceasing administration of tolebrutinib or pharmaceutically acceptable salt thereof to the patient if the patient experiences one or more of fatigue, nausea, vomiting, right upper quadrant pain or tenderness, fever, rash, and eosinophilia >5%; and optionally (e) monitoring the level of ALT in the patient; and (f) resuming administration of a therapeutically effective amount of tolebrutinib or pharmaceutically acceptable salt thereof to the patient when the patient's level of ALT is determined to be <1.5×ULN.
Embodiment 53. The method of any one of embodiments 41-52, wherein the level of ALT in step (b) is determined at least monthly.
Embodiment 54. The method of any one of embodiments 41-52, wherein the level of ALT in step (d) is monitored at least weekly.
Embodiment 55. The method of any one of embodiments 41-52, wherein the level of ALT in step (d) is monitored every 2 to 3 days.
Embodiment 56. The method of any one of embodiments 1-55, wherein the patient is not receiving potent and moderate inducers of cytochrome P450 3A (CYP3A) or potent inhibitors of CYP2C8 hepatic enzymes.
Embodiment 57. The method of any one of embodiments 1-56, further comprising the steps of: (a) advising the patient to limit alcohol consumption during treatment; and (b) administering a therapeutically effective amount of tolebrutinib or pharmaceutically acceptable salt thereof to the patient, wherein the patient is female and is advised to limit alcohol consumption to 14 grams/day or less, or the patient is male and is advised to limit alcohol consumption to 28 grams/day or less.

### VI. Additional Enumerated Embodiments

The present disclosure relates to therapeutic tyrosine kinase inhibitors, in particular, Bruton tyrosine kinase (“BTK”) inhibitors (e.g., tolebrutinib), for reducing the risk of confirmed disability worsening (CDW) patients with a relapsing form of multiple sclerosis (RMS). For example, embodiments of the disclosure include the following:

Embodiment 1. A method of reducing the risk of confirmed disability worsening (CDW) in a patient with a relapsing form of multiple sclerosis (RMS) in need thereof, comprising administering to the patient in need thereof a therapeutically effective amount of tolebrutinib or pharmaceutically acceptable salt thereof.
Embodiment 2. The method of embodiment 1, wherein the CDW is measured over at least three months, such as at least six months.
Embodiment 3. The method of embodiment 1 or 2, wherein CDW comprises: (a) increasing at least 1.5 point from a baseline expanded disability status scale score (EDSS) when the baseline score is 0; (b) increasing at least 1.0 point from the baseline EDSS when the baseline score is greater than or equal to 0.5 and less than or equal to 5.5; or (c) increasing at least 0.5 points when the baseline EDSS score is greater than 5.5.
Embodiment 4. The method of any one of embodiments 1-3, wherein the patient has an RMS diagnosis with an EDSS less than or equal to 5.5 prior to initial administration of tolebrutinib, and one or more of the following: (a) one or more documented relapses in the previous year; (b) two or more documented relapses in the previous 2 years; and (c) one or more Gd-enhancing lesions on an MRI scan in the previous year.
Embodiment 5. The method of embodiment any one of embodiments 1-4, wherein the risk reduction is relative to treatment with teriflunomide.
Embodiment 6. A method of improving time to onset of confirmed disability worsening (CDW) in a patient with a relapsing form of multiple sclerosis (RMS) in need thereof, comprising administering to the patient in need thereof a therapeutically effective amount of tolebrutinib or pharmaceutically acceptable salt thereof.
Embodiment 7. The method of embodiment 6, wherein the CDW is measured over at least three months, such as at least six months.
Embodiment 8. The method of embodiment 6 or 7, wherein CDW comprises: (a) increasing at least 1.5 point from a baseline expanded disability status scale score (EDSS) when the baseline score is 0; (b) increasing at least 1.0 point from the baseline EDSS when the baseline score is greater than or equal to 0.5 and less than or equal to 5.5; or (c) increasing at least 0.5 points when the baseline EDSS score is greater than 5.5.
Embodiment 9. The method of any one of embodiments 6-8, wherein the patient has an RMS diagnosis with an EDSS less than or equal to 5.5 at the first visit, and one or more of the following: (a) one or more documented relapses in the previous year; (b) two or more documented relapses in the previous 2 years; and (c) one or more Gd-enhancing lesions on an MRI scan in the previous year.
Embodiment 10. A method of improving percent change in brain volume loss (BVL) as detected by brain MRI in a patient with relapsing forms of multiple sclerosis (RMS), comprising administering to the patient in need thereof a therapeutically effective amount of tolebrutinib or pharmaceutically acceptable salt thereof.
Embodiment 11. The method of embodiment 10, wherein the patient has an RMS diagnosis with an EDSS less than or equal to 5.5 at the first visit, and one or more of the following: (a) one or more documented relapses in the previous year; (b) two or more documented relapses in the previous 2 years; and (c) one or more Gd-enhancing lesions on an MRI scan in the previous year.
Embodiment 12. The method of embodiment 10 or embodiment 11, wherein the percent change BVL improvement is relative to treatment with teriflunomide.
Embodiment 13. The method of any one of embodiments 1-12, further comprising determining whether the patient has elevated transferrin or elevated ferritin levels and when the patient is found to not have elevated transferrin or elevated ferritin levels, administering to the patient a therapeutically effective amount of tolebrutinib or pharmaceutically acceptable salt thereof.
Embodiment 14. The method of any one of embodiments 1-12, further comprising determining the patient's iron panel, and when the patient is found to have a suitable iron panel, administering to the patient a therapeutically effective amount of tolebrutinib or pharmaceutically acceptable salt thereof.
Embodiment 15. The method of any one of embodiments 1-12, further comprising administering to a patient in need thereof a therapeutically effective amount of tolebrutinib or pharmaceutically acceptable salt thereof, wherein the patient does not have elevated transferrin or elevated ferritin levels.
Embodiment 16. The method of any one of embodiments 1-12, further comprising administering to a patient in need thereof a therapeutically effective amount of tolebrutinib or pharmaceutically acceptable salt thereof, wherein the patient has a suitable iron level.
Embodiment 17. The method of any one of embodiments 1-12, further comprising the steps of: (a) performing an iron panel test in a patient's blood or serum; (b) detecting levels of the iron panel test that are within normal ranges; and (c) administering a therapeutically effective amount of tolebrutinib or pharmaceutically acceptable salt thereof to the patient, wherein the iron panel test measures any one or more of levels of iron, ferritin, transferrin saturation, and total iron-binding capacity (TIBC) in a patient's blood or serum and wherein the normal ranges of the iron panel test include one or more of (i) an iron level of 60 to 170 μg/dL, (ii) a ferritin level of ≤500 g/L (iii) a transferrin saturation level ≤50% in a male patient or ≤40% in a female patient, and (iv) a TIBC of 240 to 450 μg/dL.
Embodiment 18. The method of any one of embodiments 1-12, further comprising the steps of: (a) detecting a level of transferrin saturation in a patient's blood or serum that is within normal range; and (b) administering a therapeutically effective amount of tolebrutinib or pharmaceutically acceptable salt thereof to the patient, wherein the transferrin saturation level that is within normal range in the blood or serum of a male patient is a transferrin saturation of ≤50%, and the transferrin saturation level that is within normal range in the blood or serum of a female patient is a transferrin saturation of ≤40%.
Embodiment 19. The method of any one of embodiments 1-12, further comprising the steps of: (a) detecting a level of ferritin in a patient's blood or serum that is within normal range; and (b) administering a therapeutically effective amount of tolebrutinib or pharmaceutically acceptable salt thereof to the patient, wherein the ferritin level that is within normal range in the blood or serum of the patient is ≤500 g/L.
Embodiment 20. The method of any one of embodiments 1-12, further comprising the steps of: (a) performing liver function tests in a patient; (b) detecting suitable liver function in the patient; and (c) administering a therapeutically effective amount of tolebrutinib or pharmaceutically acceptable salt thereof to the patient, wherein the liver function tests measure one or more of the levels of aspartate transaminase (AST), alanine transaminase (ALT), albumin, alkaline phosphatase, total and direct bilirubin, and total protein in a patient's blood, and wherein the patient having a suitable liver function has one or more of ALT≤1.5×upper limit of normal (ULN), AST levels of ≤1.5×ULN, alkaline phosphatase ≤2×ULN (unless caused by non-liver related disorder or explained by a stable chronic liver disorder) and total bilirubin ≤1.5×ULN (unless due to Gilbert syndrome or non-liver-related disorder).
Embodiment 21. The method of any one of embodiments 1-12, further comprising the steps of: (a) administering a therapeutically effective amount of tolebrutinib or pharmaceutically acceptable salt thereof to a patient in need thereof; (b) measuring the level of alanine aminotransferase (ALT) in the patient; (c) detecting a level of ALT of >8×upper limit of normal (ULN); (d) ceasing administration of tolebrutinib or pharmaceutically acceptable salt thereof to the patient; and optionally (e) monitoring the level of ALT in the patient; and (f) resuming administration of a therapeutically effective amount of tolebrutinib or pharmaceutically acceptable salt thereof to the patient when the patient's level of ALT is determined to be <1.5×ULN.
Embodiment 22. The method of any one of embodiments 1-12, further comprising the steps of: (a) administering a therapeutically effective amount of tolebrutinib or pharmaceutically acceptable salt thereof to a patient in need thereof; (b) measuring the level of alanine aminotransferase (ALT) in the patient; (c) detecting a level of ALT of >5×upper limit of normal (ULN) during a period of at least two weeks; (d) ceasing administration of tolebrutinib or pharmaceutically acceptable salt thereof to the patient; and optionally (e) monitoring the level of ALT in the patient; and (f) resuming administration of a therapeutically effective amount of tolebrutinib or pharmaceutically acceptable salt thereof to the patient when the patient's level of ALT is determined to be <1.5×ULN.
Embodiment 23. The method of any one of embodiments 1-12, further comprising the steps of: (a) administering a therapeutically effective amount of tolebrutinib or pharmaceutically acceptable salt thereof to a patient in need thereof; (b) measuring the level of alanine aminotransferase (ALT) in the patient; (c) detecting a level of ALT of >3×upper limit of normal (ULN); (d) measuring one or more of total bilirubin and international normalized ratio (INR) in a patient; (e) detecting one or more of total bilirubin >2×ULN and INR>1.5; (f) ceasing administration of tolebrutinib or pharmaceutically acceptable salt thereof to the patient; and optionally (g) monitoring the level of ALT in the patient; and (h) resuming administration of a therapeutically effective amount of tolebrutinib or pharmaceutically acceptable salt thereof to the patient when the patient's level of ALT is determined to be <1.5×ULN.
Embodiment 24. The method of any one of embodiments 1-12, further comprising the steps of: (a) administering a therapeutically effective amount of tolebrutinib or pharmaceutically acceptable salt thereof to a patient in need thereof; (b) measuring the level of alanine aminotransferase (ALT) in the patient; (c) detecting a level of ALT of >3×upper limit of normal (ULN); (d) ceasing administration of tolebrutinib or pharmaceutically acceptable salt thereof to the patient if the patient experiences one or more of fatigue, nausea, vomiting, right upper quadrant pain or tenderness, fever, rash, and eosinophilia >5%; and optionally (e) monitoring the level of ALT in the patient; and (f) resuming administration of a therapeutically effective amount of tolebrutinib or pharmaceutically acceptable salt thereof to the patient when the patient's level of ALT is determined to be <1.5×ULN.
Embodiment 25. The method of any one of embodiments 13-24, wherein the level of ALT in step (b) is determined at least monthly.
Embodiment 26. The method of any one of embodiments 13-24, wherein the level of ALT in step (d) is monitored at least weekly.
Embodiment 27. The method of any one of embodiments 13-24, wherein the level of ALT in step (d) is monitored every 2 to 3 days.
Embodiment 28. The method of any one of embodiments 1-27, further comprising administering a therapeutically effective amount of tolebrutinib or pharmaceutically acceptable salt thereof to the patient, wherein the patient is not receiving potent and moderate inducers of cytochrome P450 3A (CYP3A) or potent inhibitors of CYP2C8 hepatic enzymes.
Embodiment 29. The method of any one of embodiments 1-27, further comprising the steps of: (a) advising the patient to limit alcohol consumption during treatment; and (b) administering a therapeutically effective amount of tolebrutinib or pharmaceutically acceptable salt thereof to the patient, wherein the patient is female and is advised to limit alcohol consumption to 14 grams/day or less, or the patient is male and is advised to limit alcohol consumption to 28 grams/day or less.

### VII. Further Additional Enumerated Embodiments

Disclosed herein is a method of reducing disability accumulation independent of relapse activity in a patient with secondary progressive MS in need thereof comprising administering to the patient in need thereof a therapeutically effective amount of tolebrutinib or pharmaceutically acceptable salt thereof.

Embodiment 1. A method of treating a patient with non-relapsing secondary progressive multiple sclerosis (nrSPMS) in need thereof, comprising administering to the patient in need thereof a therapeutically effective amount of tolebrutinib or pharmaceutically acceptable salt thereof, wherein at least one outcome chosen from the following occurs: (a) the risk of confirmed disability progression (CDP) is reduced; (b) the total number of new and/or enlarging T2-hyperintense lesions as detected by MRI, defined as the sum of the individual number of new and/or enlarging T2 lesions, are reduced; (c) the risk of sustained 20% increase in the timed 25-foot walk (T25-FW) test confirmed over at least 3 months is reduced; and (d) the chance of confirmed disability improvement (CDI) is improved.
Embodiment 2. A method of treating a patient with non-relapsing secondary progressive multiple sclerosis (nrSPMS) in need thereof, comprising administering to the patient in need thereof a therapeutically effective amount of tolebrutinib or pharmaceutically acceptable salt thereof, wherein the risk of confirmed disability progression (CDP) is reduced.
Embodiment 3. The method of embodiment 2, wherein the CDP is measured over at least three months, such as at least six months.
Embodiment 4. The method of embodiment 2 or 3, wherein the confirmed disability progression (CDP) comprises: (a) increasing at least 1.0 point from a baseline expanded disability status scale score (EDSS) when the baseline score is less than or equal to 5.0; or (b) increasing at least 0.5 points when the baseline EDSS score is greater than 5.0.
Embodiment 5. The method of any one of embodiments 1-4, wherein the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.
Embodiment 6. A method of reducing the risk of confirmed disability progression (CDP) in a patient with non-relapsing secondary progressive multiple sclerosis (nrSPMS) in need thereof, comprising administering to the patient in need thereof a therapeutically effective amount of tolebrutinib or pharmaceutically acceptable salt thereof.
Embodiment 7. The method of embodiment 6, wherein the CDP is measured over at least three months, such as at least six months.
Embodiment 8. The method of embodiment 6 or 7, wherein confirmed disability progression (CDP) comprises: (a) increasing at least 1.0 point from a baseline expanded disability status scale score (EDSS) when the baseline score is less than or equal to 5.0; or (b) increasing at least 0.5 points when the baseline EDSS score is greater than 5.0.
Embodiment 9. The method of any one of embodiments 6-8, wherein the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.
Embodiment 10. The method of any one of embodiments 6-9, wherein the risk reduction is relative to placebo.
Embodiment 11. A method of reducing the total number of new and/or enlarging T2-hyperintense lesions as detected by MRI, in a patient with non-relapsing secondary progressive multiple sclerosis (nrSPMS) in need thereof, comprising administering to the patient in need thereof a therapeutically effective amount of tolebrutinib or pharmaceutically acceptable salt thereof.
Embodiment 12. The method of embodiment 11, wherein reducing comprises reducing the annualized rate of new and/or enlarging T2-hyperintense lesions.
Embodiment 13. The method of embodiment 11 or 12, wherein the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.
Embodiment 14. The method of embodiment 12, wherein the annualized rate is reduced relative to placebo.
Embodiment 15. A method of reducing the risk of sustained 20% increase in the timed 25-foot walk (T25-FW) test confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof.
Embodiment 16. The method of embodiment 15, wherein the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.
Embodiment 17. The method of embodiment 15 or 16, wherein the risk reduction is relative to placebo.
Embodiment 18. A method of reducing the risk of sustained 20% increase in the 9-hole peg test (9-HPT) confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof.
Embodiment 19. The method of embodiment 18, wherein the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.
Embodiment 20. The method of embodiment 18 or 19, wherein the risk reduction is relative to placebo.
Embodiment 21. A method of improving the chance of confirmed disability improvement (CDI) in a patient with non-relapsing secondary progressive multiple sclerosis (nrSPMS) in need thereof, comprising administering to the patient in need thereof a therapeutically effective amount of tolebrutinib or pharmaceutically acceptable salt thereof.
Embodiment 22. The method of embodiment 21, wherein the CDI is measured over at least three months, such as at least six months.
Embodiment 23. The method of embodiment 22, wherein the confirmed disability improvement (CDI) comprises decreasing at least 1.0 point from a baseline expanded disability status scale score (EDSS).
Embodiment 24. The method of embodiment 22 or 23, wherein the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.
Embodiment 25. The method of any one of embodiments 22-24, wherein the chance is improved relative to placebo.
Embodiment 26. A method of improving time to onset of confirmed disability progression (CDP) in a patient with non-relapsing secondary progressive multiple sclerosis (nrSPMS) in need thereof, comprising administering to the patient in need thereof a therapeutically effective amount of tolebrutinib or pharmaceutically acceptable salt thereof.
Embodiment 27. The method of embodiment 26, wherein the CDP is measured over at least three months, such as at least six months.
Embodiment 28. The method of embodiment 26 or 27, wherein the confirmed disability progression (CDP) comprises: (a) increasing at least 1.0 point from a baseline expanded disability status scale score (EDSS) when the baseline score is less than or equal to 5.0; or (b) increasing at least 0.5 points when the baseline EDSS score is greater than 5.0.
Embodiment 29. The method of any one of embodiments 26-28, wherein the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.
Embodiment 30. A method of improving time to onset of sustained 20% increase in the timed 25-foot walk (T25-FW) test confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof.
Embodiment 31. The method of embodiment 30, wherein the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.
Embodiment 32. A method of improving time to onset of sustained 20% increase in the 9-hole peg test (9-HPT) confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof.
Embodiment 33. The method of embodiment 32, wherein the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.
Embodiment 34. A method of improving time to onset of confirmed disability improvement (CDI) in a patient with non-relapsing secondary progressive multiple sclerosis (nrSPMS) in need thereof, comprising administering to the patient in need thereof a therapeutically effective amount of tolebrutinib or pharmaceutically acceptable salt thereof.
Embodiment 35. The method of embodiment 34, wherein the time to onset of CDI is measured over at least three months, such as at least six months.
Embodiment 36. The method of embodiment 34 or 35, wherein the confirmed disability improvement (CDI) comprises decreasing at least 1.0 point from a baseline expanded disability status scale score (EDSS).
Embodiment 37. The method of any one of embodiments 34-36, wherein the patient with nrSPMS has: (a) a SPMS diagnosis with an expanded disability status scale score (EDSS) between 3.0 and 6.5; (b) no clinical relapse in the previous 24 months; and (c) disability accumulation in the previous 12 months.
Embodiment 38. Tolebrutinib or a pharmaceutically acceptable salt thereof for use in treating a patient with non-relapsing secondary progressive multiple sclerosis (nrSPMS) in the method of any one of embodiments 1-5.
Embodiment 39. Tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof reduces the risk of confirmed disability progression (CDP) in the method of any one of embodiments 6-10.
Embodiment 40. Tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof reduces the total number of new and/or enlarging T2-hyperintense lesions as detected by MRI in the method of any one of embodiments 11-14.
Embodiment 41. Tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof reduces the risk of sustained 20% increase in the timed 25-foot walk (T25-FW) test confirmed over at least 3 months in the method of any one of embodiments 15-17.
Embodiment 42. Tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof reduces the risk of sustained 20% increase in the 9-hole peg test (9-HPT) confirmed over at least 3 months in the method of any one of embodiments 18-20.
Embodiment 43. Tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves the chance of confirmed disability improvement (CDI) in the method of any one of embodiments 21-25.
Embodiment 44. Tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves time to onset of confirmed disability progression (CDP) in the method of any one of embodiments 26-29.
Embodiment 45. Tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves time to onset of sustained 20% increase in the timed 25-foot walk (T25-FW) test confirmed over at least 3 months in the method of any one of embodiments 30-31.
Embodiment 46. Tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves time to onset of sustained 20% increase in the 9-hole peg test (9-HPT) confirmed over at least 3 months in the method of any one of embodiments 32-33.
Embodiment 47. Tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves time to onset of confirmed disability improvement (CDI) in the method of any one of embodiments 34-37.
Embodiment 48. A method of reducing the risk of confirmed disability worsening (CDW) in a patient with a relapsing form of multiple sclerosis (RMS) in need thereof, comprising administering to the patient in need thereof a therapeutically effective amount of tolebrutinib or pharmaceutically acceptable salt thereof.
Embodiment 49. The method of embodiment 48, wherein the CDW is measured over at least three months, such as at least six months.
Embodiment 50. The method of embodiment 48 or 49, wherein CDW comprises: (a) increasing at least 1.5 point from a baseline expanded disability status scale score (EDSS) when the baseline score is 0; (b) increasing at least 1.0 point from the baseline EDSS when the baseline score is greater than or equal to 0.5 and less than or equal to 5.5; or (c) increasing at least 0.5 points when the baseline EDSS score is greater than 5.5.
Embodiment 51. The method of any one of embodiments 48-50, wherein the patient has an RMS diagnosis with an EDSS less than or equal to 5.5 prior to initial administration of tolebrutinib, and one or more of the following: (a) one or more documented relapses in the previous year; (b) two or more documented relapses in the previous 2 years; and (c) one or more Gd-enhancing lesions on an MRI scan in the previous year.
Embodiment 52. The method of any one of embodiments 48-51, wherein the risk reduction is relative to treatment with teriflunomide.
Embodiment 53. A method of improving time to onset of confirmed disability worsening (CDW) in a patient with a relapsing form of multiple sclerosis (RMS) in need thereof, comprising administering to the patient in need thereof a therapeutically effective amount of tolebrutinib or pharmaceutically acceptable salt thereof.
Embodiment 54. The method of embodiment 53, wherein the CDW is measured over at least three months, such as at least six months.
Embodiment 55. The method of embodiment 53 or 54, wherein CDW comprises: (a) increasing at least 1.5 point from a baseline expanded disability status scale score (EDSS) when the baseline score is 0; (b) increasing at least 1.0 point from the baseline EDSS when the baseline score is greater than or equal to 0.5 and less than or equal to 5.5; or (c) increasing at least 0.5 points when the baseline EDSS score is greater than 5.5.
Embodiment 56. The method of any one of embodiments 53-55, wherein the patient has an RMS diagnosis with an EDSS less than or equal to 5.5 at the first visit, and one or more of the following: (a) one or more documented relapses in the previous year; (b) two or more documented relapses in the previous 2 years; and (c) one or more Gd-enhancing lesions on an MRI scan in the previous year.
Embodiment 57. A method of improving percent change in brain volume loss (BVL) as detected by brain MRI in a patient with relapsing forms of multiple sclerosis (RMS), comprising administering to the patient in need thereof a therapeutically effective amount of tolebrutinib or pharmaceutically acceptable salt thereof.
Embodiment 58. The method of embodiment 57, wherein the patient has an RMS diagnosis with an EDSS less than or equal to 5.5 at the first visit, and one or more of the following: (a) one or more documented relapses in the previous year; (b) two or more documented relapses in the previous 2 years; and (c) one or more Gd-enhancing lesions on an MRI scan in the previous year.
Embodiment 59. The method of embodiment 57 or 58, wherein the percent change BVL improvement is relative to treatment with teriflunomide.
Embodiment 60. Tolebrutinib or a pharmaceutically acceptable salt thereof for use in treating a patient with a relapsing form of multiple sclerosis (RMS), wherein tolebrutinib or a pharmaceutically acceptable salt thereof reduces the risk of confirmed disability worsening (CDW) in the method of any one of clams 48-52.
Embodiment 61. Tolebrutinib or a pharmaceutically acceptable salt thereof for use in treating a patient with a relapsing form of multiple sclerosis (RMS), wherein tolebrutinib or a pharmaceutically acceptable salt thereof improves time to onset of confirmed disability worsening (CDW) in the method of any one of clams 53-56.
Embodiment 62. Tolebrutinib or a pharmaceutically acceptable salt thereof for use in treating a patient with a relapsing form of multiple sclerosis (RMS), wherein tolebrutinib or a pharmaceutically acceptable salt thereof improves percent change in brain volume loss (BVL) as detected by brain MRI in the method of any one of clams 57-59.
Embodiment 63. A method of treating a patient with primary progressive multiple sclerosis (PPMS) in need thereof, comprising administering to the patient in need thereof a therapeutically effective amount of tolebrutinib or pharmaceutically acceptable salt thereof, wherein at least one outcome chosen from the following occurs: (a) the risk of composite confirmed disability progression (cCDP) is reduced, wherein the cCDP comprises progression in at least one of confirmed disability progression (CDP), sustained 20% increase in the timed 25-foot walk (T25-FW) test, and sustained 20% increase in the 9-hole peg test (9-HPT); (b) the risk of confirmed disability progression (CDP) is reduced; (c) the total number of new and/or enlarging T2-hyperintense lesions as detected by MRI, defined as the sum of the individual number of new and/or enlarging T2 lesions, are reduced; (e) the risk of sustained 20% increase in the timed 25-foot walk (T25-FW) test is reduced; (e) the risk of sustained 20% increase in the 9-hole peg test (9-HPT) is reduced; (f) the chance of confirmed disability improvement (CDI) is improved; and (g) the percent change in brain volume loss (BVL) as detected by brain MRI is improved.
Embodiment 64. A method of treating a patient with primary progressive multiple sclerosis (PPMS) in need thereof, comprising administering to the patient in need thereof a therapeutically effective amount of tolebrutinib or pharmaceutically acceptable salt thereof, wherein the risk of confirmed disability progression (CDP) or composite confirmed disability progression (cCDP) is reduced, wherein the cCDP comprises progression in at least one of: (a) CDP; (b) sustained 20% increase in the timed 25-foot walk (T25-FW) test; and (c) sustained 20% increase in the 9-hole peg test (9-HPT).
Embodiment 65. The method of embodiment 63 or 64, wherein the CDP, cCDP, sustained 20% increase in the timed 25-foot walk (T25-FW) test, or sustained 20% increase in the 9-hole peg test (9-HPT) is measured over at least three months, such as at least six months.
Embodiment 66. The method of embodiment any one of embodiments 63-65, wherein the CDP comprises: (a) increasing at least 1.0 point from a baseline expanded disability status scale score (EDSS) when the baseline score is less than or equal to 5.5; or (b) increasing at least 0.5 points when the baseline EDSS score is greater than 5.5.
Embodiment 67. The method of any one of embodiments 63-66, wherein the patient with PPMS has: (a) a PPMS diagnosis with an expanded disability status scale score (EDSS) between 2.0 and 6.5; and (b) a cerebrospinal fluid (CSF) analysis that is positive for isoelectric focusing evidence of oligoclonal bands or elevated IgG index.
Embodiment 68. A method of reducing the risk of confirmed disability progression (CDP) or composite confirmed disability progression (cCDP) in a patient with primary progressive multiple sclerosis (PPMS) in need thereof, comprising administering to the patient in need thereof a therapeutically effective amount of tolebrutinib or pharmaceutically acceptable salt thereof, wherein the cCDP comprises progression in at least one of: (a) CDP; (b) sustained 20% increase in the timed 25-foot walk (T25-FW) test; and (c) sustained 20% increase in the 9-hole peg test (9-HPT).
Embodiment 69. The method of embodiment 68, wherein the CDP or cCDP is measured over at least three months, such as at least six months.
Embodiment 70. The method of embodiment 68 or 69, wherein confirmed disability progression (CDP) comprises: (a) increasing at least 1.0 point from a baseline expanded disability status scale score (EDSS) when the baseline score is less than or equal to 5.5; or (b) increasing at least 0.5 points when the baseline EDSS score is greater than 5.5.
Embodiment 71. The method of any one of embodiments 68-70, wherein the patient with PPMS has: (a) a PPMS diagnosis with an expanded disability status scale score (EDSS) between 2.0 and 6.5; and (b) a cerebrospinal fluid (CSF) analysis that is positive for isoelectric focusing evidence of oligoclonal bands or elevated IgG index.
Embodiment 72. The method of any one of embodiments 68-71, wherein the risk reduction is relative to placebo.
Embodiment 73. A method of improving time to onset of confirmed disability progression (CDP) or composite confirmed disability progression (cCDP) in a patient with primary progressive multiple sclerosis (PPMS) in need thereof, comprising administering to the patient in need thereof a therapeutically effective amount of tolebrutinib or pharmaceutically acceptable salt thereof, wherein the cCDP comprises progression in at least one of: (a) CDP; (b) sustained 20% increase in the timed 25-foot walk (T25-FW) test; and (c) sustained 20% increase in the 9-hole peg test (9-HPT).
Embodiment 74. The method of embodiment 73, wherein the CDP or cCDP is measured over at least three months, such as at least six months.
Embodiment 75. The method of embodiment 73 or 74, wherein the CDP comprises: (a) increasing at least 1.0 point from a baseline expanded disability status scale score (EDSS) when the baseline score is less than or equal to 5.5; or (b) increasing at least 0.5 points when the baseline EDSS score is greater than 5.5.
Embodiment 76. The method of any one of embodiments 23-25, wherein the patient with PPMS has: (a) a PPMS diagnosis with an expanded disability status scale score (EDSS) between 2.0 and 6.5; and (b) a cerebrospinal fluid (CSF) analysis that is positive for isoelectric focusing evidence of oligoclonal bands or elevated IgG index.
Embodiment 77. Tolebrutinib or a pharmaceutically acceptable salt thereof for use in treating a patient with primary progressive multiple sclerosis (PPMS) in the method of any one of embodiments 63-67.
Embodiment 78. Tolebrutinib or a pharmaceutically acceptable salt thereof for use in treating a patient with primary progressive multiple sclerosis (PPMS), wherein tolebrutinib or a pharmaceutically acceptable salt thereof reduces the risk of confirmed disability progression (CDP) or composite confirmed disability progression (cCDP) in the method of any one of embodiments 68-72.
Embodiment 79. Tolebrutinib or a pharmaceutically acceptable salt thereof for use in treating a patient with primary progressive multiple sclerosis (PPMS), wherein tolebrutinib or a pharmaceutically acceptable salt thereof improves time to onset of confirmed disability progression (CDP) or composite confirmed disability progression (cCDP) in the method of any one of embodiments 73-76.
Embodiment 80. A method of treating disability accumulation in a patient with multiple sclerosis comprising administering to the patient in need thereof a therapeutically effective amount of tolebrutinib or pharmaceutically acceptable salt thereof.
Embodiment 81. A method of treating disability progression independent of relapse activity in a patient with multiple sclerosis comprising administering to the patient in need thereof a therapeutically effective amount of tolebrutinib or pharmaceutically acceptable salt thereof.
Embodiment 82. The method of embodiment 80 or 81, wherein treating disability accumulation or disability progression independent of relapse activity comprises at least one of: (a) reducing the risk of disability accumulation in the patient in need thereof; (b) delaying disability accumulation in the patient in need thereof; or (c) improving time to onset of disability accumulation in the patient in need thereof.
Embodiment 83. The method of any one of embodiments 80-82, wherein the disability accumulation or disability progression independent of relapse activity is confirmed disability accumulation (CDA).
Embodiment 84. The method of embodiment 83, wherein the confirmed disability accumulation (CDA) is confirmed disability progression (CDP).
Embodiment 85. The method of embodiment 83, wherein the confirmed disability accumulation (CDA) is confirmed disability worsening (CDW).
Embodiment 86. The method of embodiment 83, wherein the confirmed disability accumulation (CDA) is a composite confirmed disability accumulation (cCDA) comprising progression in one or more of: (a) CDW or CDP; (b) sustained 20% increase in the timed 25-foot walk test (T25-FW); and (c) sustained 20% increase in the 9-hole peg test (9-HPT).
Embodiment 87. The method of embodiment 84 or 86, wherein the CDP comprises: (a) increasing at least 1.0 point from a baseline expanded disability status scale score (EDSS) when the baseline score is less than or equal to 5.0; or (b) increasing at least 0.5 points when the baseline EDSS score is greater than 5.0.
Embodiment 88. The method of embodiment 85 or 86, wherein the CDW comprises: (a) increasing at least 1.5 point from a baseline expanded disability status scale score (EDSS) when the baseline score is 0; (b) increasing at least 1.0 point from the baseline EDSS when the baseline score is greater than or equal to 0.5 and less than or equal to 5.5; or (c) increasing at least 0.5 points when the baseline EDSS score is greater than 5.5.
Embodiment 89. The method of any one of embodiments 83-88, wherein the CDA or cCDA is measured over at least 3 months, such as at least 6 months.
Embodiment 90. The method of any one of embodiments 80-90, wherein the patient in need thereof has secondary progressive multiple sclerosis (SPMS).
Embodiment 91. The method of embodiment 91, wherein the SPMS is non-relapsing secondary progressive multiple sclerosis (nrSPMS).
Embodiment 92. The method of embodiment 91, wherein the SPMS is relapsing secondary progressive multiple sclerosis (R-SPMS).
Embodiment 93. The method of any one of embodiments 80-89, wherein the patient in need thereof has a relapsing form of multiple sclerosis (RMS).
Embodiment 94. The method of any one of embodiments 80-89, wherein the patient in need thereof has primary progressive multiple sclerosis (PPMS).
Embodiment 95. Tolebrutinib or a pharmaceutically acceptable salt thereof for use in treating disability accumulation in a patient with multiple sclerosis in the method of any one of embodiments 80 or 82-97.
Embodiment 96. Tolebrutinib or a pharmaceutically acceptable salt thereof for use in treating disability progression independent of relapse activity in a patient with multiple sclerosis in the method of any one of embodiments 81-97.
Embodiment 97. The method, or tolebrutinib or pharmaceutically acceptable salt thereof for use, of any one of embodiments 1-5 or 38, wherein one or more of the reduction in risk of CDP, reduction in total number of new and/or enlarging T2-hyperintense lesions, reduction in risk of sustained 20% increase in T25-FW test, or the improvement of chance of CDI is relative to placebo.
Embodiment 98. The method, or tolebrutinib or pharmaceutically acceptable salt thereof for use, of any one of embodiments 26-37 or 44-47, wherein the time to onset is improved relative to placebo.
Embodiment 99. The method, or tolebrutinib or pharmaceutically acceptable salt thereof for use, of any one of embodiments 53-56 or 61, wherein the time to onset is improved relative to treatment with teriflunomide.
Embodiment 100. The method, or tolebrutinib or pharmaceutically acceptable salt thereof for use, of any one of embodiments 63-67 or 77-78, wherein one or more of the reduction in risk of CDP or cCDP, the reduction in total number of new and/or enlarging T2-hyperintense lesions, the reduction in risk of sustained 20% increase in T25-FW test, the reduction in risk of sustained 20% increase in the 9-hole peg test (9-HPT), the improvement of chance of CDI, or the improvement percent change in BVL is relative to placebo.
Embodiment 101. The method, or tolebrutinib or pharmaceutically acceptable salt thereof for use, of any one of embodiments 73-76 or 79, wherein the time to onset is improved relative to placebo.
Embodiment 102. The method, or tolebrutinib or pharmaceutically acceptable salt thereof for use, of any one of embodiments 82-96, wherein one or more of the reduction of risk, delay, or improvement of time to onset of disability accumulation is relative to placebo.
Embodiment 103. The method, or tolebrutinib or pharmaceutically acceptable salt thereof for use, of any one of embodiments 82-96, wherein one or more of the reduction of risk, delay, or improvement of time to onset of disability accumulation is relative to treatment with teriflunomide.
Embodiment 104. The method, or tolebrutinib or pharmaceutically acceptable salt thereof for use, of any one of embodiments 1-47, 80-98, or 102, wherein the patient in need thereof has one or more Gd-enhancing T1-hyperintense lesions as detected by MRI before administration of tolebrutinib.
Embodiment 105. The method, or tolebrutinib or pharmaceutically acceptable salt thereof for use, of any one of embodiments 1-47, 80-98, or 102, wherein the patient in need thereof:
(a) has no Gd-enhancing T1-hyperintense lesions as detected by MRI before administration of tolebrutinib; and
(b) had no relapse in the 2 years before administration of tolebrutinib.
   Embodiment 106. The method, or tolebrutinib or pharmaceutically acceptable salt thereof for use, of any one of embodiments 1-47, 80-98, or 102, wherein the patient in need thereof has an EDSS score of 4.5 or less before administration of tolebrutinib.
   Embodiment 107. The method, or tolebrutinib or pharmaceutically acceptable salt thereof for use, of any one of embodiments 1-47, 80-98, or 102, wherein the patient in need thereof has an EDSS score of 5.5 or less before administration of tolebrutinib.
   Embodiment 108. The method, or tolebrutinib or pharmaceutically acceptable salt thereof for use, of any one of embodiments 1-47, 80-98, or 102, wherein the patient in need thereof has received 1 or fewer disease modifying therapies for multiple sclerosis before administration of tolebrutinib.
   Embodiment 109. The method, or tolebrutinib or pharmaceutically acceptable salt thereof for use, of embodiment 108, wherein the patient in need thereof has not received a disease modifying therapy before administration of tolebrutinib.
   Embodiment 110. The method, or tolebrutinib or pharmaceutically acceptable salt thereof for use, of embodiment 108, wherein the patient in need thereof has received one prior disease modifying therapy selected from the group consisting of an interferon, glatiramer acetate, dimethyl fumarate, ocrelizumab, teriflunomide, natalizumab, fingolimod, or rituximab.
   Embodiment 111. The method, or tolebrutinib or pharmaceutically acceptable salt thereof for use, of any one of embodiments 1-47, 80-98, or 102, wherein the patient in need thereof had onset of RMS symptoms 10 years or less before administration of tolebrutinib, such as 5 years or less before administration of tolebrutinib or more than 5 and 10 years or less before administration of tolebrutinib.
   Embodiment 112. The method, or tolebrutinib or pharmaceutically acceptable salt thereof for use, of any one of embodiments 48-62, 80-96, 99, or 103, wherein the patient in need thereof has an EDSS score of less than 4 before administration of tolebrutinib.
   Embodiment 113. The method, or tolebrutinib or pharmaceutically acceptable salt thereof for use, of any one of embodiments 48-62, 80-96, 99, or 103, wherein the patient in need thereof has one or more Gd-enhancing T1-hyperintense lesions as detected by MRI before administration of tolebrutinib.
   Embodiment 114. The method, or tolebrutinib or pharmaceutically acceptable salt thereof for use, of any one of embodiments 48-62, 80-96, 99, or 103, wherein the patient in need thereof has not received a disease modifying therapy multiple sclerosis before administration of tolebrutinib.
   Embodiment 115. The method, or tolebrutinib or pharmaceutically acceptable salt thereof for use, of any one of embodiments 48-62, 80-96, 99, or 103, wherein the patient in need thereof had onset of RMS symptoms less than 10 years before administration of tolebrutinib, such as less than 5 years before administration of tolebrutinib or 5 or more and less than 10 years before administration of tolebrutinib.
   Embodiment 116. The method, or tolebrutinib or pharmaceutically acceptable salt thereof for use, of any one of embodiments 48-62, 80-96, 99, or 103, wherein the patient in need thereof had 2 or more relapses in the one year before administration of tolebrutinib.
   Embodiment 117. The method, or tolebrutinib or pharmaceutically acceptable salt thereof for use, of any one of embodiments 1-62 or 80-99, or 102-103, wherein the patient in need thereof has one or more phase rim lesion (PRL) as detected by MRI before administration of tolebrutinib.
   Embodiment 118. The method, or tolebrutinib or pharmaceutically acceptable salt thereof for use, of embodiment 117, wherein the patient in need thereof has one, two, or three phase rim lesions (PRL) as detected by MRI before administration of tolebrutinib.
   Embodiment 119. The method, or tolebrutinib or pharmaceutically acceptable salt thereof for use, of embodiment 117, wherein the patient in need thereof has four or more phase rim lesions (PRL) as detected by MRI before administration of tolebrutinib.
   Embodiment 120. The method, or tolebrutinib or pharmaceutically acceptable salt thereof for use, of any one of embodiments 1-119, wherein the patient in need thereof is an adult with multiple sclerosis.
   Embodiment 121. The method, or tolebrutinib or pharmaceutically acceptable salt thereof for use, of any one of embodiments 1-120, wherein the therapeutically effective amount of tolebrutinib or pharmaceutically acceptable salt thereof is about 5 mg to about 60 mg, measured in base form.
   Embodiment 122. The method, or tolebrutinib or pharmaceutically acceptable salt thereof for use, of any one of embodiments 1-120, wherein the therapeutically effective amount of tolebrutinib or pharmaceutically acceptable salt thereof is 60 mg, measured in base form.

What is claimed is:

1. A method of treating a patient with non-relapsing secondary progressive multiple sclerosis (nrSPMS) in need thereof, comprising administering to the patient in need thereof a therapeutically effective amount of tolebrutinib or pharmaceutically acceptable salt thereof, wherein at least one outcome chosen from the following occurs:
(a) the risk of confirmed disability progression (CDP) is reduced;
(b) the total number of new and/or enlarging T2-hyperintense lesions as detected by MRI, defined as the sum of the individual number of new and/or enlarging T2 lesions, are reduced;
(c) the risk of sustained 20% increase in the timed 25-foot walk (T25-FW) test confirmed over at least 3 months is reduced; and
(d) the chance of confirmed disability improvement (CDI) is improved.

2. A method of treating a patient with non-relapsing secondary progressive multiple sclerosis (nrSPMS) in need thereof, comprising administering to the patient in need thereof a therapeutically effective amount of tolebrutinib or pharmaceutically acceptable salt thereof, wherein the risk of confirmed disability progression (CDP) is reduced.

3. The method of claim 2, wherein the CDP is measured over at least three months, such as at least six months.

4. The method of claim 2 or 3, wherein the confirmed disability progression (CDP) comprises:
(a) increasing at least 1.0 point from a baseline expanded disability status scale score (EDSS) when the baseline score is less than or equal to 5.0; or
(b) increasing at least 0.5 points when the baseline EDSS score is greater than 5.0.

5. The method of any one of claims 1-4, wherein the patient with nrSPMS has:
(a) a SPMS diagnosis with an expanded disability status scale score (EDSS) between 3.0 and 6.5;
(b) no clinical relapse in the previous 24 months; and
(c) disability accumulation in the previous 12 months.

6. A method of reducing the risk of confirmed disability progression (CDP) in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

7. The method of claim 6, wherein the CDP is measured over at least three months, such as at least six months.

8. The method of claim 6 or 7, wherein confirmed disability progression (CDP) comprises:
(a) increasing at least 1.0 point from a baseline expanded disability status scale score (EDSS) when the baseline score is less than or equal to 5.0; or
(b) increasing at least 0.5 points when the baseline EDSS score is greater than 5.0.

9. The method of any one of claims 6-8, wherein the patient with nrSPMS has:
(a) a SPMS diagnosis with an expanded disability status scale score (EDSS) between 3.0 and 6.5;
(b) no clinical relapse in the previous 24 months; and
(c) disability accumulation in the previous 12 months.

10. The method of any one of claims 6-9, wherein the risk reduction is relative to placebo.

11. A method of reducing the total number of new and/or enlarging T2-hyperintense lesions as detected by MRI, in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

12. The method of claim 11, wherein reducing comprises reducing the annualized rate of new and/or enlarging T2-hyperintense lesions.

13. The method of claim 11 or 12, wherein the patient with nrSPMS has:
(a) a SPMS diagnosis with an expanded disability status scale score (EDSS) between 3.0 and 6.5;
(b) no clinical relapse in the previous 24 months; and
(c) disability accumulation in the previous 12 months.

14. The method of claim 12, wherein the annualized rate is reduced relative to placebo.

15. A method of reducing the risk of sustained 20% increase in the timed 25-foot walk (T25-FW) test confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

16. The method of claim 15, wherein the patient with nrSPMS has:
(a) a SPMS diagnosis with an expanded disability status scale score (EDSS) between 3.0 and 6.5;
(b) no clinical relapse in the previous 24 months; and
(c) disability accumulation in the previous 12 months.

17. The method of claim 15 or 16, wherein the risk reduction is relative to placebo.

18. A method of reducing the risk of sustained 20% increase in the 9-hole peg test (9-HPT) confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

19. The method of claim 18, wherein the patient with nrSPMS has:
(a) a SPMS diagnosis with an expanded disability status scale score (EDSS) between 3.0 and 6.5;
(b) no clinical relapse in the previous 24 months; and
(c) disability accumulation in the previous 12 months.

20. The method of claim 18 or 19, wherein the risk reduction is relative to placebo.

21. A method of improving the chance of confirmed disability improvement (CDI) in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

22. The method of claim 21, wherein the CDI is measured over at least three months, such as at least six months.

23. The method of claim 22, wherein the confirmed disability improvement (CDI) comprises decreasing at least 1.0 point from a baseline expanded disability status scale score (EDSS).

24. The method of claim 22 or 23, wherein the patient with nrSPMS has:
(a) a SPMS diagnosis with an expanded disability status scale score (EDSS) between 3.0 and 6.5;
(b) no clinical relapse in the previous 24 months; and
(c) disability accumulation in the previous 12 months.

25. The method of any one of claims 22-24, wherein the chance is improved relative to placebo.

26. A method of improving time to onset of confirmed disability progression (CDP) in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

27. The method of claim 26, wherein the CDP is measured over at least three months, such as at least six months.

28. The method of claim 26 or 27, wherein the confirmed disability progression (CDP) comprises:
(a) increasing at least 1.0 point from a baseline expanded disability status scale score (EDSS) when the baseline score is less than or equal to 5.0; or
(b) increasing at least 0.5 points when the baseline EDSS score is greater than 5.0.

29. The method of any one of claims 26-28, wherein the patient with nrSPMS has:
(a) a SPMS diagnosis with an expanded disability status scale score (EDSS) between 3.0 and 6.5;
(b) no clinical relapse in the previous 24 months; and
(c) disability accumulation in the previous 12 months.

30. A method of improving time to onset of sustained 20% increase in the timed 25-foot walk (T25-FW) test confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

31. The method of claim 30, wherein the patient with nrSPMS has:
(a) a SPMS diagnosis with an expanded disability status scale score (EDSS) between 3.0 and 6.5;
(b) no clinical relapse in the previous 24 months; and
(c) disability accumulation in the previous 12 months.

32. A method of improving time to onset of sustained 20% increase in the 9-hole peg test (9-HPT) confirmed over at least 3 months, in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

33. The method of claim 32, wherein the patient with nrSPMS has:
(a) a SPMS diagnosis with an expanded disability status scale score (EDSS) between 3.0 and 6.5;
(b) no clinical relapse in the previous 24 months; and
(c) disability accumulation in the previous 12 months.

34. A method of improving time to onset of confirmed disability improvement (CDI) in a patient with non-relapsing secondary progressive multiple sclerosis (nrSPMS) in need thereof, comprising administering to the patient in need thereof a therapeutically effective amount of tolebrutinib or pharmaceutically acceptable salt thereof.

35. The method of claim 34, wherein the time to onset of CDI is measured over at least three months, such as at least six months.

36. The method of claim 34 or 35, wherein the confirmed disability improvement (CDI) comprises decreasing at least 1.0 point from a baseline expanded disability status scale score (EDSS).

37. The method of any one of claims 34-36, wherein the patient with nrSPMS has:
(a) a SPMS diagnosis with an expanded disability status scale score (EDSS) between 3.0 and 6.5;
(b) no clinical relapse in the previous 24 months; and
(c) disability accumulation in the previous 12 months.

38. Tolebrutinib or a pharmaceutically acceptable salt thereof for use in treating a patient with non-relapsing secondary progressive multiple sclerosis (nrSPMS) in the method of any one of claims 1-5.

39. Tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof reduces the risk of confirmed disability progression (CDP) in the method of any one of claims 6-10.

40. Tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof reduces the total number of new and/or enlarging T2-hyperintense lesions as detected by MRI in the method of any one of claims 11-14.

41. Tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof reduces the risk of sustained 20% increase in the timed 25-foot walk (T25-FW) test confirmed over at least 3 months in the method of any one of claims 15-17.

42. Tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof reduces the risk of sustained 20% increase in the 9-hole peg test (9-HPT) confirmed over at least 3 months in the method of any one of claims 18-20.

43. Tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves the chance of confirmed disability improvement (CDI) in the method of any one of claims 21-25.

44. Tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves time to onset of confirmed disability progression (CDP) in the method of any one of claims 26-29.

45. Tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves time to onset of sustained 20% increase in the timed 25-foot walk (T25-FW) test confirmed over at least 3 months in the method of any one of claims 30-31.

46. Tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves time to onset of sustained 20% increase in the 9-hole peg test (9-HPT) confirmed over at least 3 months in the method of any one of claims 32-33.

47. Tolebrutinib or a pharmaceutically acceptable salt thereof for use in treating a patient with non-relapsing secondary progressive multiple sclerosis (nrSPMS), wherein tolebrutinib or a pharmaceutically acceptable salt thereof improves time to onset of confirmed disability improvement (CDI) in the method of any one of claims 34-37.

48. A method of reducing the risk of confirmed disability worsening (CDW) in a patient with a relapsing form of multiple sclerosis (RMS) in need thereof, comprising administering to the patient in need thereof a therapeutically effective amount of tolebrutinib or pharmaceutically acceptable salt thereof.

49. The method of claim 48, wherein the CDW is measured over at least three months, such as at least six months.

50. The method of claim 48 or 49, wherein CDW comprises:
(a) increasing at least 1.5 point from a baseline expanded disability status scale score (EDSS) when the baseline score is 0;
(b) increasing at least 1.0 point from the baseline EDSS when the baseline score is greater than or equal to 0.5 and less than or equal to 5.5; or
(c) increasing at least 0.5 points when the baseline EDSS score is greater than 5.5.

51. The method of any one of claims 48-50, wherein the patient has an RMS diagnosis with an EDSS less than or equal to 5.5 prior to initial administration of tolebrutinib, and one or more of the following:
(a) one or more documented relapses in the previous year;
(b) two or more documented relapses in the previous 2 years; and
(c) one or more Gd-enhancing lesions on an MRI scan in the previous year.

52. The method of any one of claims 48-51, wherein the risk reduction is relative to treatment with teriflunomide.

53. A method of improving time to onset of confirmed disability worsening (CDW) in a patient with a relapsing form of multiple sclerosis (RMS) in need thereof, comprising administering to the patient in need thereof a therapeutically effective amount of tolebrutinib or pharmaceutically acceptable salt thereof.

54. The method of claim 53, wherein the CDW is measured over at least three months, such as at least six months.

55. The method of claim 53 or 54, wherein CDW comprises:
(a) increasing at least 1.5 point from a baseline expanded disability status scale score (EDSS) when the baseline score is 0;
(b) increasing at least 1.0 point from the baseline EDSS when the baseline score is greater than or equal to 0.5 and less than or equal to 5.5; or
(c) increasing at least 0.5 points when the baseline EDSS score is greater than 5.5.

56. The method of any one of claims 53-55, wherein the patient has an RMS diagnosis with an EDSS less than or equal to 5.5 at the first visit, and one or more of the following:
(a) one or more documented relapses in the previous year;
(b) two or more documented relapses in the previous 2 years; and
(c) one or more Gd-enhancing lesions on an MRI scan in the previous year.

57. A method of improving percent change in brain volume loss (BVL) as detected by brain MRI in a patient with relapsing forms of multiple sclerosis (RMS), comprising administering to the patient in need thereof a therapeutically effective amount of tolebrutinib or pharmaceutically acceptable salt thereof.

58. The method of claim 57, wherein the patient has an RMS diagnosis with an EDSS less than or equal to 5.5 at the first visit, and one or more of the following:
(a) one or more documented relapses in the previous year;
(b) two or more documented relapses in the previous 2 years; and
(c) one or more Gd-enhancing lesions on an MRI scan in the previous year.

59. The method of claim 57 or 58, wherein the percent change BVL improvement is relative to treatment with teriflunomide.

60. Tolebrutinib or a pharmaceutically acceptable salt thereof for use in treating a patient with a relapsing form of multiple sclerosis (RMS), wherein tolebrutinib or a pharmaceutically acceptable salt thereof reduces the risk of confirmed disability worsening (CDW) in the method of any one of claims 48-52.

61. Tolebrutinib or a pharmaceutically acceptable salt thereof for use in treating a patient with a relapsing form of multiple sclerosis (RMS), wherein tolebrutinib or a pharmaceutically acceptable salt thereof reduces improves time to onset of confirmed disability worsening (CDW) in the method of any one of claims 53-56.

62. Tolebrutinib or a pharmaceutically acceptable salt thereof for use in treating a patient with a relapsing form of multiple sclerosis (RMS), wherein tolebrutinib or a pharmaceutically acceptable salt thereof reduces improves percent change in brain volume loss (BVL) as detected by brain MRI in the method of any one of claims 57-59.

63. A method of treating a patient with primary progressive multiple sclerosis (PPMS) in need thereof, comprising administering to the patient in need thereof a therapeutically effective amount of tolebrutinib or pharmaceutically acceptable salt thereof, wherein at least one outcome chosen from the following occurs:
(a) the risk of composite confirmed disability progression (cCDP) is reduced, wherein the cCDP comprises progression in at least one of confirmed disability progression (CDP), sustained 20% increase in the timed 25-foot walk (T25-FW) test, and sustained 20% increase in the 9-hole peg test (9-HPT);
(b) the risk of confirmed disability progression (CDP) is reduced;
(c) the total number of new and/or enlarging T2-hyperintense lesions as detected by MRI, defined as the sum of the individual number of new and/or enlarging T2 lesions, are reduced;
(d) the risk of sustained 20% increase in the timed 25-foot walk (T25-FW) test is reduced;
(e) the risk of sustained 20% increase in the 9-hole peg test (9-HPT) is reduced;
(f) the chance of confirmed disability improvement (CDI) is improved; and
(g) the percent change in brain volume loss (BVL) as detected by brain MRI is improved.

64. A method of treating a patient with primary progressive multiple sclerosis (PPMS) in need thereof, comprising administering to the patient in need thereof a therapeutically effective amount of tolebrutinib or pharmaceutically acceptable salt thereof, wherein the risk of confirmed disability progression (CDP) or composite confirmed disability progression (cCDP) is reduced, wherein the cCDP comprises progression in at least one of:
(a) CDP;
(b) sustained 20% increase in the timed 25-foot walk (T25-FW) test; and
(c) sustained 20% increase in the 9-hole peg test (9-HPT).

65. The method of claim 63 or 64, wherein the CDP, cCDP, sustained 20% increase in the timed 25-foot walk (T25-FW) test, or sustained 20% increase in the 9-hole peg test (9-HPT) is measured over at least three months, such as at least six months.

66. The method of claim any one of claims 63-65, wherein the CDP comprises:
(a) increasing at least 1.0 point from a baseline expanded disability status scale score (EDSS) when the baseline score is less than or equal to 5.5; or
(b) increasing at least 0.5 points when the baseline EDSS score is greater than 5.5.

67. The method of any one of claims 63-66, wherein the patient with PPMS has:
(a) a PPMS diagnosis with an expanded disability status scale score (EDSS) between 2.0 and 6.5; and
(b) a cerebrospinal fluid (CSF) analysis that is positive for isoelectric focusing evidence of oligoclonal bands or elevated IgG index.

68. A method of reducing the risk of confirmed disability progression (CDP) or composite confirmed disability progression (cCDP) in a patient with primary progressive multiple sclerosis (PPMS) in need thereof, comprising administering to the patient in need thereof a therapeutically effective amount of tolebrutinib or pharmaceutically acceptable salt thereof, wherein the cCDP comprises progression in at least one of:
(a) CDP;
(b) sustained 20% increase in the timed 25-foot walk (T25-FW) test; and
(c) sustained 20% increase in the 9-hole peg test (9-HPT).

69. The method of claim 68, wherein the CDP or cCDP is measured over at least three months, such as at least six months.

70. The method of claim 68 or 69, wherein confirmed disability progression (CDP) comprises:
(a) increasing at least 1.0 point from a baseline expanded disability status scale score (EDSS) when the baseline score is less than or equal to 5.5; or
(b) increasing at least 0.5 points when the baseline EDSS score is greater than 5.5.

71. The method of any one of claims 68-70, wherein the patient with PPMS has:
(a) a PPMS diagnosis with an expanded disability status scale score (EDSS) between 2.0 and 6.5; and
(b) a cerebrospinal fluid (CSF) analysis that is positive for isoelectric focusing evidence of oligoclonal bands or elevated IgG index.

72. The method of any one of claims 68-71, wherein the risk reduction is relative to placebo.

73. A method of improving time to onset of confirmed disability progression (CDP) or composite confirmed disability progression (cCDP) in a patient with primary progressive multiple sclerosis (PPMS) in need thereof, comprising administering to the patient in need thereof a therapeutically effective amount of tolebrutinib or pharmaceutically acceptable salt thereof, wherein the cCDP comprises progression in at least one of:
(a) CDP;
(b) sustained 20% increase in the timed 25-foot walk (T25-FW) test; and
(c) sustained 20% increase in the 9-hole peg test (9-HPT).

74. The method of claim 73, wherein the CDP or cCDP is measured over at least three months, such as at least six months.

75. The method of claim 73 or 74, wherein the CDP comprises:
(a) increasing at least 1.0 point from a baseline expanded disability status scale score (EDSS) when the baseline score is less than or equal to 5.5; or
(b) increasing at least 0.5 points when the baseline EDSS score is greater than 5.5.

76. The method of any one of claims 23-25, wherein the patient with PPMS has:
(a) a PPMS diagnosis with an expanded disability status scale score (EDSS) between 2.0 and 6.5; and
(b) a cerebrospinal fluid (CSF) analysis that is positive for isoelectric focusing evidence of oligoclonal bands or elevated IgG index.

77. Tolebrutinib or a pharmaceutically acceptable salt thereof for use in treating a patient with primary progressive multiple sclerosis (PPMS) in the method of any one of claims 63-67.

78. Tolebrutinib or a pharmaceutically acceptable salt thereof for use in treating a patient with primary progressive multiple sclerosis (PPMS), wherein tolebrutinib or a pharmaceutically acceptable salt thereof reduces the risk of confirmed disability progression (CDP) or composite confirmed disability progression (cCDP) in the method of any one of claims 68-72.

79. Tolebrutinib or a pharmaceutically acceptable salt thereof for use in treating a patient with primary progressive multiple sclerosis (PPMS), wherein tolebrutinib or a pharmaceutically acceptable salt thereof improves time to onset of confirmed disability progression (CDP) or composite confirmed disability progression (cCDP) in the method of any one of claims 73-76.

80. A method of treating disability accumulation in a patient with multiple sclerosis comprising administering to the patient in need thereof a therapeutically effective amount of tolebrutinib or pharmaceutically acceptable salt thereof.

81. A method of treating disability progression independent of relapse activity in a patient with multiple sclerosis comprising administering to the patient in need thereof a therapeutically effective amount of tolebrutinib or pharmaceutically acceptable salt thereof.

82. The method of claim 80 or 81, wherein treating disability accumulation or disability progression independent of relapse activity comprises at least one of:
(a) reducing the risk of disability accumulation in the patient in need thereof,
(b) delaying disability accumulation in the patient in need thereof; or
(c) improving time to onset of disability accumulation in the patient in need thereof.

83. The method of any one of claims 80-82, wherein the disability accumulation or disability progression independent of relapse activity is confirmed disability accumulation (CDA).

84. The method of claim 83, wherein the confirmed disability accumulation (CDA) is confirmed disability progression (CDP).

85. The method of claim 83, wherein the confirmed disability accumulation (CDA) is confirmed disability worsening (CDW).

86. The method of claim 83, wherein the confirmed disability accumulation (CDA) is a composite confirmed disability accumulation (cCDA) comprising progression in one or more of:
(a) CDW or CDP;
(b) sustained 20% increase in the timed 25-foot walk test (T25-FW); and
(c) sustained 20% increase in the 9-hole peg test (9-HPT).

87. The method of claim 84 or 86, wherein the CDP comprises:
(a) increasing at least 1.0 point from a baseline expanded disability status scale score (EDSS) when the baseline score is less than or equal to 5.0; or
(b) increasing at least 0.5 points when the baseline EDSS score is greater than 5.0.

88. The method of claim 85 or 86, wherein the CDW comprises:
(a) increasing at least 1.5 point from a baseline expanded disability status scale score (EDSS) when the baseline score is 0;
(b) increasing at least 1.0 point from the baseline EDSS when the baseline score is greater than or equal to 0.5 and less than or equal to 5.5; or
(c) increasing at least 0.5 points when the baseline EDSS score is greater than 5.5.

89. The method of any one of claims 83-88, wherein the CDA or cCDA is measured over at least 3 months, such as at least 6 months.

90. The method of any one of claims 80-90, wherein the patient in need thereof has secondary progressive multiple sclerosis (SPMS).

91. The method of claim 91, wherein the SPMS is non-relapsing secondary progressive multiple sclerosis (nrSPMS).

92. The method of claim 91, wherein the SPMS is relapsing secondary progressive multiple sclerosis (R-SPMS).

93. The method of any one of claims 80-89, wherein the patient in need thereof has a relapsing form of multiple sclerosis (RMS).

94. The method of any one of claims 80-89, wherein the patient in need thereof has primary progressive multiple sclerosis (PPMS).

95. Tolebrutinib or a pharmaceutically acceptable salt thereof for use in treating disability accumulation in a patient with multiple sclerosis in the method of any one of claims 80 or 82-94.

96. Tolebrutinib or a pharmaceutically acceptable salt thereof for use in treating disability progression independent of relapse activity in a patient with multiple sclerosis in the method of any one of claims 81-94.

97. The method, or tolebrutinib or pharmaceutically acceptable salt thereof for use, of any one of claims 1-5 or 38, wherein one or more of the reduction in risk of CDP, reduction in total number of new and/or enlarging T2-hyperintense lesions, reduction in risk of sustained 20% increase in T25-FW test, or the improvement of chance of CDI is relative to placebo.

98. The method, or tolebrutinib or pharmaceutically acceptable salt thereof for use, of any one of claims 26-37 or 44-47, wherein the time to onset is improved relative to placebo.

99. The method, or tolebrutinib or pharmaceutically acceptable salt thereof for use, of any one of claims 53-56 or 61, wherein the time to onset is improved relative to treatment with teriflunomide.

100. The method, or tolebrutinib or pharmaceutically acceptable salt thereof for use, of any one of claims 63-67 or 77-78, wherein one or more of the reduction in risk of CDP or cCDP, the reduction in total number of new and/or enlarging T2-hyperintense lesions, the reduction in risk of sustained 20% increase in T25-FW test, the reduction in risk of sustained 20% increase in the 9-hole peg test (9-HPT), the improvement of chance of CDI, or the improvement percent change in BVL is relative to placebo.

101. The method, or tolebrutinib or pharmaceutically acceptable salt thereof for use, of any one of claims 73-76 or 79, wherein the time to onset is improved relative to placebo.

102. The method, or tolebrutinib or pharmaceutically acceptable salt thereof for use, of any one of claims 82-96, wherein one or more of the reduction of risk, delay, or improvement of time to onset of disability accumulation is relative to placebo.

103. The method, or tolebrutinib or pharmaceutically acceptable salt thereof for use, of any one of claims 82-96, wherein one or more of the reduction of risk, delay, or improvement of time to onset of disability accumulation is relative to treatment with teriflunomide.

104. The method, or tolebrutinib or pharmaceutically acceptable salt thereof for use, of any one of claims 1-47, 80-98, or 102, wherein the patient in need thereof has one or more Gd-enhancing T1-hyperintense lesions as detected by MRI before administration of tolebrutinib.

105. The method, or tolebrutinib or pharmaceutically acceptable salt thereof for use, of any one of claims 1-47, 80-98, or 102, wherein the patient in need thereof:
(a) has no Gd-enhancing T1-hyperintense lesions as detected by MRI before administration of tolebrutinib; and
(b) had no relapse in the 2 years before administration of tolebrutinib.

106. The method, or tolebrutinib or pharmaceutically acceptable salt thereof for use, of any one of claims 1-47, 80-98, or 102, wherein the patient in need thereof has an EDSS score of 4.5 or less before administration of tolebrutinib.

107. The method, or tolebrutinib or pharmaceutically acceptable salt thereof for use, of any one of claims 1-47, 80-98, or 102, wherein the patient in need thereof has an EDSS score of 5.5 or less before administration of tolebrutinib.

108. The method, or tolebrutinib or pharmaceutically acceptable salt thereof for use, of any one of claims 1-47, 80-98, or 102, wherein the patient in need thereof has received 1 or fewer disease modifying therapies for multiple sclerosis before administration of tolebrutinib.

109. The method, or tolebrutinib or pharmaceutically acceptable salt thereof for use, of claim 108, wherein the patient in need thereof has not received a disease modifying therapy before administration of tolebrutinib.

110. The method, or tolebrutinib or pharmaceutically acceptable salt thereof for use, of claim 108, wherein the patient in need thereof has received one prior disease modifying therapy selected from the group consisting of an interferon, glatiramer acetate, dimethyl fumarate, ocrelizumab, teriflunomide, natalizumab, fingolimod, and rituximab.

111. The method, or tolebrutinib or pharmaceutically acceptable salt thereof for use, of any one of claims 1-47, 80-98, or 102, wherein the patient in need thereof had onset of RMS symptoms 10 years or less before administration of tolebrutinib, such as 5 years or less before administration of tolebrutinib or more than 5 and 10 years or less before administration of tolebrutinib.

112. The method, or tolebrutinib or pharmaceutically acceptable salt thereof for use, of any one of claims 48-62, 80-96, 99, or 103, wherein the patient in need thereof has an EDSS score of less than 4 before administration of tolebrutinib.

113. The method, or tolebrutinib or pharmaceutically acceptable salt thereof for use, of any one of claims 48-62, 80-96, 99, or 103, wherein the patient in need thereof has one or more Gd-enhancing T1-hyperintense lesions as detected by MRI before administration of tolebrutinib.

114. The method, or tolebrutinib or pharmaceutically acceptable salt thereof for use, of any one of claims 48-62, 80-96, 99, or 103, wherein the patient in need thereof has not received a disease modifying therapy multiple sclerosis before administration of tolebrutinib.

115. The method, or tolebrutinib or pharmaceutically acceptable salt thereof for use, of any one of claims 48-62, 80-96, 99, or 103, wherein the patient in need thereof had onset of RMS symptoms less than 10 years before administration of tolebrutinib, such as less than 5 years before administration of tolebrutinib or 5 or more and less than 10 years before administration of tolebrutinib.

116. The method, or tolebrutinib or pharmaceutically acceptable salt thereof for use, of any one of claims 48-62, 80-96, 99, or 103, wherein the patient in need thereof had 2 or more relapses in the one year before administration of tolebrutinib.

117. The method, or tolebrutinib or pharmaceutically acceptable salt thereof for use, of any one of claims 1-62 or 80-99, or 102-103, wherein the patient in need thereof has one or more phase rim lesion (PRL) as detected by MRI before administration of tolebrutinib.

118. The method, or tolebrutinib or pharmaceutically acceptable salt thereof for use, of claim 117, wherein the patient in need thereof has one, two, or three phase rim lesions (PRL) as detected by MRI before administration of tolebrutinib.

119. The method, or tolebrutinib or pharmaceutically acceptable salt thereof for use, of claim 117, wherein the patient in need thereof has four or more phase rim lesions (PRL) as detected by MRI before administration of tolebrutinib.

120. The method, or tolebrutinib or pharmaceutically acceptable salt thereof for use, of any one of claims 1-119, wherein the patient in need thereof is an adult with multiple sclerosis.

121. The method, or tolebrutinib or pharmaceutically acceptable salt thereof for use, of any one of claims 1-120, wherein the therapeutically effective amount of tolebrutinib or pharmaceutically acceptable salt thereof is about 5 mg to about 60 mg, measured in base form.

122. The method, or tolebrutinib or pharmaceutically acceptable salt thereof for use, of any one of claims 1-120, wherein the therapeutically effective amount of tolebrutinib or pharmaceutically acceptable salt thereof is 60 mg, measured in base form.
